(19) United States
(12) Patent Application Publication
(10) Pub. No.: US 2023/0235082 A1
(43) Pub. Date: Jul. 27, 2023

(54) SITE-SPECIFIC ANTIBODY CONJUGATES AND THE METHODS FOR PREPARATION OF THE SAME

(71) Applicant: GLYCO-THERAPY BIOTECHNOLOGY CO., LTD., Hangzhou, Zhejiang (CN)

(72) Inventor: Yi YANG, Hangzhou, Zhejiang (CN)

(21) Appl. No.: 18/010,634

(22) PCT Filed: Aug. 20, 2021
(86) PCT No.: PCT/CN2021/113692

§ 371 Date: Dec. 15, 2022

(30) Foreign Application Priority Data
Aug. 21, 2020 (WO) PCT/CN2020/110607

(51) Int. Cl. C07K 16/30; A61P 35/00

(52) U.S. Cl. CPC C07K 16/3015; A61P 35/00; C07K 2317/41

(57) ABSTRACT

The present disclosure provides a site-specific protein conjugate and the method for preparation of the same. The protein conjugate comprising a protein and an oligosaccharide, wherein said oligosaccharide comprises

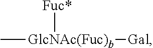

wherein: said GlcNAc is directly or indirectly linked to an amino acid of said protein; said Gal is a galactose; said (Fuc) is a fucose, b is 0 or 1; said Fuc* comprises a fucose or a fucose derivative linked to a molecule of interest (MOI), said protein comprises an antigen binding fragment and/or a Fc fragment.

### CROSS-REFERENCE TO RELATED APPLICATIONS

This application claims the benefit of priority of PCT application NO. PCT/CN2020/110607 filed on Aug. 21, 2020 and entitled “Antibody conjugates and chemoenzymatic N-glycan editing strategies for preparation of the antibody conjugates”, the entirety of which is incorporated herein by reference.

### BACKGROUND OF THE INVENTION

Antibody-conjugates, i.e., antibodies conjugated to a molecule of interest (MOI) via a linker, are known in the art. Their unique property combines the specificity of monoclonal antibodies (mAbs) and the activity, such as the toxicity, of MOIs. Antibody-conjugates serve as powerful agents to deliver highly potent drugs to tumors with minimal off-target toxicity. There are approximately several hundreds of antibody-conjugates approved by FDA or under clinical/pre-clinical evaluations.

Antibody-conjugates known in the art generally suffer from several disadvantages. For example, most of ADCs approved by FDA or under clinical evaluations are conjugated with payloads through modifications of naturally available amino acid side-chains (lysine, cysteine), leading to a stochastic distribution of drug-antibody ratio (DAR). Such heterogeneous mixtures of ADCs have been demonstrated to show low efficacy and narrow therapeutic windows. In addition, such kind of linkers are not stable, leading to the dissociation of the high toxic payloads into the plasma. Different approaches have been developed to obtain site-specific and stable ADCs efficiently. However, the majority of these methods still requires antibodies to be modified either by site-directed mutations or the introduction of genetically encoded tags. Genetic re-engineering of antibodies is a laborious task and sometimes results in compromised expression yields, which has a negative impact on the cost of goods of the ADC. Additionally, the position of to-be-introduced amino acids or amino acid sequences needs to be carefully optimized.

Recently, the modification of the pendant glycans of antibodies has become a convenient way for site-specific conjugation without additional genetical re-engineering. However, glycosylation is a highly heterogeneous post-translational modification, rendering the generation of homogeneous glycans for chemical modification a formidable challenge. Most of therapeutic mAbs have a single N-linked biantennary carbohydrate structures at Asn297 with heterogeneity in core α-1,6-fucosylation, terminal sialylation and galactosylation, which is located in both heavy chains in the Fc region of the molecule. Several attempts have been made to remodel the N297 glycan part to construct antibody conjugates on native antibodies (Bertozzi C. R. et al., Bioconjugate Chem. 2015, 26, 176-192).

Through a metabolic incorporating strategy, Okeley N. M. et al. were able to incorporate 6-thiofucose onto IgG glycans at a percentage of 60-70% (Okeley N. M. et al., Bioconjugate Chem. 2013, 24, 1650-1655). Following conjugation with a maleimide-linked MMAE yielded a conjugate with a DAR of 1.3. However, the incorporation ratio of the unnatural 6-thiofucose were difficult to control, leading to heterogeneous antibodies conjugates.

Several strategies were developed by using endoglycosidases and their mutants to install reaction handles on the N-Glycan of the Fc domains of antibodies. In general, N297 glycans were first trimmed by a endoglycosidase to leave the core N-acetylglucosamine (GlcNAc) moiety with or without core-fucoslylation. Then, endoglycosidases mutants were used to transfer oligosaccharide bearing alkyne or azido groups to the trimmed antibody. Wang L. et al. reported the transfer of a tetrasaccharide oxazoline containing two 6-azido mannose moieties on rituximab by using EndoS and its mutant, resulting in a antibody molecule containing four azido groups (Wang L. et al., J. Am. Chem. Soc. 2012, 134, 12308-12318). Similarly, Huang W. et al. reported the transfer of non-natural egg-yolk sialylglycopeptide (SGP) derivatives carrying azido tags to trastuzumab, following reaction with DBCO conjugated cytotoxin enables the generation of homogeneous antibody drug conjugates with a DAR of 4 (Huang W. et al., Org. Biomol. Chem., 2016, 14, 9501-9518). However, a disadvantage of the glycosynthase strategies is the lengthy and complex synthesis of the required oligosaccharide derivatives.

Zhou Q. et al. using galactosyl and sialyltransferases to introduce terminal sialic acids on the native glycans of N297 on the antibody (Zhou Q. et al., Bioconjugate Chem. 2014, 25, 510-520). Periodate oxidation of these sialic acids yielded aldehyde groups which were subsequently used to conjugate aminooxy functionalized cytotoxins via oxime ligation. This strategy enables the incorporation of an average of 1.6 cytotoxins per antibody molecule. Similarly, by treating co-fucosylated IgG with sodium periodate, Neri D. et al. were able to oxidize the core-fucose residues to an aldehyde, which were further used to prepare hydrazone conjugates with fluorophores and a dolastatin analogue (Neri D. et al., Chem. Commun., 2012, 48, 7100-7102). However, the use of periodate oxidation may lead to the oxidative damage to the antibodies.

Dimitrov D. S et al. employed the bovine GalT-Y289L galatosyltransferase mutant to transfer the a galactose moiety comprising a C2-substituted keto group onto the terminal GlcNAc of a degalatosylated GoF glycoform of an intact antibody (Dimitrov D. S et al., mAbs, 2014, 6, 1190-1200). Following oxime ligation reaction enables the installation of cytotoxins to the modified antibody. Boons G. et al. exploited the ST6Gal1 to incorporate a sialic acid derivative modified with an azide at the C9 position into the terminal galactose of an intact antibody, leading to a four azido groups modified antibody (Boons G. et al., Angew. Chem. Int. Ed. 2014, 53, 7179-7182). van Delft F. L. et al. employed a mutant of bovine galactosyltransferase (GalT-Y289L) to transfer azido-tagged N-Acetylgalactosamine (UDP-GalNAz) onto a core-fucosylated as well as nonfucosylated core-GlcNAc-Fc domain of intact antibodies, resulted in a antibody molecule containing two azido groups, following reaction with the bicyclononyne (BCN) conjugated cytotoxins enables the generation of homogeneous ADCs with a DAR of 2 (van Delft F. L. et al., Bioconjugate Chem. 2015, 26, 2233-2242). However, only very limited groups (typically azido group and ketone group) were modified to the antibodies by these strategies, leading to very finite reactions could be applied to introduce a molecule of interest to the antibodies by a second ligation step. Thus, a glyco-editing strategy that enables the introducing of multiple functional reaction groups into the antibodies, or the directly introducing of a molecule of interest (MOI) into the antibodies without a second step ligation reactions is highly desirable. However, development of such a robust conjugation strategy to generate effective and safe ADCs remains high challenging due many factors, including the minimal disturbance of affinity of the antibody, the number and the sites of the installed payloads, the linkage stability, and the homogeneity of the conjugates are difficult to control. Thus, an antibody conjugate with well-defined DAR, high homogeneity and high stablility, and a method for preparing the antibody conjugates are urgently needed.

### SUMMARY OF THE INVENTION

The present disclosure provides a protein conjugate and a method for making the same. The protein conjugate of the present disclosure has at least one of the following characteristics: (a) well-controlled and defined conjugation sites; (b) well defined molecule of interest (MOI)-to-antibody ratio (MAR) (c) high homogeneity; (d) negligible influence of the binding affinity of the antibody; (e) high stability (for example, the conjugation linkage is stable in human plasma for at least one day, e.g., two days, three days, four days, five days, six days, seven days, eight days or more); (f) high reactivity or good efficacy.

With the method developed in this invention, a variety of functional reaction groups (e.g., azide, BCN, TCO, MCP and Tz) could be transferred to the antibodies using an α-1,3-fucotrasferase and a GDP-(F)ₘ-(L)ₙ-Y₁ to generated the antibody-functional group-conjugates with high reactivity. Multiple ligation reaction could be applied to install a pharmaceutically active substance P (e.g. a cytotoxin) to the antibody to generate the antibody conjugates (the “two-step” process). Furthermore, a pharmaceutically active substance P (e.g. a cytotoxin) could be directly transferred to the antibodies using an α-1,3-fucotrasferase and the GDP-(F)ₘ-(L)ₙ-P to make the antibody conjugates (the “one-step” process). With either the “one-step” or the “two-step” process, we were able to make highly homogeneous and stable antibody conjugates, with excellent efficacy and negligible influence of the binding affinity of the antibody.

In one aspect, the present disclosure provides a protein conjugate, comprising a protein and an oligosaccharide, wherein said oligosaccharide comprises a structure of:

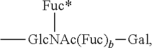

wherein: said GlcNAc is directly or indirectly linked to an amino acid of said protein; said Gal is a galactose; said (Fuc) is a fucose, b is 0 or 1; said Fuc* comprises a fucose or a fucose derivative linked to a molecule of interest (MOI), said protein comprises an antigen binding fragment and/or a Fc fragment.

In some embodiments, the oligosaccharide is an N-linked oligosaccharide. In some embodiments, the oligosaccharide is an O-linked oligosaccharide.

In some embodiments, the oligosaccharide is linked to an Asn residue of said protein.

In some embodiments, the GlcNAc in said oligosaccharide is directly linked to an Asn residue of said protein.

In some embodiments, the GlcNAc in said oligosaccharide is linked to a saccharide of said oligosaccharide.

In some embodiments, the GlcNAc in said oligosaccharide is linked to a mannose of said oligosaccharide.

In some embodiments, the protein comprises a Fc fragment.

In some embodiments, the protein comprises a Fc fragment and the oligosaccharide is linked to said Fc fragment.

In some embodiments, the oligosaccharide is linked to the CH₂ domain of said Fc fragment.

In some embodiments, the oligosaccharide is linked to the Asn297 position of said Fc fragment, numbered according to the Kabat numbering system.

In some embodiments, the protein is an antibody.

In some embodiments, the protein is an antibody, and the protein conjugate is capable of binding to an antigen. In some embodiments, the protein is an antibody, and the protein conjugate has the similar binding affinity towards an antigen, compared to the corresponding antibody. In some embodiments, the protein is an antibody, and the protein conjugate has a considerable binding affinity towards an antigen, compared to the corresponding antibody.

In some embodiments, the protein conjugate is capable of binding to an antigen and the binding affinity of said protein conjugate is about 0.1% to about 100000% of the binding affinity of the corresponding antibody.

In some embodiments, the protein conjugate is capable of binding to an antigen and the binding affinity of said protein conjugate is about 1% to about 10000% of the binding affinity of the corresponding antibody.

In some embodiments, the protein conjugate is capable of binding to an antigen and the binding affinity of said protein conjugate is about 10% to about 1000% of the binding affinity of the corresponding antibody.

In some embodiments, the protein conjugate is capable of binding to an antigen and the binding affinity of said protein conjugate is about 50% to about 200% of the binding affinity of the corresponding antibody.

In some embodiments, the antibody is a monoclonal antibody.

In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, the antibody is a chimeric antibody. In some embodiments, the antibody is a humanized antibody. In some embodiments, the antibody is a fully human antibody.

In some embodiments, the fucose or fucose derivative of said Fuc* is linked to said GlcNAc through an Fuc*α1,3GlcNAc linkage.

In some embodiments, the Gal is linked to said GlcNAc through a Galβ1,4GlcNAc linkage.

In some embodiments, the fucose of (Fuc) is linked to said GlcNAc through an α1,6 linkage.

In some embodiments, the MOI of Fuc* comprises an active moiety.

In some embodiments, the active moiety is a chemically active moiety, an enzymatically active moiety, a biologically active moiety, and/or a pharmaceutically active moiety.

In some embodiments, the active moiety comprises a chemically active moiety and/or an enzymatically active moiety.

In some embodiments, the said active moiety comprises a functional group Y₁ capable of participating in a ligation reaction.

In some embodiments, the Y₁ comprises a functional moiety capable of participating in a bioorthogonal reaction.

In some embodiments, the Y₁ comprises a functional moiety selected from the group consisting of azide, terminal alkyne, cyclic alkyne, tetrazine, 1,2,4-trazine, terminal alkene, transcyclooctene, cyclopropene, norbornene, keto, aldehyde, aminooxy, thiol, maleimide and their derivatives thereof.

In some embodiments, the Y₁ comprises a functional moiety selected from the group consisting of

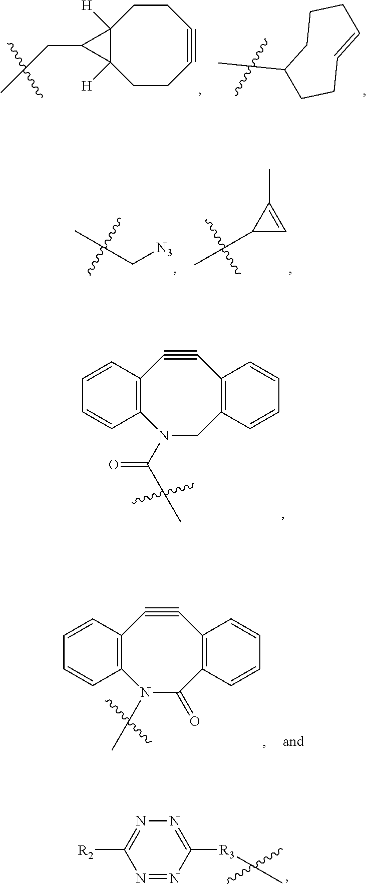

wherein each of R₁ and R₂ is independently selected from the group consisting of hydrogen, halogen, C₁-C₂₂ alkyl group, C₅-C₂₂ (hetero)aryl group, C₇-C₂₂ alkyl(hetero)aryl group and C₇-C₂₂ (hetero)arylalkyl group, wherein each of said alkyl group optionally is interrupted by one or more hetero-atom selected from the group consisting of O, N, and S, and wherein each of the alkyl group, (hetero)aryl group, alkyl(hetero)aryl group and (hetero)arylalkyl groups is independently optionally substituted.

In some embodiments, the Y₁ comprises a functional moiety selected from the group consisting of

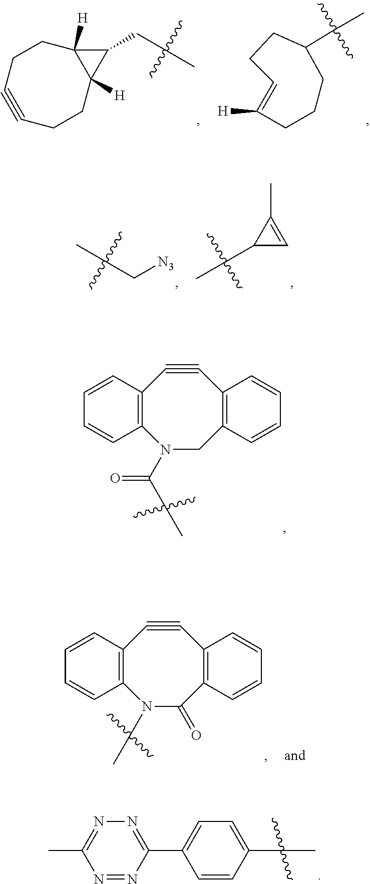

In some embodiments, the active moiety of MOI comprises a P, and the P is a biologically and/or a pharmaceutically active substance.

In some embodiments, the P is a pharmaceutically active substance.

In some embodiments, the P comprises a cytotoxin, an agonist, an antagonist, an antiviral agent, an antibacterial agent, a radioisotope or radionuclide, a metal chelator, an oligonucleotide, a peptide, a polypeptide, a protein, or any combination thereof.

In some embodiments, the P comprises a cytotoxin, an agonist, an antagonist, an antiviral agent, an antibacterial agent, an oligonucleotide, a peptide, a polypeptide or any combination thereof.

In some embodiments, the P comprises a cytotoxin, an agonist, an antagonist, or any combination thereof.

In some embodiments, the P comprises an anti-tumor agent. In some embodiments, the P comprises a substance which results in cell damage or cell death.

In some embodiments, the P comprises a cytotoxin. In some embodiments, the P comprises a cytotoxin selected from the group consisting of a DNA damaging agent, a topoisomerase inhibitor and a microtubule inhibitor.

In some embodiments, the P comprises a cytotoxin selected from the group consisting of pyrrolobenzodiazepine (PBD), auristatin (e.g., MMAE, or MMAF), maytansinoids (Maytansine, DM1, or DM4), duocarmycin, tubulysin, enediyene (e.g. Calicheamicin), doxorubicin (PNUs), pyrrole-based kinesin spindle protein (KSP) inhibitor, calicheamicin, amanitin (e.g. a-Amanitin), camptothecin (e.g. exatecan, deruxtecan).

In some embodiments, the P comprises a cytotoxin selected from the group consisting of MMAE, DXd, MMAF, seco-DUBA and DM4.

In some embodiments, the MOI may further comprise a remaining group Y₁Y₂ after a ligation reaction between said Y₁ and a functional group Y₂.

In some embodiments, the Y₁Y₂ is between said fucose or fucose derivative of Fuc* and said P.

In some embodiments, the Y₂ comprises a functional moiety capable of participating in a bioorthogonal reaction.

In some embodiments, the Y₂ comprises a functional moiety selected from the group consisting of azide, terminal alkyne, cyclic alkyne, tetrazine, 1,2,4-trazine, terminal alkene, transcyclooctene, cyclopropene, norbornene, keto, aldehyde, aminooxy, thiol, maleimide and their derivatives thereof.

In some embodiments, the Y₂ comprises a functional moiety selected from the group consisting of

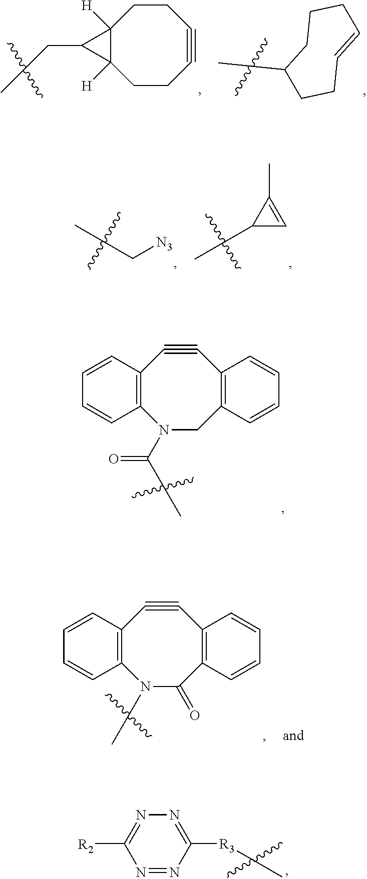

wherein each of R₁ and R₂ is independently selected from the group consisting of hydrogen, halogen, C₁-C₂₂ alkyl group, C₅-C₂₂ (hetero)aryl group, C₇-C₂₂ alkyl(hetero)aryl group and C₇-C₂₂ (hetero)arylalkyl group, wherein each of said alkyl group optionally is interrupted by one or more hetero-atom selected from the group consisting of O, N, and S, and wherein each of the alkyl group, (hetero)aryl group, alkyl(hetero)aryl group and (hetero)arylalkyl groups is independently optionally substituted.

In some embodiments, the Y₂ comprises a functional moiety selected from the group consisting of

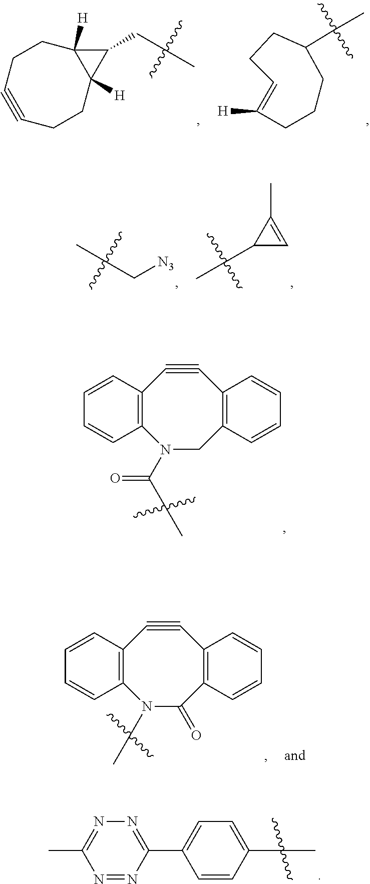

In some embodiments, the group Y₁Y₂ is selected from the group consisting of

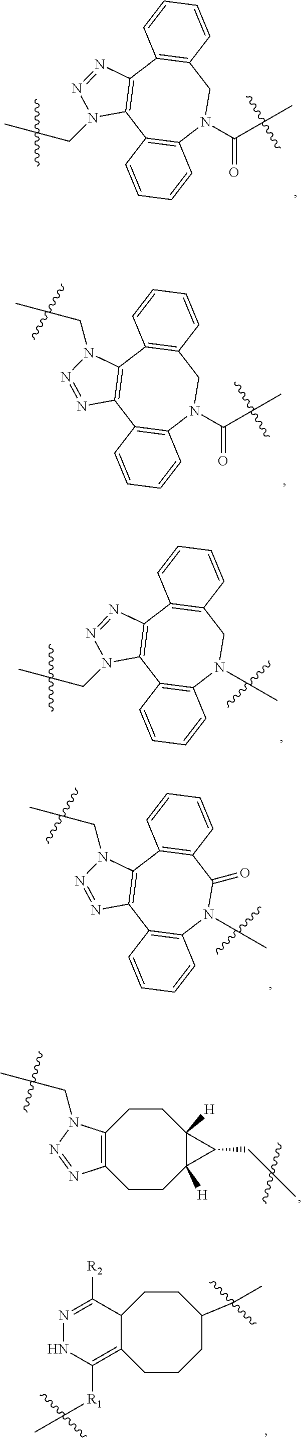

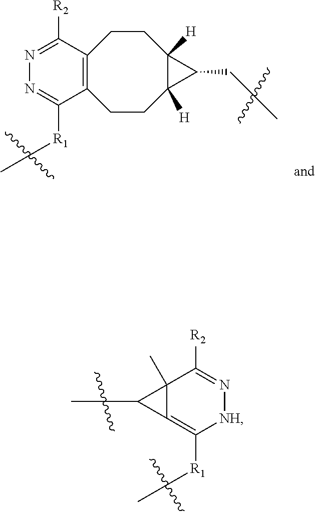

wherein R₁ and R₂ are defined as above.

In some embodiments, the Y₁ and the Y₂ comprise the functional moiety selected from the group consisting of: a) Y₁ comprises

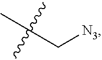 [SMILES: *CN=[N+]=[N-]]

and Y₂ comprises

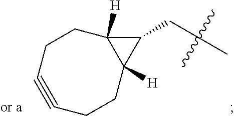 [SMILES: *C[C@H]1[C@H]2CCC#CCC[C@@H]12]

b) Y₁ comprises

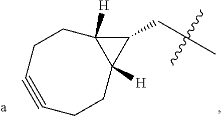 [SMILES: *C[C@H]1[C@H]2CCC#CCC[C@@H]12]

and Y₂ comprises

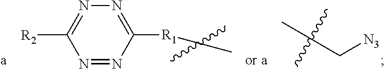

c) Y₁ comprises

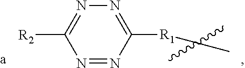

and Y₂ comprises

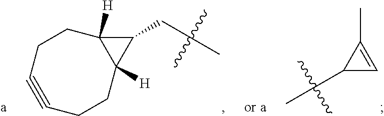

and d) Y₁ comprises

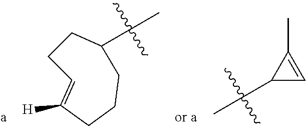

and Y₂ comprises

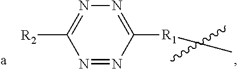

wherein R₁ and R₂ are defined as above.

In some embodiments, the MOI of Fuc* may further comprise a L, and L is a linker. The linker can be a cleavable linker or a non-cleavable linker.

In some embodiments, the linker L is a cleavable linker (e.g., susceptible to cleavage under intracellular conditions). In some embodiments, cleavable linker can be selectively cleaved by a chemical or biological process and upon cleavage separate the P.

In some embodiments, the L is an acid-labile linker, a redox-active linker, a photo-active linker and/or a proteolytically cleavable linker.

In some embodiments, the L is a vc-PAB linker, a GGFG linker or a dislufo linker.

In some embodiments, the L is between said fucose or fucose derivative of Fuc* and said P. In some embodiments, the L is between said fucose or fucose derivative of Fuc* and said Y₁. In some embodiments, the L is between said fucose or fucose derivative of Fuc* and said Y₁Y₂.

In some embodiments, the MOI of Fuc* may further comprise a F, and F is a connector.

In some embodiments, the F is

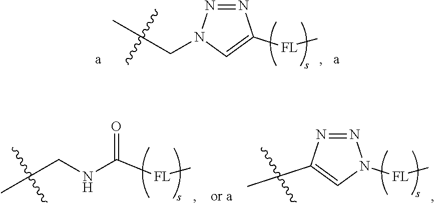

wherein said FL is a spacer and s is 0 or 1. In some embodiments, s is 1.

In some embodiments, the F is

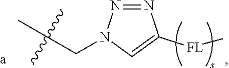

wherein said FL is a spacer and s is 0 or 1. In some embodiments, s is 1.

In some embodiments, the F is

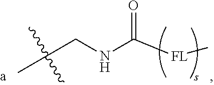

Wherein said FL is a spacer and s is 0 or 1. In some embodiments, s is 1.

In some embodiments, the FL is a polypeptide, a PEG, an alkyl and/or their derivatives or combination thereof.

In some embodiments, the FL has a structure selected from the group consisting of:

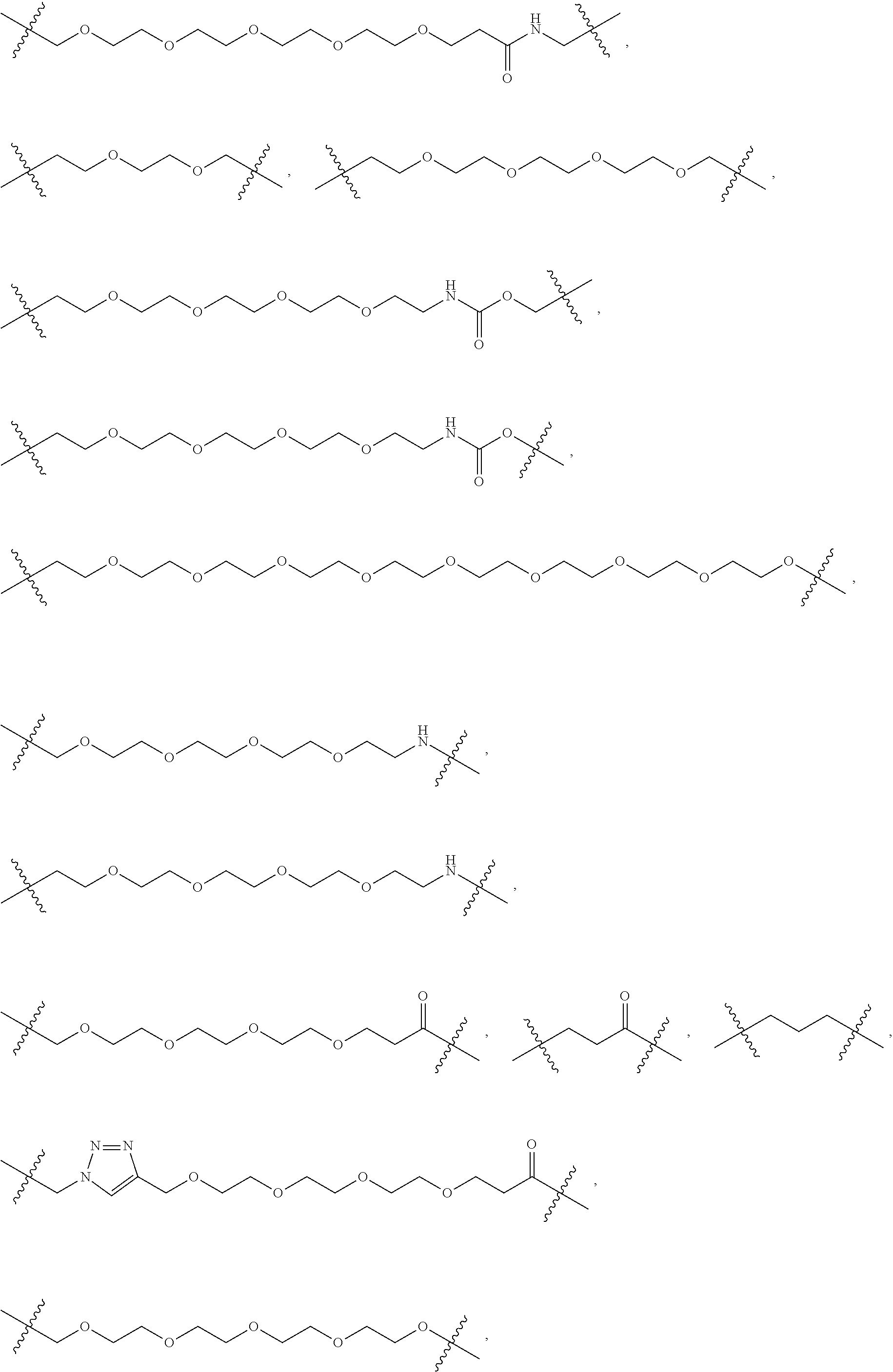

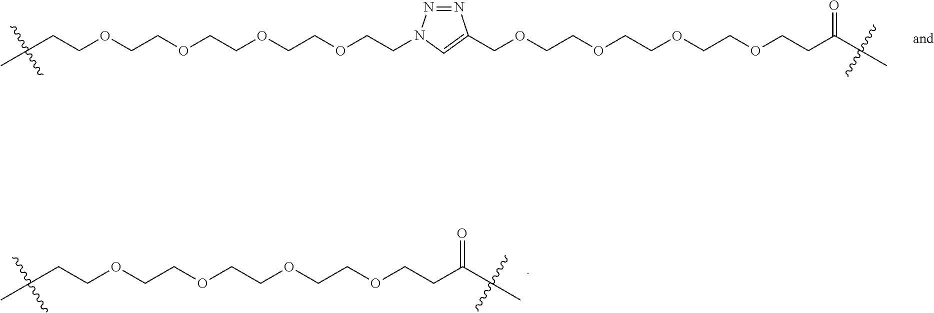 [SMILES: *CCOCCOCCOCCOCCn1cc(COCCOCCOCCOCCC(*)=O)nn1 ; *CCOCCOCCOCCOCCC(*)=O]

For example, the fucoses or fucose derivative, e.g., the Fuc, is linked to the left terminus of the structure of the FL.

In some embodiments, the F is between said fucose or fucose derivative of Fuc* and said P. In some embodiments, the F is between said fucose or fucose derivative of Fuc* and said Y₁. In some embodiments, the F is between said fucose or fucose derivative of Fuc* and said Y₁Y₂.

In some embodiments, Fuc* is Fuc-(F)ₘ-(L)ₙ-Y₁, Fuc-(F)ₘ-(L)ₙ-P, or Fuc-(F)ₘ-(L)ₙ-Y₁Y₂-(FL′)_{m′}-(L′)_{n′}-P, wherein Fuc is said fucose or fucose derivative of the Fuc*, F is the connector, L is the linker, P is the biologically and/or a pharmaceutically active substance, Y₁ is the functional group, FL′ is a spacer defined as the same as FL, L′ is a linker defined as the same as L, m is 0 or 1, n is 0 or 1, m′ is 0 or 1, and n′ is 0 or 1. For example, Fuc* is Fuc-Y₁. For example, Fuc* is Fuc-F-Y₁. For example, Fuc* is Fuc-L-Y₁. For example, Fuc* is Fuc-F-L-P. For example, Fuc* is Fuc-P For example, Fuc* is Fuc-F-P. For example, Fuc* is Fuc-L-P. For example, Fuc* is Fuc-Y₁Y₂-L′-P. For example, Fuc* is Fuc-Y₁Y₂-FL′-L′-P. For example, Fuc* is Fuc-Y₁Y₂-FL′-P. For example, Fuc* is Fuc-Y₁Y₂-P. For example, Fuc* is Fuc-F-Y₁Y₂-FL′-L′-P. For example, Fuc* is Fuc-F-Y₁Y₂-FL′-P. For example, Fuc* is Fuc-F-Y₁Y₂-P. For example, Fuc* is Fuc-F-Y₁Y₂-L′-P.

In some embodiments, the Fuc is according to the formula

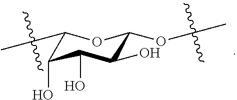

In some embodiments, the Fuc* is according to the formula

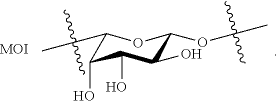

In some embodiments, the protein conjugate comprises 1-20

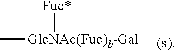

In some embodiments, the protein conjugate comprises 2 or 4

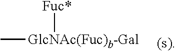

In some embodiments, in the protein conjugate, the Fuc* is linked to the GlcNAc of a terminal LacNAc of the

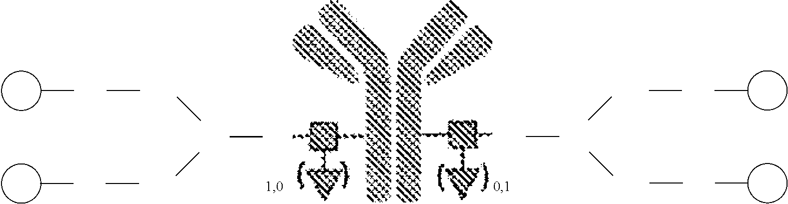

wherein [symbol] is a GlcNAc, [symbol] is the fucose of (Fuc) linked a core GlcNAc through an α1,6 linkage, [symbol] is a mannose, ◯ is a galactose linked to a GlcNAc through a Galβ1,4GlcNAc linkage, and [symbol] is an antibody or a Fc-fusion protein. For example, the Fuc* is linked to the GlcNAc of a terminal LacNAc of an antibody-G₂F. For example, the Fuc* is linked to the GlcNAc of a terminal LacNAc of a Fc-fusion protein-G₂F.

In some embodiments, the Fuc* is linked to the core GlcNAc of

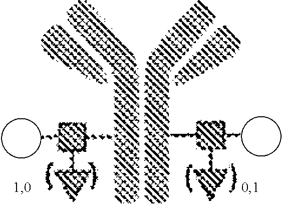

through an Fuc*α1,3GlcNAc linkage, wherein [symbol] is a GlcNAc, [symbol] is the fucose of (Fuc) linked the core GlcNAc through an α1,6 linkage, ◯ is a galactose linked to a GlcNAc through a Galβ1,4GlcNAc linkage, and [symbol] is an antibody or a Fc-fusion protein. For example, the Fuc* is linked to the core GlcNAc of the -((Fuc)α1,6)GlcNAc-Gal of an antibody. For example, Fuc* is linked to the core GlcNAc of the -((Fuc)α1,6)GlcNAc-Gal of an Fc-fusion protein. For example, the Fuc* is linked to the core GlcNAc of the -GlcNAc-Gal of an antibody. For example, Fuc* is linked to the core GlcNAc of the -GlcNAc-Gal of an Fc-fusion protein.

In some embodiments, the protein conjugate is according to the formula

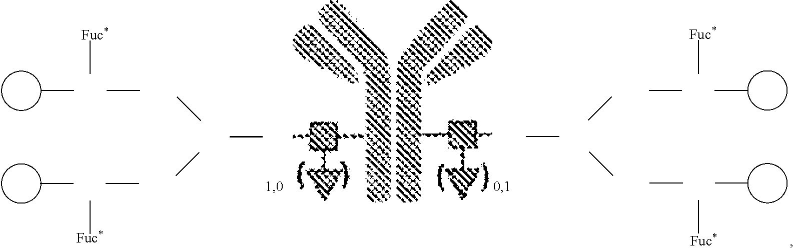

wherein [symbol] is the GlcNAc, [symbol] is the fucose of (Fuc) linked the GlcNAc through an α-1,6 linkage, [symbol] is the mannose, ◯ is the galactose linked to the GlcNAc through a Galβ1,4GlcNAc linkage, Fuc* is linked to the GlcNAc through an Fuc*α1,3GlcNAc linkage, and [symbol] is an antibody or a Fc fusion protein. In some embodiments, the oligosaccharide is linked to the N297 position of a Fc part of the antibody or the Fc fusion protein. For example, the protein is an antibody. For example, the antibody conjugate is for treating disease. For example, when the Fuc* comprise the pharmaceutically active substance P, the antibody conjugate is for treating disease. For example, when the Fuc* comprise the functional group Y₁, the antibody conjugate is for making an agent for treating disease. For example, the protein conjugate may be capable of binding to an antigen. For example, the protein conjugate has a similar binding affinity as the corresponding antibody towards an antigen.

In some embodiments, the protein conjugate is according to the formula

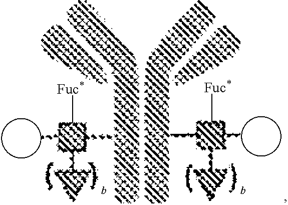

wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody or a Fc fusion protein, and b is 0 or 1. In some embodiments, the GlcNAc is linked to the N297 position of the Fc part. For example, b is 0. For example, b is 1. For example, the antibody conjugate is for treating disease. For example, when the Fuc* comprise the pharmaceutically active substance P, the antibody conjugate is for treating disease. For example, when the Fuc* comprise the functional group Y₁, the antibody conjugate is for making an agent for treating disease. For example, the protein conjugate may be capable of binding to an antigen. For example, the protein conjugate has a similar binding affinity as the corresponding antibody towards an antigen.

In some embodiments, the protein conjugate is an antibody-drug conjugate. In some embodiments, the protein conjugate is a protein conjugate for treating disease.

In some embodiments, when the Fuc* of the protein conjugate comprises the functional group Y₁, the antibody conjugate is for making an agent for treating disease.

In some embodiments, the protein conjugate has one or more of the following properties: (1) having a MAR of 2 or 4, (2) capable of binding to an antigen, (3) capable of binding to an antigen, with a similar binding affinity as a corresponding antibody, (4) stable in plasma for at least one day (e.g., two days, three days, four days, five days, six days, seven days, eight days or more), as measured in mass spectrometry analysis, (5) the linkage between the Fuc of Fuc* and the GlcNAc of the -GlcNAc(Fuc)_{b}-Gal are stable in plasma for at least 1 day (e.g., two days, three days, four days, five days, six days, seven days, eight days or more), as measured in mass spectrometry analysis, b is 0 or 1. (6) having a high reactive activity, and/or (7) capable of inhibiting tumor growth and/or tumor cell proliferation.

In another aspect, the present disclosure provides a composition comprising the protein conjugate of the present disclosure. In some embodiments, the present disclosure provides a composition comprising the protein conjugate according to the formula

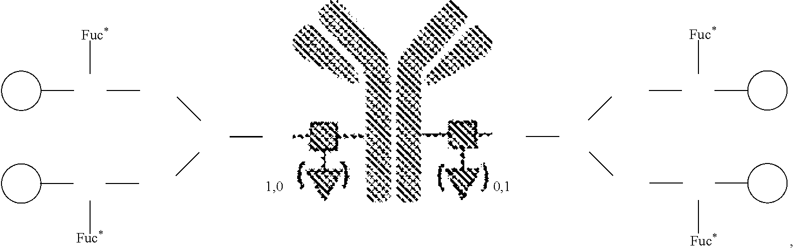

wherein [symbol] is the GlcNAc, [symbol] is the fucose of (Fuc) linked the GlcNAc through an α-1,6 linkage, [symbol] is the mannose, ◯ is the galactose linked to the GlcNAc through a Galβ1,4GlcNAc linkage, Fuc* is linked to the GlcNAc through an Fuc*α1,3GlcNAc linkage, and [symbol] is an antibody or a Fc fusion protein. In some embodiments, the oligosaccharide is linked to the N297 position of a Fc domain of the antibody or the Fc fusion protein. In some embodiments, the composition has an average MOI-to-antibody ratio (MAR) of about 2.4-4. In some embodiments, the composition has an average MAR of about 2.8-4. In some embodiments, the composition has an average MAR of about 3-4. In some embodiments, the composition has an average MAR of about 3.5-4. In some embodiments, the composition has an average MAR of about 3.8-4. In some embodiments, the composition has an average MAR of about 4.

In another aspect, the present disclosure provides a composition comprising the protein conjugate of the present disclosure. In some embodiments, the present disclosure provides a composition comprising the protein conjugate according to the formula

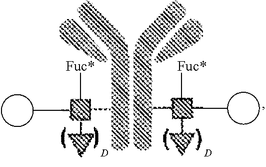

wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody or a Fc fusion protein, and b is 0 or 1. In some embodiments, the GlcNAc is linked to the N297 position of a Fe domain of the antibody or the Fc fusion protein. In some embodiments, b is 0. In some embodiments, b is 1. In some embodiments, the composition has an average MOI-to-antibody ratio (MAR) of about 0.5-2. In some embodiments, the composition has an average MAR of about 1-2. In some embodiments, the composition has an average MAR of about 1.5-2. In some embodiments, the composition has an average MAR of about 1.8-2. In some embodiments, the composition has an average MAR of about 2.

In another aspect, the present disclosure provides a method for preparing the protein conjugate and/or the composition of the present disclosure.

In another aspect, the present disclosure provides a method for preparing a protein conjugate, wherein the method comprises a step (a), and step (a) comprises contacting a fucose derivative donor Q-Fuc*′ to a protein comprising an oligosaccharide in the presence of a catalyst, wherein the oligosaccharide comprise -GlcNAc(Fuc)_{b}-Gal, to obtain a protein conjugate comprising

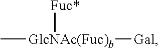

wherein the GlcNAc is directly or indirectly linked to an amino acid of the protein; the Gal is a galactose; the (Fuc) is a fucose, b is 0 or 1; the Fuc*′ comprises a fucose or fucose derivative linked to a molecule of interest (MOI′); the protein comprises an antigen binding fragment and/or a Fc fragment; the Q-Fuc*′ is a molecule comprises the Fuc*′.

In some embodiments, the method comprises buffer exchange of the obtained protein conjugate comprising

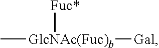

into a buffer. In some embodiments, the buffer is a formulation buffer. In some embodiments, the buffer is a storage buffer.

In some embodiments, the catalyst is a fucosyltransferase or a functional variant or fragment thereof. In some embodiments, the fucosyltransferase is an α-1,3-fucosyltransferase or a functional variant or fragment thereof.

In some embodiments, the fucosyltransferase is derived from a bacterium, a nematode, a trematode, or a mammal. In some embodiments, the fucosyltransferase is derived from Helicobacter pylori. In some embodiments, the fucosyltransferase comprises an amino acid sequence as set forth in GenBank Accession No. AF008596.1, GenBank Accession No. AAD07447.1, GenBank Accession No. AAD07710.1, GenBank Accession No. AAF35291.2, or GenBank Accession No. AAB93985.1, or their functional variant or fragment thereof. In some embodiments, the fucosyltransferase comprises an amino acid sequence as set forth in GenBank Accession No. AAD07710.1, or a functional variant or fragment thereof. In some embodiments, the fucosyltransferase comprises an amino acid sequence as set forth in SEQ ID NO: 3 or SEQ ID NO: 4.

In some embodiments, the fucosyltransferase is derived from human. In some embodiments, the fucosyltransferase comprises an amino acid sequence as set forth in Uniprot Accession No. P51993, or a functional variant or fragment thereof.

In some embodiments, the oligosaccharide is an N-linked oligosaccharide.

In some embodiments, the oligosaccharide is linked to an Asn residue of said protein.

In some embodiments, the GlcNAc in said oligosaccharide is directly linked to an Asn residue of said protein.

In some embodiments, the GlcNAc in said oligosaccharide is linked to a saccharide of said oligosaccharide.

In some embodiments, the GlcNAc in said oligosaccharide is linked to a mannose of said oligosaccharide.

In some embodiments, the protein comprises a Fc fragment.

In some embodiments, the oligosaccharide is linked to the Fc fragment.

In some embodiments, the oligosaccharide is linked to the CH₂ domain of said Fc fragment.

In some embodiments, the oligosaccharide is linked to the Asn297 position of said Fc fragment, numbered according to the Kabat numbering system.

In some embodiments, the protein is an antibody. In some embodiments, the protein is an antibody, and the protein conjugate is capable of binding to an antigen.

In some embodiments, the protein is an antibody, and the protein conjugate has a similar binding affinity towards an antigen, compared to the corresponding antibody. In some embodiments, the protein is an antibody, and the protein conjugate has a considerable binding affinity towards an antigen, compared to the corresponding antibody.

In some embodiments, the obtained protein conjugate is capable of binding to an antigen and the binding affinity is about 0.1% to about 100000% of the binding affinity of the corresponding antibody.

In some embodiments, the obtained protein conjugate is capable of binding to an antigen and the binding affinity is about 1% to about 10000% of the binding affinity of the corresponding antibody.

In some embodiments, the obtained protein conjugate is capable of binding to an antigen and the binding affinity is about 10% to about 1000% of the binding affinity of the corresponding antibody.

In some embodiments, the obtained protein conjugate is capable of binding to an antigen and the binding affinity is about 50% to about 200% of the binding affinity of the corresponding antibody.

In some embodiments, the antibody is a monoclonal antibody.

In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, the antibody is a chimeric antibody. In some embodiments, the antibody is a humanized antibody. In some embodiments, the antibody is a fully human antibody.

In some embodiments, the Gal is linked to said GlcNAc through a Galβ1,4GlcNAc linkage.

In some embodiments, the fucose of the (Fuc) is linked to said GlcNAc through an α1,6 linkage.

In some embodiments, the Q-Fuc*′ comprises a ribonucleotide diphosphate.

In some embodiments, the Q-Fuc*′ comprises uridine diphosphate (UDP), guanosine diphosphate (GDP) or cytidine diphosphate (CDP). In some embodiments, the Q-Fuc*′ is GDP-Fuc*′

In some embodiments, the MOI′ of Fuc*′ comprises an active moiety.

In some embodiments, the active moiety of the MOI′ comprises a chemically active moiety, an enzymatically active moiety, a biologically active moiety, and/or a pharmaceutically active moiety.

In some embodiments, the active moiety of the MOI′ comprises a chemically active moiety and/or an enzymatically active moiety active moiety.

In some embodiments, the active moiety of the MOI′ comprises a functional group Y₁ capable of participating in a ligation reaction.

In some embodiments, the Y₁ comprises a functional moiety capable of participating in a bioorthogonal reaction.

In some embodiments, the Y₁ comprises a functional moiety selected from the group consisting of azide, terminal alkyne, cyclic alkyne, tetrazine, 1,2,4-trazine, terminal alkene, transcyclooctene, cyclopropene, norbornene, keto, aldehyde, aminooxy, thiol, maleimide and their derivatives thereof.

In some embodiments, the Y₁ comprises a functional moiety selected from the group consisting of

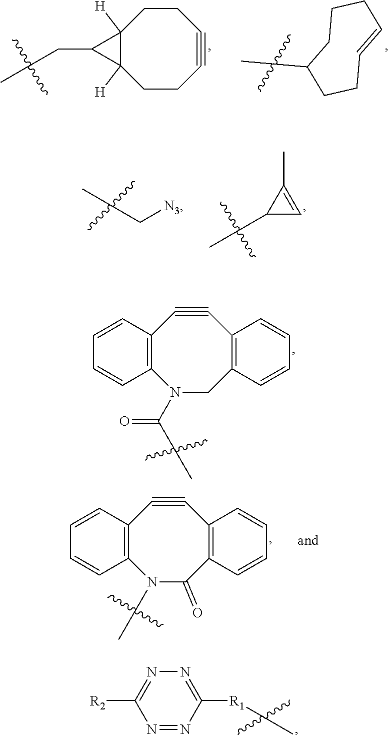

wherein each of R₁ and R₂ is independently selected from the group consisting of hydrogen, halogen, C₁-C₂₂ alkyl group, C₅-C₂₂ (hetero)aryl group, C₇-C₂₂ alkyl(hetero)aryl group and C₇-C₂₂ (hetero)arylalkyl group, wherein each of said alkyl group optionally is interrupted by one or more hetero-atom selected from the group consisting of O, N, and S, and wherein each of the alkyl group, (hetero)aryl group, alkyl(hetero)aryl group and (hetero)arylalkyl groups is independently optionally substituted.

In some embodiments, the Y₁ comprises a functional moiety selected from the group consisting of

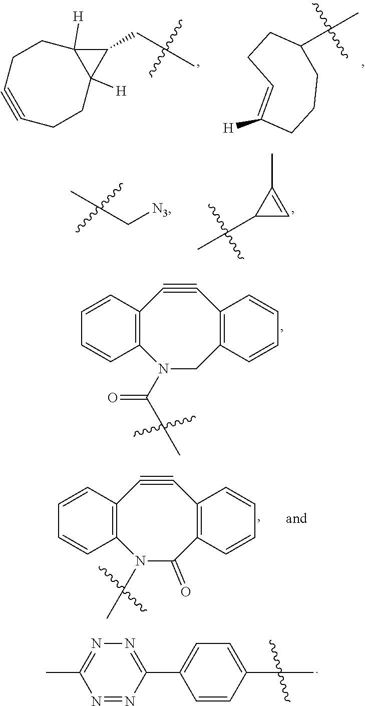

In some embodiments, the method comprises contacting the Q-Fuc*′ with the protein to obtain a protein conjugate comprising

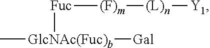

wherein Q-Fuc*′ is Q-Fuc-(F)ₘ-(L)ₙ-Y₁, Fuc is the fucose or fucose derivative of Fuc*′, F is a connector, L is a linker, b is 0 or 1, m is 0 or 1, and n is 0 or 1. For example, m is 1 and n is 0. For example, m is 0 and n is 1. For example, m is 1 and n is 1.

In some embodiments, the active moiety of the MOI′ comprises a P, and P is a biologically and/or a pharmaceutically active substance.

In some embodiments, the method comprises contacting said Q-Fuc*′ with said protein to obtain a protein conjugate comprising

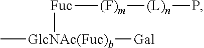

wherein Q-Fuc*′ is Q-Fuc-(F)ₘ-(L)ₙ-P, Fuc is the fucose or fucose derivative of Fuc*′, F is a connector, L is a linker, and P is a biologically and/or pharmaceutically active substance, b is 0 or 1, m is 0 or 1, and n is 0 or 1. For example, m is 0 and n is 1. For example, m is 1 and n is 0. For example, m is 1 and n is 1.

In some embodiments, the method further comprises a step (b) contacting said protein conjugate comprising

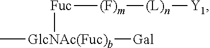

to a Y₂-(FL′)_{m′}-(L′)_{n′}-P, to obtain a protein conjugate comprising

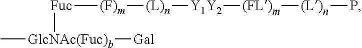

wherein Y₁Y₂ is a remaining group after a ligation reaction between said Y₁ and a functional group Y₂ comprising a functional moiety capable of reacting with Y₁, FL′ is a spacer defined as the same as FL, L′ is a linker defined the same as L, b is 0 or 1, m is 0 or 1, n is 0 or 1, m′ is 0 or 1, n′ is 0 or 1, and P is a biologically and/or pharmaceutically active substance. For example, m is 1 and n is 0. For example, m is 1 and n is 1.

In some embodiments, the P is a pharmaceutically active substance.

In some embodiments, the P a cytotoxin, an agonist, an antagonist, an antiviral agent, an antibacterial agent, a radioisotope or radionuclide, a metal chelator, an oligonucleotide, a peptide, a polypeptide, a protein, or any combination thereof.

In some embodiments, the P comprises a cytotoxin, an agonist, an antagonist, an antiviral agent, an antibacterial agent, an oligonucleotide, a peptide, a polypeptide or any combination thereof.

In some embodiments, the P comprises a cytotoxin, an agonist, an antagonist or any combination thereof.

In some embodiments, the P comprises an anti-tumor agent.

In some embodiments, the comprises a substance which results in cell damage or cell death.

In some embodiments, the P comprises a cytotoxin.

In some embodiments, the P comprises a cytotoxin selected from the group consisting of a DNA damaging agent, a topoisomerase inhibitor and a microtubule inhibitor.

In some embodiments, the P comprises a cytotoxin selected from the group consisting of pyrrolobenzodiazepine (PBD), auristatin (e.g., MMAE, or MMAF, maytansinoids (Maytansine, DM1, or DM4), duocarmycin, tubulysin, enediyene (e.g. Calicheamicin), doxorubicin (PNUs,), pyrrole-based kinesin spindle protein (KSP) inhibitor, calicheamicin, amanitin (e.g. a-Amanitin), camptothecin (e.g. exatecan, deruxtecan).

In some embodiments, the P comprises a cytotoxin selected from the group consisting of MMAE, DXd, MMAF, seco-DUBA and DM4.

In some embodiments, the linker L is a cleavable linker.

In some embodiments, the L is an acid-labile linker, a redox-active linker, a photo-active linker and/or a proteolytically cleavable linker.

In some embodiments, the L is a vc-PAB linker, a GGFG linker or a disulfo linker.

In some embodiments, the linker L′ is a cleavable linker.

In some embodiments, the L′ is an acid-labile linker, a redox-active linker, a photo-active linker and/or a proteolytically cleavable linker.

In some embodiments, the L′ is a vc-PAB linker, a GGFG linker or a disulfo linker.

In some embodiments, the connector F is is

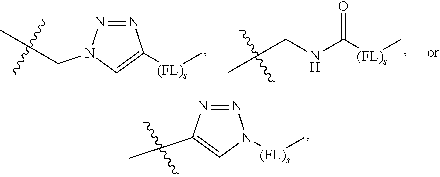

wherein said FL is a spacer and s is 0 or 1. For example, s is 1.

In some embodiments, the connector F is

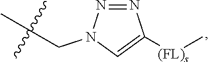

wherein said FL is a spacer and s is 0 or 1. For example, s is 1.

In some embodiments, the connector F is

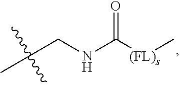

wherein said FL is a spacer and s is 0 or 1. For example, s is 1.

In some embodiments, the spacer FL is a polypeptide, a PEG, an alkyl and/or their derivatives or combination thereof.

In some embodiments, the spacer FL is selected from the group consisting of:

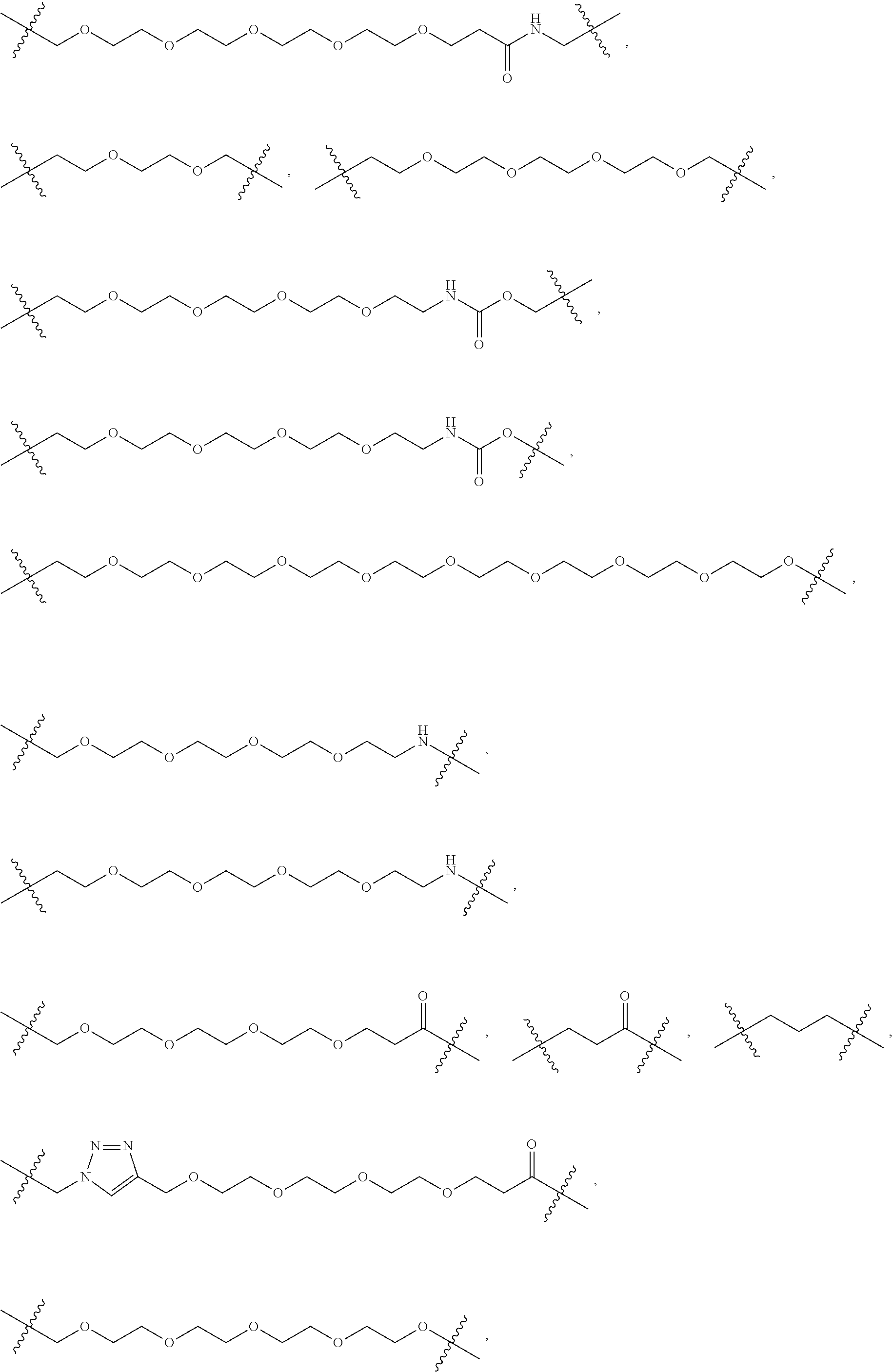

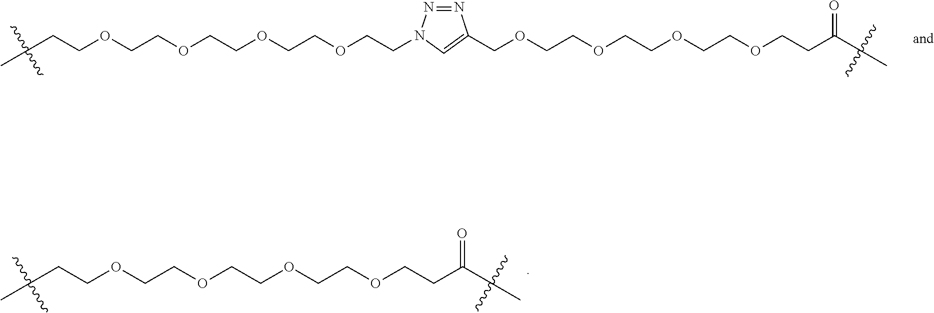 [SMILES: *CCOCCOCCOCCOCCn1cc(COCCOCCOCCOCCC(*)=O)nn1 ; *CCOCCOCCOCCOCCC(*)=O]

In some embodiments, the spacer FL′ is a polypeptide, a PEG, an alkyl and/or their derivatives or combination thereof.

In some embodiments, the spacer FL′ is selected from the group consisting of:

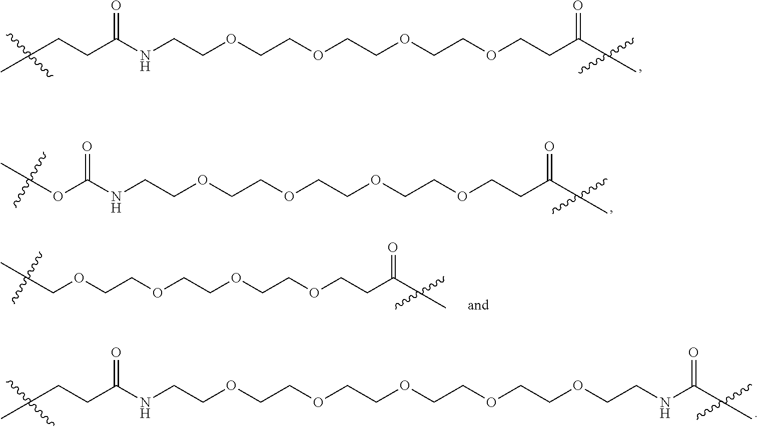 [SMILES: *CCC(=O)NCCOCCOCCOCCOCCC(*)=O ; *OC(=O)NCCOCCOCCOCCOCCC(*)=O ; *COCCOCCOCCOCCC(*)=O ; *CCC(=O)NCCOCCOCCOCCOCCOCCNC(*)=O]

In some embodiments, the Y₂ comprises a functional moiety capable of participating in a bioorthogonal reaction.

In some embodiments, the Y₂ comprises a functional moiety selected from the group consisting of azide, terminal alkyne, cyclic alkyne, tetrazine, 1,2,4-trazine, terminal alkene, transcyclooctene, cyclopropene, norbornene, keto, aldehyde, aminooxy, thiol, maleimide and their derivatives thereof.

In some embodiments, the Y₂ comprises a functional moiety selected from the group consisting of

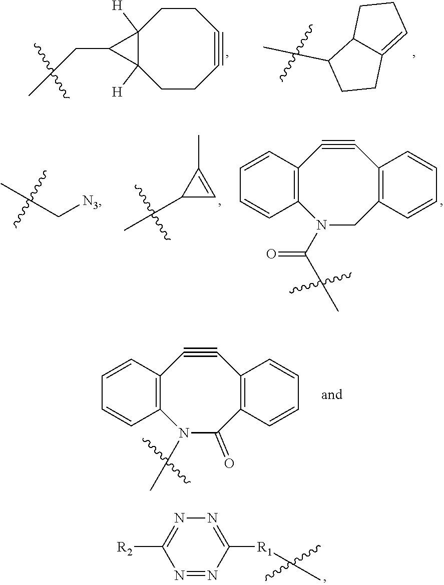

wherein each of R₁ and R₂ is independently selected from the group consisting of hydrogen, halogen, C₁-C₂₂ alkyl group, C₅-C₂₂ (hetero)aryl group, C₇-C₂₂ alkyl(hetero)aryl group and C₇-C₂₂ (hetero)arylalkyl group, wherein each of said alkyl group optionally is interrupted by one or more hetero-atom selected from the group consisting of O, N, and S, and wherein each of the alkyl group, (hetero)aryl group, alkyl(hetero)aryl group and (hetero)arylalkyl groups is independently optionally substituted.

In some embodiments, the Y₂ comprises a functional moiety selected from the group consisting of

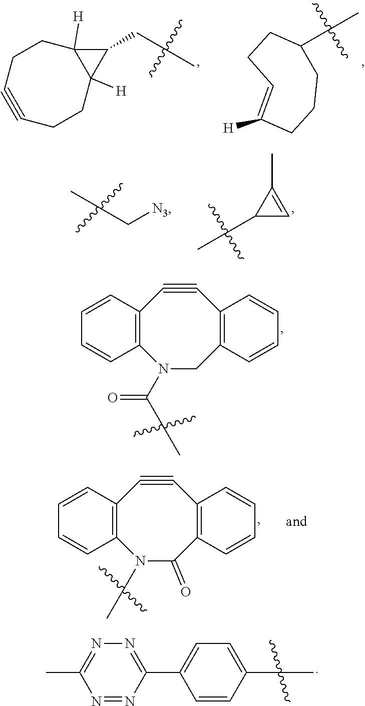

In some embodiments, the group Y₁Y₂ is selected from the group consisting of

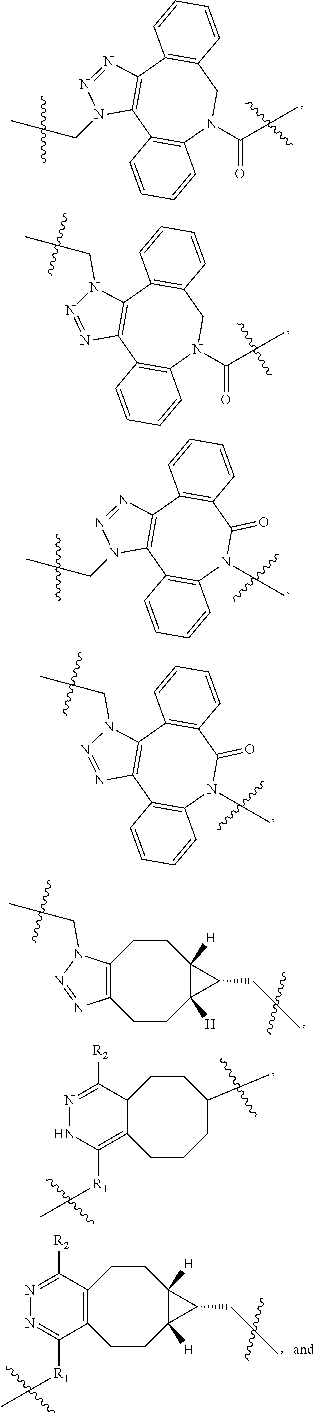

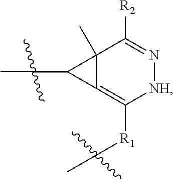

wherein said R₁ and R₂ are defined as above.

In some embodiments, the Y₁ and the Y₂ comprise the functional moiety selected from the group consisting of a) Y₁ comprises

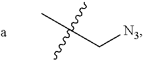 [SMILES: *CN=[N+]=[N-]]

and Y₂ comprises

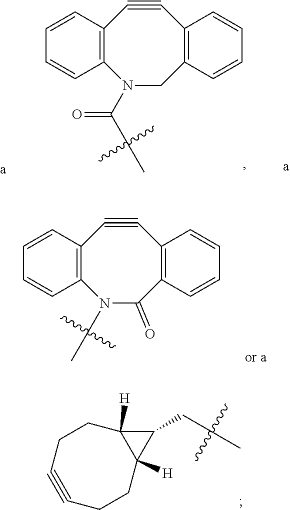 [SMILES: *C(=O)N1Cc2ccccc2C#Cc2ccccc21 ; *N1C(=O)c2ccccc2C#Cc2ccccc21 ; *C[C@H]1[C@H]2CCC#CCC[C@@H]12]

b) Y₁ comprises

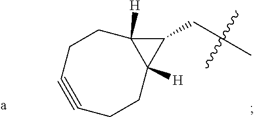 [SMILES: *C[C@H]1[C@H]2CCC#CCC[C@@H]12]

and Y₂ comprises

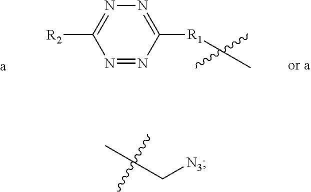

c) Y₁ comprises

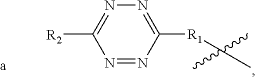

and Y₂ comprises

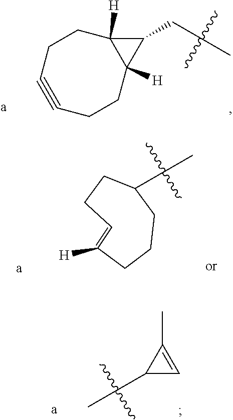

and d) Y₁ comprises

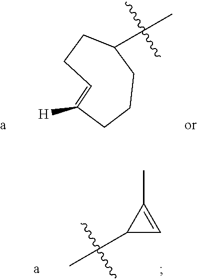

and Y₂ comprises

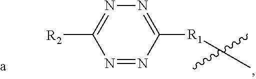

wherein said R₁ and R₂ are defined as above.

In some embodiments, the Q-Fuc*′ has a structure of

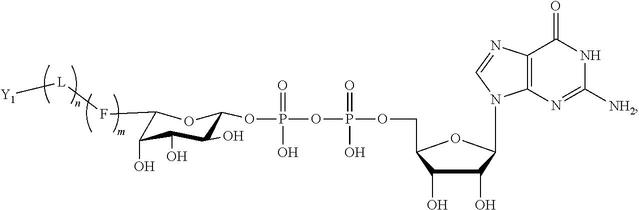

wherein said the F is the connector, L is the linker, Y₁ is the functional group, m is 0 or 1 and n is 0 or 1. In some embodiments m is 1 and n is 0.

In some embodiments, the Q-Fuc*′ is selected from the group consisting of

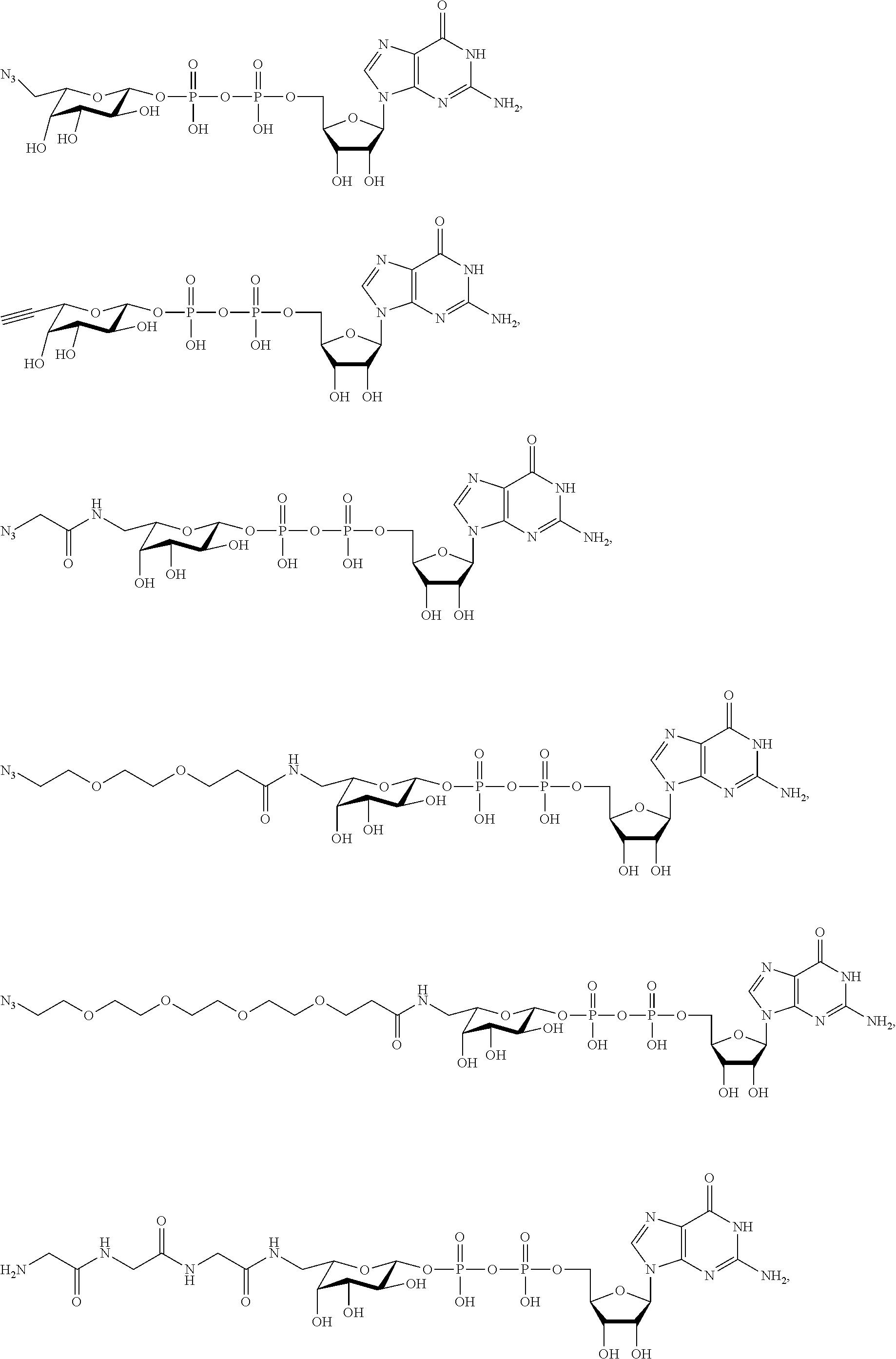

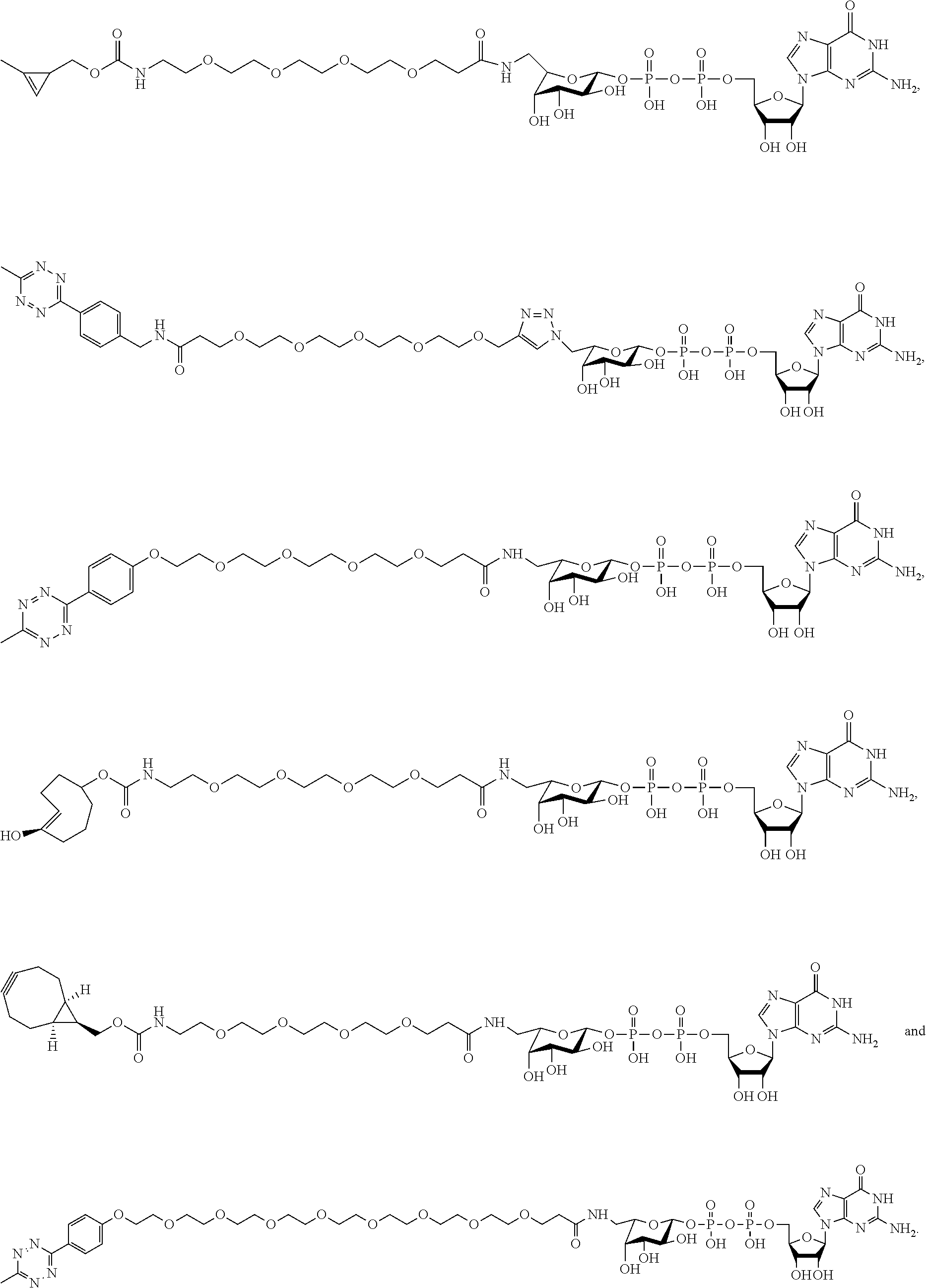

In some embodiment, the Q-Fuc*′ is according to the formula

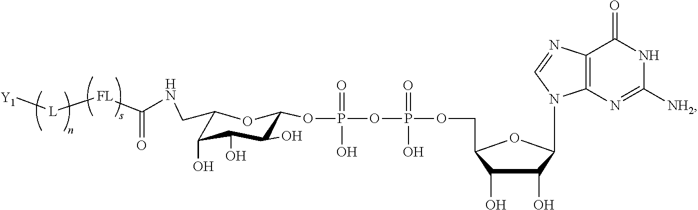

wherein said the FL is the spacer, L is the linker, Y₁ is the functional group, s is 0 or 1 and n is 0 or 1. In some embodiment, s is 1 and n is 0.

In some embodiments, Q-Fuc*′ is selected from the group consisting of

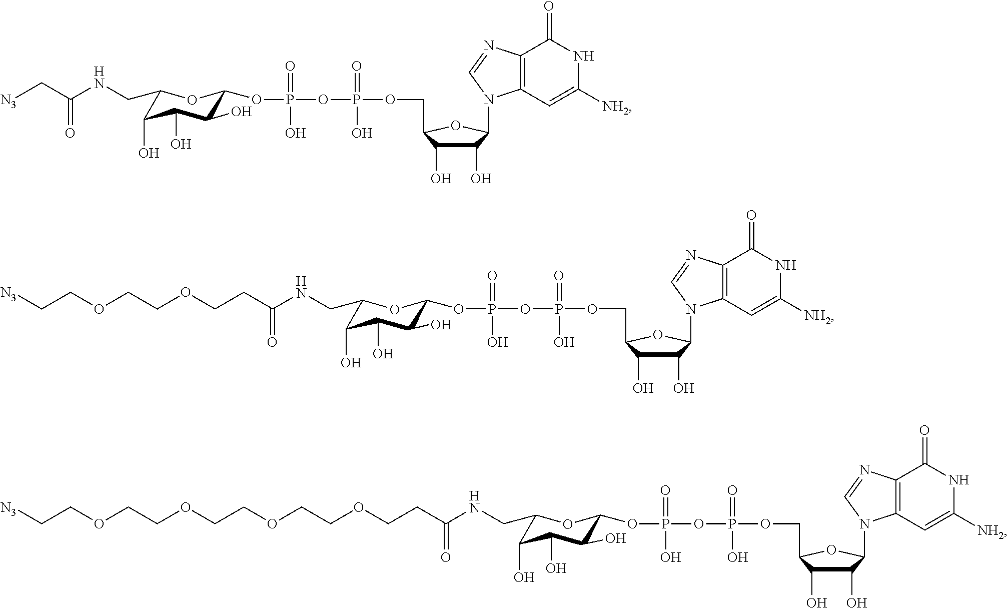

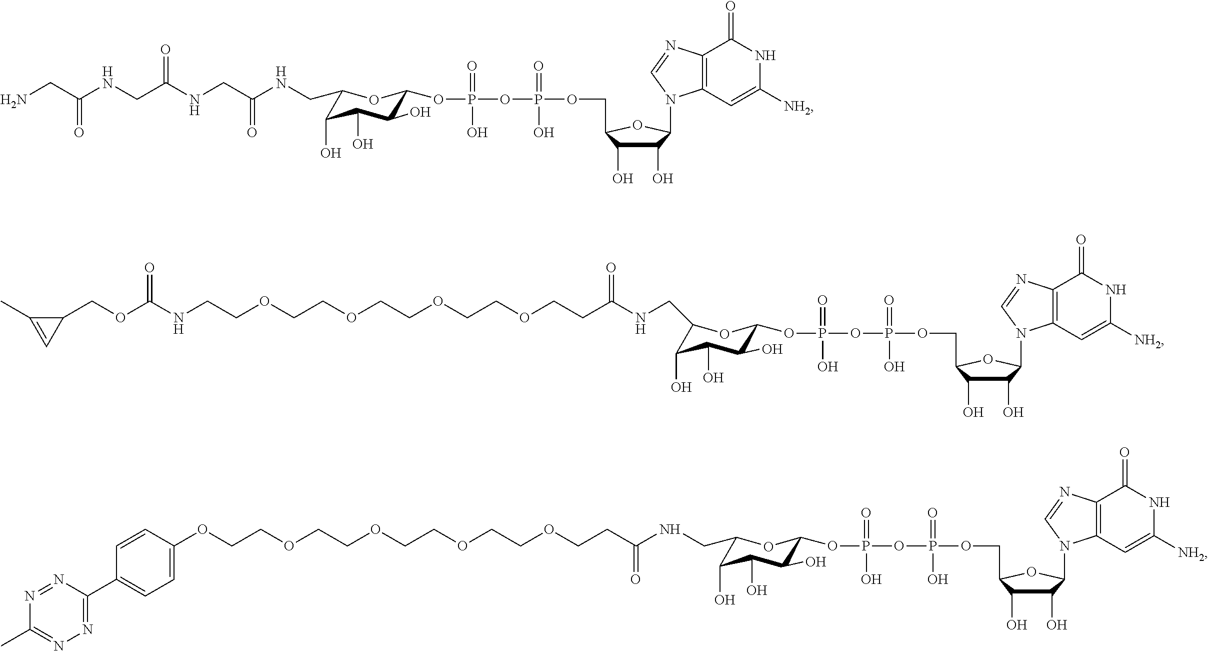

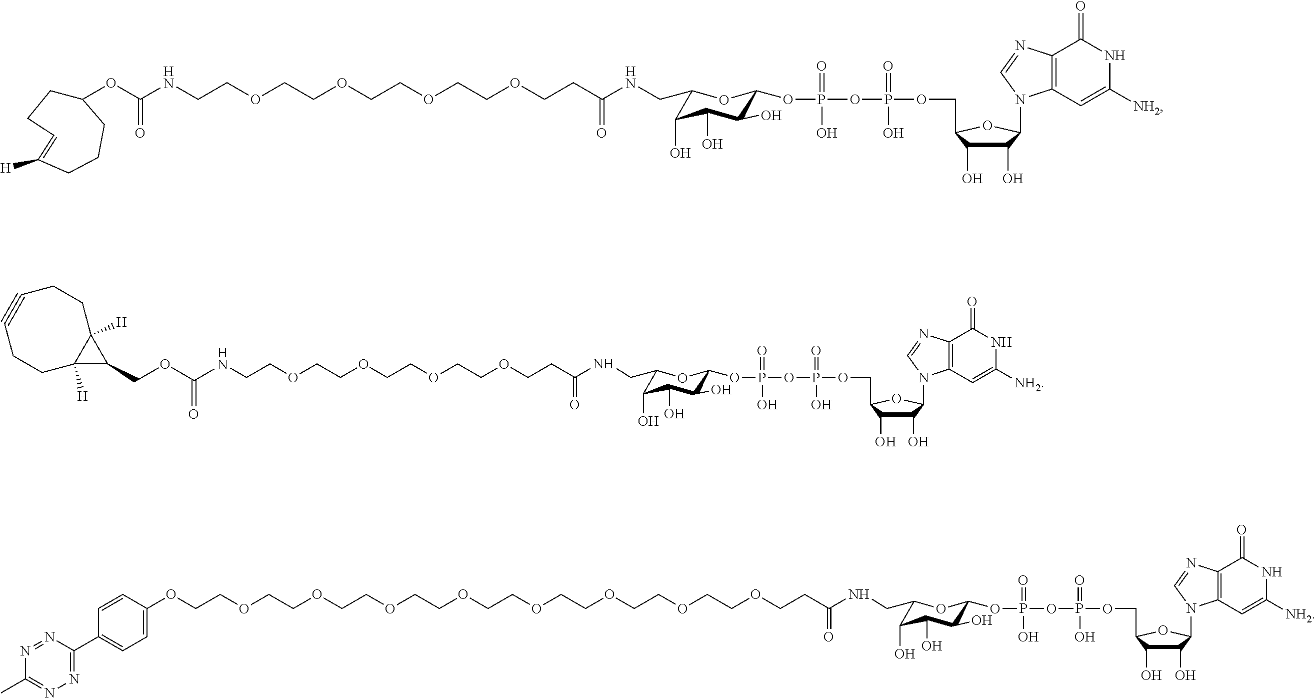

In some embodiments, the Q-Fuc*′ has a structure of

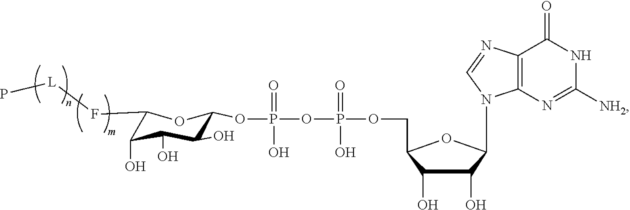

wherein said the F is the connector, L is the linker, P is the biologically and/or pharmaceutically active substance, m is 0 or 1 and n is 0 or 1. In some embodiments, m is 1 and n is 1. In some embodiments, m is 1 and n is 0.

In some embodiments, the Q-Fuc*′ is selected from the group consisting of

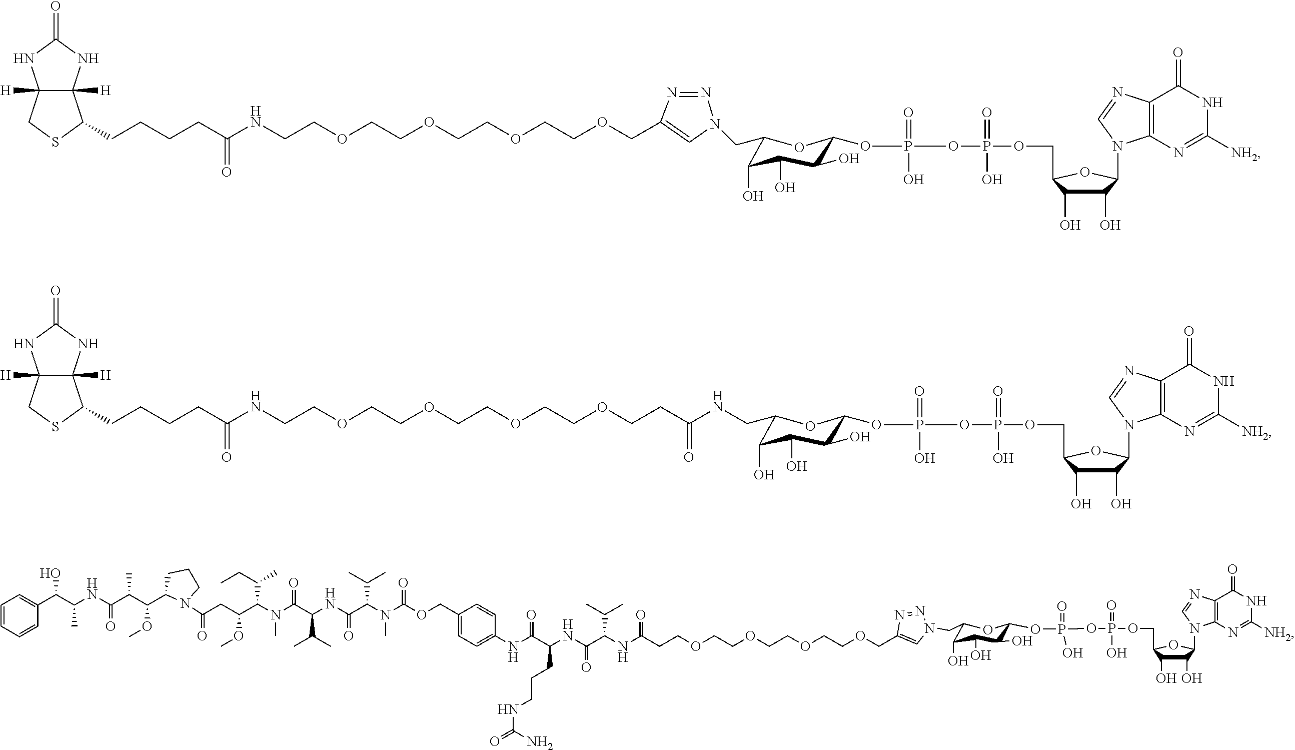

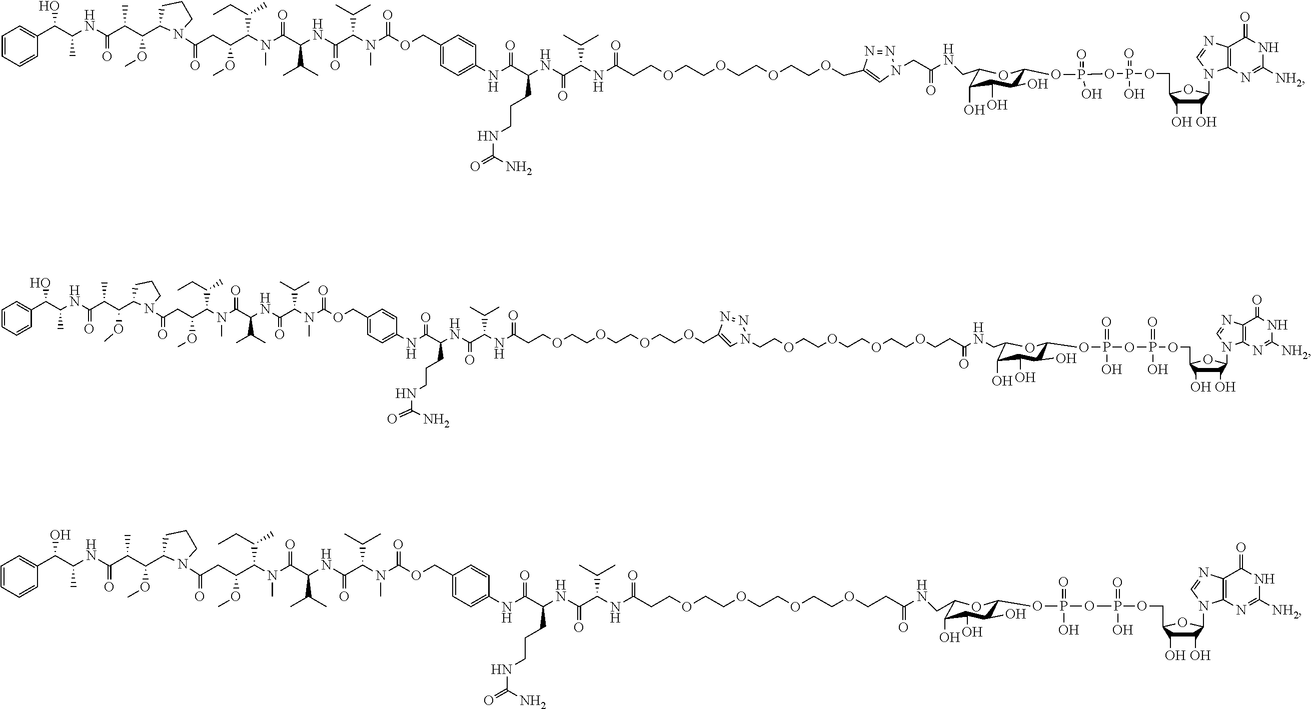

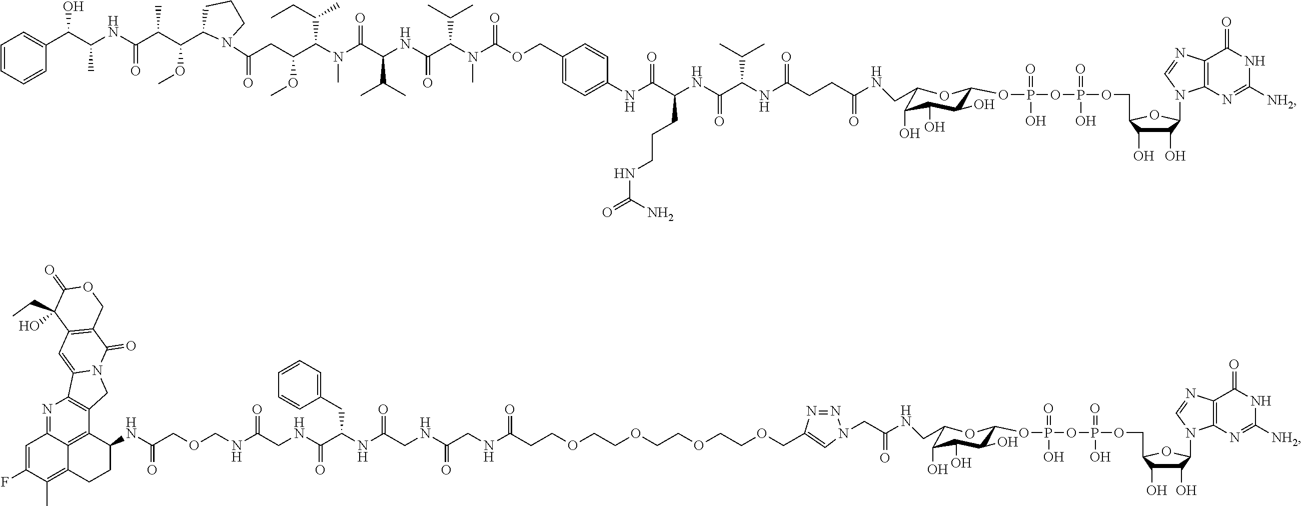

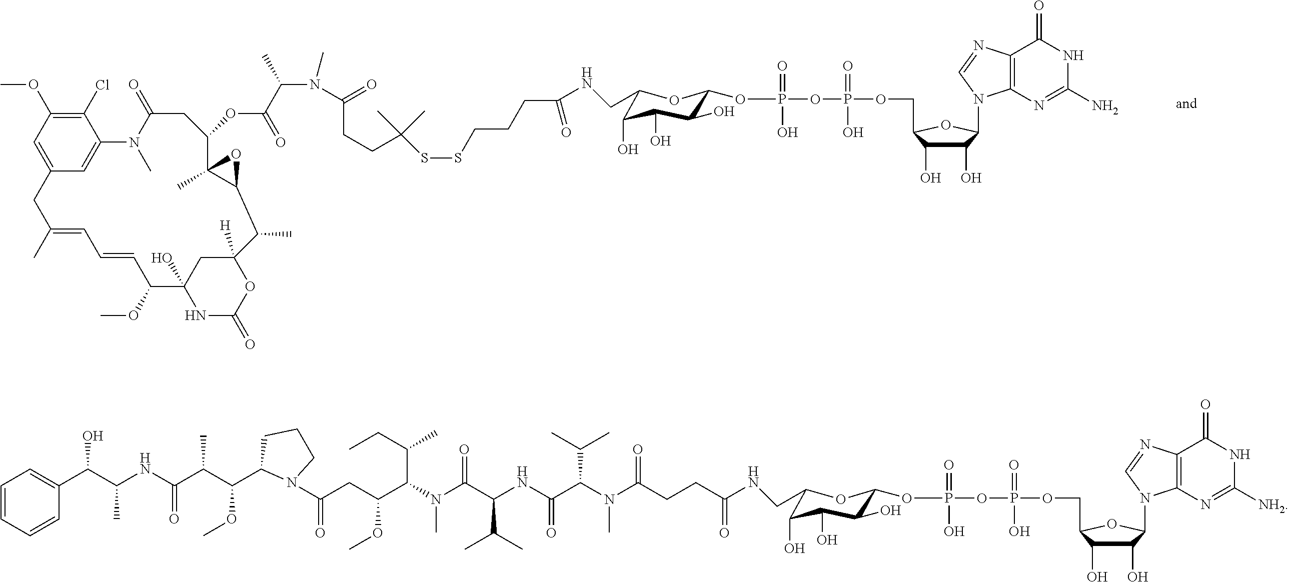

In some embodiments, the Q-Fuc*′ has a structure of

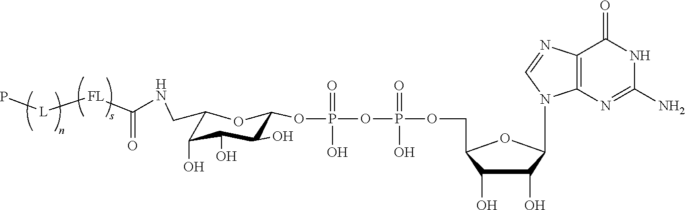

wherein said the FL is the spacer, L is the linker, P is the biologically and/or pharmaceutically active substance, s is 0 or 1 and n is 0 or 1. In some embodiments, s is 1 and n is 1. In some embodiments, s is 1 and n is 0. In some embodiments, P is a pharmaceutically active substance. In some embodiments, P is a cytotoxin.

In some embodiments, the Q-Fuc*′ is selected from the group consisting of

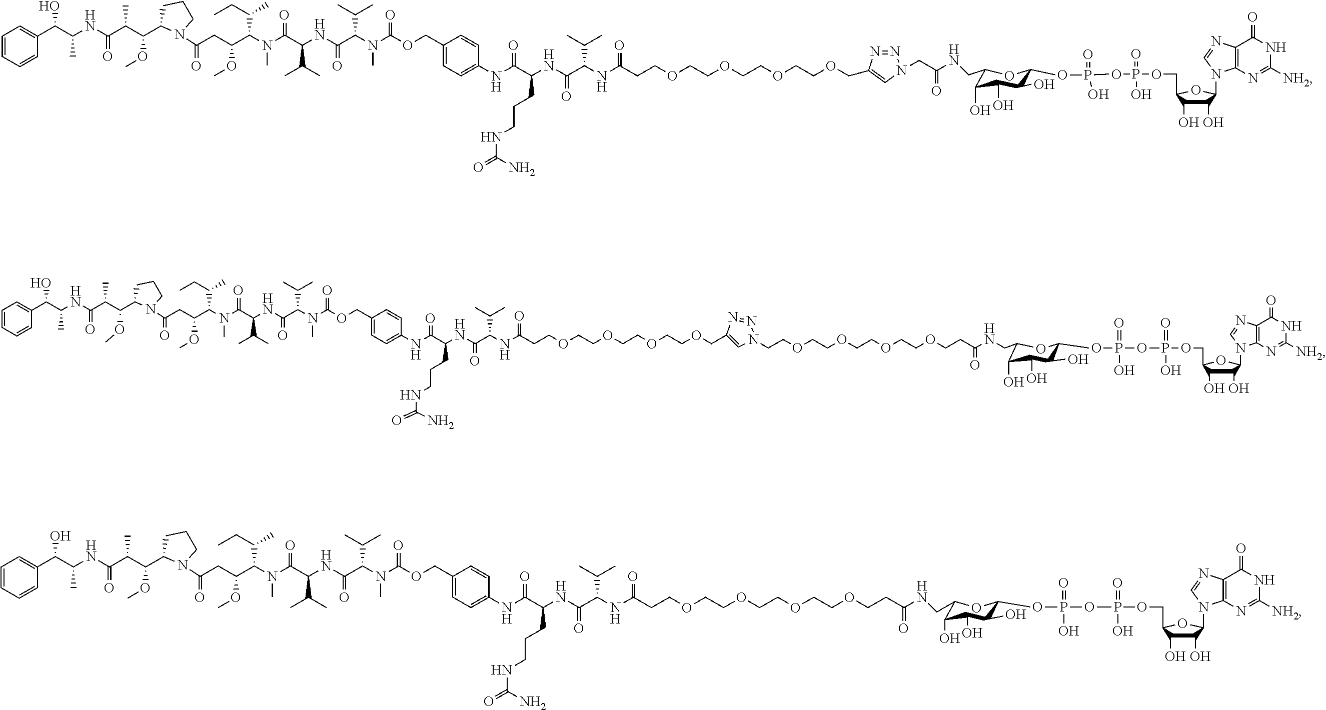

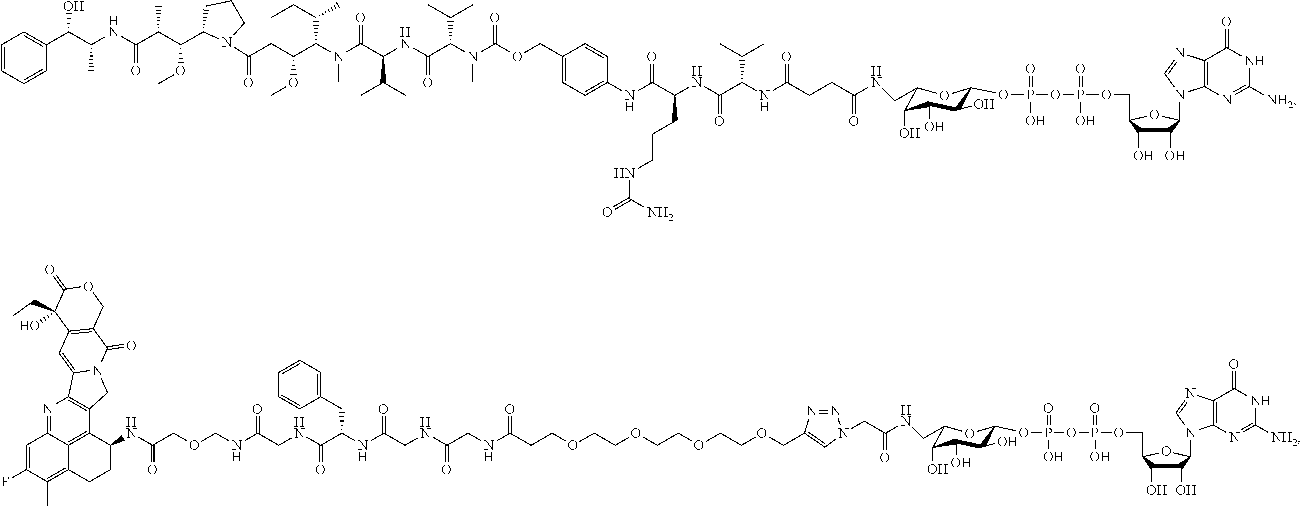

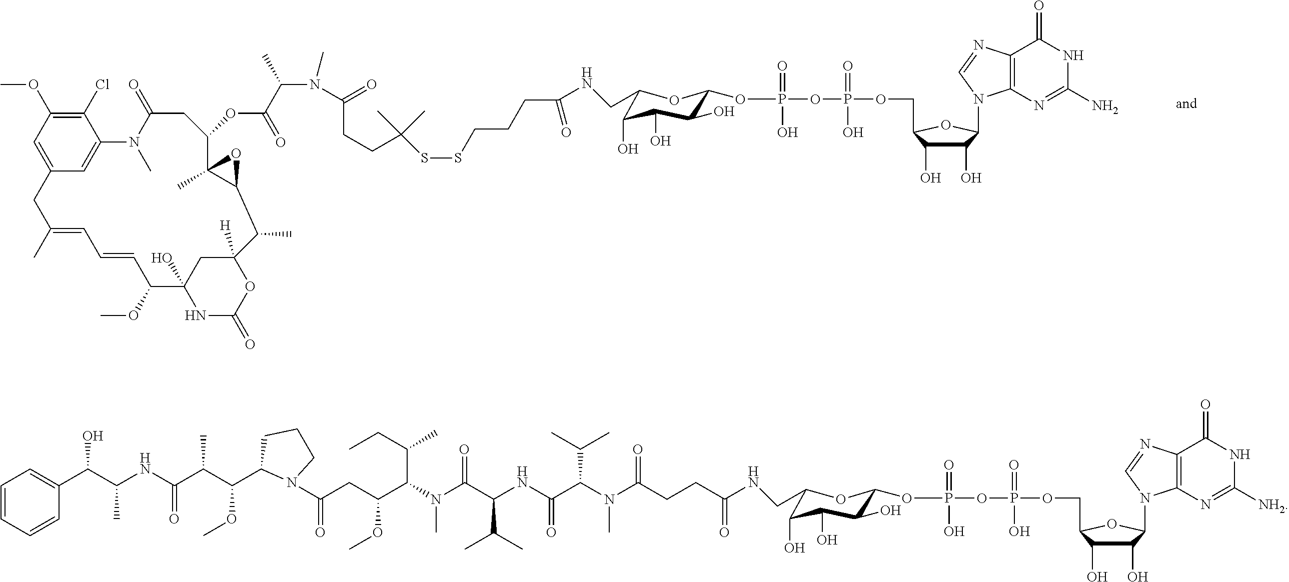

In some embodiments, the Fuc is according to the formula

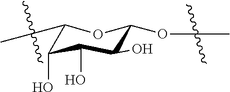

and Fuc*′ is according to the formula

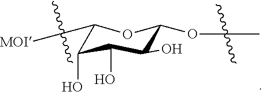

In some embodiments, in the protein conjugate, said fucose or fucose derivative of said Fuc*′ is linked to said GlcNAc through an Fuc*′α1,3 linkage.

In some embodiments, the protein comprises 1 to 20-GlcNAc(Fuc)_{b}-Gal (s).

In some embodiments, the protein comprises 2 or 4-GlcNAc(Fuc)_{b}-Gal (s).

In some embodiments, the protein comprising the oligosaccharide is according to the formula

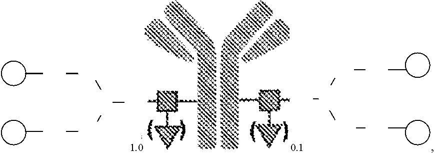

wherein [symbol] is the GlcNAc, [symbol] is the fucose of (Fuc) linked the GlcNAc through an α-1,6 linkage, [symbol] is the mannose, ◯ is the galactose linked to the GlcNAc through a Galβ1,4GlcNAc linkage, and [symbol] is an antibody or a Fc fusion protein. For example, the oligosaccharide is linked to the N297 position of the Fc fragment. For example, the protein is an antibody. For example, the antibody conjugate comprises

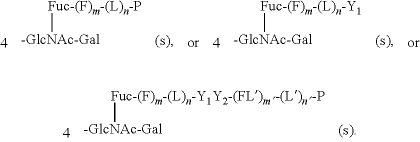

wherein, Fuc is the fucose or fucose derivative, Y₁Y₂ is the remaining group, Y₁ is the functional group, L is the linker, F is the connecter, L′ is the linked defined as the same as the L, FL′ is the spacer defined as the same as the FL, P is the biologically and/or pharmaceutically active substance, m is 0 or 1, n is 0 or 1, m′ is 0 or 1 and n′ is 0 or 1. For example, the protein conjugate is for treating disease. For example, when the protein conjugate comprises the pharmaceutically active substance P, the antibody conjugate is for treating disease. For example, when the protein conjugate comprises the functional group Y₁, the protein conjugate is for making an agent for treating disease. For example, when the protein is an antibody, the protein conjugate has a similar binding affinity as the corresponding antibody towards an antigen.

In some embodiments, the protein comprising the oligosaccharide is according to the formula

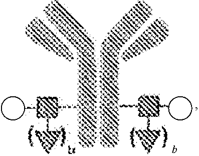

wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, [symbol] is an antibody or a Fc fusion protein and b is 0 or 1. In some embodiments, the GlcNAc is linked to the N297 position of the Fc fragment. In some embodiments, the antibody conjugate comprises

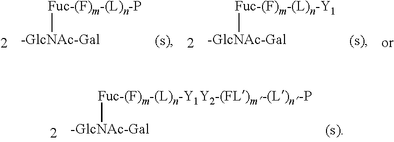

wherein, Fuc is the fucose or fucose derivative, Y₁Y₂ is the remaining group, Y₁ is the functional group, L is the linker, F is the connecter, L′ is the linker defined as the same as the L, FL′ is the spacer defined as the same as the FL, P is the biologically and/or pharmaceutically active substance, m is 0 or 1, n is 0 or 1, m′ is 0 or 1 and n′ is 0 or 1. In some embodiments, the antibody conjugate comprises

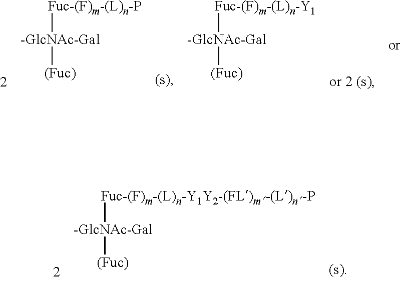

wherein, Fuc is the fucose or fucose derivative, Y₁Y₂ is the remaining group, Y₁ is the functional group, L is the linker, F is the connecter, L′ is the linker defined as the same as the L, FL′ is the spacer defined as the same as the FL, P is the biologically and/or pharmaceutically active substance, (Fuc) is the fusoce linked to the GlcNAc through an α1,6 linkage, m is 0 or 1, n is 0 or 1, m′ is 0 or 1 and n′ is 0 or 1. For example, the protein conjugate is for treating disease. For example, when the protein conjugate comprises the pharmaceutically active substance P, the antibody conjugate is for treating disease. For example, when the protein conjugate comprises the functional group Y₁, the protein conjugate is for making an agent for treating disease. For example, when the protein is an antibody, the protein conjugate has a similar binding affinity as the corresponding antibody towards an antigen.

In some embodiments, the method comprises a step (c): contacting a protein comprising an oligosaccharide comprising an oligosaccharide comprising the -GlcNAc(Fuc)_{b} with a UDP-galactose in the presence of a catalyst, to obtain said protein comprising -GlcNAc(Fuc)_{b}-Gal, wherein Gal is a galactose, b is 0 or 1. For example b is 0. For example, b is 1.

In some embodiments, the catalyst is a galactosyltransferase or a functional variant or fragment thereof.

In some embodiments, the catalyst is a β1,4-galactosyltransferase or a functional variant or fragment thereof.

In some embodiments, the catalyst is a bovine β1,4-galactosyltransferase, a human β1,4-galactosyltransferase, or a functional variant or fragment thereof.

In some embodiments, the catalyst is a human β(1,4)-GalT1 with a mutation of Y285L or a bovine β(1,4)-GalT1 with a mutation of Y289L.

In some embodiments, the catalyst comprises an amino acid as set forth in SEQ ID NO: 1, SEQ ID NO 2 or SEQ ID NO 16.

In some embodiments, the step (c) is before step (a).

In some embodiments, the method does not comprise a purification process between step (c) and step (a).

In some embodiments, step (a) and step (c) is performed in the same reaction vessel.

In some embodiments, the step (a) and step (c) are performed simultaneously.

In some embodiments, the method further comprises a step (d) modifying a protein comprising an oligosaccharide to a protein comprises a core -((Fuc)α1,6)_{b} GlcNAc, wherein b is 0 or 1. For example, b is 0. For example, b is 1.

In some embodiments, the step (d) is performed in the presence of an endoglycosidase or a functional variant or fragment thereof.

In some embodiments, the step (d) is performed in the presence of EndoS or a functional variant or fragment thereof.

In some embodiments, the EndoS comprises an amino acid as set forth in SEQ ID NO 6.

In some embodiments, the step (d) is before the step (c).

In some embodiments, the method comprises a step (e): modifying a protein comprising the core -((Fuc)α1,6) GlcNAc to a protein comprises a core -GlcNAc.

In some embodiments, the step (e) is performed in the presence of a core-α1,6 fucosidase or a functional variant or fragment thereof.

In some embodiments, the core-α1,6 fucosidase is Alfc or a functional variant or fragment thereof.

In some embodiments, the Alfc comprises an amino acid as set forth in SEQ ID NO 7 or SEQ ID NO 18.

In some embodiments, the step (e) is performed behind step (d) and before the step (c).

In some embodiments, the step (d) and step (e) are performed simultaneously.

In some embodiments, the step (d) and step (e) are performed in the same reaction vessel.

In some embodiments, the method does not comprise a purification process among step (a), step (c), step (d) and step (e).

In some embodiments, the step (a), step (c), step (d) and step (e) are performed in the same reaction vessel.

In some embodiments, the protein conjugate is a protein conjugate for treating disease.

In some embodiments, when the MOI′ comprises the Y₁, the protein conjugate is for making an agent for treating disease.

In another aspect, the present disclosure provides a method for preparation of a composition comprising the protein conjugate.

In some embodiments, the present disclosure provide a method for preparation of a composition comprising the protein conjugate comprising

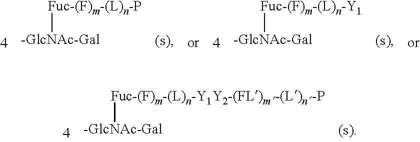

wherein, Fuc is the fucose or fucose derivative, Y₁Y₂ is the remaining group, Y₁ is the functional group, L is the linker, F is the connecter, L′ is the linker defined as the same as the L, FL′ is the spacer defined as the same as the FL, P is the biologically and/or pharmaceutically active substance, m is 0 or 1, n is 0 or 1, m′ is 0 or 1 and n′ is 0 or 1. For example, the protein is an antibody. For example, the the GlcNAc is linked to the mannose of

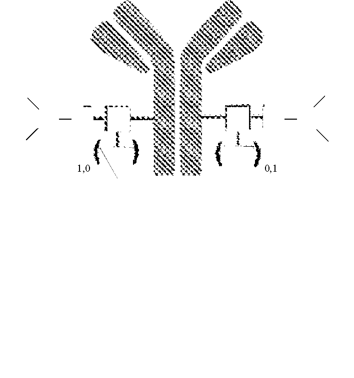

For example, the protein conjugate has the similar binding affinity as the corresponding antibody towards an antigen. For example, the protein conjugate is for treating disease. For example, the protein conjugate is for making an agent for treating disease. For example, the composition has a average MAR of about 2.4-4. For example, the composition has a average MAR of about 2.8-4. For example, the composition has a average MAR of about 3.2-4. For example, the composition has a average MAR of about 3.6-4. For example, the composition has a average MAR of about 3.8-4. For example, the composition has a average MAR of about 4.

In some embodiments, the present disclosure provide a method for preparation of a composition comprising the protein conjugate.comprises

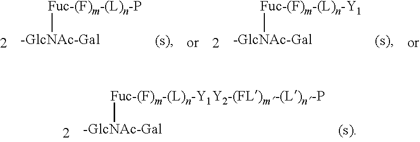

In some embodiments, the present disclosure provide a method for preparation of a composition comprising the protein conjugate comprises

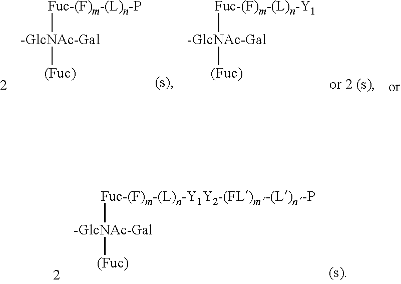

wherein, (Fuc) is the fusoce linked to the GlcNAc through an α1,6 linkage, Fuc is the fucose or fucose derivative, Y₁Y₂ is the remaining group, Y₁ is the functional group, L is the linker, F is the connecter, L′ is the linker defined as the same as the L, FL′ is the spacer defined as the same as the FL, P is the biologically and/or pharmaceutically active substance, m is 0 or 1, n is 0 or 1, m′ is 0 or 1 and n′ is 0 or 1. For example, the protein is an antibody. For example, the GlcNAc is linked directly to an Asn of the antibody. For example, the protein conjugate has the similar binding affinity as the corresponding antibody towards an antigen. For example, the protein conjugate is for making an agent for treating disease. For example, the composition has a average MAR of about 0.5-2. For example, the composition has a average MAR of about 1-2. For example, the composition has a average MAR of about 1.5-2. For example, the composition has a average MAR of about 1.8-2. For example, the composition has a average MAR of about 2.

In another aspect, the present disclosure provides a protein conjugate, which is obtained from the method of the present disclosure.

In another aspect, the present disclosure provides a composition, which is obtained from the method of the present disclosure.

In another aspect, the present disclosure provides use of the Q-Fuc*′ of the present disclosure in preparation of said protein conjugate.

In another aspect, the present disclosure provides a pharmaceutical composition, comprising the protein conjugate of the present disclosure and optionally a pharmaceutically acceptable carrier.

In another aspect, the present disclosure provides a pharmaceutical composition, comprising the composition of the present disclosure and optionally a pharmaceutically acceptable carrier.

In another aspect, the present disclosure provides a method for alleviating, preventing or treating disease, comprising administrating the protein conjugate of the present disclosure and/or the pharmaceutical composition of the present disclosure.

In another aspect, the present disclosure provides a method for alleviating, preventing or treating disease, comprising administrating the composition of the present disclosure and/or the pharmaceutical composition of the present disclosure. In some embodiments, the disease is a tumor. In some embodiments, the tumor is a solid tumor. In some embodiments, the tumor comprises breast carcinoma and/or gastric carcinoma.

In another aspect, the present disclosure provides the use of the protein conjugate and/or the pharmaceutical composition, in preparation of a medicament for alleviating, preventing or treating disease. In some embodiments, the disease is a tumor. In some embodiments, the tumor is a solid tumor. In some embodiments, the tumor comprises breast carcinoma and/or gastric carcinoma.

In another aspect, the present disclosure provides the use of the composition and/or the pharmaceutical composition, in preparation of a medicament for alleviating, preventing or treating disease. In some embodiments, the disease is a tumor. In some embodiments, the tumor is a solid tumor. In some embodiments, the tumor comprises breast carcinoma and/or gastric carcinoma.

In another aspect, the present disclosure provides the protein conjugate and/or the pharmaceutical composition, for use in preventing or treating disease. In some embodiments, the disease is a tumor. In some embodiments, the tumor is a solid tumor. In some embodiments, the tumor comprises breast carcinoma and/or gastric carcinoma.

In another aspect, the present disclosure provides the composition and/or the pharmaceutical composition, for use in alleviating, preventing or treating disease. In some embodiments, the disease is a tumor. In some embodiments, the tumor is a solid tumor. In some embodiments, the tumor comprises breast carcinoma and/or gastric carcinoma.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure”, “FIG.” and “FIG.” herein), of which:

FIG. 1 illustrates a preferred embodiment process for the preparation of antibody-Fuc* conjugates using GDP-Fuc-MOI (GDP-Fuc derivatives bearing a molecule of interest) and an α1,3-FucT. MOI: molecule of interest.

FIGS. 2A and 2B illustrates the structure of exemplary GDP-Fuc-MOI (GDP-Fuc derivatives bearing a molecule of interest).

FIG. 3 illustrates the reaction scheme for the synthesis of the GDP-Fuc-MOI (GDP-Fuc derivatives bearing a molecule of interest).

FIG. 4 illustrates the structure of the compounds used for the synthesis of the GDP-Fuc-MOI (GDP-Fuc derivatives bearing a molecule of interest).

FIG. 5 illustrates the structure of the compounds used for the generation of antibody conjugates from the “two-step” process.

FIG. 6 illustrates the MS analysis of the transform of commercialized trastuzumab to tratuzumab-FAzs and the transform of tratuzumab-G₂F to tratuzumab-G₂F-FAz respectively.

FIG. 7 illustrates a preferred embodiment process for the preparation of antibody-G₂F-Fuc* conjugates using GDP-Fuc-MOI (GDP-Fuc derivatives bearing a molecule of interest) and an α1,3-FucT.

FIG. 8 illustrates the MS analysis of some exemplary antibody-G₂(F)-Fuc* conjugates of the present disclosure generated from the “one-step” process.

FIG. 9 illustrates a preferred embodiment of a “two-step” processes for the preparation of an antibody conjugates and the exemplary combination of Y₁ and Y₂ groups. A reaction handle Y₁ was first installed to the antibody through α1,3-FucT catalyzed reaction to generate an antibody-Y₁ conjugate. Then the antibody-Y₁ conjugate was subjected to react with the complementary reaction handle Y₂ linked to a X moiety comprising an active substance to generate the antibody conjugates.

FIG. 10 illustrates the MS-analysis of trastuzumab-G₂F-GGG conjugates generated from the “two-step” process.

FIG. 11 illustrates the MS analysis of the trastuzumab-G₂F-FAzP₄MMAE and the trastuzumab-G₂F-FAzDBCO-MMAE generated from the “one-step” and the “two-step” process respectively.

FIG. 12 illustrates the in vitro cytotoxicity of trastuzumab, trastuzumab-G₂F-FAzP₄MMAE and trastuzumab-G₂F-FAzDBCO-MMAE on SK-Br-3 (Her2+) cell line and MDA-MB-231(Her2−) cell line respectively.

FIG. 13 illustrates a preferred embodiment process for the preparation of antibody-((Fuc)α1,6)_{0,1}(Galβ1,4)GlcNAc-Fuc* conjugates using GDP-Fuc-MOI (GDP-Fuc derivatives bearing a molecule of interest) and an α1,3-FucT.

FIG. 14 illustrates the MS analysis of some exemplary antibody-((Fuc)α1,6)(Galβ1,4)GlcNAc-Fuc* conjugates of the present disclosure.

FIG. 15 illustrates the MS analysis of some exemplary antibody-(Galβ1,4)GlcNAc-Fuc* conjugates of the present disclosure.

FIG. 16 illustrates the comparison of the catalytic efficiency of Hp-α(1,3)-FucT towards GDP-FAzX derivatives and GDP-FAmX derivatives on the antibody-G₂F and the antibody-(Galβ1,4)GlcNAc respectively. Hp-α(1,3)-FucT display significant higher catalytic efficiency towards the GDP-FAmX derivatives than the GDP-FAzX. A) trastuzumab-G₂F or trastuzumab-(Galβ1,4)GlcNAc were treated Hp-α(1,3)-FucT in the presence of GDP-FAzP₄Biotin or GDP-FAmP₄Biotin for indicated time (for 6 h of trastuzumab-G₂F and 2 h of trastuzumab-(Galβ1,4)GlcNAc) and measured by LC-MS. B) trastuzumab-(Galβ1,4)GlcNAc were treated with Hp-α(1,3)-FucT in the presence of GDP-FAzP₄MMAE or GDP-FAmP₄MMAE for indicated time for 2 hours and measured by LC-MS. For antibody-G₂F, % of conversion=average MAR/4*100%, for antibody-(Galβ1,4)GlcNAc, % of conversion=average MAR/2*100%).

FIG. 17 illustrates MS analysis of some exemplary antibody-drug conjugates (DAR 2 or DAR 4) generated from the “one-step” or “two-step” process.

FIG. 18 illustrates the catalytic efficiency of Hp-α(1,3)-FucT and Human FT6 in transferring the antibody to the GDP-FAmP₄Biotin.

FIG. 19 illustrates the reactivities of different trastuzumab-G₂F-Az conjugates and trastuzumab-(Galβ1,4)GlcNAc-Az conjugates towards DBCO-PEG₄-vc-PAB-MMAE respectively, and the reactivities of trastuzumab-G₂F-FAmP₄Tz and trastuzumab-(Galβ1,4)GlcNAc-FAmP₄Tz towards TCO-PEG₄-vc-PAB-MMAE respectively.

FIG. 20 illustrates binding affinity analysis of some exemplary DAR 2 or DAR 4 trastuzumab-drug conjugates generated from A) the “two-step” process or B) the “one-step” process compared with trastuzumab.

FIG. 21 illustrates HIC-HPLC analysis of some exemplary antibody-drug conjugates generated from the “two-step” process and the “one-step” process.

FIG. 22 illustrates plasma stability analysis of some exemplary antibody-drug conjugates (DAR 2 or DAR 4) generated from the “one-step” and the “two-step” process.

FIG. 23 illustrates the in vitro cytotoxicity of some exemplary trastuzumab-MMAE or trastuzumab-MMAF conjugates on SK-Br-3 (Her2+) cell line, BT474 (Her2+) cell line and MDA-MB-231 (Her2−) cell lines respectively.

FIG. 24 illustrates the in vitro cytotoxicity of the trastuzumab-seco-DUBA conjugate (trastuzumab-(Galβ1,4)GlcNAc-FAmP₄AzDBCO-seco-DUBA) on NCI-N87 (Her2+) cell line, SKBr3 (Her2+) cell line, BT474 (Her2+) cell line and MDA-MB-231 (Her2−) cell line respectively.

FIG. 25 illustrates the in vitro cytotoxicity of some exemplary trastuzumab-drug conjugates on NCI-N87 (Her2+) cell line and MDA-MB-231(Her2−) cell line respectively.

FIG. 26 illustrates the in vitro cytotoxicity of an exemplary anti-Trop2-MMAE conjugate (hRS7-(Galβ1,4)GlcNAc-FAmSucMMAE) on JIMT-1 (trop2 high expression) cell line and MDA-MB-231 (trop2 low expression) cell line respectively.

FIG. 27 illustrates the in vivo efficacy of trastuzumab-drug conjugates on a nude mouse (BALB/c) human gastric NCI-N87(Her2+) xenograft model. A) antitumor activity of trastuzumab-G₂F-FAmSucMMAE compared with trastuzumab and Kadcyla®. B) The bodyweight of the mice after the single injection. The black arrow at day 0 represents the single i.v. injection. (n=6 mice per group).

FIG. 28 illustrates the in vivo efficacy of anti-Trop2-drug conjugates on a nude mouse (BALB/c) human breast cancer JIMT-1 (Trop2 high expression) xenograft model A) antitumor activity of hRS7-(Galβ1,4)GlcNAc-FAmSucMMAE compared with hRS7. B) The bodyweight of the mice after the single injection. The black arrow at day 0 represents the single i.v. injection. (n=6 mice per group).

FIG. 29 illustrates the G₀, GoF, G₁, G₁F, G₂ and G₂F glycoforms of antibodies. The G₀(F) form lacks both galactose (Gal) residues at the ends of the biantennary chains. G₁(F) are biantennary positional isomers carrying one Gal residue attached to the mannose GlcNAc branch. In G₂(F), both branches carry a Gal residue. In GoF, G₁F and G₂F, the core-fucose were attached to the core-GlcNAc in an α-1,6 linkage. Both G₁F and G₂F structures contain the disaccharide N-acetyllactosamine (LacNAc, Galβ1,4GlcNAc) unit.

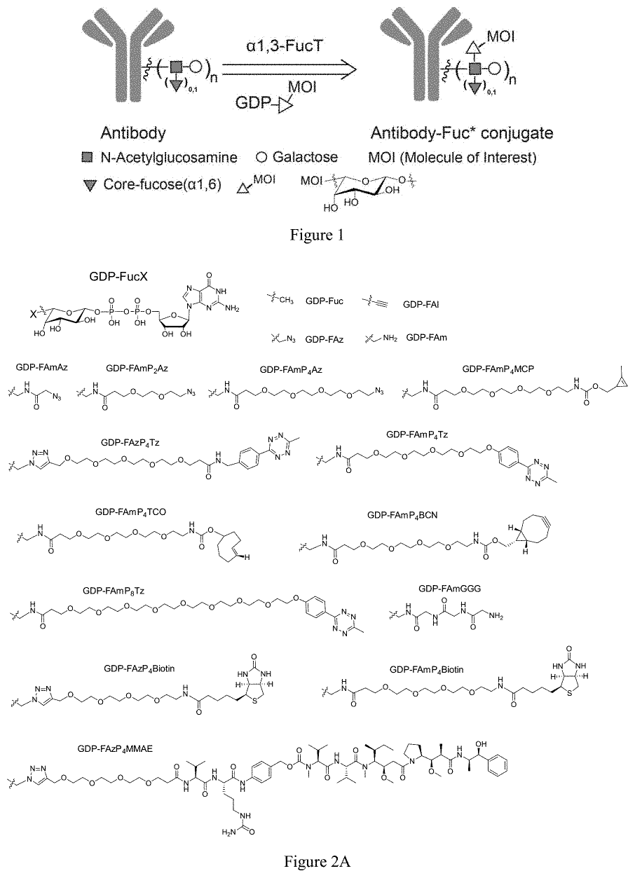

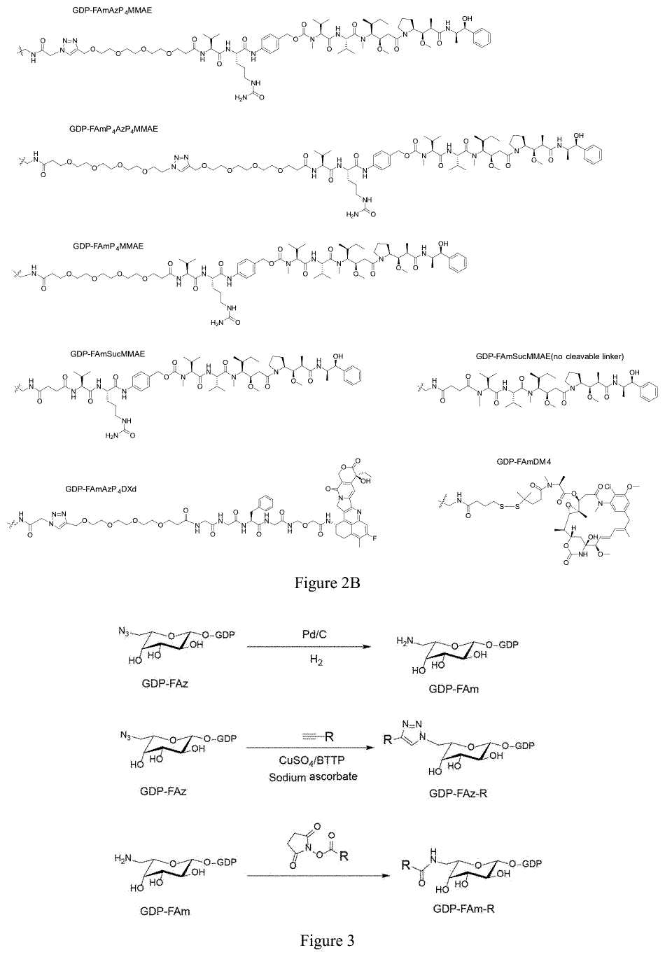

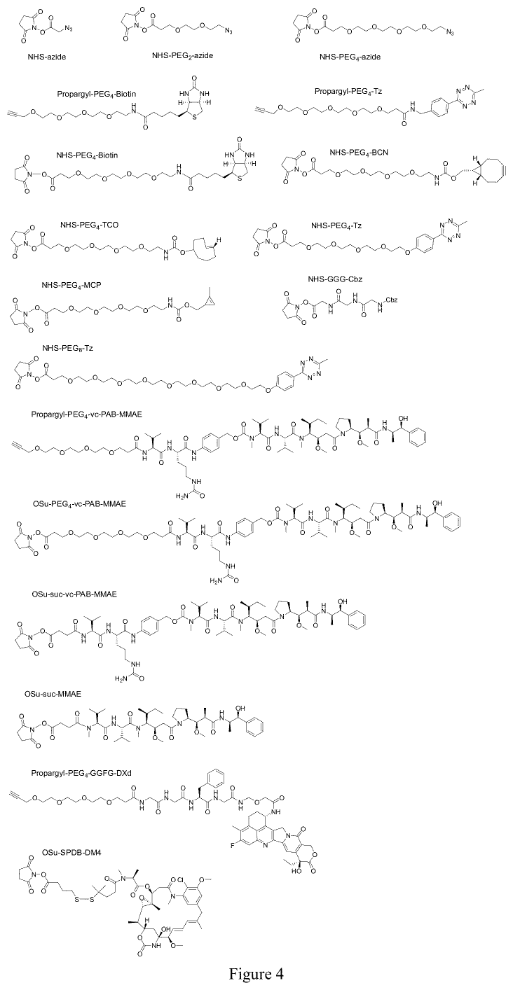

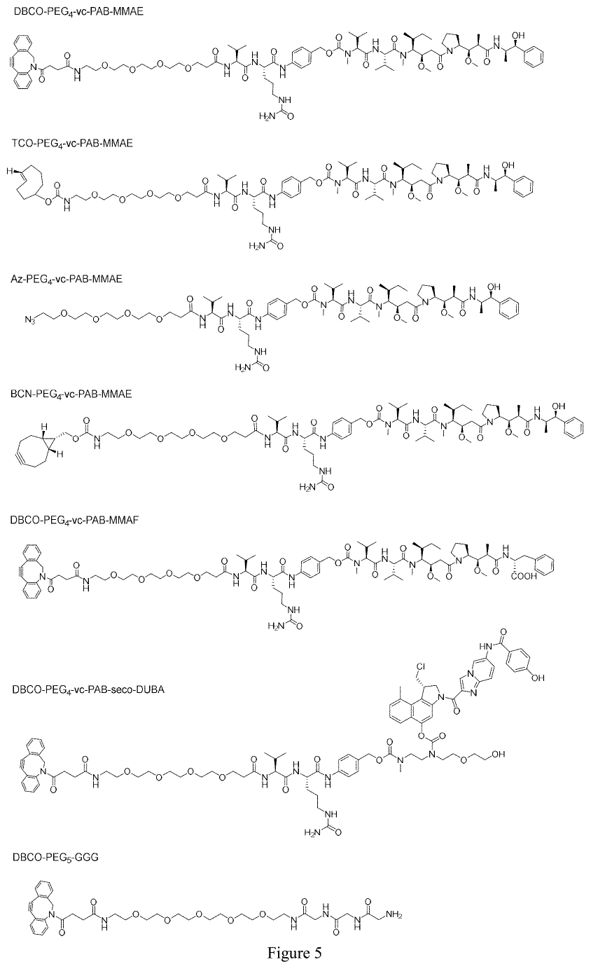

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “conjugate”, as used herein, generally refers to any substance formed from the joining together of separate parts. In the conjugate, the separate parts may be joined at one or more active site with each other. Moreover, the separate parts may be covalently or non-covalently associated with, or linked to, each other and exhibit various stoichiometric molar ratios. The conjugate may comprise peptides, polypeptides, proteins, prodrugs which are metabolized to an active agent in vivo, polymers, nucleic acid molecules, small molecules, binding agents, mimetic agents, synthetic drugs, inorganic molecules, organic molecules and radioisotopes.

The term “Fc fragment”, as used herein, generally refers to a portion of an antibody constant region. Traditionally, the term Fe domain refers to a protease (e.g., papain) cleavage product encompassing the paired CH₂, CH₃ and hinge regions of an antibody. In the context of this disclosure, the term Fc domain or Fc refers to any polypeptide (or nucleic acid encoding such a polypeptide), regardless of the means of production, that includes all or a portion of the CH₂, CH₃ and hinge regions of an immunoglobulin polypeptide.

The term “antigen binding fragment”, as used herein, generally refers to a peptide fragment capable of binding antigen. The antigen binding fragment may be a fragment of an immunoglobulin molecule. An antigen-binding fragment may comprise one light chain and part of a heavy chain with a single antigen-binding site. An antigen-binding fragment may be obtained by papain digestion of an immunoglobulin molecule. For example, an antigen-binding fragment may be composed of one constant and one variable domain of each of the heavy and the light chain. The variable domain may contain the paratope (the antigen-binding site), comprising a set of the complementarity determining regions, at the amino-terminal end of the immunoglobulin molecule. For example, the antigen binding fragment may be a Fab, a F(ab)₂, F(ab′), a F(ab′)₂, a ScFv, and/or a nanobody.

The term “Fc-fusion protein”, as used herein, generally refers to a protein which are composed of the Fc domain of IgG genetically linked to a peptide or protein of interest.

The term “directly linked” as used herein, generally refers to that a moiety is linked to another moiety without any intermediate moiety or linker. For example, a GlcNAc is directly linked to an amino acid residue of an antibody generally refers to that the GlcNAc is bonded via a covalent bond to an amino acid residue of the antibody, for example, via an N-glycosidic bond to an amide nitrogen atom in a side chain of an amino acid (e.g., an asparagine amino acid) of the antibody. In the present disclosure, when a GlcNAc is “indirectly linked” to an amino acid of the protein, there are usually at least one monosaccharide moiety between the GlcNAc and the amino acid of the protein.

The term “GlcNAc”, or “N-acetylglucosamine”, can be used interchangeably, generally refers to an amide derivative of the monosaccharide glucose that usually polymerizes linearly through β-(1,4) linkages.

Glycosylation generally refers to the reaction in which a carbohydrate, i.e., a glycosyl donor, is attached to a hydroxyl or other functional group of another molecule (a glycosyl acceptor). In some embodiments, glycosylation mainly refers in particular to the enzymatic process that attaches glycans to proteins, or other organic molecules. The glycosylation in protein can be modified in glycosylation linkage, glycosylation structure, glycosylation composition and/or glycosylation length. Glycosylation can comprise N-linked glycosylation, O-linked glycosylation, phosphoserine glycosylation, C-mannosylation, formation of GPI anchors (glypiation), and/or chemical glycosylation. Correspondingly, a glycosylated oligosaccharide of a protein can be a N-linked oligosaccharide, O-linked oligosaccharide, phosphoserine oligosaccharide, C-mannosylated oligosaccharide, glypiated oligosaccharide, and/or chemical oligosaccharide.

The term “antibody”, as used herein, generally refers to a polypeptide or a protein complex that specifically binds an epitope of an antigen or mimotope thereof. An antibody includes an intact antibody, or a binding fragment thereof that competes with the intact antibody for specific binding and includes chimeric, humanized, fully human, and bispecific antibodies. Binding fragments include, but are not limited to, Fab, Fab′, F(ab′)₂, Fv, single-chain antibodies, nanobodies. In some embodiments, an antibody is referred to as an immunoglobulin and include the various classes and isotypes, such as IgA (IgA1 and IgA2), IgD, IgE, IgM, and IgG (IgG1, IgG3 and IgG4) etc. in some embodiments the term “antibody” as used herein refers to polyclonal and monoclonal antibodies and functional fragments thereof. An antibody includes modified or derivatized antibody variants that retain the ability to specifically bind an epitope. Antibodies are capable of selectively binding to a target antigen or epitope. Antibodies may include, but are not limited to polyclonal antibodies, monoclonal antibodies (mAbs), humanized and other chimeric antibodies, nanobodies, single chain antibodies (scFvs), Fab fragments, F(ab′)₂ fragments and disulfide-linked Fvs (sdFv) fragments. In some embodiments, the antibody is from any origin, such as mouse or human, including a chimeric antibody thereof. In some embodiments, the antibody is humanized.

The term “monoclonal antibody”, as used herein, generally refers to an antibody obtained from a population of substantially homogeneous antibodies, i.e., the individual antibodies comprising the population are identical except for possible naturally occurring mutations and/or post-translational modifications (e.g., isomerizations, amidations) that may be present in minor amounts.

The term “IgG”, as used herein, generally refers to various broad classes of polypeptides or proteins that can be distinguished biochemically. Those skilled in the art will appreciate that immunoglobulin heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgG, or IgE, respectively. The immunoglobulin subclasses (subtypes) e.g., IgGi, IgG2, IgG3, IgG4, IgAi, IgA2, etc. are well characterized and are known to confer functional specialization. Human IgG is typically characterized by glycosylation at position Asn297 (numbering according to Kabat) in the heavy chain CH₂ region of the Fc region.

In the present disclosure, “Asn297”, or “N297”, can be used interchangeably, is the Asparagine at site 297 (numbered according to the Kabat numbering system) of an antibody Fc fragment. Asn297 may be attached with one or more oligosaccharide.

The term “humanized antibody”, as used herein, generally refers to containing the antibody from some or all CDR of nonhuman animal antibody, and the framework of antibody and constant region contain the amino acid residue of derived from human antibody sequence.

The term “Fuc*α1,3GlcNAc linkage”, as used herein, generally refers to a linkage between a fucose or fucose derivative Fuc of the Fuc* and a GlcNAc.

The term “Galβ1,4GlcNAc linkage”, as used herein, generally refers to a linkage between a galactose and a GlcNAc.

The term “N-linked oligosaccharide”, as used herein, generally refers to the attachment of an oligosaccharide to a nitrogen atom. In some embodiments, the oligosaccharide may comprise a carbohydrate consisting of several sugar molecules, sometimes also referred to as glycan. In some embodiments, the nitrogen atom is an amide nitrogen of an amino acid residue of a protein, for example, an asparagine (Asn) of a protein.

The term “molecule of interest (MOI)”, as used herein, generally refers to a molecule with a desired characteristic. The desired characteristic may be a physical characteristic or a chemical characteristic, for example, reactive activity, stability, solubility, binding activity, inhibiting activity, toxicity or degradability. A MOI may comprise any substances possessing a desired biological activity and/or a reactive functional group that may be used to incorporate a drug into the protein conjugate of the disclosure. For example, a MOI may comprise an active substance. For example, the active substance may be a therapeutical agent, a diagnosis agent, a pharmacological agent and/or a biological agent, e.g., a cytotoxin, a cytostatic agent, a radioisotope or radionuclide, a metal chelator, an oligonucleotide, an antibiotic, a fluorophore, a biotin tag, a peptide, a protein, or any combination thereof. In some cases, an active substance could be a chemically active substance. For example, a chemically active substance may be a chemically functional moiety that could reacted with another chemically functional moiety to form a covalent bond. For example, a chemically active substance may be able to participate in a ligation reaction. In some cases, an active substance could be an enzymatically active substance that could be reacted with complementary functional moiety to form a covalent bond in the presence of an enzyme. For example, an enzymatically active substance may be an N-terminal peptide tag GGG (NH₂-GGG) which could react with a C terminal peptide tag LPETGG (LPETGG-COOH) (SEQ.ID NO. 23) in the presence of a sortase ligase to form a covalent bond.

The term “functional group”, as used herein, generally refers to a group capable of reacting with another group. A functional group can be used to incorporate an agent (e.g., an agent without a reactive activity or with a low reactive activity) into the protein conjugate of the disclosure. For example, the agent may be a pharmaceutically active substance (e.g. a cytotoxin). A functional group may be a chemical group or a residue having chemical and/or enzymatic reactivity. In some embodiments, a functional group may be a group capable of reacting in a ligation reaction. A functional group usually comprises a functional moiety, and the functional group may react with another group due to the functional moiety.

The term “ligation reaction”, as used herein, generally refers to a chemically and/or enzymatically reaction in which a molecule is capable of linked to another molecule. This binding may be driven by the functional groups of the reactive molecules.

The term “fucosyltransferase”, as used herein, generally refers to an enzyme or a functional fragment or a variant thereof that can transfer a L-fucose sugar from a fucose donor substrate (such as, guanosine diphosphate-fucose) to an acceptor substrate. The acceptor substrate can be another sugar such as a sugar comprising a GlcNAc-Gal (LacNAc), as in the case of N-glycosylation, or in the case of O-linked glycosylation. The term “fucosyltransferase” may comprise any functional fragments, or a catalytic domain thereof, and functional variants (such, mutant, isoform) with a catalytic activity domain. The example of fucosyltransferase may be an α-1,3 fucosyltransferase. The term “fucosyltransferase” may derived from various species, such as mammals (e.g., humans), bacteria, nematodes or trematodes. In some embodiments, the fucosyltransferase is derived from Bacteroides fragilis. In some embodiments, the fucosyltransferase comprises an amino acid sequence as set forth in GenBank Accession No. YP_213065.1, or a functional variant or fragment thereof. In some embodiments, the fucosyltransferase is derived from Helicobacter pylori. In some embodiments, wherein said fucosyltransferase comprises an amino acid sequence as set forth in GenBank Accession No. AF008596.1, GenBank Accession No. AAD07447.1, GenBank Accession No. AAD07710.1, GenBank Accession No. AAF35291.2, or GenBank Accession No. AAB93985.1, or their functional variant or fragment thereof. In some embodiments, the fucosyltransferase comprises an amino acid sequence as set forth in GenBank Accession No. AAD07710.1, or a functional variant or fragment thereof. For example, the fucosyltransferase may comprise an amino acid sequence as set forth in GenBank Accession No. AAD07710.1, or the fucosyltransferase may comprise an amino acid sequence with an identity of more than 80% (e.g., more than 83%, more than 88%, more than 90%, more than 95%, more than 96%, more than 97%, more than 98%, more than 99%, or more) of an amino acid sequence as set forth in GenBank Accession No. AAD07710.1 or a functional variant or fragment thereof. For another example, the fucosyltransferase may comprise an amino acid sequence as set forth in SEQ ID NO: 3 or 4, or the fucosyltransferase may comprise an amino acid sequence with an identity of more than 80% (e.g., more than 88%, more than 88%, more than 90%, more than 95%, more than 96%, more than 97%, more than 98%, more than 99%, or more) of an amino acid sequence as set forth in SEQ ID NO: 3 or SEQ ID NO: 4.

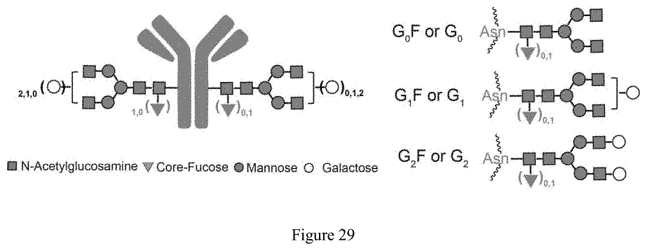

The term G₂, G₁ or G₀, as used herein, generally refers to a glycoform of G₂, G₁ or G₀ as shown in FIG. 29. The term G₂F G₁F G₀F, as used herein, generally refers to a glycoform of G₂F, G₁F, GoF as shown in FIG. 29. The term G₂(F) as used here generally refers to a G₂ glycoform with a optional core α1,6-fucose. The “antibody-G₂F, as used herein, generally refers to an antibody with a G₂F glycoform. The antibody-G₂(F), antibody-G₁(F) and antibody-G₀(F) as used herein, generally refers to an antibody with a G₂(F), G₁(F) and G₀(F) glycoform respectively.

The term “(Fuc)” as used herein, generally refers to a fucose linked with a GlcNAc, wherein the GlcNAc is directly linked to an amino acid of a protein (e.g., an antibody or a fragment thereof). For example, the “(Fuc)” is linked with the GlcNAc through a α1,6 linkage. The fucose of (Fuc) is different with the fucose or fucose derivative of the Fuc* of the present disclosure. In the present disclosure, the “Fuc” represents the fucose or fucose derivative of the Fuc*.

The term “first part”, as used herein, generally refers to a part which comprises a GlcNAc-Gal (i.e. LacNAc) in the conjugate of the present disclosure. In some embodiments, said first part may be an isolated protein with a GlcNAc-Gal (i.e. LacNAc). The term “second part”, as used herein, generally refers to a part which comprise a fucose or fucose derivative Fuc in the conjugate of the present disclosure. The first part may connect with the second part via a covalent bond between the GlcNAc and the fucose or fucose derivative Fuc. The term “first molecule”, as used herein, generally refers to, an isolated molecule with a LacNAc, especially a protein, such as an antibody or a fragment with a Fc domain. The term “second molecule”, as used herein, generally refers to a molecule comprising a fucose or fucose derivative Fuc and an active moiety. The first molecule and the second molecule are able to react with each other to form a conjugate. And in the conjugate, the part derived from the first molecule can be the first part, and the part derived from the second molecule can be the second part.

The term “linking units” W, refer to a moiety existed between the fucose or fucose derivative Fuc and the active moiety. The linking unit W links the Fuc to the active moiety. In some embodiments, the “linking units” W may comprise may comprise a polypeptide, PEG, alkyl and/or derivatives thereof.

The term “pharmaceutically active substance”, as used herein, generally refers to any substance being pharmaceutically useful or having a pharmaceutical effect. In the present disclosure, a pharmaceutically active substance may not comprise a detectable agent (e.g., an agent with a detectable physical or a chemical moiety, and/or only be used for detective purpose in the present disclosure). In the present disclosure, a fluorescent label may be not a pharmaceutically active substance. For example, a pharmaceutically active substance may be an agent capable of alleviating, treating, preventing a disease, or delaying a disease process. The disease may be a disease associated with abnormal cell proliferation and/or cellular dysfunction. The disease may be a tumor and/or an immune disease.

A pharmaceutically active substance may comprise a compound useful in the characterization of tumors or other medical condition, for example, diagnosis, characterization of the progression of a tumor, and assay of the factors secreted by tumor cells. For example, the pharmaceutically active substance may be a radioisotope or radionuclide. For example, the pharmaceutically active substance may be a PET imaging agent.

A pharmaceutically active substance may be a cytotoxin. A cytotoxin may comprise any agents capable of damaging to cell proliferation and/or differentiation. A cytotoxin may have a cytotoxic effect on tumors including the depletion, elimination and/or the killing of tumor cells.

The term a “corresponding antibody”, as used herein, generally refers to the antibody from which a protein conjugate can be obtained after some modifications, e.g., glycosylation modification or conjugation, especially after performing the method of the present disclosure. A protein conjugate may be capable of binding to the same antigen or the same antigen epitome with its corresponding antibody. A corresponding antibody can be conjugated with a molecule of interest to become a protein conjugate. For example, in the protein conjugate of the present, which may comprise an antibody and an oligosaccharide, wherein said oligosaccharide comprises a structure of:

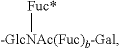

b is 0 or 1, a corresponding antibody is the antibody not modified with the Fuc*. For example, in the present disclosure, in the step (a) of the method, the protein comprising an oligosaccharide may be an antibody, and the antibody can be reacted with a Q-Fuc*′, to obtained a protein conjugate. In that situation, the antibody can be the “corresponding antibody” to the obtained protein conjugate. For example, in the present disclosure, a corresponding antibody may be an antibody comprising -GlcNAc(Fuc)_{b}-Gal, b is 0 or 1. The corresponding antibody may be modified through a step (a) to obtain a protein conjugate. The corresponding antibody may be modified through a step (a) and a step (b) to obtain a protein conjugate. In those conditions, the antibody comprising -GlcNAc(Fuc)_{b} can be the “corresponding antibody” of those protein conjugates. For example, in the present disclosure, a corresponding antibody may be an antibody comprising -GlcNAc(Fuc)_{b}, b is 0 or 1. The corresponding antibody may be modified through a step (c) and a step (a) to obtain a protein conjugate. The corresponding antibody may be modified through a step (c), a step (a) and a step (b) to obtain the protein conjugate. In those conditions, the antibody comprising -GlcNAc(Fuc)_{b} can be the corresponding antibody of those protein conjugates. For example, in the present disclosure, a corresponding antibody may be an antibody comprising heterogenous glycoforms (e.g. a mixture of G₂(F), G₁(F) and G₀(F)). The antibody may be modified through a step (c) and a step (a) to obtain a protein conjugate. The antibody may be modified through a step (c), a step (a) and a step (b) to obtain a protein conjugate. In those conditions, the antibody comprising heterogenous glycoforms (e.g. a mixture of G₂(F), G₁(F) and G₀(F)) can be the corresponding antibody of those protein conjugates. For example, in the present disclosure, a corresponding antibody may be an antibody comprising heterogenous glycoforms (e.g. a mixture of G₂(F), G₁(F) and G₀(F)). The antibody may be modified through a step (d), a step (c) and a step (a) to obtain a protein conjugate. The antibody may be modified through a step (d), a step (c), a step (a) and a step (b) to obtain a protein conjugate. The antibody may be modified through a step (d), a step (e), a step (c) and a step (a) to obtain a protein conjuagte. The antibody may be modified through a step (d), a step (e), a step (c), a step (a) and a step (b) to obtain a protein conjugate. In those conditions, the antibody comprising heterogenous glycoforms can be the corresponding antibody of those protein conjugates. For example, the corresponding antibody of trastuzumab-G₂F-Fuc* conjugates prepared from both the “one-step process” and the “two-step” process could be a trastuzumab with heterogenous glycoforms (e.g. a mixture of G₂(F), G₁(F) and G₀(F)). For example, the corresponding antibody of trastuzumab-(Galβ1,4)GlcNAc-Fuc* prepared from both the “one-step process” and the “two-step” process could be a trastuzumab with heterogenous glycoforms (e.g. a mixture of G₂(F), G₁(F) and G₀(F)). FIG. 20 showed that the trastuzumab-G₂F-Fuc* conjugates and the trastuzumab-(Galβ1,4)GlcNAc-Fuc* conjugates prepared from both the “one-step” process and the “two-step” process had a similar binding affinity as their corresponding antibody (i.e., the trastuzumab with a heterogenous glycoforms) towards a Her2 antigen.

The terms “comprise” and variations thereof do not have a limiting meaning where these terms appear in the description and claims.

Unless otherwise specified, “a,” “an,” “the,” and “at least one” are used interchangeably and mean one or more than one.

Protein Conjugate

In the present disclosure, the present disclosure provides a protein conjugate, and the protein conjugate comprises a protein and an oligosaccharide, wherein the oligosaccharide comprises

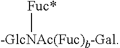

For example, the oligosaccharide may comprise

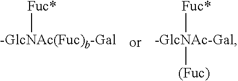

wherein (Fuc) below GlcNAc is a fucose. When the oligosaccharide comprises the

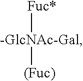

the (Fuc) may be linked to the GlcNAc through an α1,6 linkage.

For example, the Gal may be linked to said GlcNAc through a Galβ1,4GlcNAc linkage.

In the

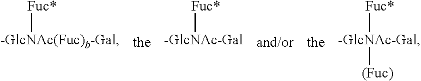

the GlcNAc is directly or indirectly linked to an amino acid of said protein; said Gal is a galactose; said (Fuc) is a fucose, b is 0 or 1; said Fuc* comprises a fucose or a fucose derivative linked to a molecule of interest (MOI), said protein comprises an antigen binding fragment and/or a Fc fragment. In the protein conjugate, the molecule of interest may be conjugated to the protein through the oligosaccharide.

In the present disclosure, the oligosaccharide may be an N-linked oligosaccharide. In the present disclosure, the oligosaccharide may be linked to a nitrogen atom of an amino acid residue of the protein. In the present disclosure, the oligosaccharide may be linked to an asparagine (Asn) residue of the protein.

For example, the GlcNAc of the

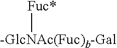

may be directly linked to an amino acid residue of the protein. For example, the GlcNAc of the

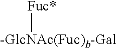

may be directly linked to an asparagine (Asn) residue of the protein. For example, the oligosaccharide may be

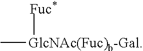

For example, the GlcNAc of the

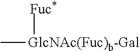

may be indirectly linked to an amino acid residue of the protein. For example, a saccharide may be between the GlcNAc of the

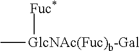

and an amino acid residue of the protein. For example, the saccharide may comprise mannoses. For example, the saccharide may be a

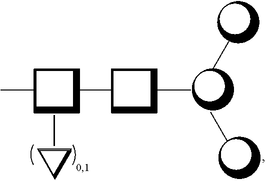

wherein [symbol] is a GlcNAc, [symbol] is a fucose, and [symbol] is a mannose.

For example, the GlcNAc of the

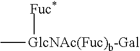

may be indirectly linked to an asparagine (Asn) residue of the protein. For example, a saccharide may be between the GlcNAc of the

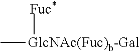

and an Asn of the protein. For example, the saccharide may comprise mannoses. For example, the saccharide may be

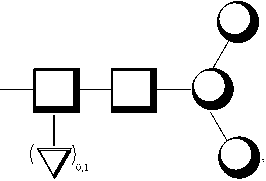

wherein [symbol] is a GlcNAc, [symbol] is a fucose, and [symbol] is a mannose.

For example, the GlcNAc of the

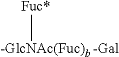

may be linked to a mannose of the saccharide and the oligosaccharide comprising the

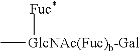

may be

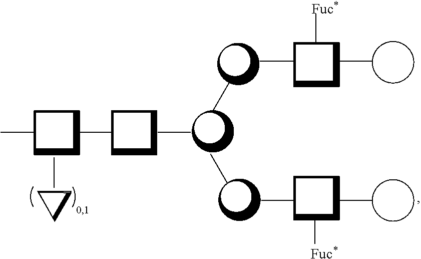

wherein [symbol] is a GlcNAc (wherein the [symbol] linked with Fuc* is the GlcNAc of the

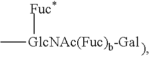

[symbol]

is a fucose, and [symbol] is a mannose, ◯ is a galactose.

In the present disclosure, the protein of the protein conjugate may comprise a Fc fragment. In the present disclosure, the oligosaccharide comprising the

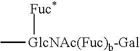

may be located in the Fc fragment. When the protein of the protein conjugate comprises a Fc fragment, the oligosaccharide comprising the

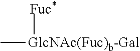

may be located in the CH₂ domain of the Fc fragment. For example, the oligosaccharide comprising the

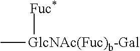

may be linked to the Asn297 of said Fc fragment, numbered according to the Kabat numbering system.

For example, the protein of the protein conjugate may be a Fc fusion protein. The protein of the protein conjugate may comprise a Fc fragment and a biologically active protein. For example, the biological active protein may be a therapeutic protein. For example, the biological active protein may be derived from a non-immunoglobulin. For example, the biological active protein may be a cytokine, a complement, and/or an antigen, or a fragment thereof.

In the present disclosure, the protein of the protein conjugate may comprise an antigen binding fragment. In the present disclosure, the oligosaccharide comprising the

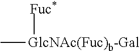

may located in the antigen binding fragment. For example, the protein of the protein conjugate may comprise nanobody, ScFv, Fab, F(ab)₂, F(ab′) and/or F(ab′)₂.

In the present disclosure, the protein of the protein conjugate may comprise a Fc fragment and an antigen binding fragment. In the present disclosure, the protein may be an antibody or a fragment thereof. In one aspect of the application, the antibody may recognize a target antigen. In some embodiments, the target antigen is a tumor antigen and may be localized to a tumor cell's surface. In some cases, the antibody bound to the target antigen can be internalized after binding to the tumor cell. When the antibody is covalently linked to a molecule of interest, the molecule of interest can be released into the cell after internalization. For example, when the functionalized antibody is linked to a cytotoxic drug, the cytotoxic drug can be released into the cell after internalization, resulting in cell death. In some cases, the target antigen displays differential expression between normal cells and tumor cells, displaying increased expression on tumor cells. For example, the target antigen may be selected from the group consisting of Trop2, Her2, CD20 and VEGF.

In the present disclosure, the protein may be an antibody or a fragment thereof. The antibody or antibody fragment can be of any class, such as an IgM, IgA, IgD, IgE, or IgG class, or subclass of immunoglobulin molecule. In some embodiments, the antibody or antibody fragment is of the IgG class. The antibody or antibody fragment can be from the IgGl, IgG2, IgG3, and/or IgG4 subclasses. In some embodiments, the antibody or antibody fragment is from the IgGl subclass. In some embodiments, the antibody or antibody fragment have a conserved asparagine at position 297 of the heavy chain as defined by the Kabat numbering system (Kabat et al., Sequences of Proteins of Immunological Interest, Vol. 1, 5th Ed. U.S. Public Health Service, National Institutes of Health. NIH Publication No. 91-3242; Copyright 1991).

In the present disclosure, the protein may be an antibody or a fragment thereof. The antibody or antibody fragment may be derived from a human, a mouse, a rat, or another mammal. The antibody or antibody fragment may also be a hybridization of antibodies from human, mouse, rat, and/or other mammals. In some embodiments, the antibody or antibody fragment is derived from a human. The antibody or antibody fragment may be produced by hybridoma cells or cell lines. The antibody or antibody fragment may be humanized. For example, the antibody could be but not limited trastuzumab, bevacizumab, rituximab, durvalumab, pertuzumabetc, raxibacumab, dinutuximab, ixekizumab, labetuzumab, odesivimab. risankizumab, dinutuximab, adalimumab, cetuximab, daratumumab, tocilizumab, and etc. For example, the antibody may be trastuzumab, rituximab, bevacizumab or hRS7. For example, the heavy chain of trastuzumab may comprise the amino acid sequence as set forth in SEQ ID NO: 9, and the light chain of trastuzumab may comprise the amino acid sequence as set forth in SEQ ID NO: 8. For example, the heavy chain of rituximab may comprise the amino acid sequence as set forth in SEQ ID NO: 11, and the light chain of rituximab may comprise the amino acid sequence as set forth in SEQ ID NO: 10. For example, the heavy chain of bevacizumab may comprise the amino acid sequence as set forth in SEQ ID NO: 13, and the light chain of bevacizumab may comprise the amino acid sequence as set forth in SEQ ID NO: 12. For example, the heavy chain of hRS7 may comprise the amino acid sequence as set forth in SEQ ID NO: 15, and the light chain of hRS7 may comprise the amino acid sequence as set forth in SEQ ID NO: 14.

In the present disclosure, the protein conjugate may comprise an antibody. For example, the protein may be an antibody, and the protein conjugate may have the similar binding affinity towards an antigen, compared to the corresponding antibody. For example, the protein may be an antibody, and the protein conjugate may have a comparable binding activity towards an antigen, compared to the corresponding antibody.

For example, the binding activity or binding affinity to an antigen of the protein conjugate in the present disclosure may be about 0.1% to about 100000% (e.g., about 1%-10000%, about 10%-1000%, or about 50%-200%) of the binding activity or binding affinity of the corresponding antibody. The binding activity to an antigen may be compared by a quantitative or a non-quantitative method. In some cases, the binding activity or binding affinity can be qualified. For example, by ELISA, under a condition that allows the protein conjugate and/or its corresponding antigen to bind to a target (e.g., an antigen), contact the protein conjugate and/or its corresponding antigen with the target (eg, antigen), determine whether a complex is formed between the protein conjugate and the target (e.g., an antigen), and determine whether a complex is formed between the corresponding and the target (e.g., an antigen). For example, the binding activity or binding affinity to a target (e.g., an antigen) may be quantified by a value. For example, the value is a Kd value. For example, the value is an OD value. For example, the value is an absorbance value. The binding affinity can be qualified by the value (e.g., OD value, KD value, or absorbance value) after statistical analysis, in which the binding affinity of the corresponding antibody may be set as 100%.

In the present disclosure, a variety of methods can be used to determine the binding activity of the protein conjugate and its corresponding antigen.

The binding activity can be determined by, for example, ELISA, isothermal titration calorimetry, surface plasmon resonance, and/or biolayer interferometry. For example, the binding activity of the corresponding antibody can be set as 100%.

In the present disclosure, the Fuc* may be Fuc-MOI, wherein said Fuc is a fucose or fucose derivative. The fucose of Fuc* connects the MOI and the GlcNAc of the oligosaccharide. For example, the fucose or fucose derivative of the Fuc* may be linked to the GlcNAc through an Fuc*α1,3GlcNAc linkage.

In the present disclosure, the MOI of Fuc* may comprise an active moiety. One or more desirable characteristics can be introduced to the protein conjugate with the MOI.

In the present disclosure, the active moiety may comprise a functional group Y₁. The functional group Y₁ may be capable of participating in a ligation reaction. For example, the functional group Y₁ may be able to connect a molecule linked with Y₁ to another molecule linked with another functional group, which is capable of reacting with Y₁.

For example, the functional group Y₁ comprises a functional moiety capable of participating in a bioorthogonal reaction. For example, the functional moiety of Y₁ may be selected from the group consisting of azide, terminal alkyne, cyclic alkyne, tetrazine, 1,2,4-trazine, terminal alkene, transcyclooctene, cyclopropene, norbornene, keto, aldehyde, aminooxy, thiol, and maleimide. For example, the functional moiety of Y₁ may be selected from the group consisting of azide derivative, terminal alkyne derivative, cyclic alkyne derivative, tetrazine derivative, 1,2,4-trazine derivative, terminal alkene derivative, transcyclooctene derivative, cyclopropene derivative, norbornene derivative, keto derivative, aldehyde derivative, aminooxy derivative, thiol derivative, and maleimide derivative.

For example, the functional group Y₁ comprises a functional moiety capable of participating in a bioorthogonal reaction. For example, the functional moiety of Y₁ may be selected from the group consisting of

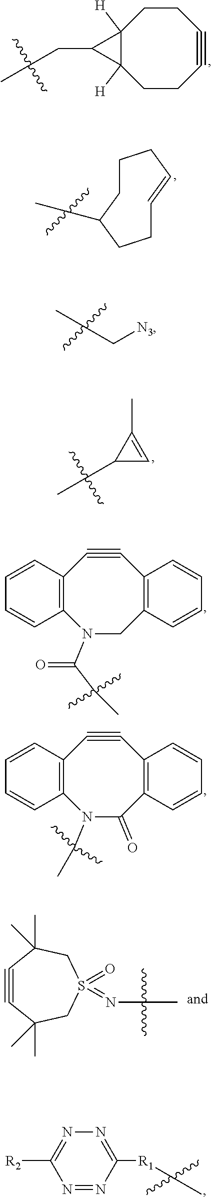

wherein each of R₁ and R₂ is independently selected from the group consisting of hydrogen, halogen, C₁-C₂₂ alkyl group, C₅-C₂₂ (hetero)aryl group, C₇-C₂₂ alkyl(hetero)aryl group and C₇-C₂₂ (hetero)arylalkyl group, wherein each of said alkyl group optionally is interrupted by one or more hetero-atom selected from the group consisting of O, N, and S, and wherein each of the alkyl group, (hetero)aryl group, alkyl(hetero)aryl group and (hetero)arylalkyl groups is independently optionally substituted.

For example, the functional group Y₁ comprises a functional moiety capable of participating in a bioorthogonal reaction. For example, the functional moiety of Y₁ may be selected from the group consisting of

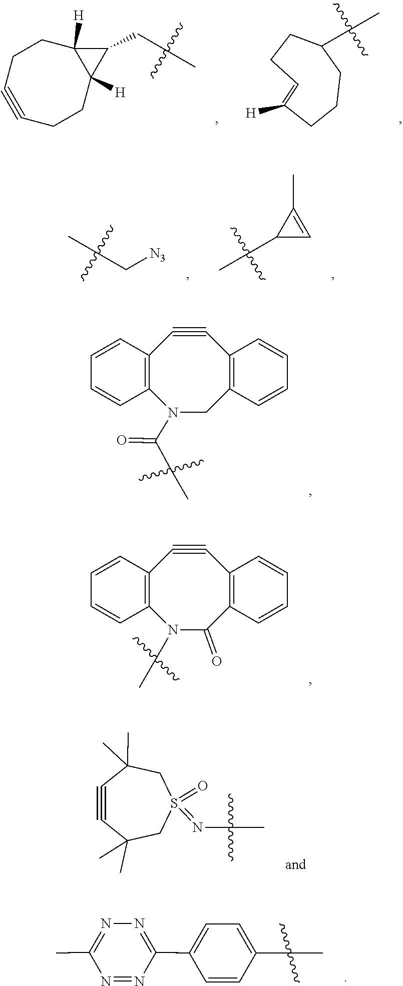

In the present disclosure, the MOI of Fuc* may further comprise a connector F. In some circumstance, the connector F is necessary in an enzymic reaction, glycosyl transfer reaction, and or a ligand reaction (e.g., biorthogonal reaction). For example, the connector F may comprise a, a or a. For example, the connector F may comprise a spacer FL. For example, the connector F may comprise a, a or a, and a spacer FL. And the spacer FL may be a polypeptide, a PEG, an alkyl and/or their derivatives or combination thereof. For example, the spacer FL may be selected from the group consisting of

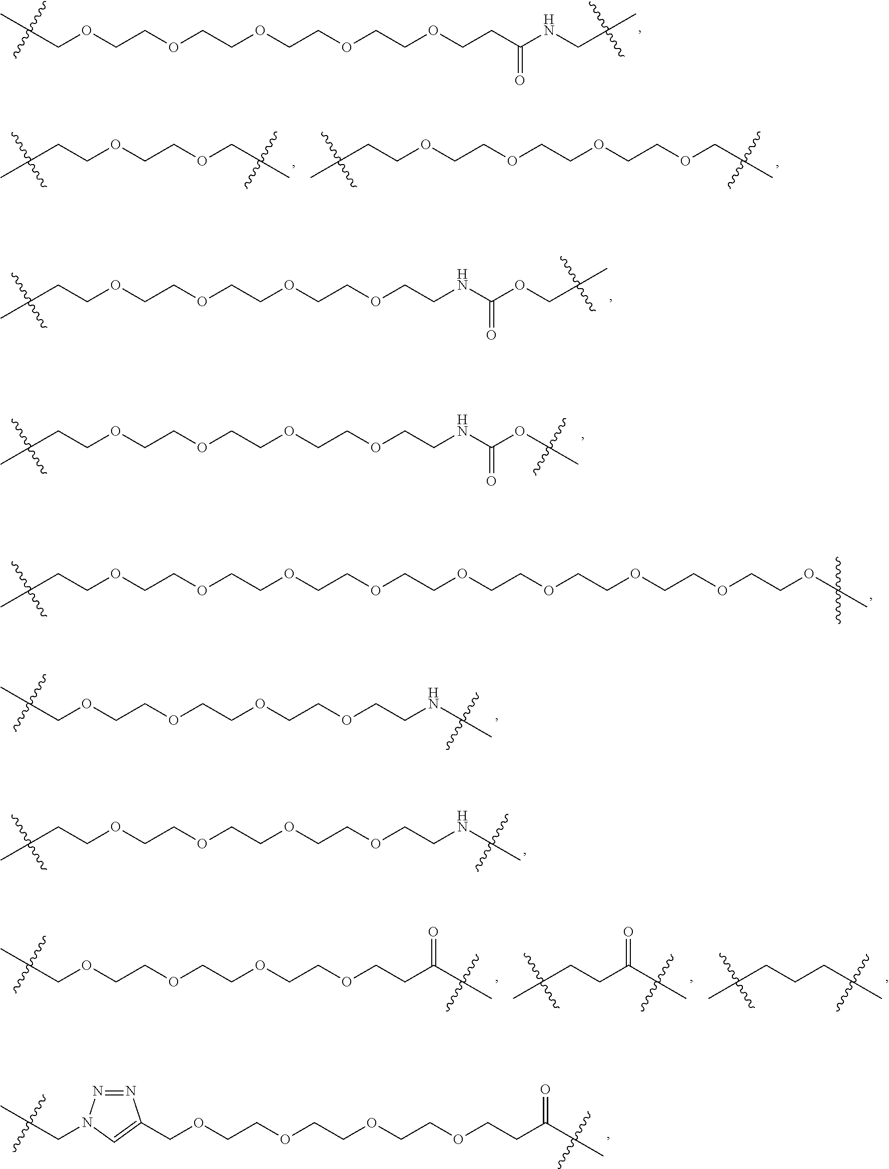

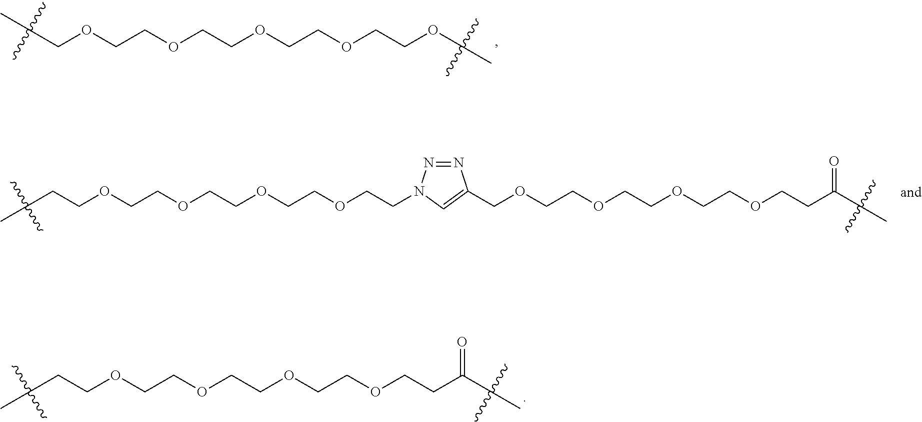 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

For example, the connector F may be a

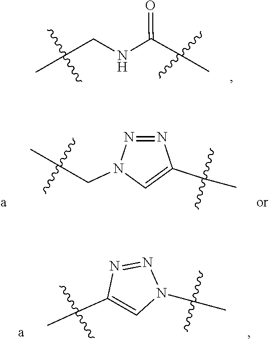 [SMILES: *CNC(*)=O ; *Cn1cc(*)nn1 ; *c1cn(*)nn1]

or the combination of thereof. For example, the connector F may be a

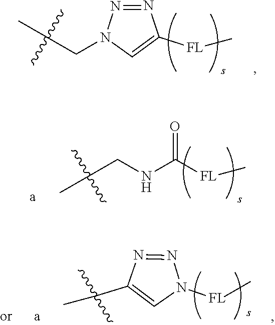

or the combination of thereof, wherein the FL is a spacer and s is 1

For example, the connector F is a

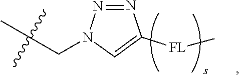

wherein said FL is a polypeptide, a PEG, an alkyl and/or their derivatives or combination thereof, and s is 1.

For example, the connector F is a

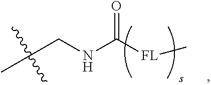

wherein said FL is a polypeptide, a PEG, an alkyl and/or their derivatives or combination thereof, and s is 1. For example, FL is

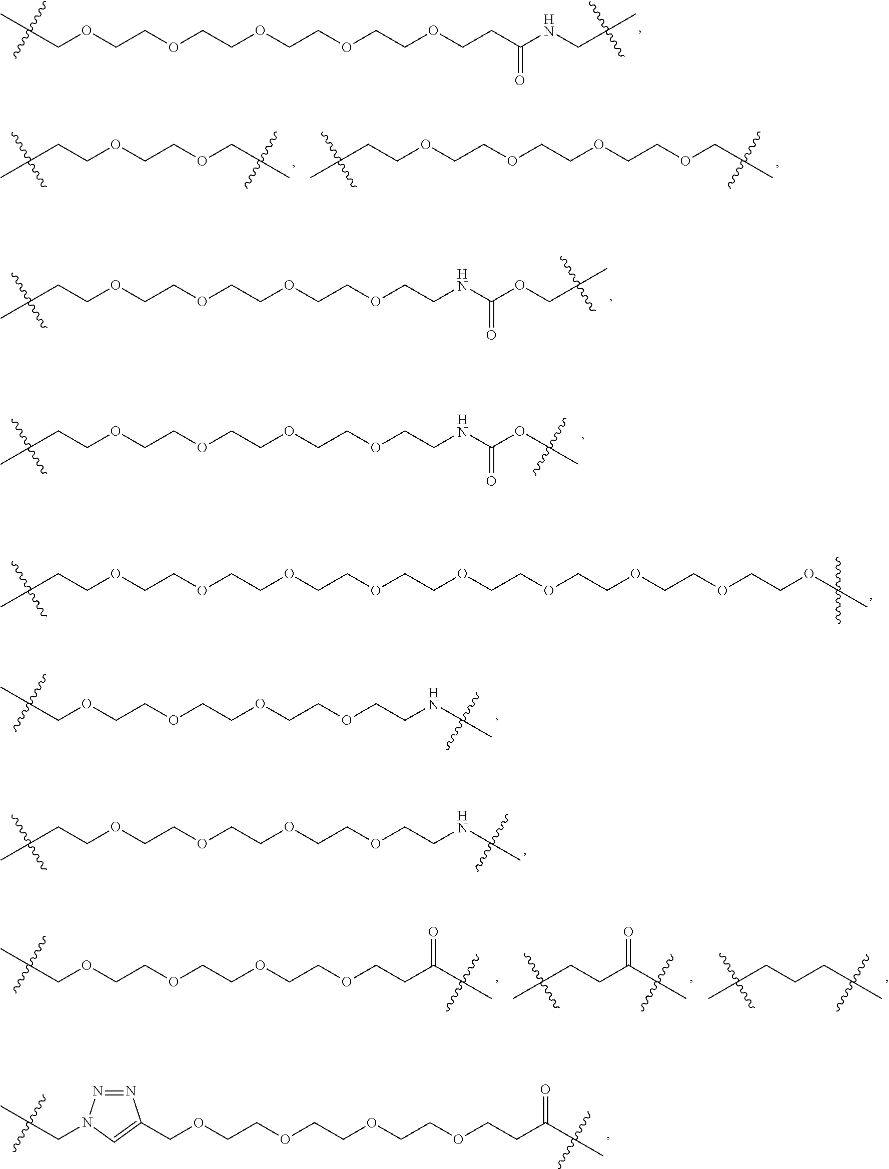

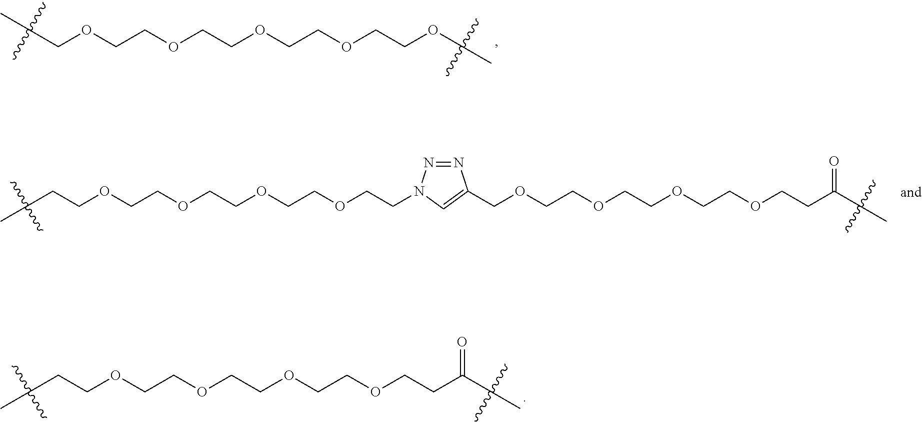 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

In the present disclosure, the MOI of Fuc* may further comprise a linker L. In some circumstances, e.g., in a special range of pH, in a special range of temperature, or in presence of an enzyme, the linker may be cleaved.

In the present disclosure, the Fuc* may have the structure of Fuc-(F)ₘ-(L)ₙ-Y₁, wherein Fuc is a fucose or fucose derivative, F is the connector, L is a linker, Y₁ is the functional group, m, n is 0 or 1. For example, Fuc* may have the structure of Fuc-Y₁, Fuc-L-Y₁, Fuc-F-Y₁ or Fuc-F-L-Y₁, wherein the Fuc, F, and Y₁ are defined as above.

For example, the Fuc* may have the structure of Fuc-F-L-Y₁, wherein the F is a

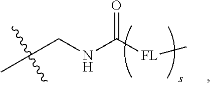

s is 1, and FL may be selected from the group consisting of

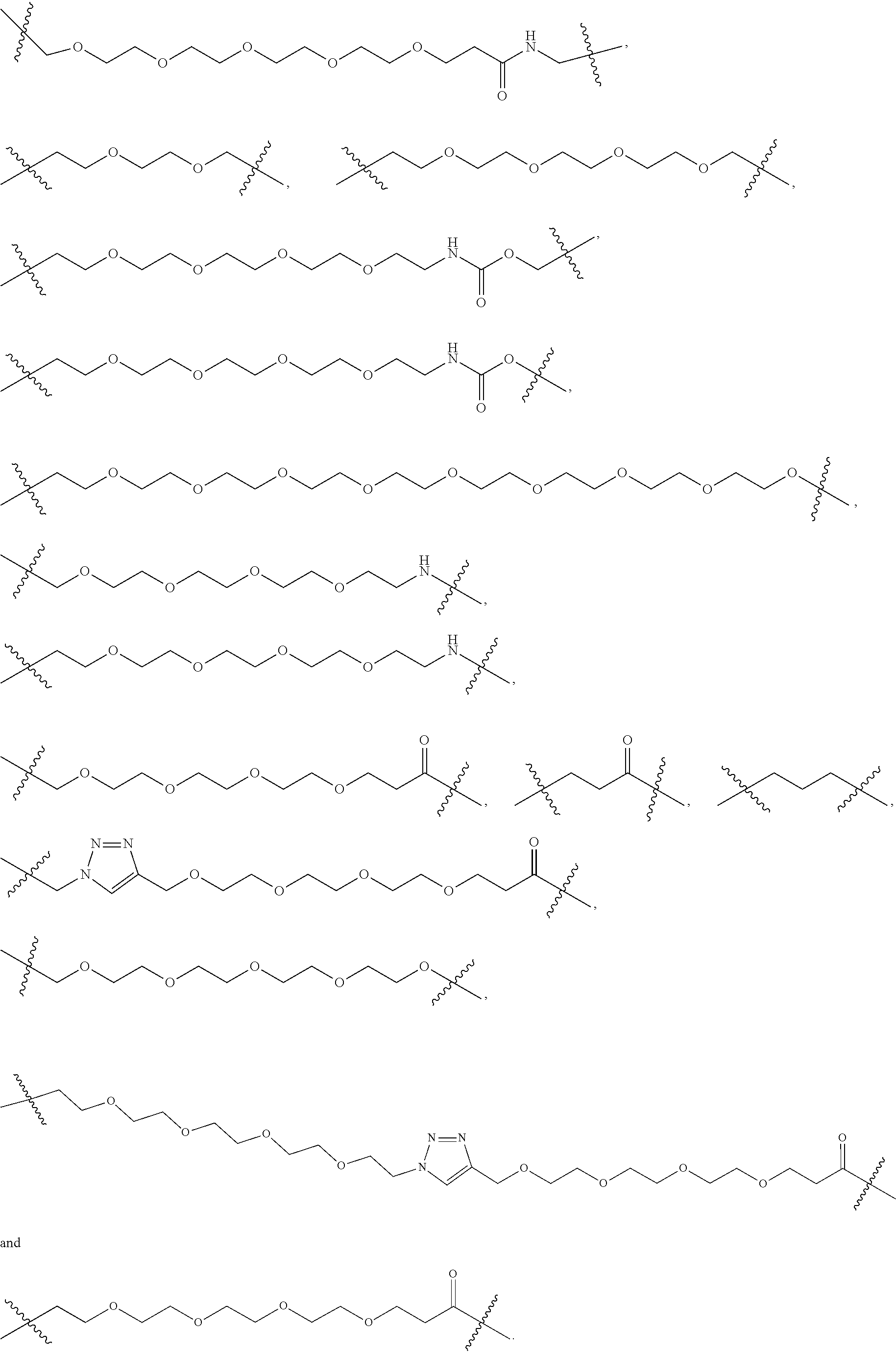

For example the Fuc* may comprise a structure of

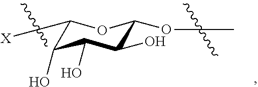

wherein the X is selected from:

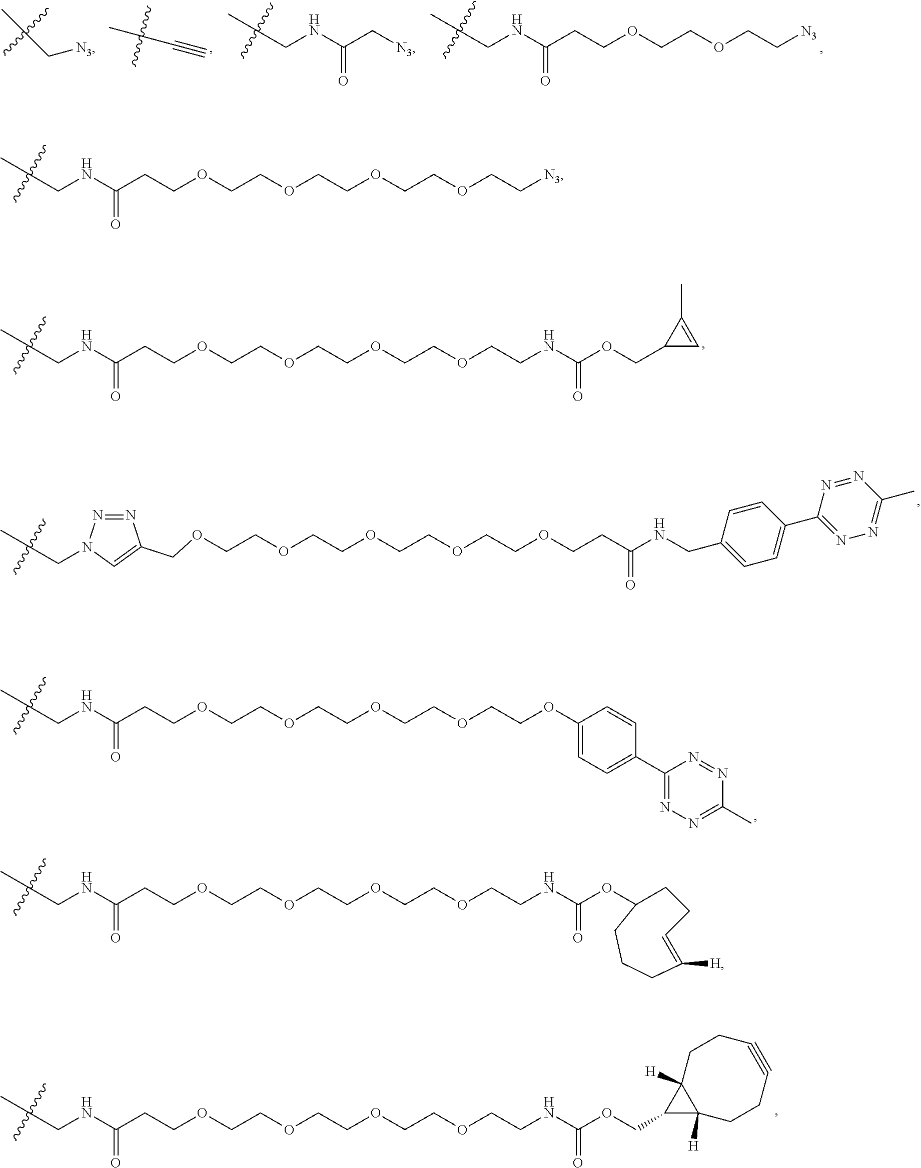

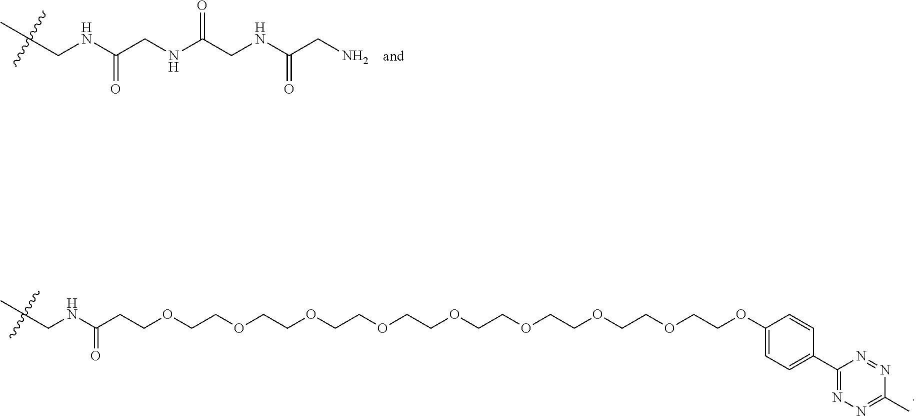 [SMILES: *CNC(=O)CNC(=O)CNC(=O)CN ; "and" ; *CNC(=O)CCOCCOCCOCCOCCOCCOCCOCCOCCOc1ccc(-c2nnc(C)nn2)cc1]

For example, the Fuc* may comprise a structure of

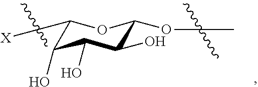

wherein the X is selected from:

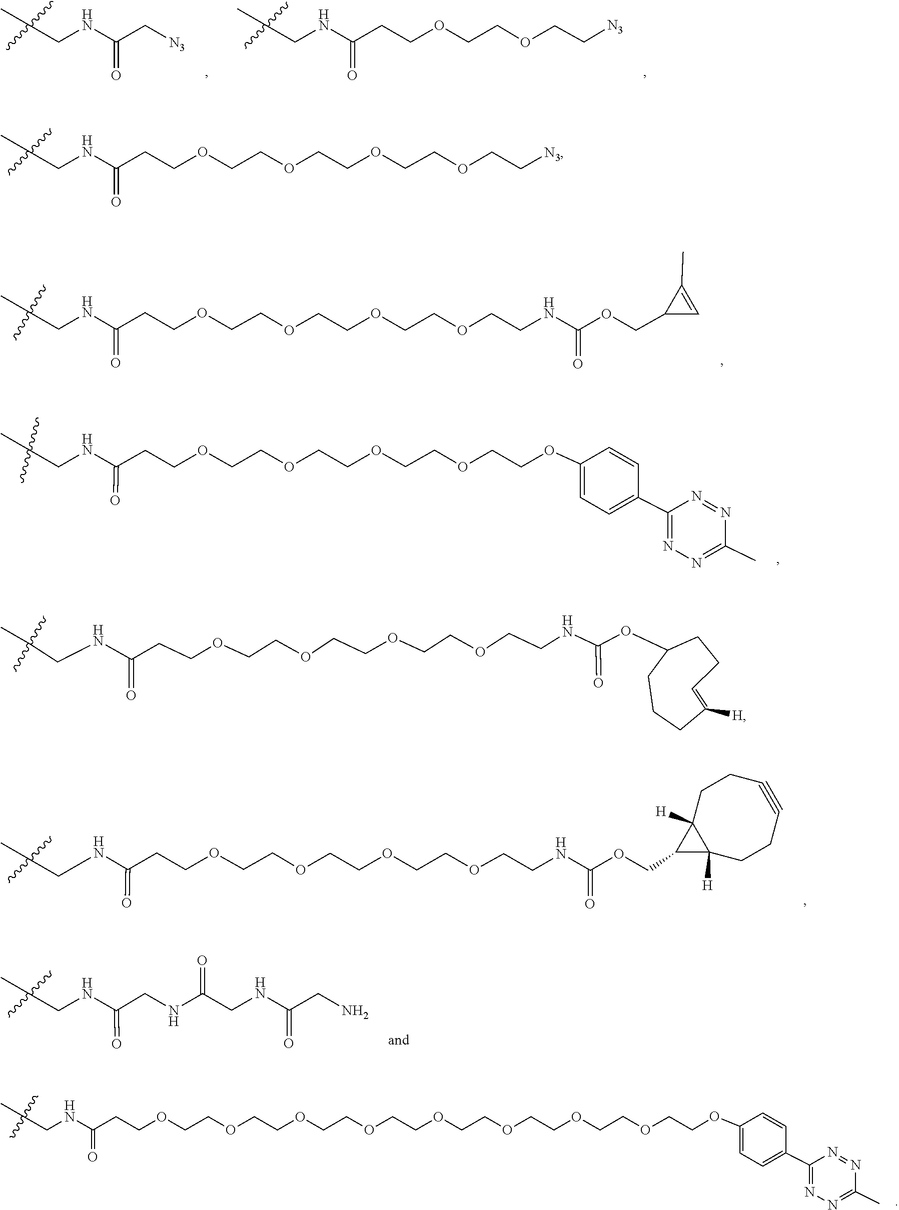

In the present disclosure, the active moiety may comprise a biologically and/or a pharmaceutically active substance P. The biologically and/or pharmaceutically active substance P itself may not participate in a ligation reaction. The P may induce a biologically and/or pharmaceutically activity to the protein conjugate. For example, the P may comprise a cytotoxin, a cytostatic agent, a radioisotope or radionuclide, a metal chelator, an oligonucleotide, an antibiotic, a fluorophore, a biotin tag, a peptide, or a protein, or any combination thereof. For example, the P may comprise a pharmaceutically active substance selected from a cytotoxin, a cytostatic agent, a radioisotope or radionuclide, a metal chelator, an oligonucleotide, an antibiotic, a peptide, or a protein, and/or any combination thereof.

For example, the P may be toxin, cytokine, growth factor, radionuclide, hormone, anti-viral agent, anti-bacterial agent, fluorescent dye, agent, half-life increasing moiety, solubility increasing moiety, a polymer-toxin conjugate, a nucleic acid, a biotin or streptavidin moiety, a vitamin, a target binding moiety, and/or, anti-inflammatory agent. For example, the P may be toxin, cytokine, growth factor, radionuclide, hormone, anti-viral agent, anti-bacterial agent, half-life increasing moiety, solubility increasing moiety, a polymer-toxin conjugate, a nucleic acid, a vitamin, a target binding moiety, and/or, anti-inflammatory agent.

For example, the P may be a therapeutically active moiety, which can be used in preventing, treating and/or relieving a disease. For example, the P may be an anti-tumor agent, which may be selected from chemical therapy agent, and/or a targeting therapy agent. For example, the P may be a substance which results in cell damage or cell death, e.g, a cytotoxin. For example, the P comprises a cytotoxin selected from the group consisting of pyrrolobenzodiazepine (PBD), auristatin (e.g., MMAE, or MMAF, maytansinoids (Maytansine, DM1, or DM4), duocarmycin, tubulysin, enediyene (e.g. Calicheamicin), doxorubicin (PNUs), pyrrole-based kinesin spindle protein (KSP) inhibitor, calicheamicin, amanitin (e.g. a-Amanitin), and camptothecin (e.g. exatecan, deruxtecan).

For example, the P may comprise a cytotoxin. For example, the P may comprise MMAE, DXd, MMAF, seco-DUBA or DM4.

In the present disclosure, the MOI of Fuc* may further comprise a linker L. In some circumstances, e.g., in a special range of pH, in a special range of temperature, or in presence of an enzyme, the linker may be cleaved, and the P of MOI can exert a biologically and/or pharmaceutically activity in vivo or in vitro, depended on where the protein of the protein conjugate are. For example, L is a cleavable linker. A lot of type of cleavable linkers in the art can be used in the present disclosure. For example, the L may be an acid-labile linker, a redox-active linker, a photo-active linker and/or a proteolytically cleavable linker. For example, the L may be a vc-PAB linker, a GGFG linker or a dislufo linker.

In the present disclosure, the MOI of Fuc* may further comprise a connector F. In some circumstance, the connector F is necessary in an enzymic reaction, glycosyl transfer reaction, and or a ligand reaction (e.g., biorthogonal reaction). For example, the connector F may comprise a

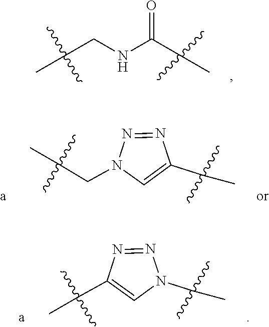 [SMILES: *CNC(*)=O ; *Cn1cc(*)nn1 ; *c1cn(*)nn1]

For example, the connector F may comprise a spacer FL. For example, the connector F may comprise a

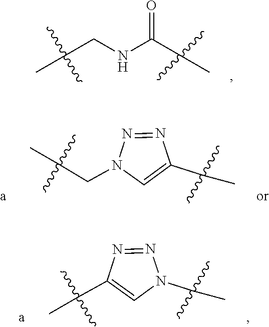 [SMILES: *CNC(*)=O ; *Cn1cc(*)nn1 ; *c1cn(*)nn1]

and a spacer FL. And the spacer FL may be a polypeptide, a PEG, an alkyl and/or their derivatives or combination thereof. For example, the spacer FL may be selected from the group consisting of

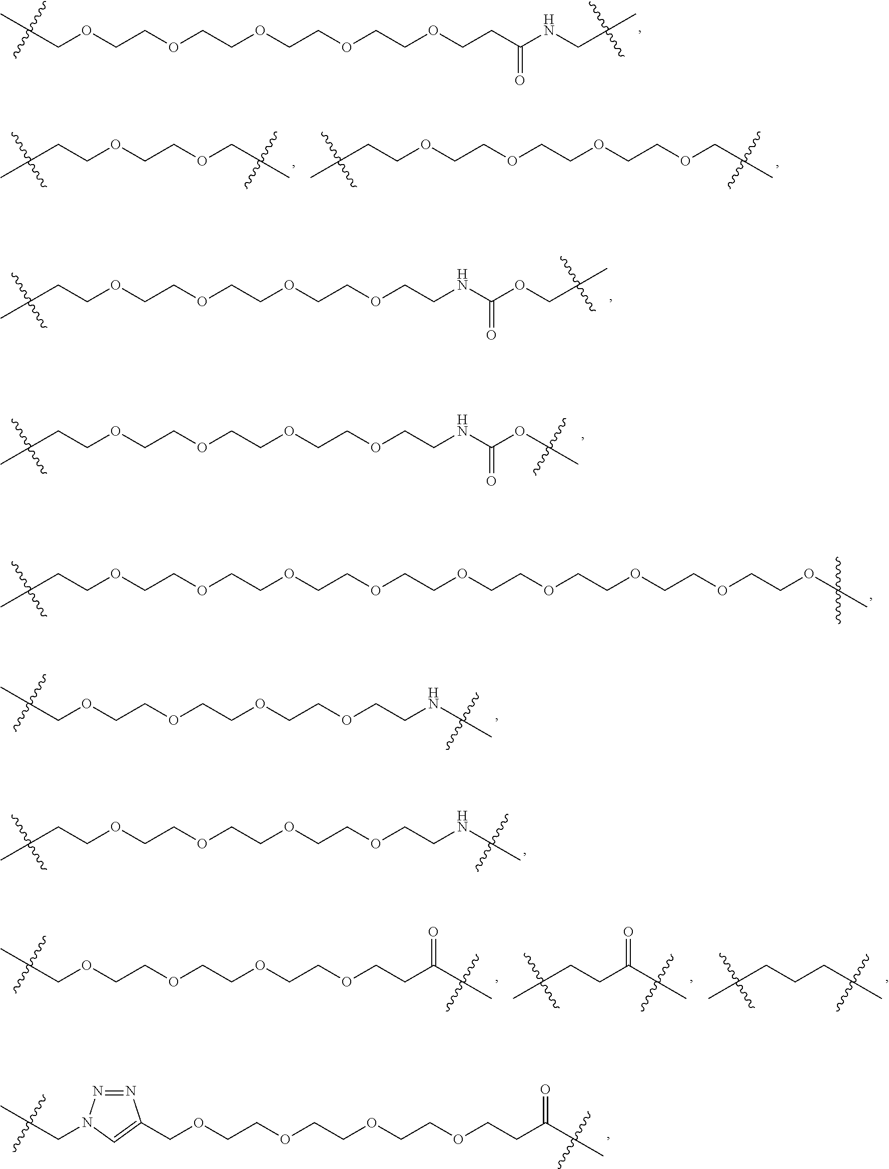

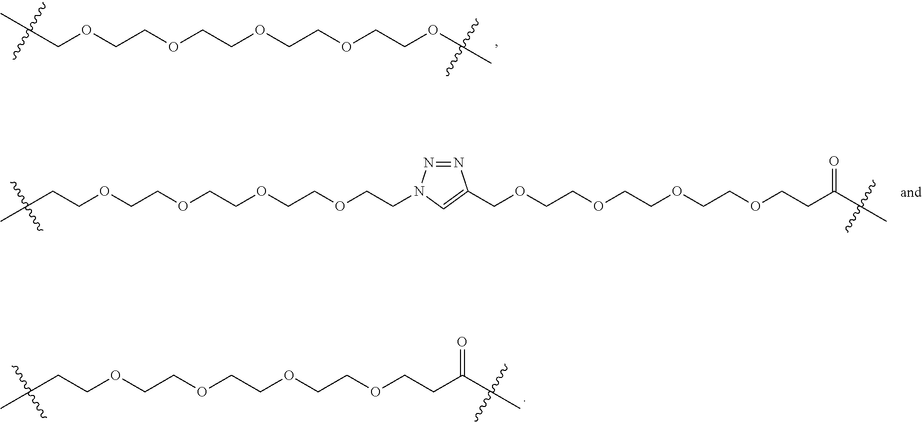 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

For example, the connector F may be a

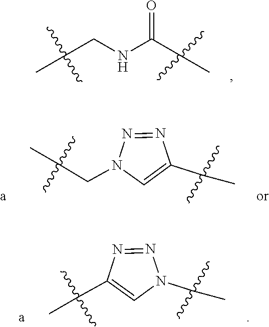 [SMILES: *CNC(*)=O ; *Cn1cc(*)nn1 ; *c1cn(*)nn1]

or the combination of thereof. For example, the connector F is a

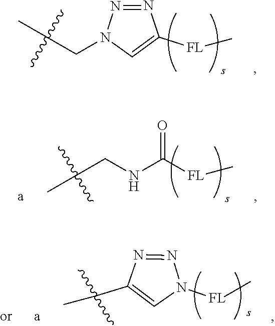

or the combination of thereof, wherein the FL is a spacer and s is 1.

For example, the connector F is a

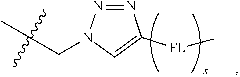

wherein said FL is a polypeptide, a PEG, an alkyl and/or their derivatives or combination thereof, and m is 0 or 1. For example, FL may be selected from the group consisting of

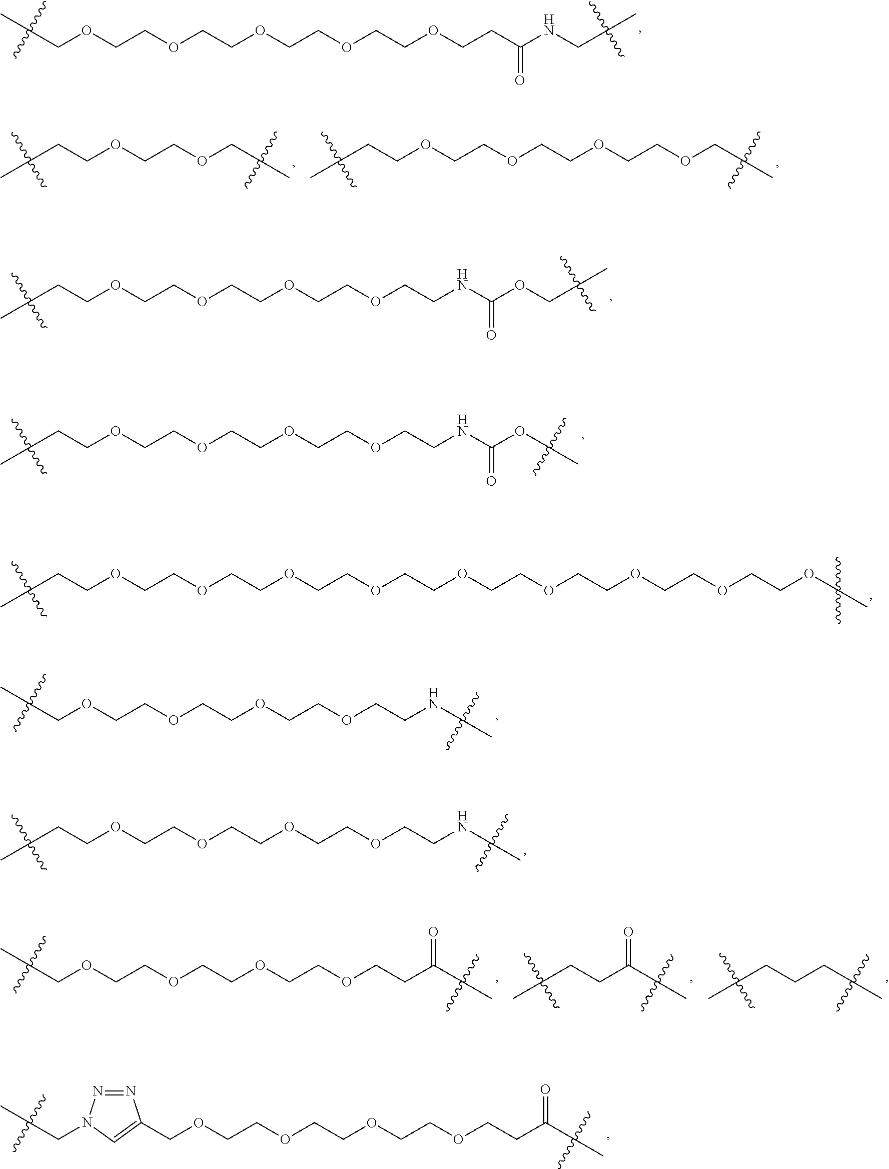

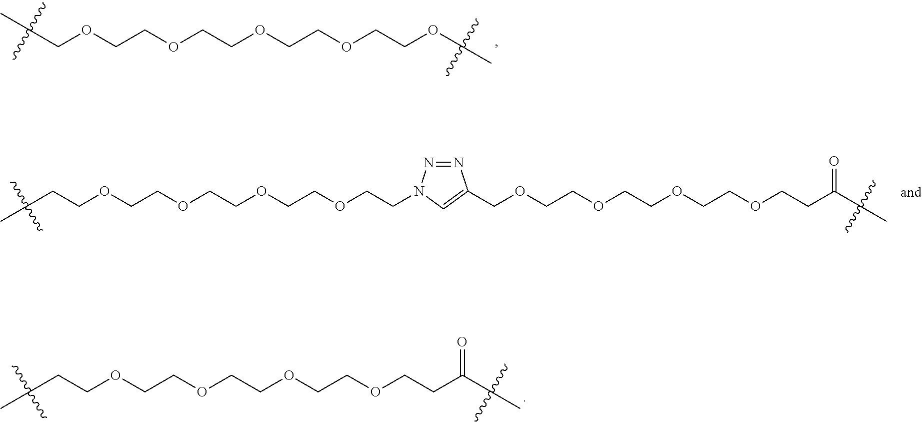 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

For example, the connector F is a

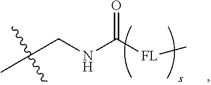

wherein said FL is a polypeptide, a PEG, an alkyl and/or their derivatives or combination thereof, and s is 0 or 1. For example, FL may be selected from the group consisting of

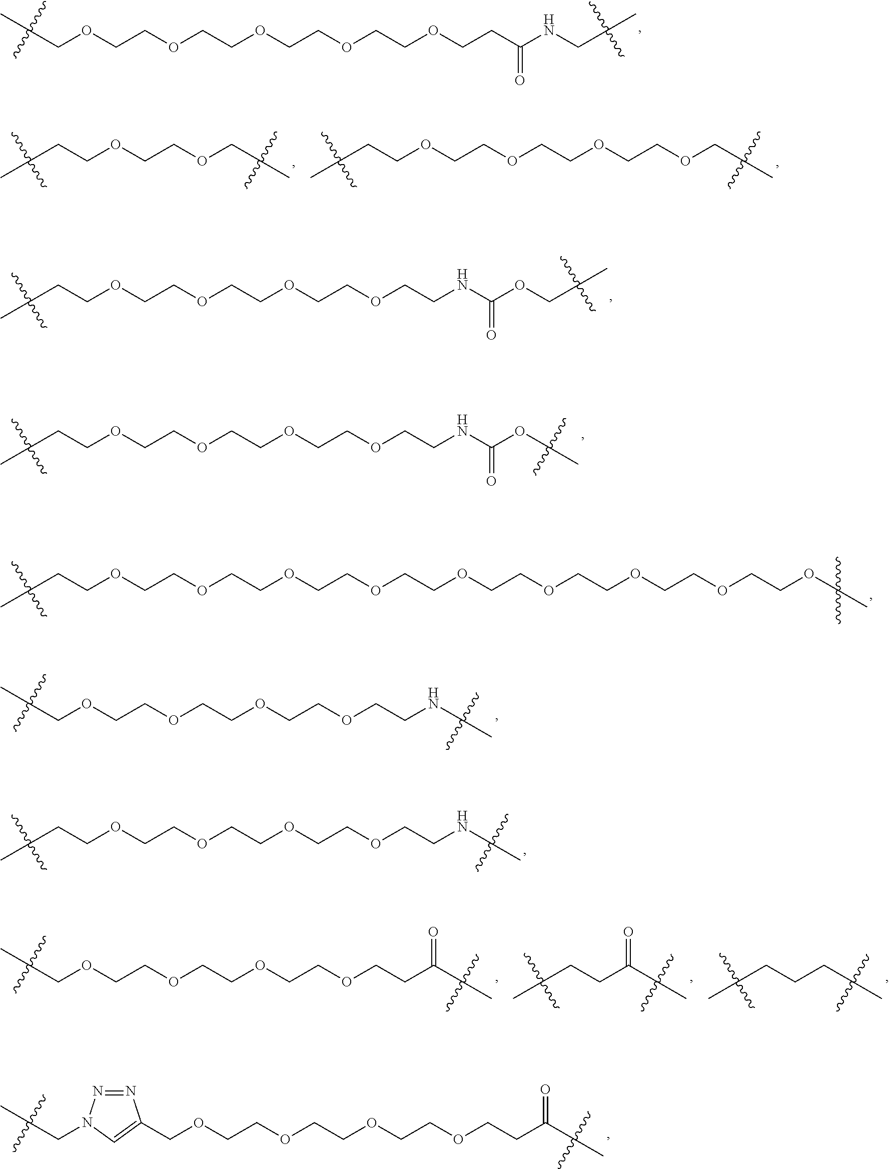

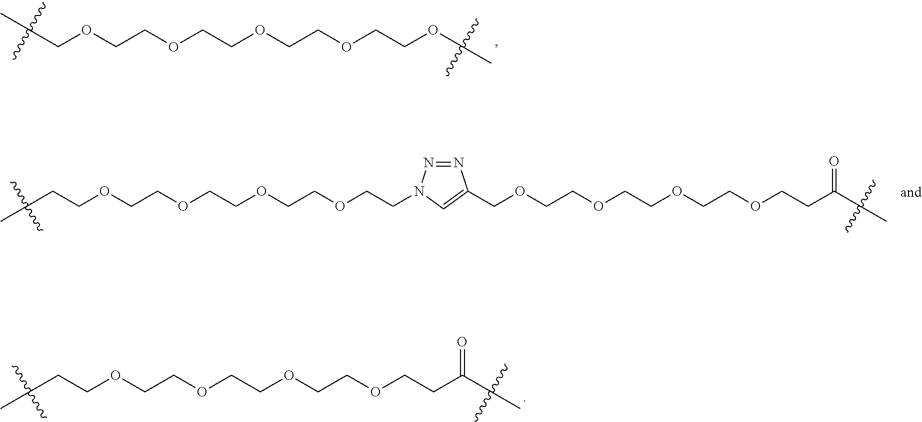 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

In the present disclosure, the Fuc* may have the structure of Fuc-(F)ₘ-(L)ₙ-P, wherein Fuc is a fucose or fucose derivative, F is the connector, L is the linker, P is the biologically and/or pharmaceutically active substance, m is 0 or 1, n is 0 or 1. For example, Fuc* may have the structure of Fuc-P, Fuc-F-P, Fuc-L-P, Fuc-F-L-P, wherein the Fuc, F, L and P are defined as above.

For example, the Fuc* may have the structure of Fuc-F-L-P, wherein the F is a

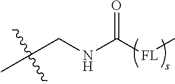

s is 1, and FL may be selected from the group consisting of

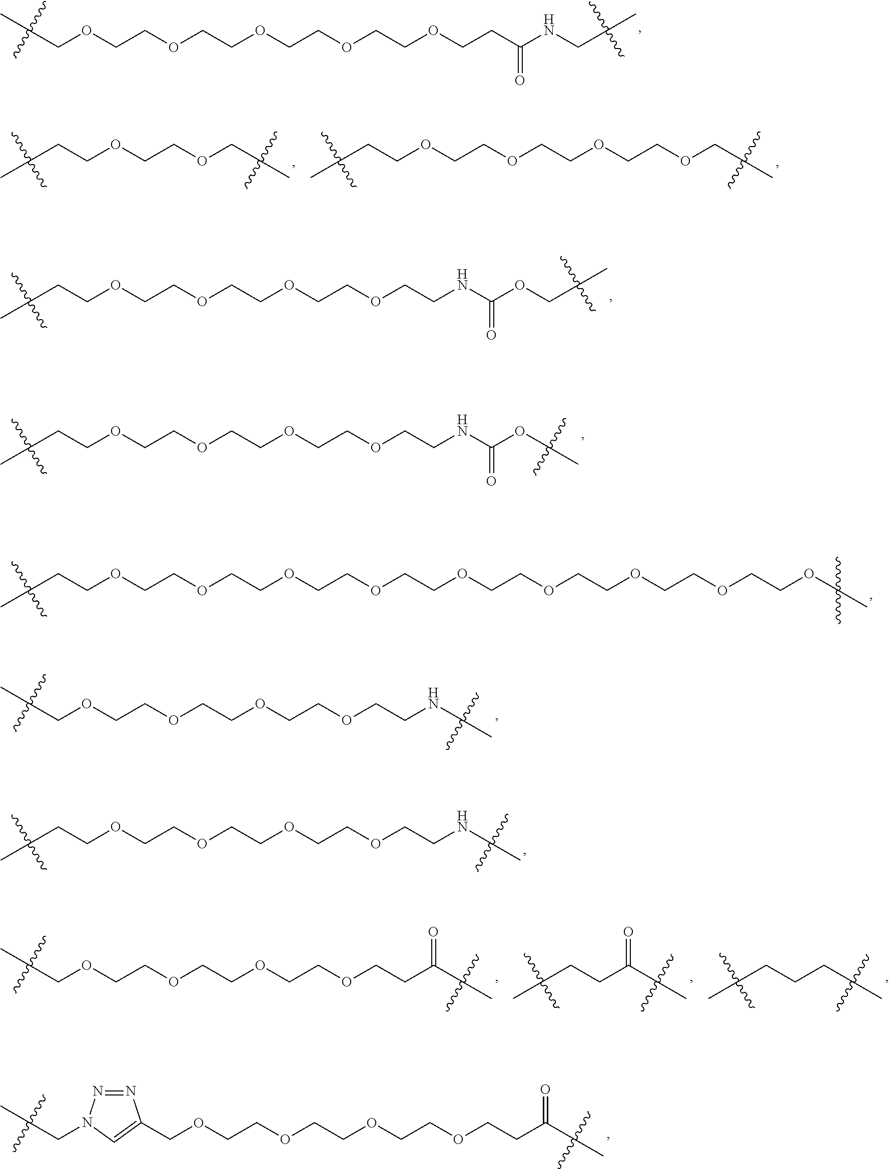

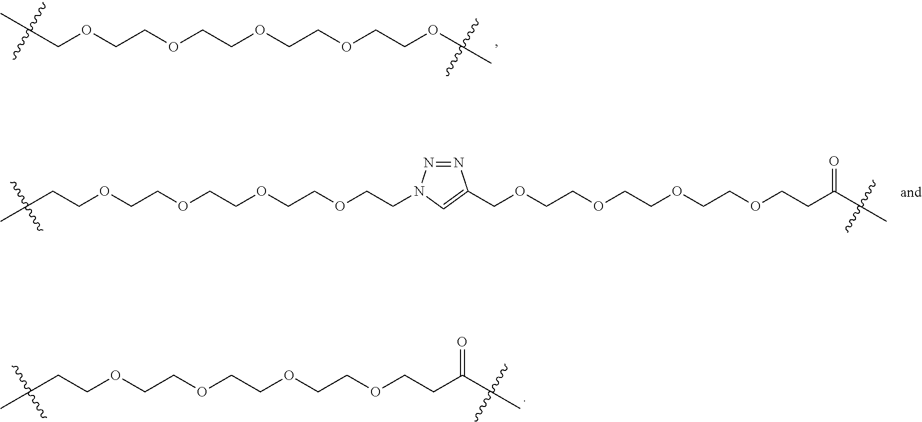 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

For example the Fuc* may comprise a structure of a

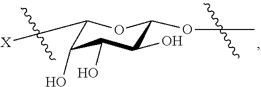

wherein the X is selected from:

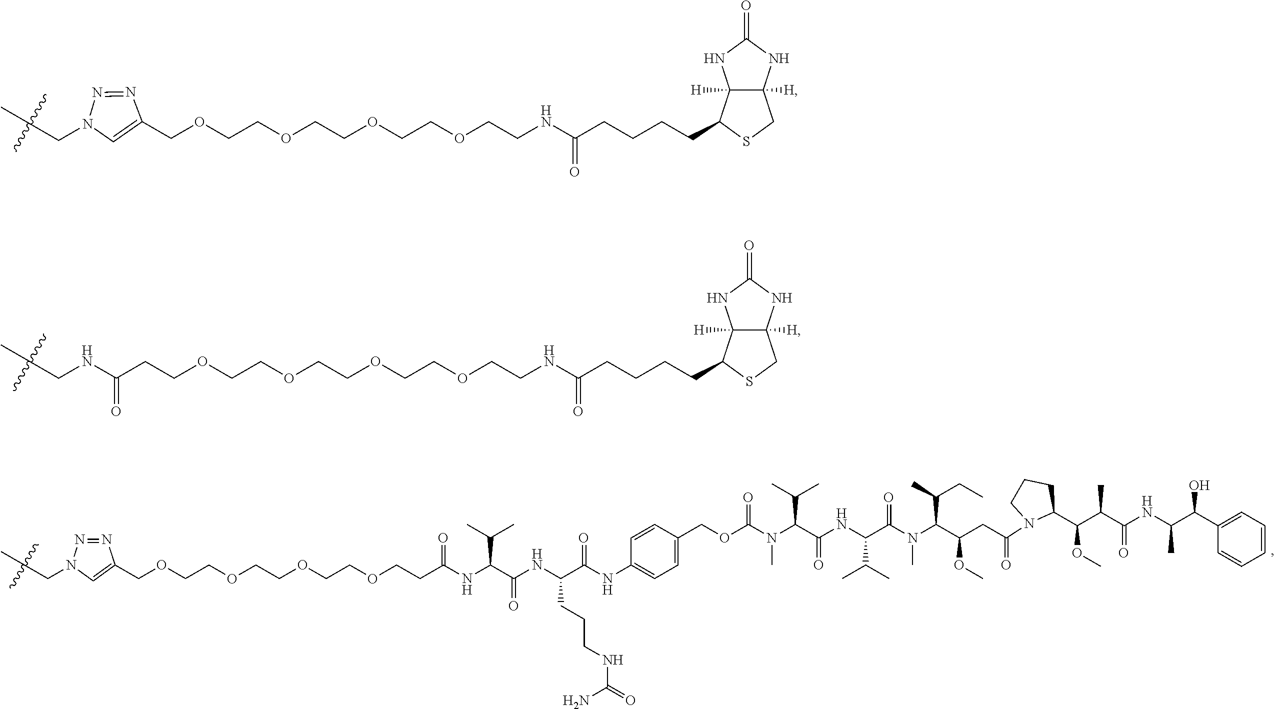

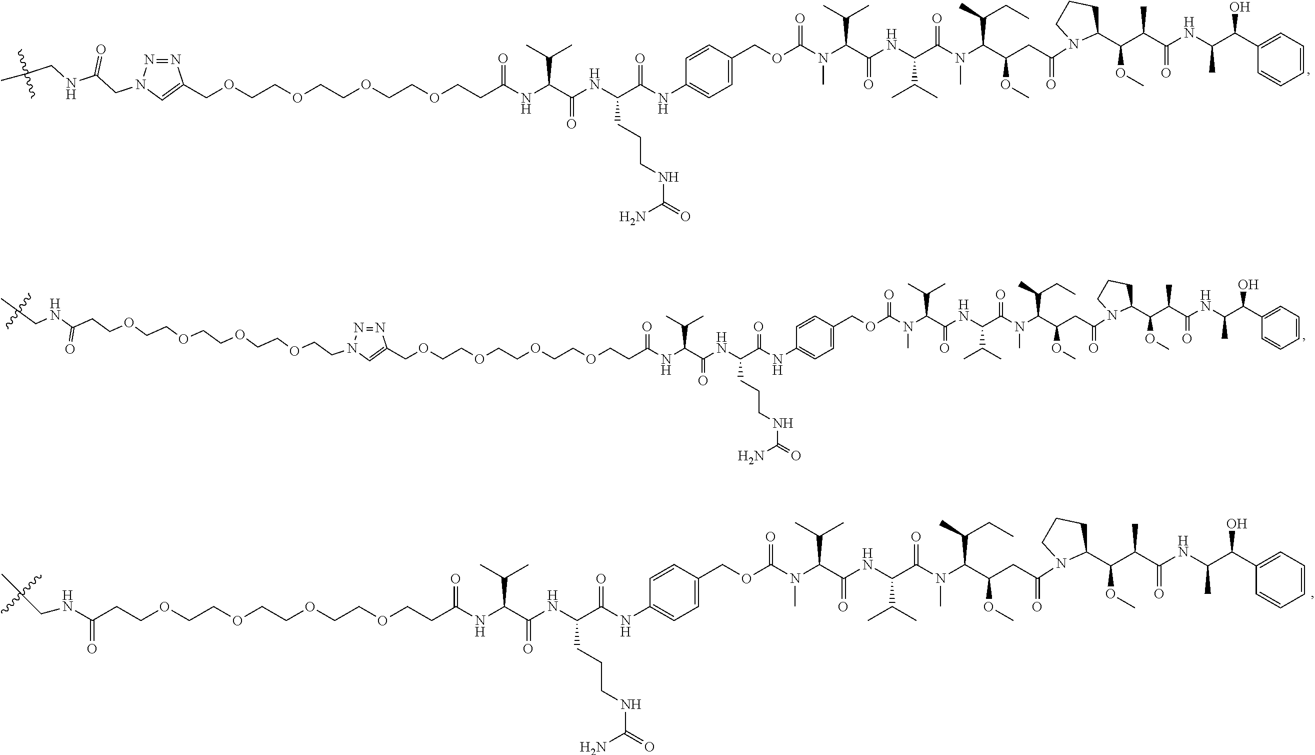

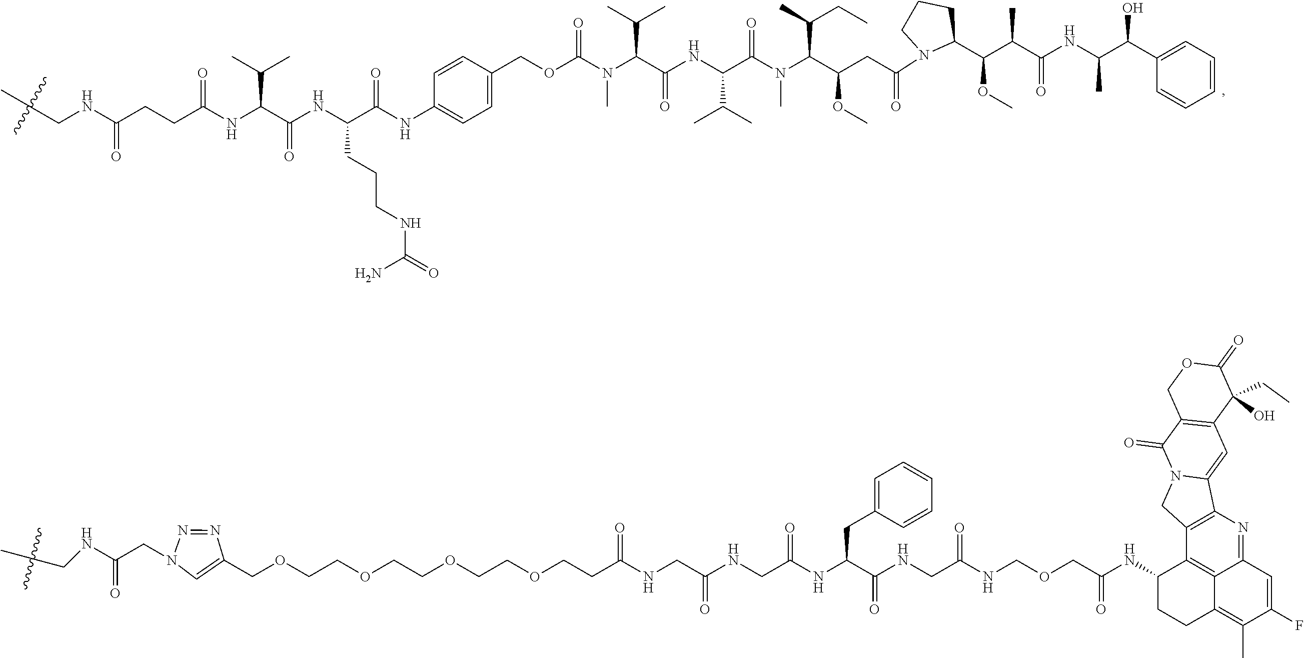 [SMILES: *CNC(=O)CCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C ; *CNC(=O)Cn1cc(COCCOCCOCCOCCC(=O)NCC(=O)NCC(=O)N[C@@H](Cc2ccccc2)C(=O)NCC(=O)NCOCC(=O)N[C@H]2CCc3c(C)c(F)cc4nc5c(c2c34)Cn2c-5cc3c(c2=O)COC(=O)[C@]3(O)CC)nn1]

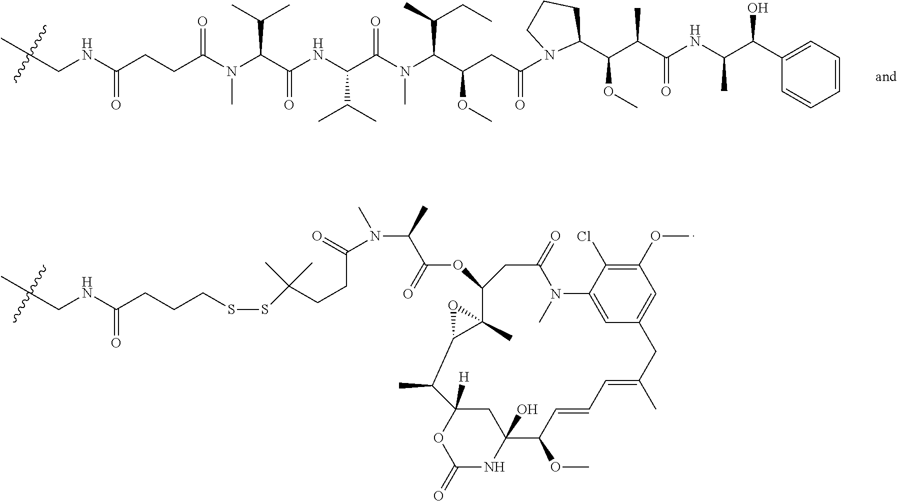

When in this case, the protein of the protein conjugate may be an antibody, and the protein conjugate may have the similar binding affinity towards an antigen, compared to the corresponding antibody.

For example, the binding affinity of said obtained protein conjugate may be about 0.1% to about 100000% (e.g., about 1%-10000%, about 10%-1000%, or about 50%-200%) of the binding affinity of the corresponding antibody.

For example, the Fuc* may comprise a structure of

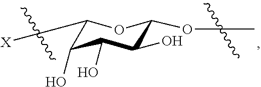

wherein the X is selected from:

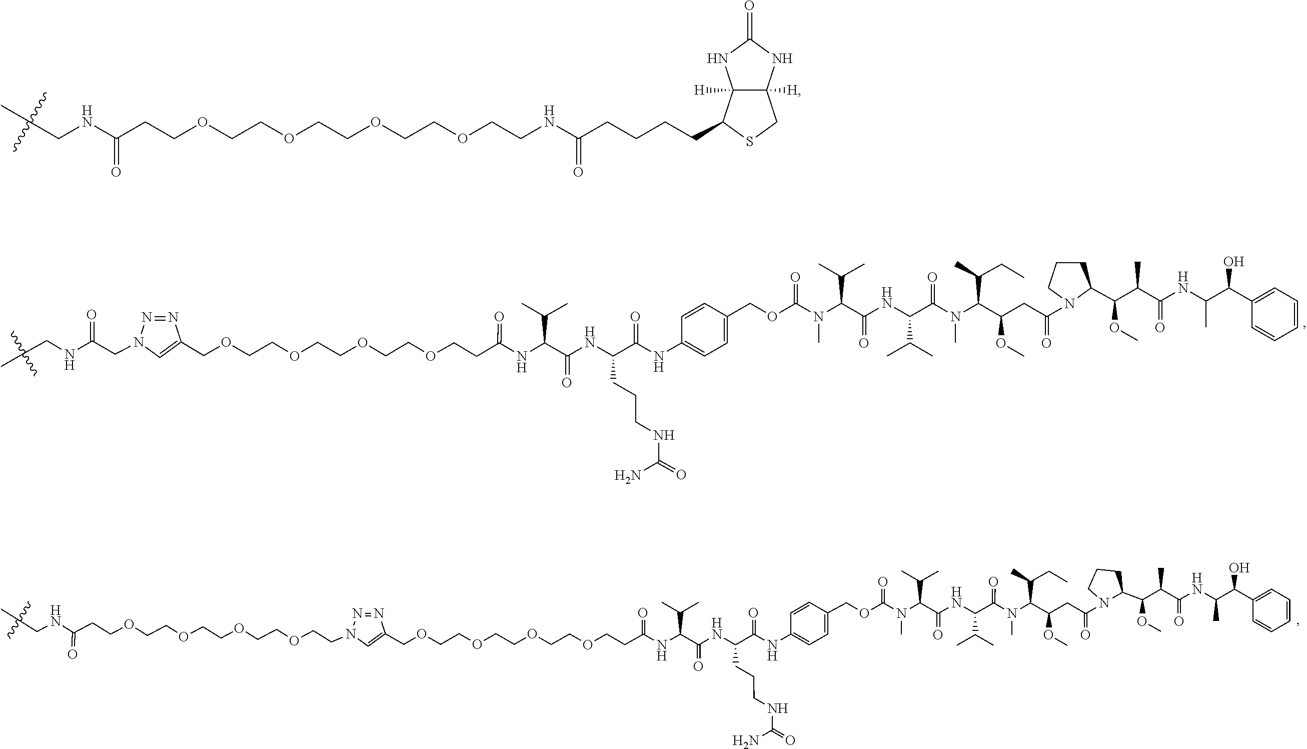

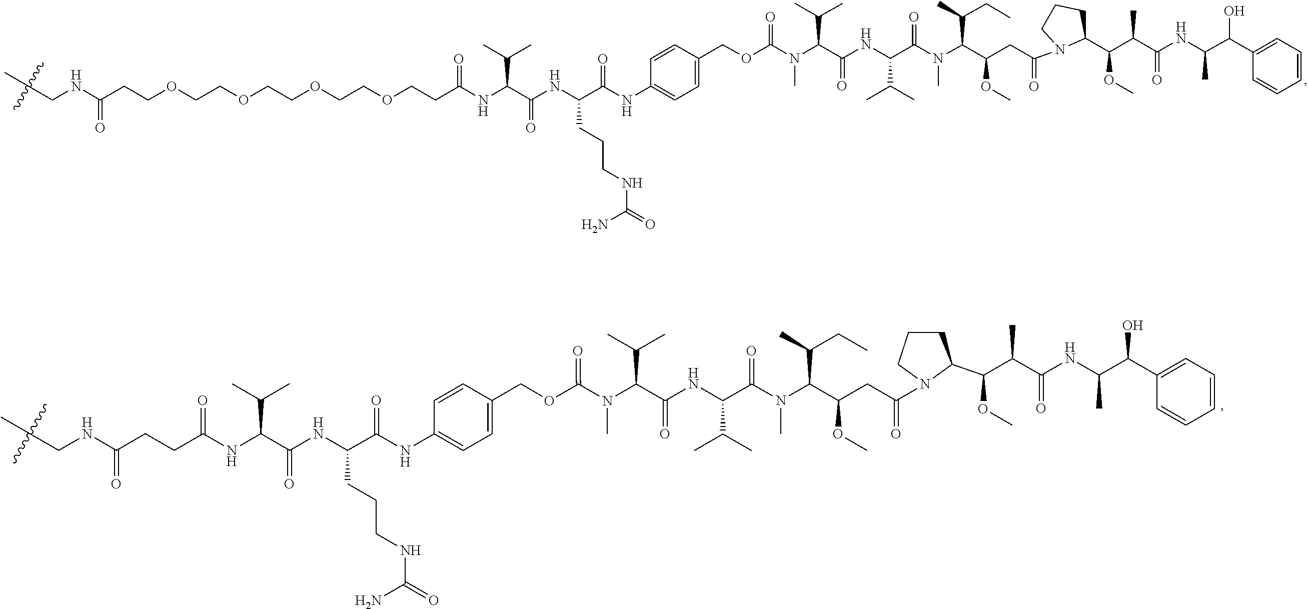 [SMILES: *CNC(=O)CCOCCOCCOCCOCCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)C(O)c2ccccc2)OC)C(C)C)C(C)C)cc1)C(C)C ; *CNC(=O)CCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C]

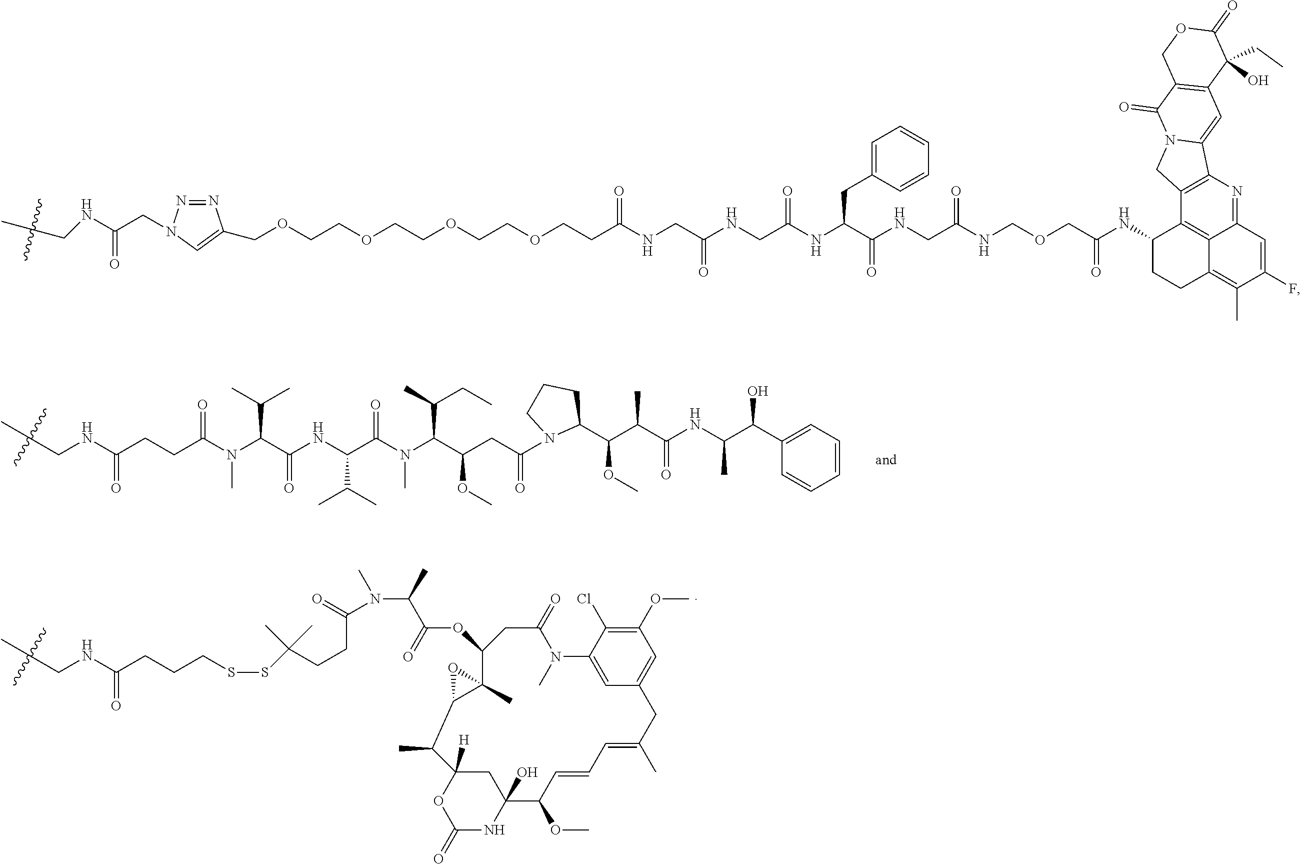

When in this case, the protein of the protein conjugate may be an antibody, and the protein conjugate may have the similar binding affinity towards an antigen, compared to the correspond antibody.

For example, the Fuc* may comprise a structure of

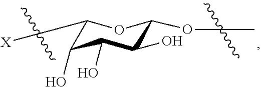

wherein the X is selected from:

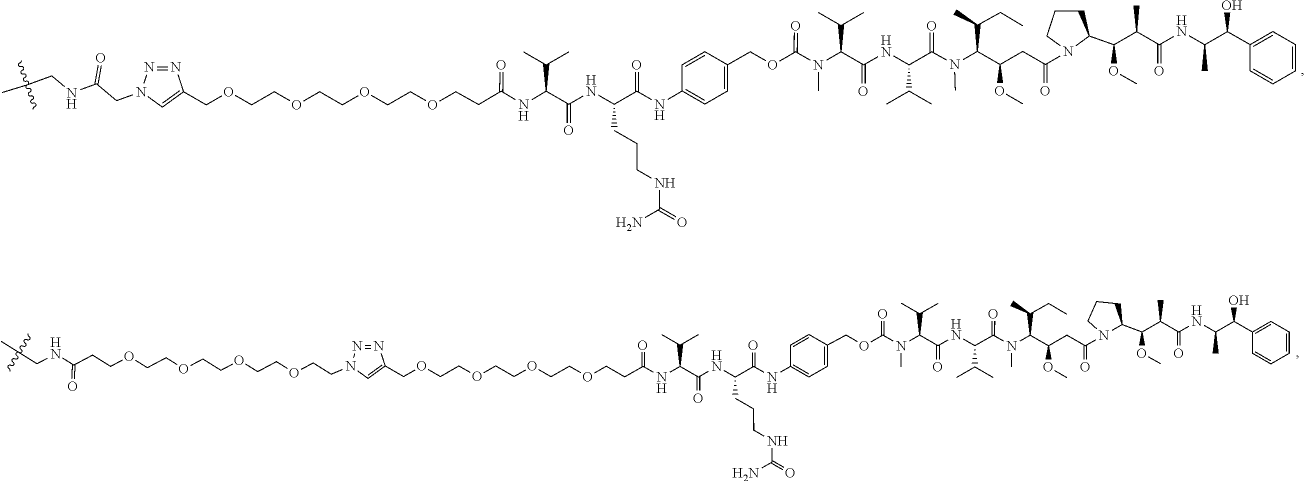 [SMILES: *CNC(=O)Cn1cc(COCCOCCOCCOCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)nn1 ; *CNC(=O)CCOCCOCCOCCOCCn1cc(COCCOCCOCCOCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)nn1]

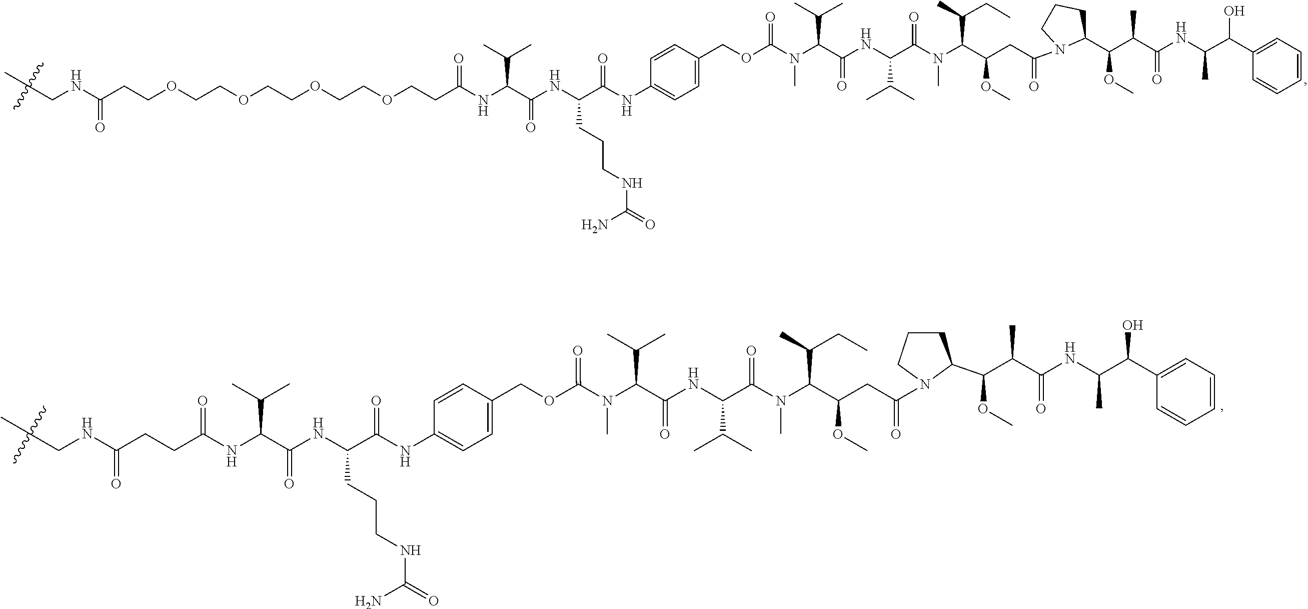 [SMILES: *CNC(=O)CCOCCOCCOCCOCCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)C(O)c2ccccc2)OC)C(C)C)C(C)C)cc1)C(C)C ; *CNC(=O)CCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C]

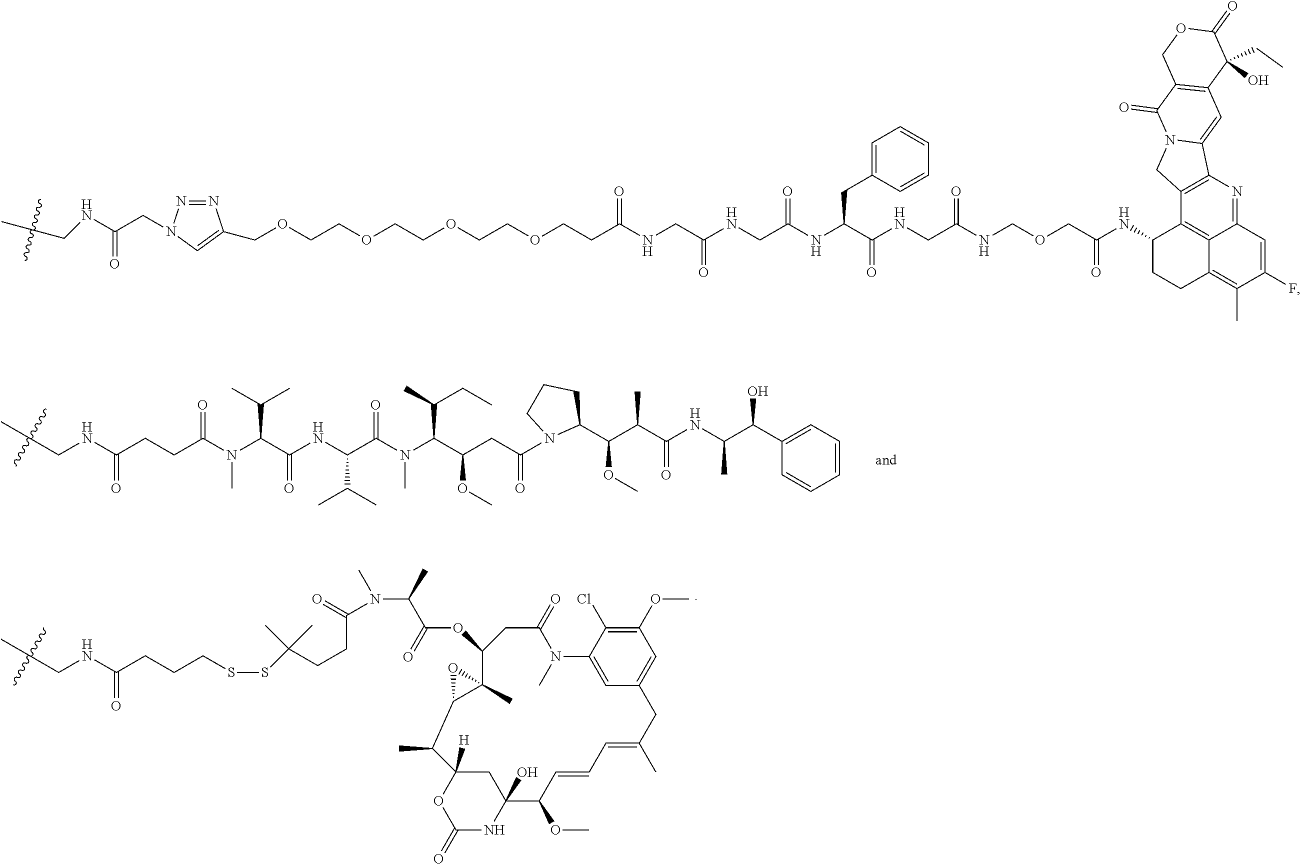

When in this case, the protein of the protein conjugate may be an antibody, and the protein conjugate may have the similar binding affinity towards an antigen, compared to the corresponding antibody.

For example, the Fuc* may comprise a structure of

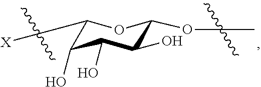

wherein the X is selected from:

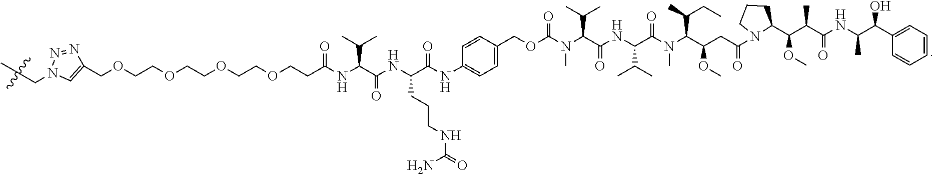 [SMILES: *Cn1cc(COCCOCCOCCOCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)nn1]

When in this case, the protein of the protein conjugate may be an antibody, and the protein conjugate may have the similar binding affinity towards an antigen, compared to the corresponding antibody.

In the present disclosure, a group Y₁Y₂ may be between the fucose of the Fuc* and the P. the group Y₁Y₂ may remained after a ligation reaction between the Y₁ and a functional group Y₂. The ligation reaction conjugates the P to the protein of the conjugate. For example, the ligation reaction may be a bioorthogonal reaction. For example, the Y₂ may comprise a functional moiety.

For example, the Y₂ may comprise a functional moiety selected from the group consisting of azide, terminal alkyne, cyclic alkyne, tetrazine, 1,2,4-trazine, terminal alkene, transcyclooctene, cyclopropene, norbornene, keto, aldehyde, aminooxy, thiol, and maleimide. For example, the functional moiety of Y₂ may be selected from the group consisting of azide derivative, terminal alkyne derivative, cyclic alkyne derivative, tetrazine derivative, 1,2,4-trazine derivative, terminal alkene derivative, transcyclooctene derivative, cyclopropene derivative, norbornene derivative, keto derivative, aldehyde derivative, aminooxy derivative, thiol derivative, and maleimide derivative.

In the present disclosure, the Y₂ may comprise a functional moiety selected from the group consisting of

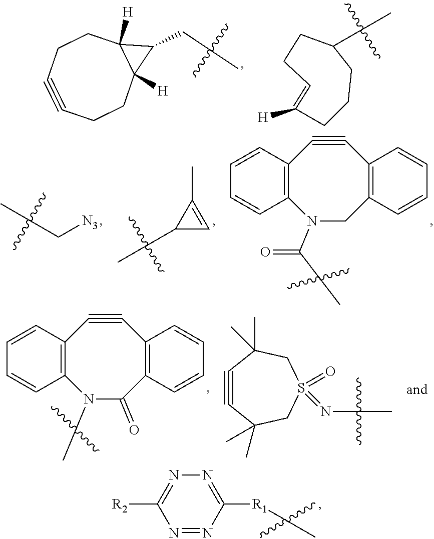

wherein each of R₁ and R₂ is independently selected from the group consisting of hydrogen, halogen, C₁-C₂₂ alkyl group, C₅-C₂₂ (hetero)aryl group, C₇-C₂₂ alkyl(hetero)aryl group and C₇-C₂₂ (hetero)arylalkyl group, wherein each of said alkyl group optionally is interrupted by one or more hetero-atom selected from the group consisting of O, N, and S, and wherein each of the alkyl group, (hetero)aryl group, alkyl(hetero)aryl group and (hetero)arylalkyl groups is independently optionally substituted.

For example, the remaining group Y₁Y₂ may be selected from the group consisting of

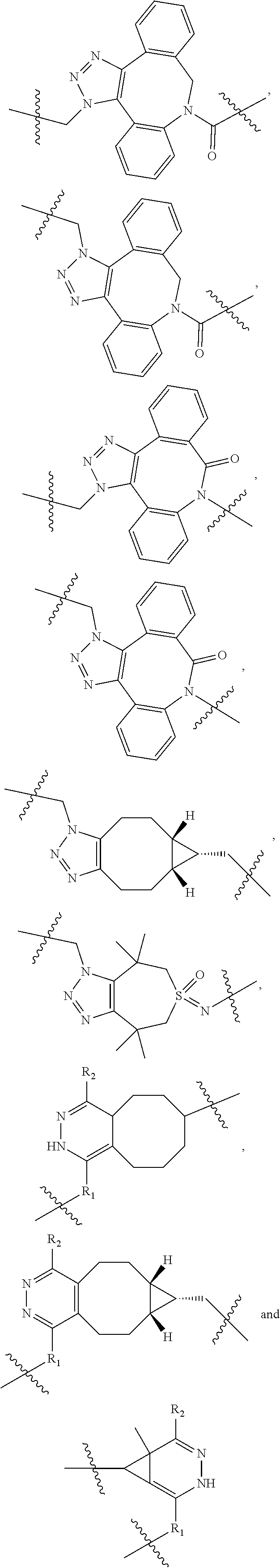

When Y₁ comprise a specific functional moiety, what the type of functional moiety of Y₂ can be selected is known to the art. For example, the Y₁ and the Y₂ may comprise the functional moiety selected from the group consisting of: a) Y₁ comprises

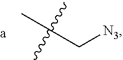 [SMILES: *CN=[N+]=[N-]]

and Y₂ comprises

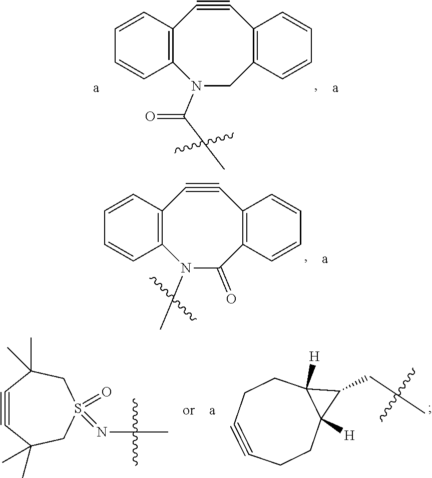 [SMILES: *C(=O)N1Cc2ccccc2C#Cc2ccccc21 ; *N1C(=O)c2ccccc2C#Cc2ccccc21 ; *N=S1(=O)CC(C)(C)C#CC(C)(C)C1 ; *C[C@H]1[C@H]2CCC#CCC[C@@H]12]

b) Y₁ comprises

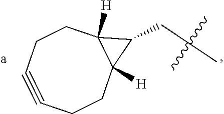 [SMILES: *C[C@H]1[C@H]2CCC#CCC[C@@H]12]

and Y₂ comprises

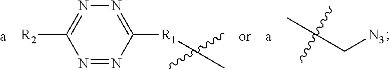

c) Y₁ comprises

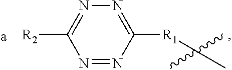

and Y₂ comprises

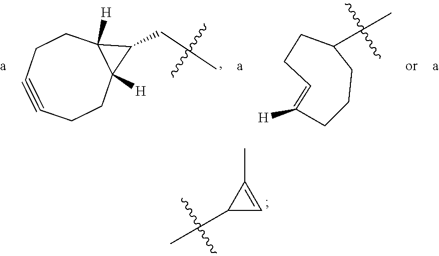

and d) Y₁ comprises

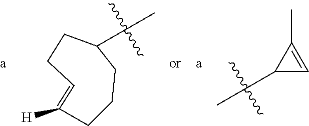

and Y₂ comprises

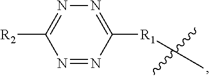

wherein each of R₁ and R₂ is independently selected from the group consisting of hydrogen, halogen, C₁-C₂₂ alkyl group, C₅-C₂₂ (hetero)aryl group, C₇-C₂₂ alkyl(hetero)aryl group and C₇-C₂₂ (hetero)arylalkyl group, wherein each of said alkyl group optionally is interrupted by one or more hetero-atom selected from the group consisting of O, N, and S, and wherein each of the alkyl group, (hetero)aryl group, alkyl(hetero)aryl group and (hetero)arylalkyl groups is independently optionally substituted.

In the present disclosure, the biologically and/or pharmaceutically active substance P may be conjugated to the protein by an enzyme catalyzed reaction.

In the present disclosure, the Fuc* may have the structure of Fuc-(F)ₘ-(L)ₙ-Y₁Y₂-(FL′)_{m′}-(L′)_{n′}-P, wherein Fuc is a fucose, F is the connector, Y₁Y₂ is the remaining group, L is the linker, L′ is a linker defined as the same as L, FL′ is a spacer defined as the same as the FL, P is the biologically and/or pharmaceutically active substance, each of the m, n, m′ and n′ is independently 0 or 1. wherein F, Y₁Y₂, L and P are defined as above.

For example, the Fuc* may have the structure of Fuc-F-L-Y₁Y₂-FL′-L′-P, wherein the F is

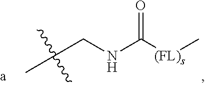

and FL may be selected from the group consisting of

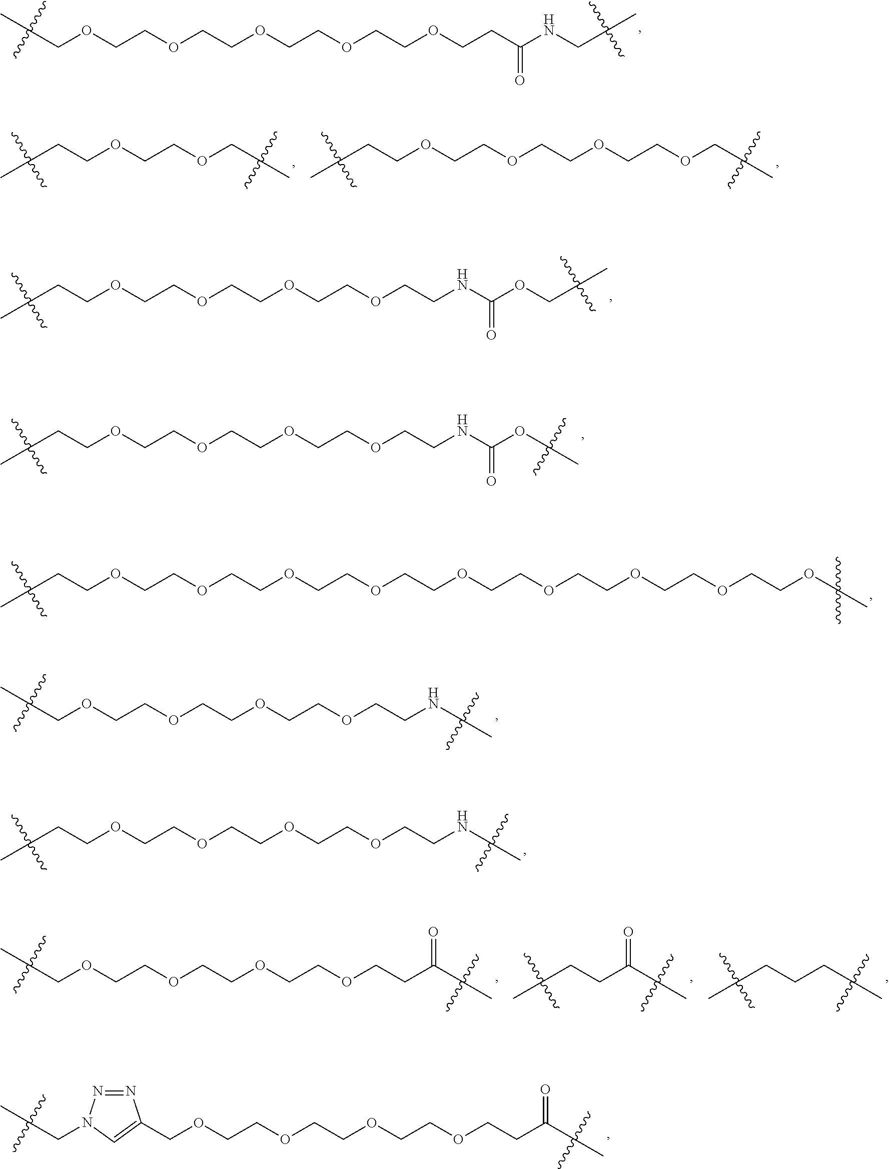

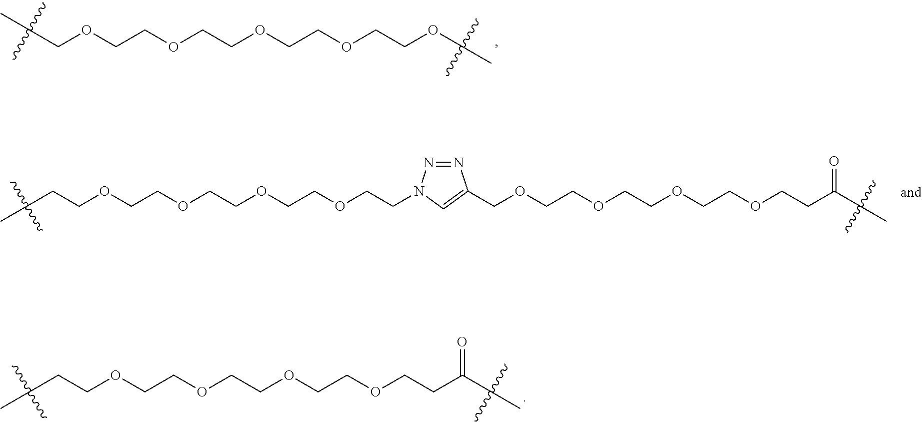 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

And FL′ may be selected from the group consisting of

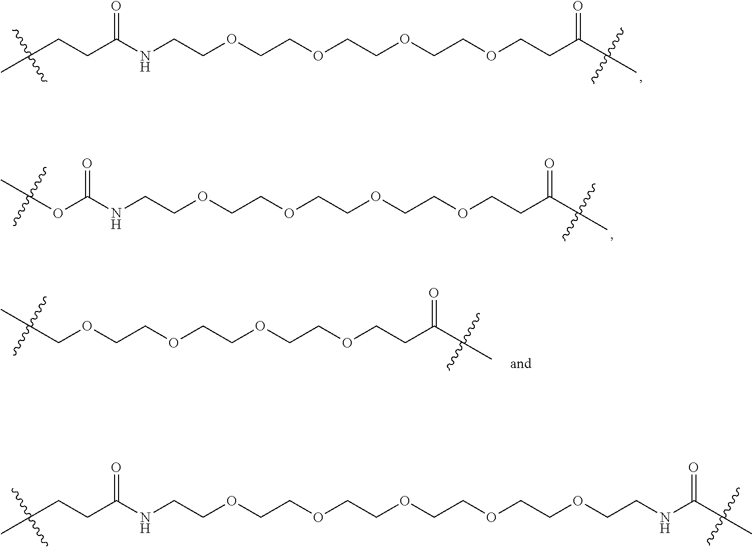 [SMILES: *CCC(=O)NCCOCCOCCOCCOCCC(*)=O ; *OC(=O)NCCOCCOCCOCCOCCC(*)=O ; *COCCOCCOCCOCCC(*)=O ; *CCC(=O)NCCOCCOCCOCCOCCOCCNC(*)=O]

For example the Fuc* may comprise a structure of

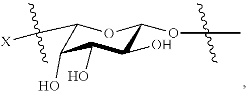

wherein the X is selected from:

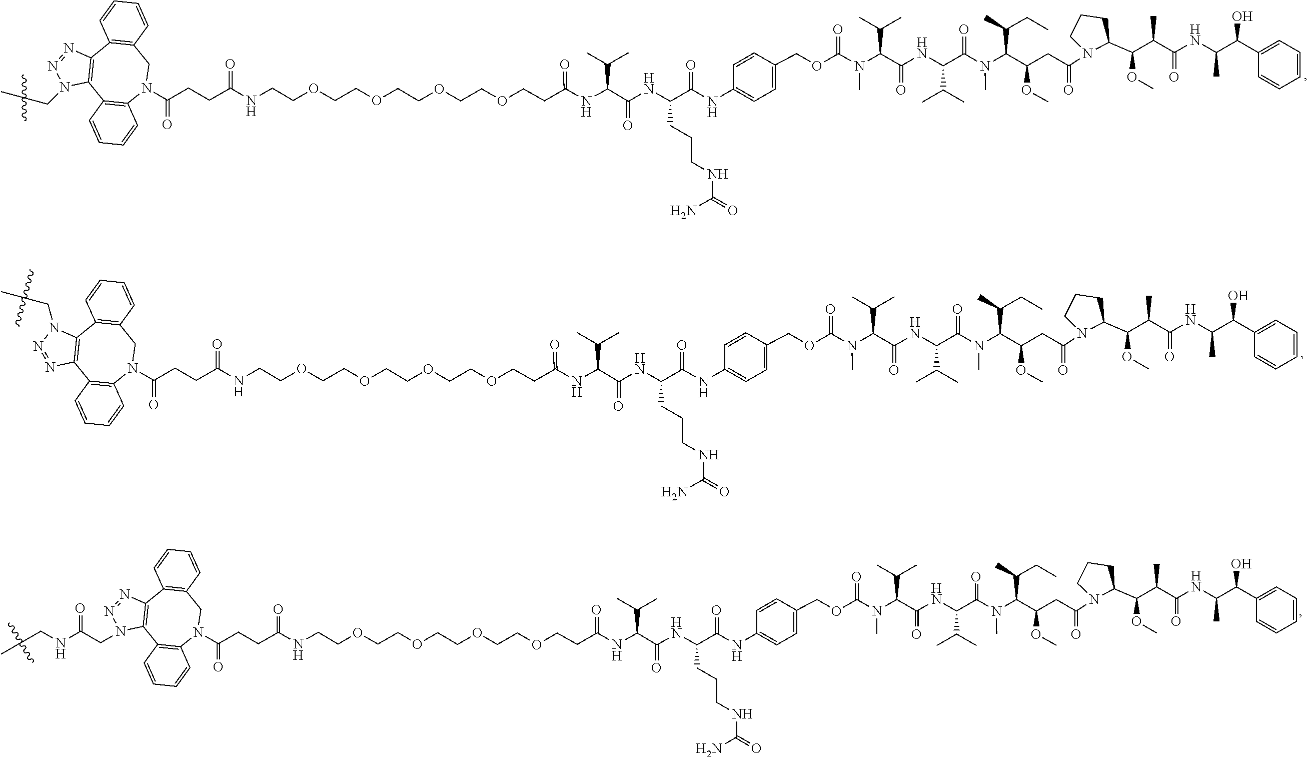

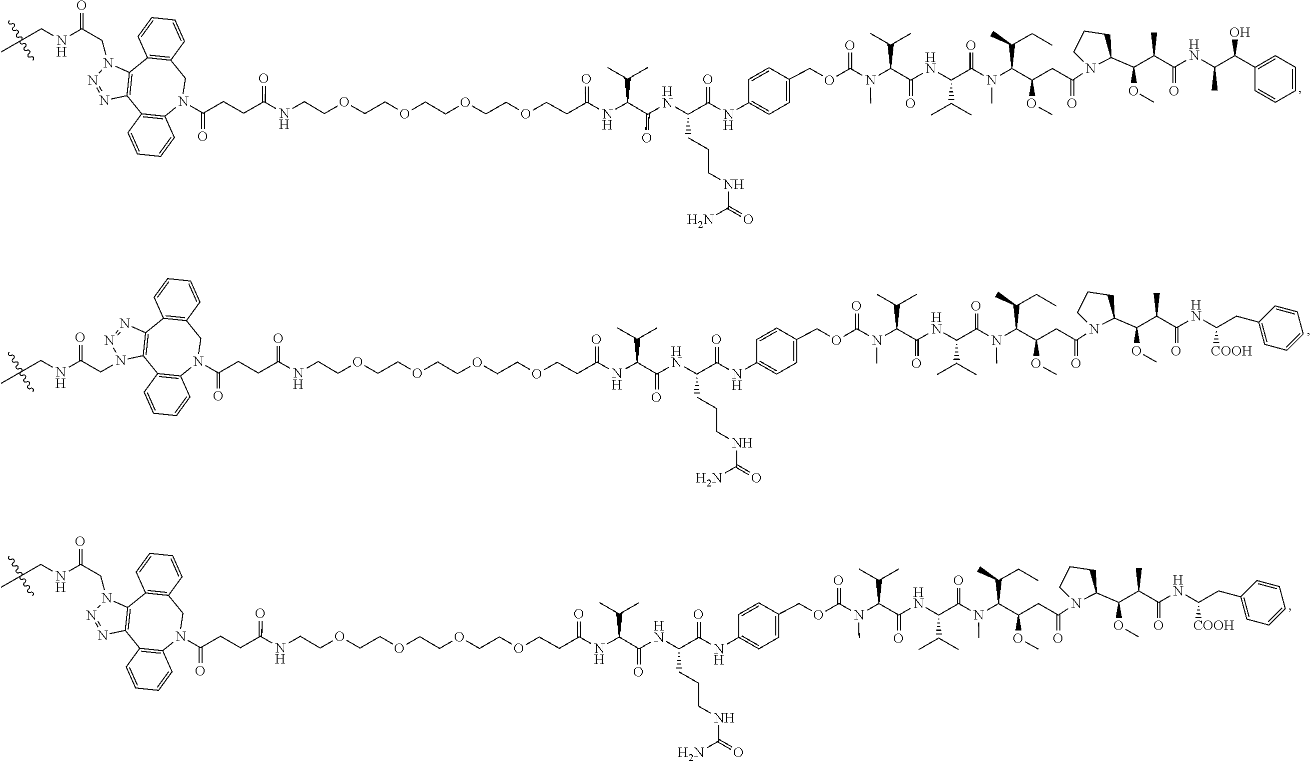

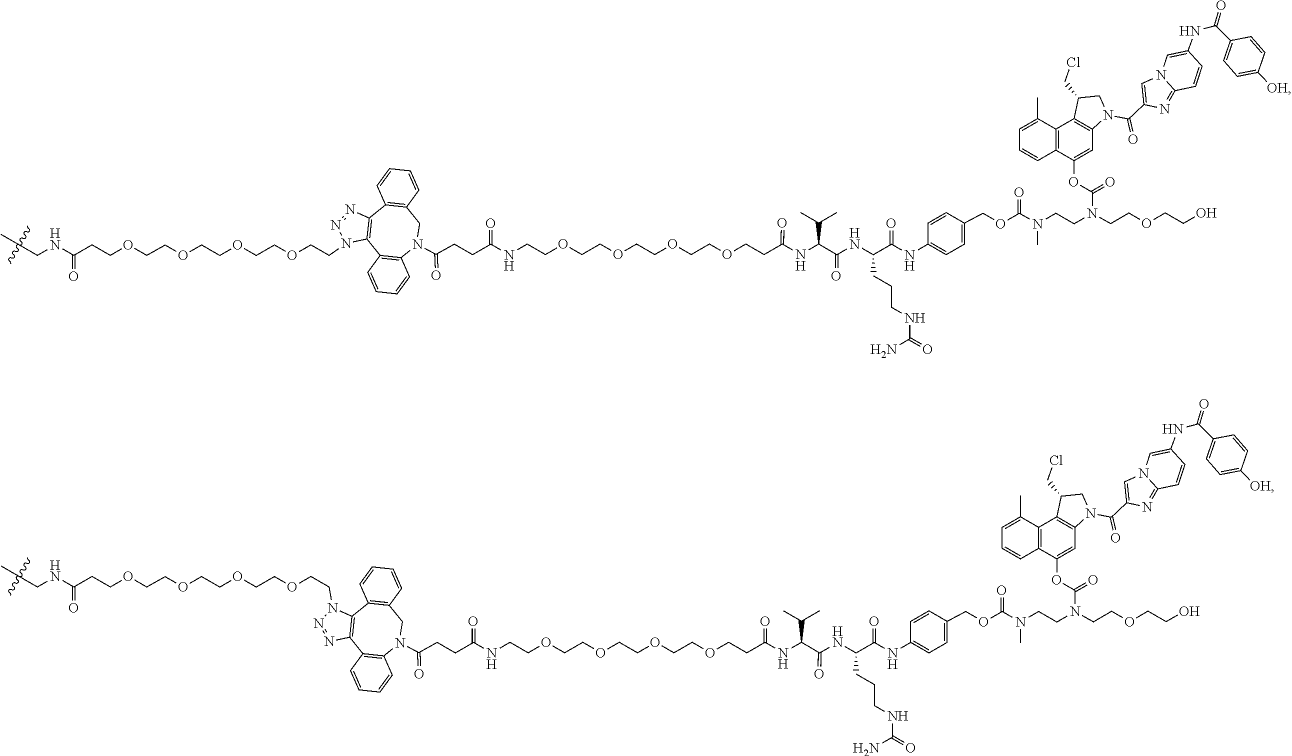 [SMILES: *CNC(=O)CCOCCOCCOCCOCCn1nnc2c1-c1ccccc1N(C(=O)CCC(=O)NCCOCCOCCOCCOCCC(=O)N[C@H](C(=O)N[C@@H](CCCNC(N)=O)C(=O)Nc1ccc(COC(=O)N(C)CCN(CCOCCO)C(=O)Oc3cc4c(c5c(C)cccc35)[C@H](CCl)CN4C(=O)c3cn4cc(NC(=O)c5ccc(O)cc5)ccc4n3)cc1)C(C)C)Cc1ccccc1-2 ; *CNC(=O)CCOCCOCCOCCOCCn1nnc2c1-c1ccccc1CN(C(=O)CCC(=O)NCCOCCOCCOCCOCCC(=O)N[C@H](C(=O)N[C@@H](CCCNC(N)=O)C(=O)Nc1ccc(COC(=O)N(C)CCN(CCOCCO)C(=O)Oc3cc4c(c5c(C)cccc35)[C@H](CCl)CN4C(=O)c3cn4cc(NC(=O)c5ccc(O)cc5)ccc4n3)cc1)C(C)C)c1ccccc1-2]

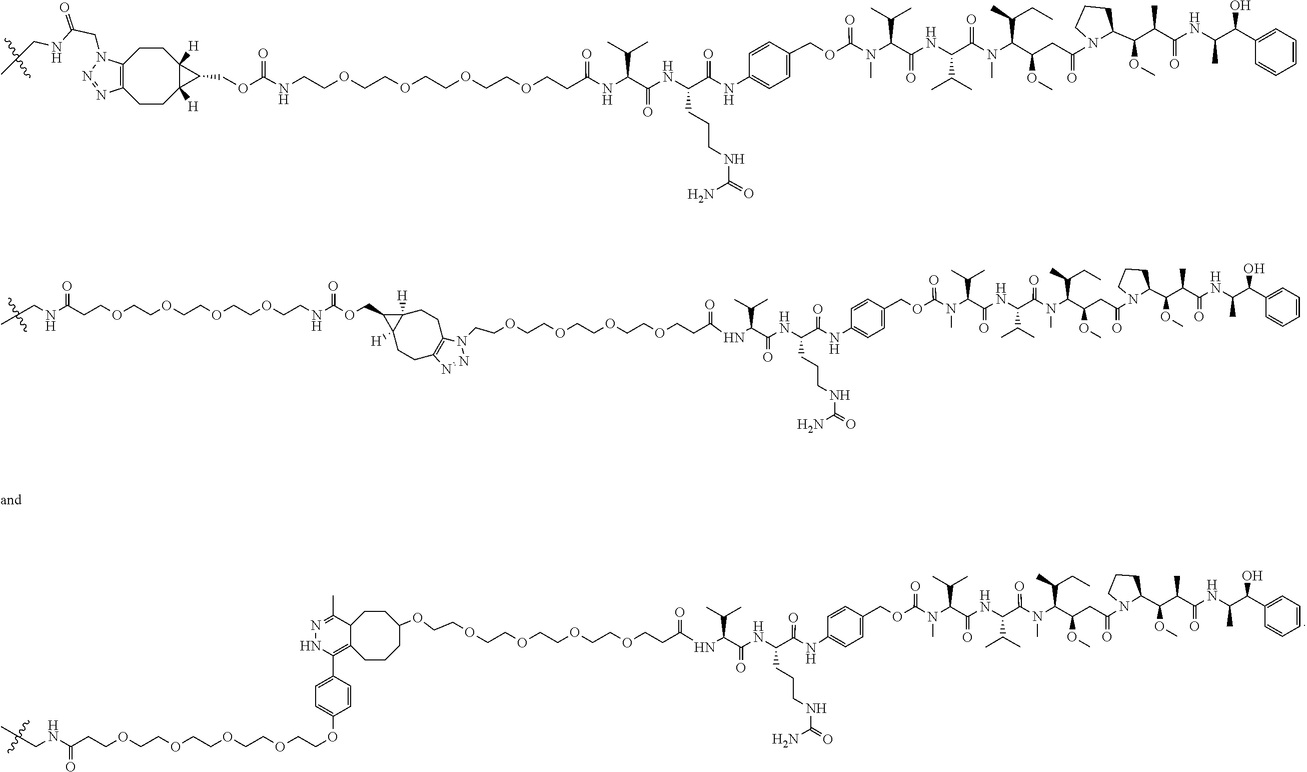

When in this case, the protein of the protein conjugate may be an antibody, and the protein conjugate may have the similar binding affinity towards an antigen, compared to the corresponding antibody. In some embodiments, the protein conjugate is for treating disease.

In the present disclosure, the Fuc* comprise a fucose or a fucose derivative. The structure of the fucose or a fucose derivative in the Fuc* may be

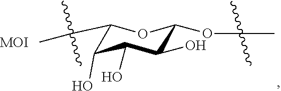

wherein the MOI is the molecule of interest as defined above.

In the present disclosure, the oligosaccharide of the protein conjugate comprises

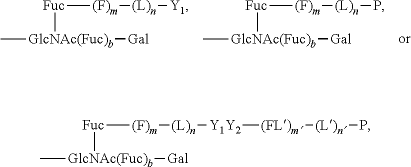

wherein, Fuc, (Fuc), F, FL′, L, L′, GlcNAc, Y₁, Y₁Y₂, P, Gal are defined as above, b is 0 or 1, m is 0 or 1, n is 0 or 1, m′ is 0 or 1, and n′ is 0 or 1.

For example, the Fuc* is linked to the GlcNAc of a terminal LacNAc of the

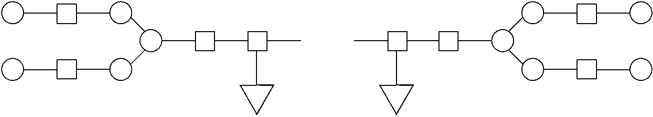

through an Fuc*α1,3GlcNAc linkage wherein [symbol] is a GlcNAc, [symbol] is the fucose of (Fuc) linked a core GlcNAc through an α1,6 linkage, [symbol] is a mannose, ◯ is a galactose linked to a GlcNAc through a Galβ1,4GlcNAc linkage, and [symbol] is an antibody or a Fc-fusion protein.

For example, the protein conjugate of the resent disclosure may be according to the formula

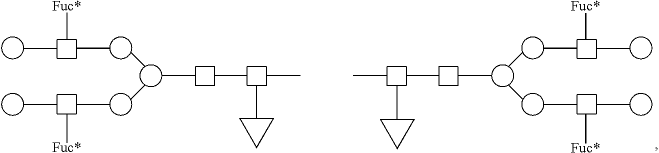

wherein [symbol] is the GlcNAc, [symbol] is the fucose of (Fuc) linked the GlcNAc through an α1,6 linkage, [symbol] is the mannose, ◯ is the galactose linked to the GlcNAc through a Galβ1,4GlcNAc linkage, Fuc* is linked to the GlcNAc through an Fuc*α1,3GlcNAc linkage, and [symbol] is an antibody or a Fc fusion protein. The oligosaccharide may be linked to the N297 position of the Fc fragment. For example, the [symbol] is an antibody. For example, the protein conjugate may have the similar binding affinity towards an antigen, compared to the corresponding antibody. For example, the protein conjugate is for treating disease. For example, when the protein conjugates comprise the Y₁, the protein conjugates is for making agents for treating disease

In the present disclosure, the protein conjugate may have the formula of

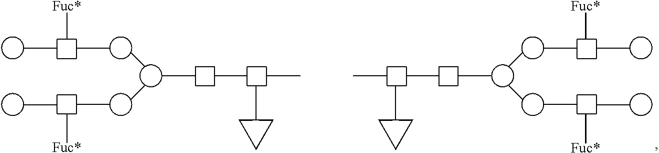

wherein, wherein [symbol] is the GlcNAc, [symbol] is the fucose of (Fuc) linked the GlcNAc through an α1,6 linkage, [symbol] is a mannose, ◯ is the galactose [symbol] linked to the GlcNAc through a Galβ1,4GlcNAc linkage, Fuc* is linked to the GlcNAc through an Fuc*α1,3GlcNAc linkage, [symbol] is an antibody or a Fc fusion protein, and Fuc* is Fuc-(F)ₘ-(L)ₙ-Y₁, wherein, Fuc is according to the formula

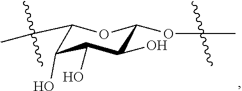

### F is

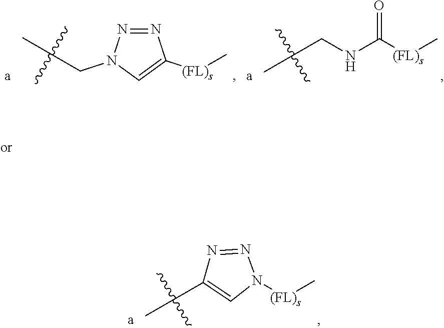

wherein FL is a polypeptide, a PEG, an alkyl and/or their derivatives or combination thereof, L is a cleavable linker, Y₁ comprises a functional moiety capable of participating in a bioorthogonal reaction, s is 0 or 1, m is 0 or 1 and n is 0 or 1. For example, the [symbol] is an antibody. For example, the protein conjugate has a similar binding affinity as its corresponding antibody towards an antigen. For example, the protein conjugate is for making an agent for treating diseases.

In the present disclosure, the protein conjugate may have the formula of

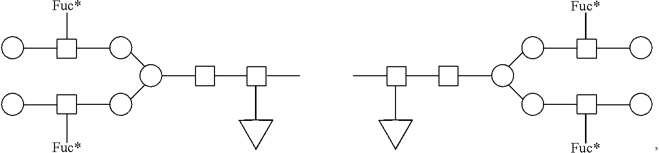

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is an antibody, and Fuc* is Fuc-F-(L)ₙ-Y₁, wherein, Fuc is according to the formula

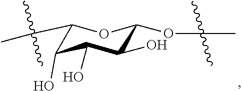

### F is

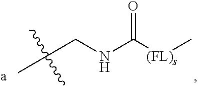

wherein FL is a polypeptide, a PEG, an alkyl and/or their derivatives or combination thereof, L is a cleavable linker, Y₁ comprises a functional moiety capable of participating in a bioorthogonal reaction, s is 0 or 1 and n is 0 or 1. For example, the protein conjugate has a similar binding affinity as its corresponding antibody towards an antigen. For example, the protein conjugate is for making agents for treating diseases.

In the present disclosure, the protein conjugate may have the formula of

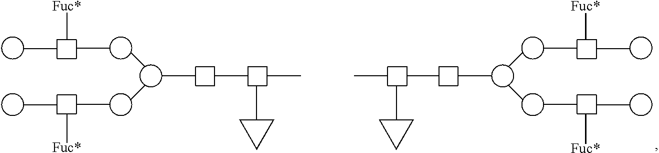

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is antibody, and Fuc* is Fuc-F-Y₁, wherein, Fuc is according to the formula

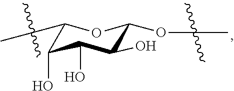

### F is

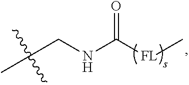

wherein FL is a polypeptide, a PEG, an alkyl and/or their derivatives or combination thereof, Y₁ comprises a functional moiety capable of participating in a bioorthogonal reaction, s is 0 or 1 and the

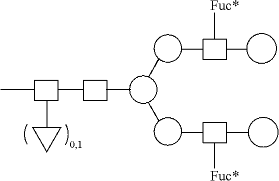

is linked to the N297 position of the antibody. For example, the protein conjugate has a similar binding affinity as its corresponding antibody towards an antigen. For example, the protein conjugate is for making an agent for treating diseases.

In the present disclosure, the protein conjugate may have the formula of

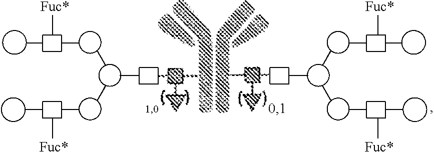

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is an antibody or a Fc fusion protein, and Fuc* is Fuc-(F)ₘ-(L)ₙ-P, wherein, Fuc is according to the formula

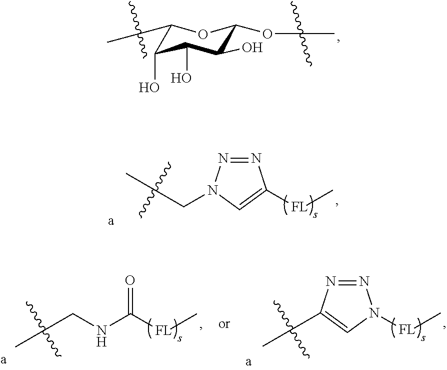

wherein FL is a polypeptide, a PEG, an alkyl and/or their derivatives or combination thereof, L is a cleavabe linker, P is a biologically and/or a pharmaceutically active substance, s is 0 or 1, m is 0 or 1, and n is 0 or 1. For example, the [symbol] is an antibody. For example, the protein conjugate has a similar binding affinity as its corresponding antibody towards an antigen. For example, the protein conjugate is for treating disease.

In the present disclosure, the protein conjugate may have the formula of

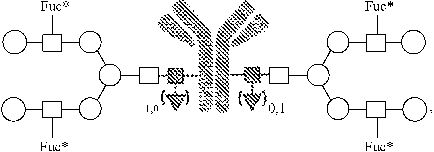

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is an antibody, and Fuc* is Fuc-F-(L)ₙ-P, wherein, Fuc is according to the formula

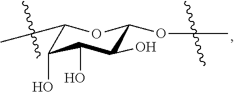

### F is

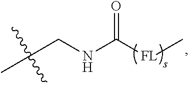

wherein FL is a polypeptide, a PEG, an alkyl and/or their derivatives or combination thereof, L is a cleavable linker, P is a pharmaceutically active substance, s is 0 or 1 and n is 0 or 1. For example, the protein conjugate has a similar binding affinity as its corresponding antibody towards an antigen. For example, the protein conjugate is for treating disease.

In the present disclosure, the protein conjugate may have the formula of

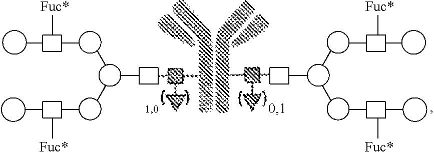

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is an antibody, and Fuc* is Fuc-F-L-P, wherein, Fuc is according to the formula

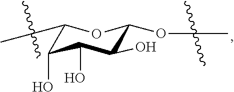

### F is a

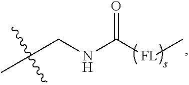

wherein FL is a polypeptide, a PEG, an alkyl and/or their derivatives or combination thereof, L is a cleavable linker, and P is a pharmaceutically active substance, s is 0 or 1 and the

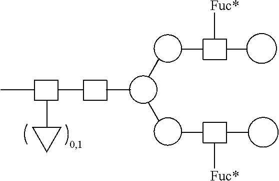

is linked to the N297 position of the antibody. For example, the protein conjugate has a similar binding affinity as its corresponding antibody towards an antigen. For example, the protein conjugate is for treating disease.

In the present disclosure, the protein conjugate may have the formula of

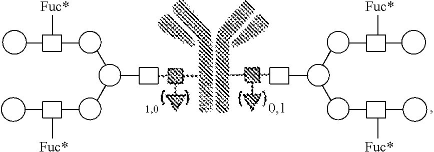

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is an antibody or a Fc fusion protein, and Fuc* is Fuc-(F)ₘ-(L)ₙ-Y₁Y₂(FL′)_{m′}-(L′)_{n′}-P, wherein, Fuc is according to the formula

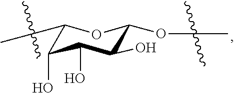

### F is a

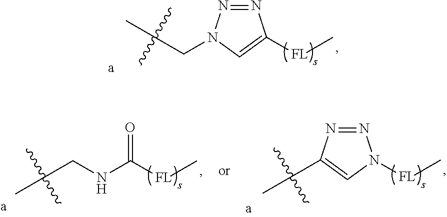

wherein FL is a polypeptide, a PEG, an alkyl and/or their derivatives or combination thereof, s is 0 or 1, L is a cleavable linker, Y₁Y₂ is a remaining group after a ligation reaction, FL′ is a polypeptide, a PEG, an alkyl and/or their derivatives or combination thereof, L is a cleavable linker, P is a biologically and/or a pharmaceutically active substance, m is 0 or 1, n is 0 or 1, m′ is 0 or 1 and n′ is 0 or 1. For example, the [symbol] is an antibody. For example, the protein conjugate has a similar binding affinity as its corresponding antibody towards an antigen. For example, the protein conjugate is for treating disease.

In the present disclosure, the protein conjugate may have the formula of

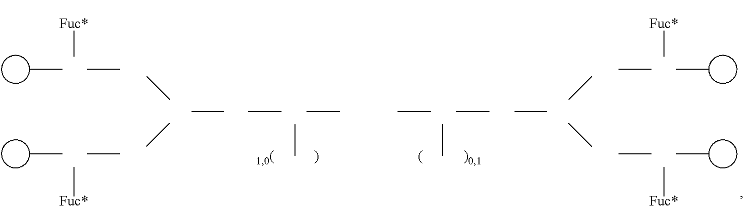

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is an antibody, and Fuc* is Fuc-Fuc-F-Y₁Y₂-(FL′)_{n′}-L′-P, wherein, Fuc is according to the formula

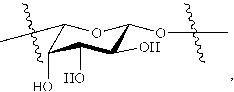

### F is a

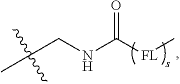

wherein FL is a polypeptide, a PEG, an alkyl and/or their derivatives or combination thereof, s is 0 or 1, Y₁Y₂ is a remaining group after a ligation reaction, L′ is a cleavable linker, P is a pharmaceutically active substance (e.g., a toxicin). and n′ is 0 or 1, and the protein conjugate has a similar binding affinity as its corresponding antibody towards an antigen. For example, the protein conjugate is for treating disease.

In the present disclosure, the protein conjugate may have the formula of

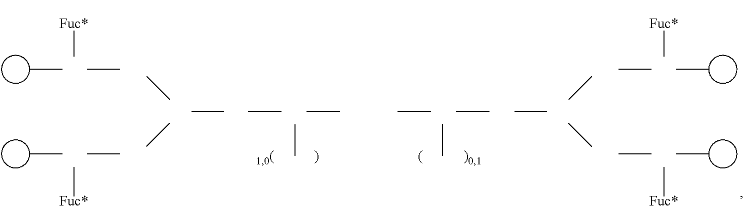

wherein, wherein [symbol] is the GlcNAc, [symbol] is the fucose of (Fuc) linked the GlcNAc through an α1,6 linkage, [symbol] is a mannose, ◯ is the galactose linked to the GlcNAc through a Galβ1,4GlcNAc linkage, Fuc* is linked to the GlcNAc through an Fuc*α1,3GlcNAc linkage, [symbol] is antibody, and Fuc* is Fuc-F-Y₁Y₂-FL′-L′-P, wherein, Fuc is according to the formula

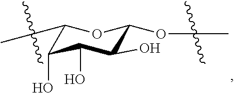

### F is a

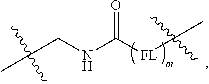

wherein FL is a polypeptide, a PEG, an alkyl and/or their derivatives or combination thereof, s is 0 or 1, Y₁Y₂ is a remaining group after a ligation reaction, FL′ is a polypeptide, a PEG, an alkyl and/or their derivatives or combination thereof, L is a cleavable linker, P is a pharmaceutically active substance (e.g., a toxicin), and the

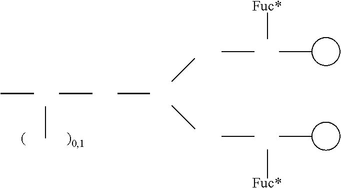

is linked to the N297 position of the antibody. For example, the protein conjugate has a similar binding affinity as its corresponding antibody towards an antigen. For example, the protein conjugate is for treating disease.

In the present disclosure, the Fuc* is linked to the core GlcNAc of

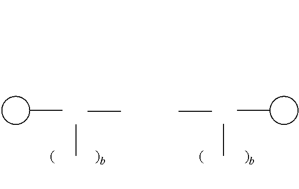

through an Fuc*α1,3GlcNAc linkage, wherein [symbol] is a GlcNAc, [symbol] is the fucose of (Fuc) linked the core GlcNAc through an α1,6 linkage, ◯ is a galactose linked to a GlcNAc through a Galβ1,4GlcNAc linkage, [symbol] is an antibody or a Fc-fusion protein and b is 0 or 1. In some embodiments, the protein conjugate may have one or two molecules (preferably, two molecules) of interest conjugated in the GlcNAc.

For example, the protein conjugate is according to the formula

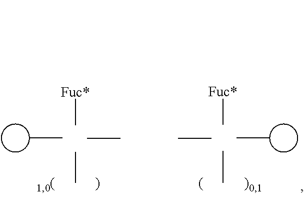

wherein said [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, and [symbol] is an antibody or a Fc fusion protein comprising a Fc fragment. The GlcNAc may be linked to the N297 position of the protein. For example, the [symbol] is an antibody. For example, the protein conjugate may have the similar binding affinity towards an antigen, compared to the corresponding antibody. For example, the protein conjugate is for treating disease. For example, when the protein conjugates comprise the Y₁, the protein conjugates is for making an agent for treating disease.

For example, the protein conjugate is according to the formula

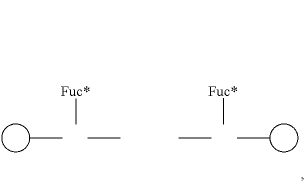

wherein said [symbol] is the GlcNAc, ◯ is the galactose linked to the GlcNAc through a Galβ1,4GlcNAc linkage, Fuc* is linked to the GlcNAc through an Fuc*α1,3GlcNAc linkage, and [symbol] is an antibody or a Fc fusion protein comprising a Fc fragment. The GlcNAc may be linked to the N297 position of the protein. For example, the [symbol] is an antibody. For example, the protein conjugate may have the similar binding affinity towards an antigen, compared to the corresponding antibody. For example, the protein conjugate is for treating disease. For example, when the protein conjugates comprise the Y₁, the protein conjugates is for making an agent for treating disease.

For example, the protein conjugate is according to the formula

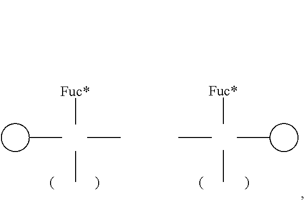

wherein said [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, and [symbol] is an antibody or a Fc fusion protein comprising a Fc fragment. The GlcNAc may be linked to the N297 position of the protein. For example, the [symbol] is an antibody. For example the protein conjugate may have the similar binding affinity towards an antigen, compared to the corresponding antibody For example, the protein conjugate is for treating disease. For example, when the protein conjugates comprise the Y₁, the protein conjugates is for making an agent for treating disease.

In the present disclosure, the protein conjugate may have the formula of

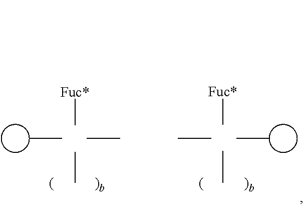

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody or a Fc fusion protein, b is 0 or 1, and Fuc* is Fuc-(F)ₘ-(L)ₙ-Y₁, wherein, Fuc is according to the formula

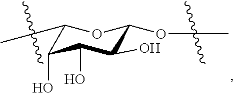

### F

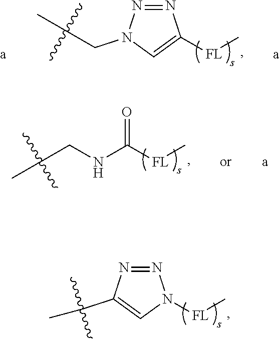

wherein FL is a polypeptide, a PEG, an alkyl and/or their derivatives or combination thereof, L is a cleavable linker, Y₁ comprises a functional moiety capable of participating in a bioorthogonal reaction, s is 0 or 1, m is 0 or 1, and n is 0 or 1. For example, the [symbol] is an antibody. For example, the protein conjugate has a similar binding affinity as its corresponding antibody towards an antigen. For example, the protein conjugate is for making an agent for treating diseases.

In the present disclosure, the protein conjugate may have the formula of

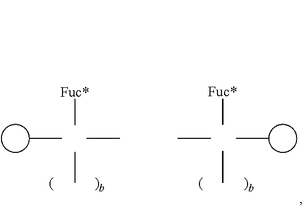

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody, b is 0 or 1 and Fuc* is Fuc-F-(L)ₙ-Y₁, wherein, Fuc is according to the formula

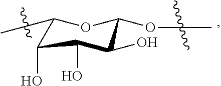

### F is a

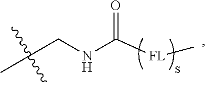

wherein FL is a polypeptide, a PEG, an alkyl and/or their derivatives or combination thereof, L is a cleavable linker, Y₁ comprises a functional moiety capable of participating in a bioorthogonal reaction, s is 0 or 1 and n is 0 or 1. For example, the protein conjugate has a similar binding affinity as its corresponding antibody towards an antigen. For example, the protein conjugate is for making an agent for treating diseases.

In the present disclosure, the protein conjugate may have the formula of

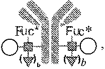

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody, and Fuc* is Fuc-F-Y₁, wherein, Fuc is according to the formula

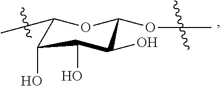

### F is a

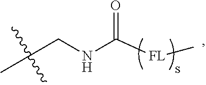

wherein FL is a polypeptide, a PEG, an alkyl and/or their derivatives or combination thereof, Y₁ comprises a functional moiety capable of participating in a bioorthogonal reaction, s is 0 or 1 and the

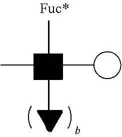

is linked to the N297 position of the antibody. For example, the protein conjugate has a similar binding affinity as its corresponding antibody towards an antigen. For example, the protein conjugate is for making an agent for treating diseases.

In the present disclosure, the protein conjugate may have the formula of

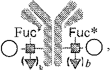

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody or a Fc fusion protein, b is 0 or 1, and Fuc* is Fuc-(F)ₘ-(L)ₙ-P, wherein, Fuc is according to the formula

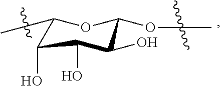

### F is

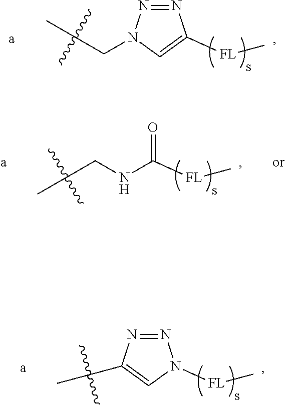

wherein FL is a polypeptide, a PEG, an alkyl and/or their derivatives or combination thereof, L is a cleavable linker, P is a biologically and/or a pharmaceutically active substance, s is 0 or 1, and n is 0 or 1. For example, the [symbol] is an antibody. For example, the protein conjugate has a similar binding affinity as its corresponding antibody towards an antigen. For example, the protein conjugate is for treating disease.

In the present disclosure, the protein conjugate may have the formula of

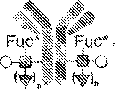

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody, b is 0 or 1 and Fuc* is Fuc-F-(L)ₙ-P, wherein, Fuc is according to the formula

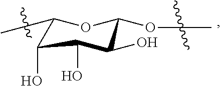

### F is a

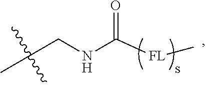

wherein FL is a polypeptide, a PEG, an alkyl and/or their derivatives or combination thereof, L is a cleavable linker, P is a pharmaceutically active substance, s is 0 or 1 and n is 0 or 1. For example, the protein conjugate has a similar binding affinity as its corresponding antibody towards an antigen. For example, the protein conjugate is for treating diseases.

In the present disclosure, the protein conjugate may have the formula of

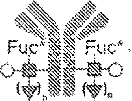

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody, b is 0 or 1 and Fuc* is Fuc-F-L-P, wherein, Fuc is according to the formula

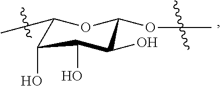

### F is a

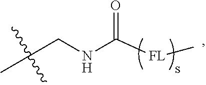

wherein FL is a polypeptide, a PEG, an alkyl and/or their derivatives or combination thereof, L is a cleavable linker, and P is a pharmaceutically active substance, and the

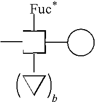

is linked to the N297 position of the antibody. For example, the protein conjugate has a similar binding affinity as its corresponding antibody towards an antigen. For example, the protein conjugate is for treating diseases.

In the present disclosure, the protein conjugate may have the formula of

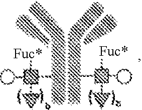

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody or a Fc fusion protein, b is 0 or 1 and Fuc* is Fuc-(F)ₘ-(L)ₙ-Y₁Y₂-(FL′)_{m′}-(L′)_{n′}-P, wherein, Fuc is according to the formula

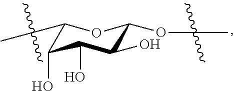

### F is a

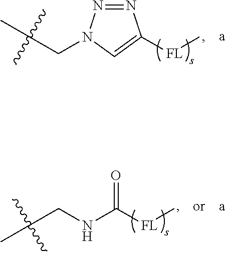

wherein FL is a polypeptide, a PEG, an alkyl and/or their derivatives or combination thereof, s is 0 or 1, L is a cleavable linker, Y₁Y₂ is a remaining group after a ligation reaction, FL′ is a polypeptide, a PEG, an alkyl and/or their derivatives or combination thereof, L′ is a cleavable linker, P is a biologically and/or a pharmaceutically active substance, m is 0 or 1, n is 0 or 1, m′ is 0 or 1 and n′ is 0 or 1. For example, the [symbol] is an antibody. For example, the protein conjugate has a similar binding affinity as its corresponding antibody towards an antigen. For example, the protein conjugate is for treating diseases.

In the present disclosure, the protein conjugate may have the formula of

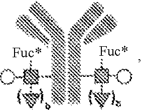

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody, b is 0 or 1 and Fuc* is Fuc-Fuc-F-Y₁Y₂-(FL′)_{n′}-L′-P, wherein, Fuc is according to the formula

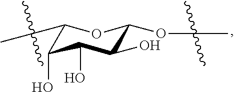

### F is

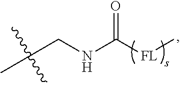

wherein FL is a polypeptide, a PEG, an alkyl and/or their derivatives or combination thereof, s is 1 or 0, Y₁Y₂ is a remaining group after a ligation reaction, L′ is a cleavable linker, P is a pharmaceutically active substance (e.g., a toxicin), and n′ is 0 or 1. For example, the protein conjugate has a similar binding affinity as its corresponding antibody towards an antigen. For example, the protein conjugate is for treating diseases.

In the present disclosure, the protein conjugate may have the formula of

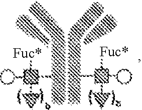

wherein, wherein [symbol] is the GlcNAc, [symbol] is the fucose of (Fuc) linked the GlcNAc through an α1,6 linkage, ◯ is the galactose linked to the GlcNAc through a Galβ1,4GlcNAc linkage, Fuc* is linked to the GlcNAc through an Fuc*α1,3GlcNAc linkage, [symbol] is an antibody, b is 0 or 1 and Fuc* is Fuc-F-Y₁Y₂-FL′-L′-P, wherein, Fuc is according to the formula

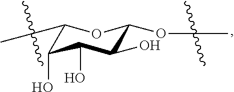

is

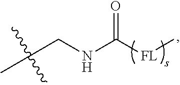

wherein FL is a polypeptide, a PEG, an alkyl and/or their derivatives or combination thereof, s is 1 or 0, Y₁Y₂ is a remaining group after a ligation reaction, FL′ is a polypeptide, a PEG, an alkyl and/or their derivatives or combination thereof, L is a cleavable linker, P is a pharmaceutically active substance (e.g., a toxicin), and the

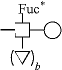

is linked to the N297 position of the antibody. For example, the protein conjugate has a similar binding affinity as its corresponding antibody towards an antigen. For example, the protein conjugate is for treating diseases.

For example, the binding affinity of some examplary antibody-G₂(F)-Fuc* conjugates and some antibody-(Galβ1,4)(GlcNAc)-Fuc* conjugates prepared from either the “one-step” process or the “two-step” process, compared with their corresponding antibody are shown in FIG. 20. FIG. 20 showed the binding affinity of some examplary trastuzumab-G₂F-Fuc* conjugates and some trastuzumab-(Galβ1,4)(GlcNAc)-Fuc* conjugates have a similar binding affinity as trasztuzumab towards Her2. The results indicating that the antibody conjugates in present disclosure has a similar binding affinity as the corresponding antibody towards. The results indicating that the conjugation methods in present disclosure have negligible influence on the binding affinity of the antibody.

The present disclosure provides a composition of the protein conjugate.

In the present disclosure, the composition of the protein conjugate may have an average MOI-antibody ratio (MAR) of about 4 (for example, range of 3.5-4). For example, when the protein conjugate has the formula of

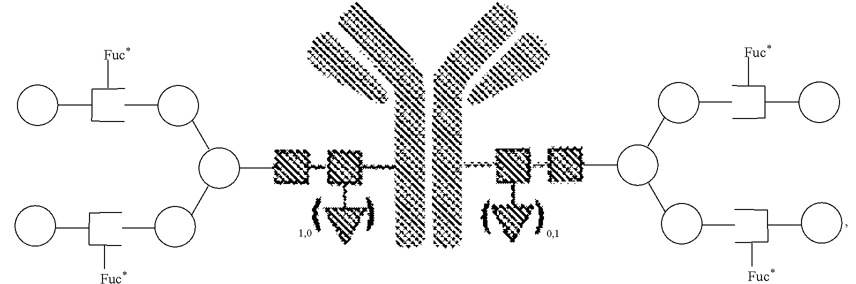

the composition of the protein conjugate may have a molecule of interest-antibody ratio (MAR) of between 2.4-4, 2.8-4, 3.2-4, 3.5-4, or 3.6-4. When the molecule of interest comprises a pharmaceutically active substance (e.i., a drug), the MAR can be drug-antibody ratio (DAR).

In the present disclosure, the composition of the protein conjugate may have a molecule of interest-to-antibody ratio (MAR) of about 2 (for example, range of 1.6-2). For example, when the protein conjugate has the formula of

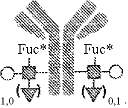

For example, the composition of the protein conjugate may have a molecule of interest-antibody ratio (MAR) of between 0.5-2, 1-2, 1.5-2, 1.6-2, 1.8-2, or 1.9-2. When the molecule of interest comprises a pharmaceutically active substance (e.i., a drug), the MAR can be drug-antibody ratio (DAR).

For example, the average MAR or DAR of some examplory antibody conjugates were listed in the examples.

In another aspect, the present disclosure provides an antibody-drug conjugate, wherein the antibody is an antibody specifically binding tumor antigen and wherein the molecule of interest is a cytotoxin, for use as a medicament. The invention also relates to an antibody-drug conjugate according to the invention, wherein the antibody is an antibody specifically binding tumor antigen and wherein the molecule of interest is a cytotoxin, for use in the treatment of cancer. In some embodiment, the tumor antigen may be Trop2, VEGF, CD20, and/or Her2. In some embodiments, the protein conjugate (e.g., the antibody-drug conjugate) is capable of treating breast cancer, lymphoma, colorectal cancer, lung cancer, kidney cancer, brain cancer and/or ovarian cancer.

In present disclosure, the linker L or L′ is a cleavable linker and may be necessary for the antibody-conjugates in present disclosure to achieve the functionality of the P part. For example, in present disclosure, the antibody-conjugates with a cleavable linker may have much higher efficacy compared to the antibody-conjugates without linker.

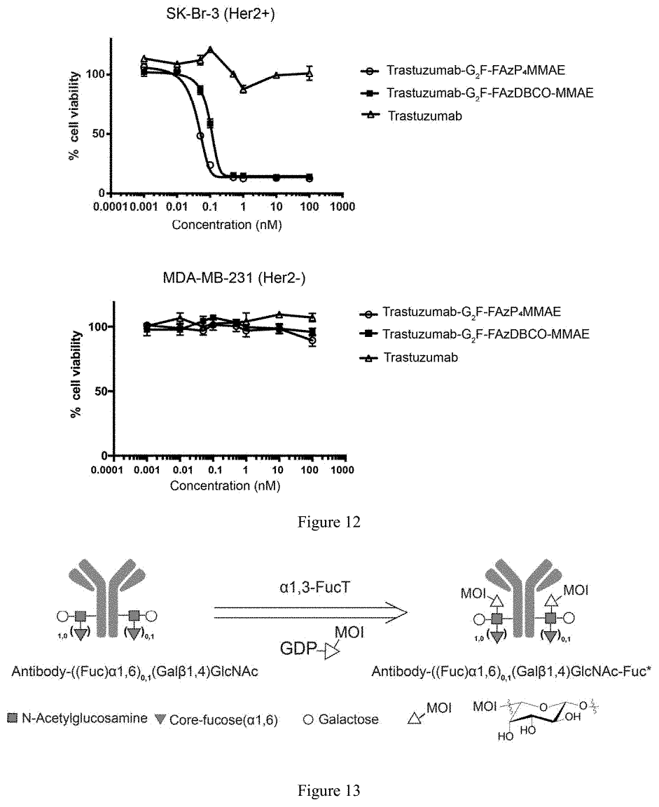

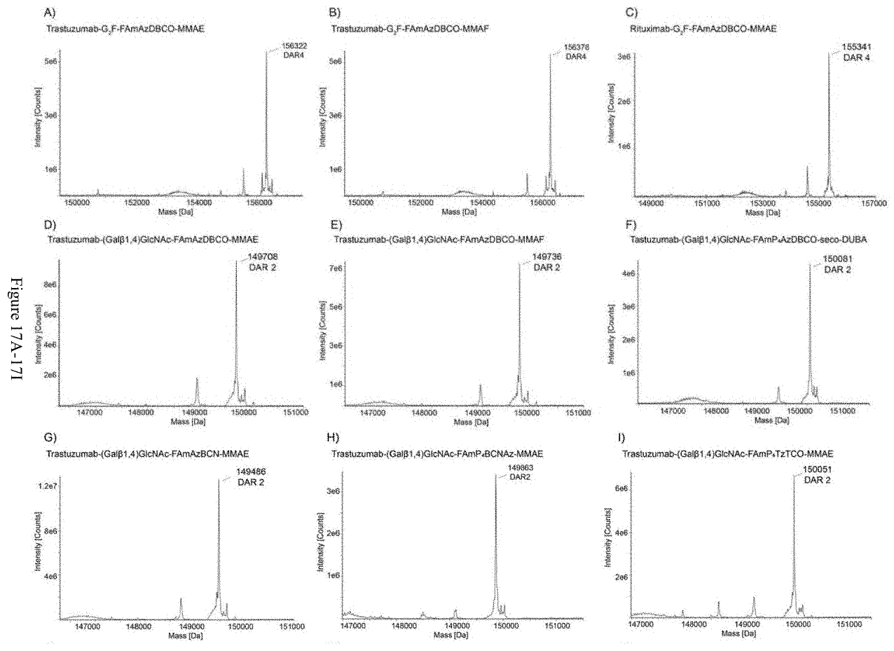

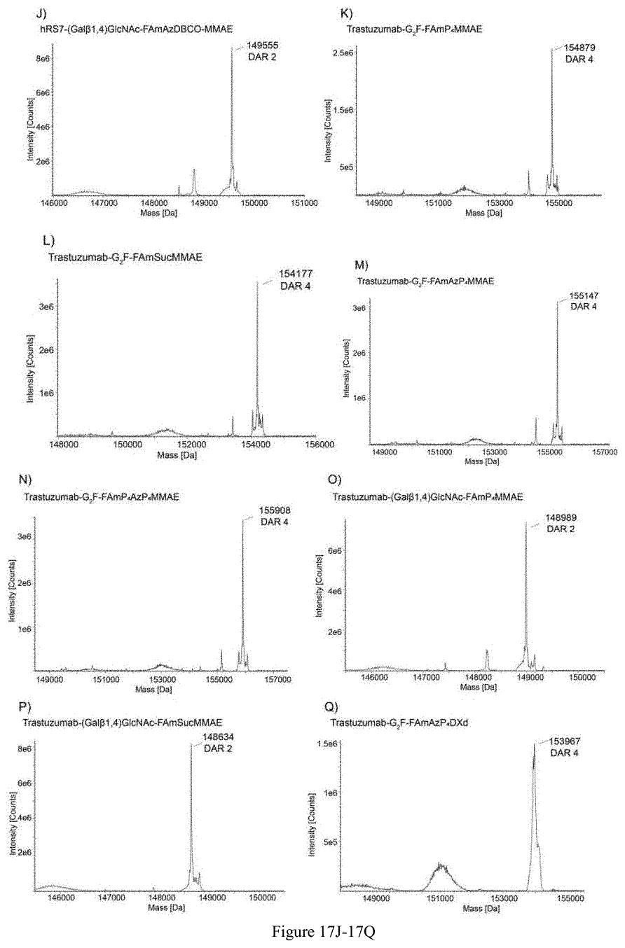

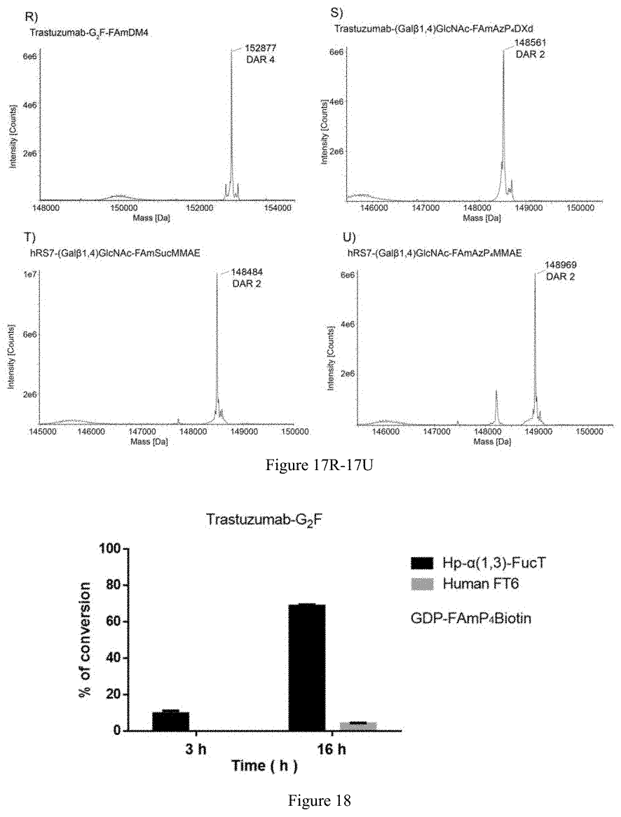

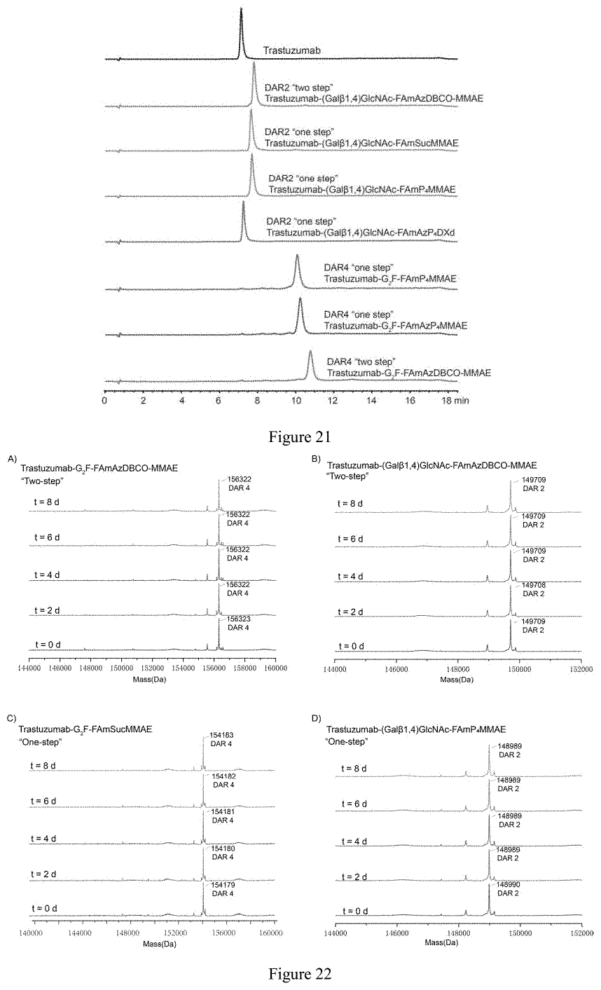

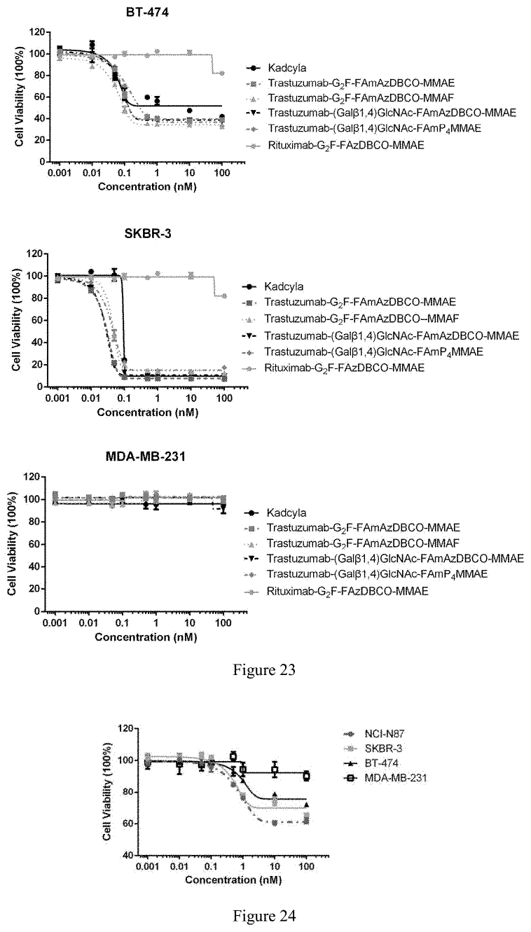

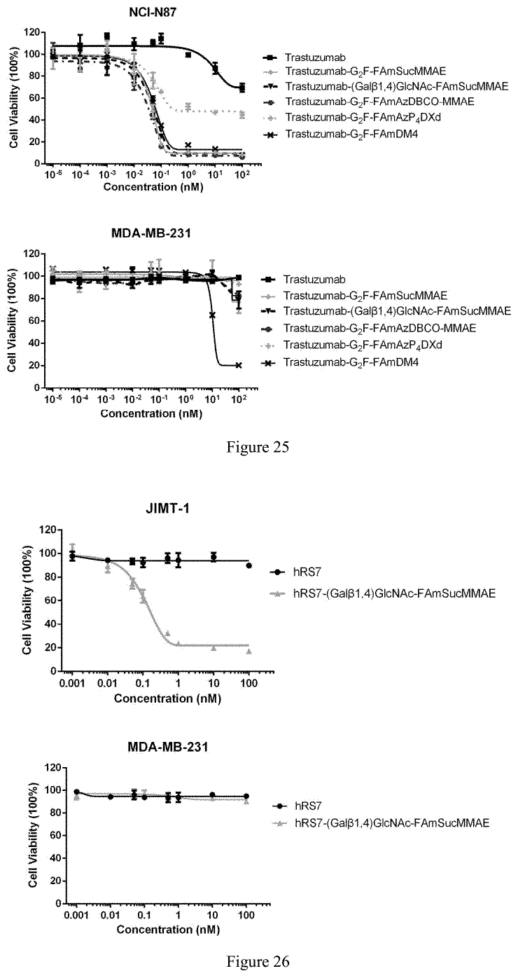

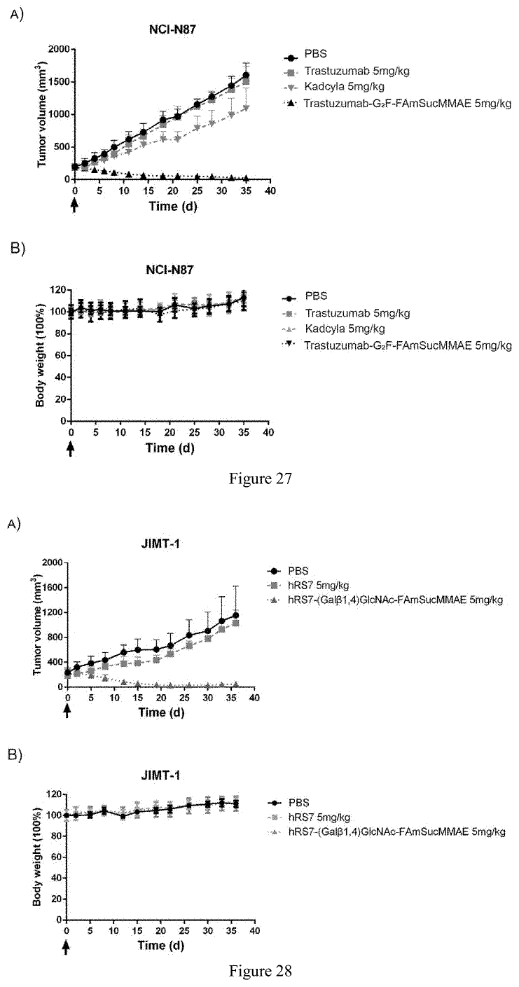

In present disclosure, the protein conjugate has at least one of the following characteristics: (a) well-controlled and defined conjugation sites; (b) well-defined and well-controlled DAR or MAR (c) high homogeneity; (d) negligible influence of the binding affinity of the antibody; (e) high stability (for example, the conjugation linkage between the Fuc of Fuc* and the GlcNAc of the -GlcNAc-Gal is stable in human plasma for at least one day); (f) good efficacy. For example, FIG. 17 showed the MS-analysis of the antibody-drug conjugates prepared from the “one-step” and the “two-step” process, illustrating well defined and well-controlled DAR or MAR, and the high homogeneity of the antibody-drug conjugates. For another example, FIG. 21 showed the HIC-HPLC analysis of some antibody-drug conjugates prepared from the “one-step” and the “two-step” process, also demonstrating the high homogeneity of the antibody-drug conjugates. For another example, FIG. 20 illustrated that the trastuzumab-drug conjugates showed the similar binding affinity as the trastuzumab toward the Her2 antigen, indicating the negligible influence of the binding affinity of the antibody. For another example, FIG. 22 shows the antibody-drug conjugates prepared from the “one-step” and the “two-step” process were stable in human plasma for at least 8 days. FIG. 22 also shows the linkage between the Fuc of Fuc* and the GlcNAc of the -GlcNAc-Gal are stable in plasma for at least 1 day (even for 8 days), as measured in mass spectrometry analysis. Antibody conjugates prepared through conventional strategies may not stable in the plasma. For example, antibody conjugates prepared through the cysteine-maleimide conjugation were not stable in the plasma and leading to significant DAR loss over time. The linkage of the cysteine-maleimide were not stable in the plasma. For example, the plasma may be human plasma. For another example, FIG. 12, FIG. 23, FIG. 24, FIG. 25, FIG. 26, FIG. 27 and FIG. 28 illustrated the antibody conjugates in present disclosure showed good in vitro and in vivo efficacy.

Compound

In another aspect, the present disclosure provides a compound, which comprises a guanosine diphosphate (GDP) and a pharmaceutically active substance (P).

For example, the P is a pharmaceutically active substance. For example, the P may be a cytotoxin, a cytostatic agent, a radioisotope or radionuclide, a metal chelator, an oligonucleotide, an antibiotic, a peptide, and/or a protein, or any combination thereof. For example, the P may be an anti-tumor agent. For example, the P may comprise a cytotoxin. For example, the P may comprise a cytotoxin selected from the group consisting of pyrrolobenzodiazepine, auristatin, maytansinoids, duocarmycin, tubulysin, enediyene, doxorubicin, pyrrole-based kinesin spindle protein inhibitor, calicheamicin, amanitin, and camptothecin. For example, the P may comprise a cytotoxin selected from the group consisting of MMAE, DXd, MMAF, seco-DUBA and DM4.

The compound may have a higher hydrophilicity than said P.

In the present disclosure the compound may have a formula of GDP-(F)ₘ-(L)ₙ-P, wherein F is a connector, L is a linker, m is 0 or 1 and n is 0 or 1.

For example, the F may be

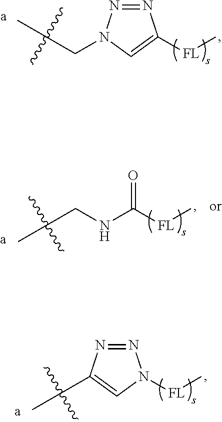

wherein said FL is a spacer and s is 0 or 1.

For example, the compound may comprise a formula (I):

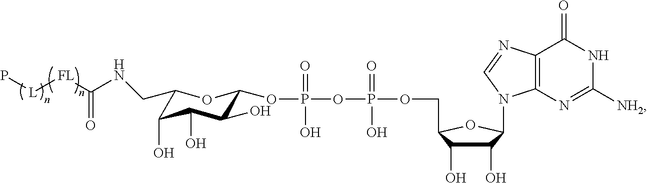

wherein P is the pharmaceutically active substance, L is the linker, FL is the spacer, n is 0 or 1, and s is 0 or 1. For example, s is 1 and n is 0. For example, s is 1 and n is 1.

For example, the L may be a cleavable linker.

For example, the L may be an acid-labile linker, a redox-active linker, a photo-active linker and/or a proteolytically cleavable linker.

For example, the L may be a vc-PAB linker, a GGFG linker or a disulfo linker.

For example, the FL may be a polypeptide, a PEG, an alkyl and/or their derivatives or combination thereof.

For example, the FL may be selected from the group consisting of:

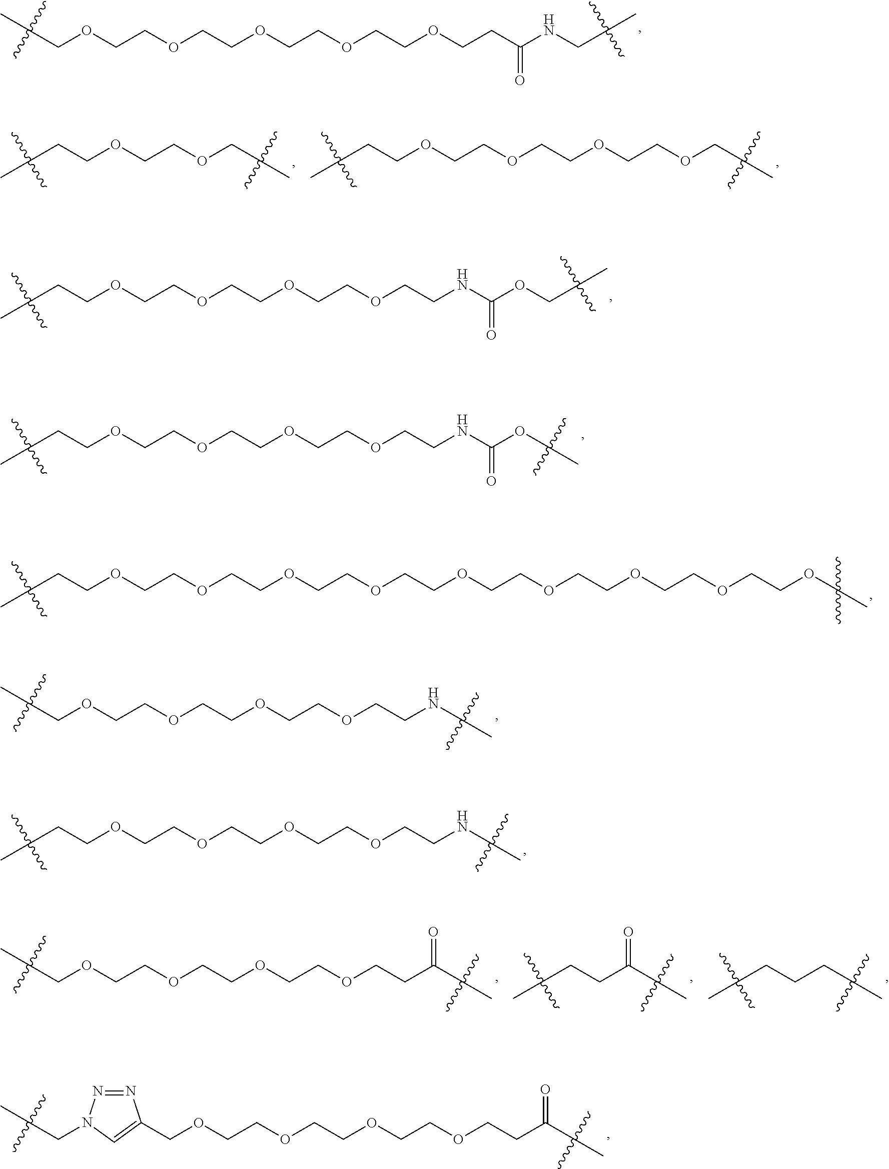

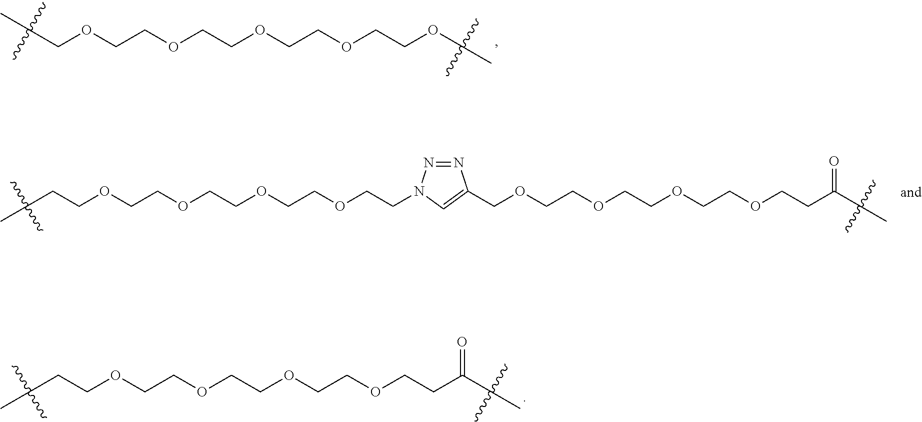 [SMILES: *COCCOCCOCCOCCO* ; *CCOCCOCCOCCOCCn1cc(COCCOCCOCCOCCC(*)=O)nn1 ; *CCOCCOCCOCCOCCC(*)=O]

For example, the compound may be selected from the group consisting of

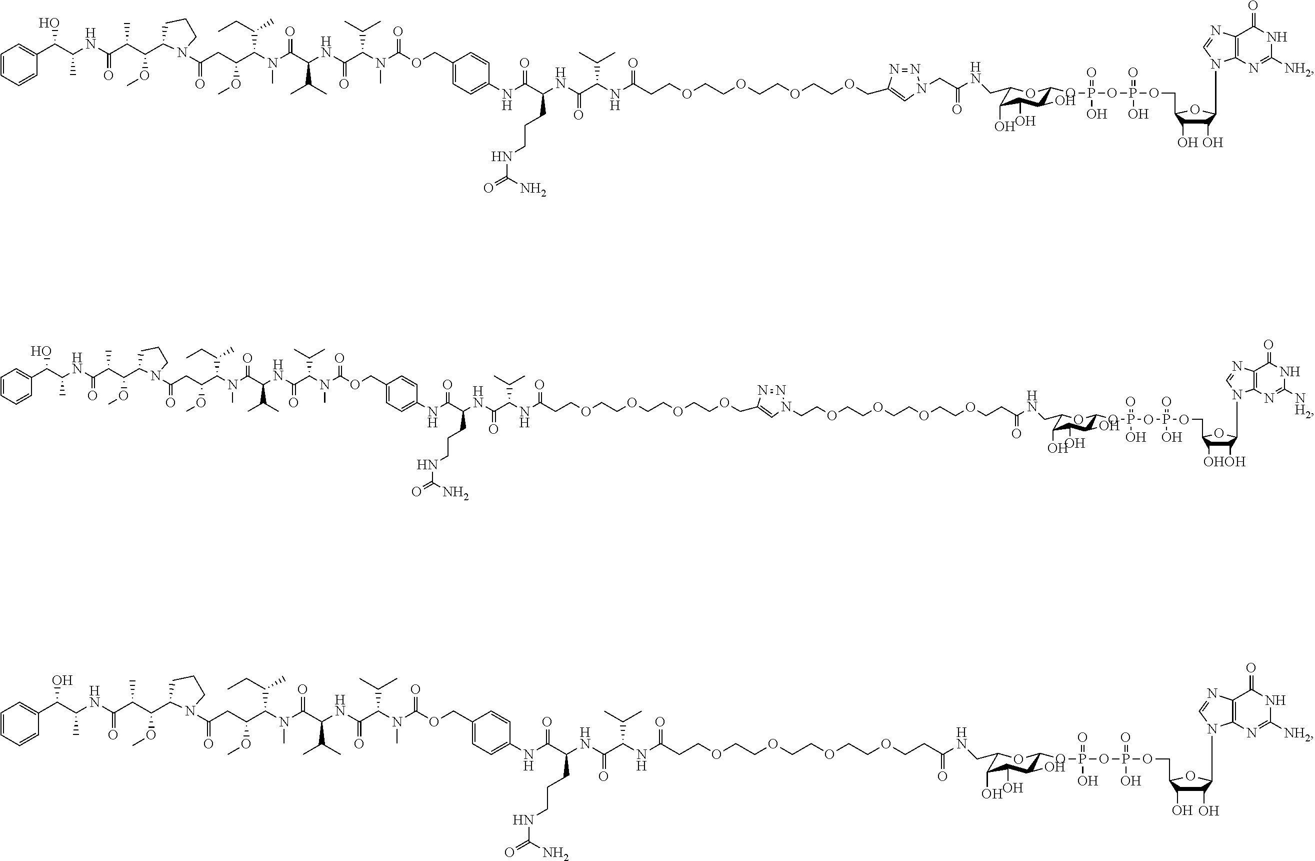

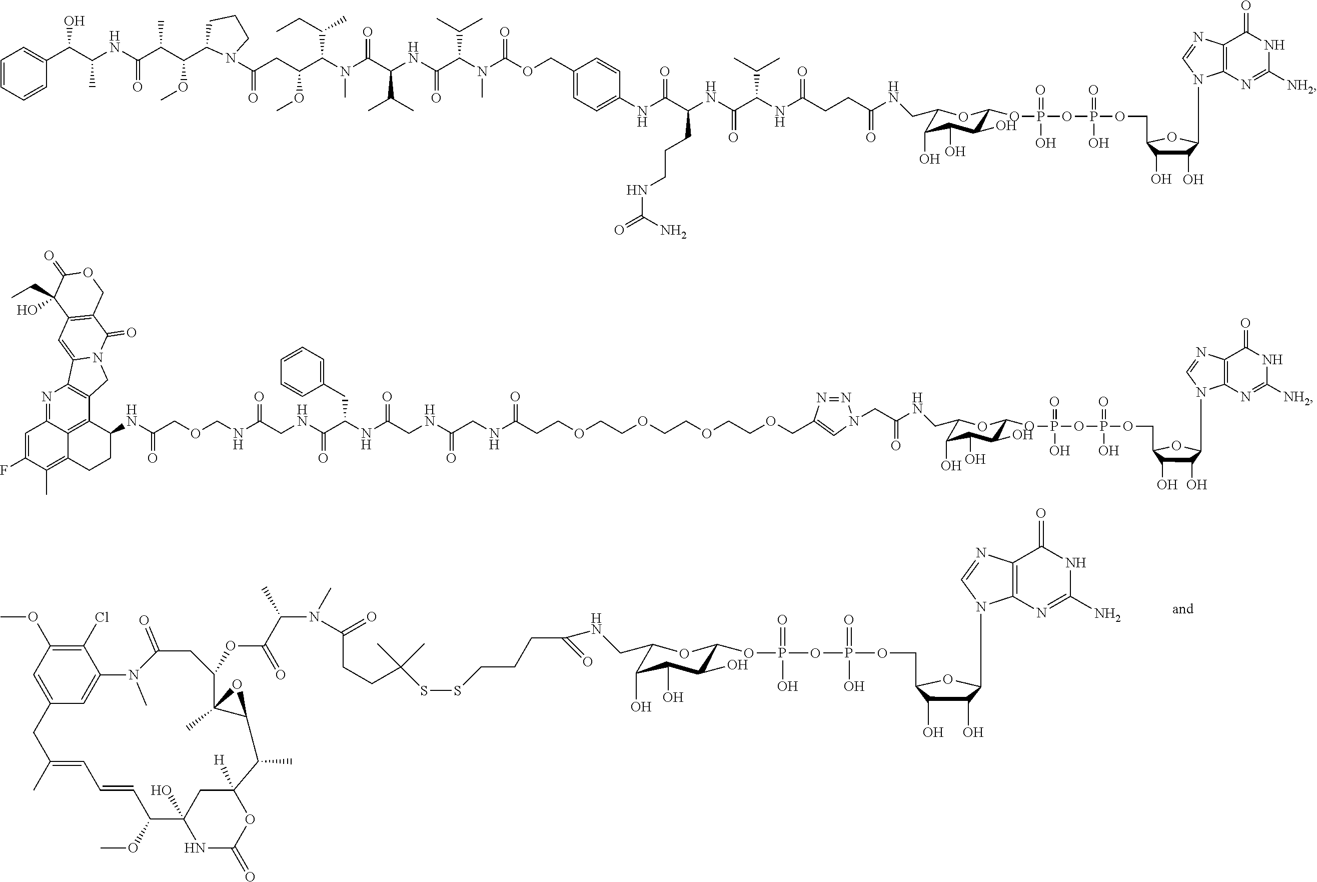

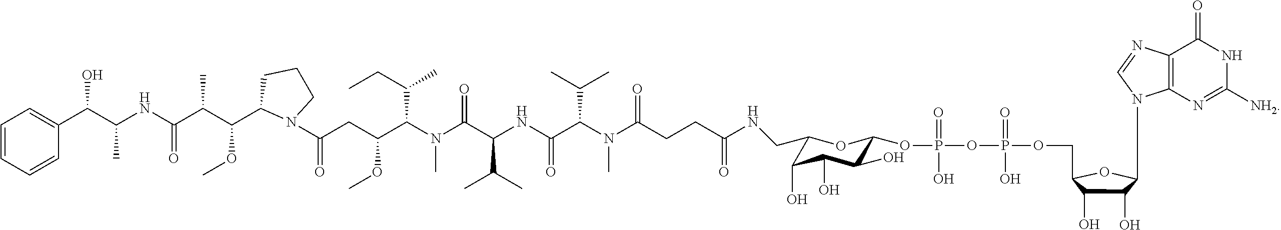

In another aspect, the present disclosure further provides a compound comprising a formula (II):

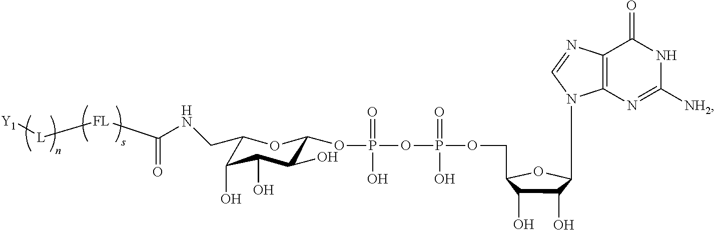

wherein Y₁ is a functional group defined as above, the L is a linker defined as above, the FL is a spacer defined as above, s is 0 or 1 and n is 0 or 1. For example, n is 0 and s is 1. For example, n is 0 and s is 0.

For example, the exemplary compound comprising the formula (II) may be selected from the group consisting of

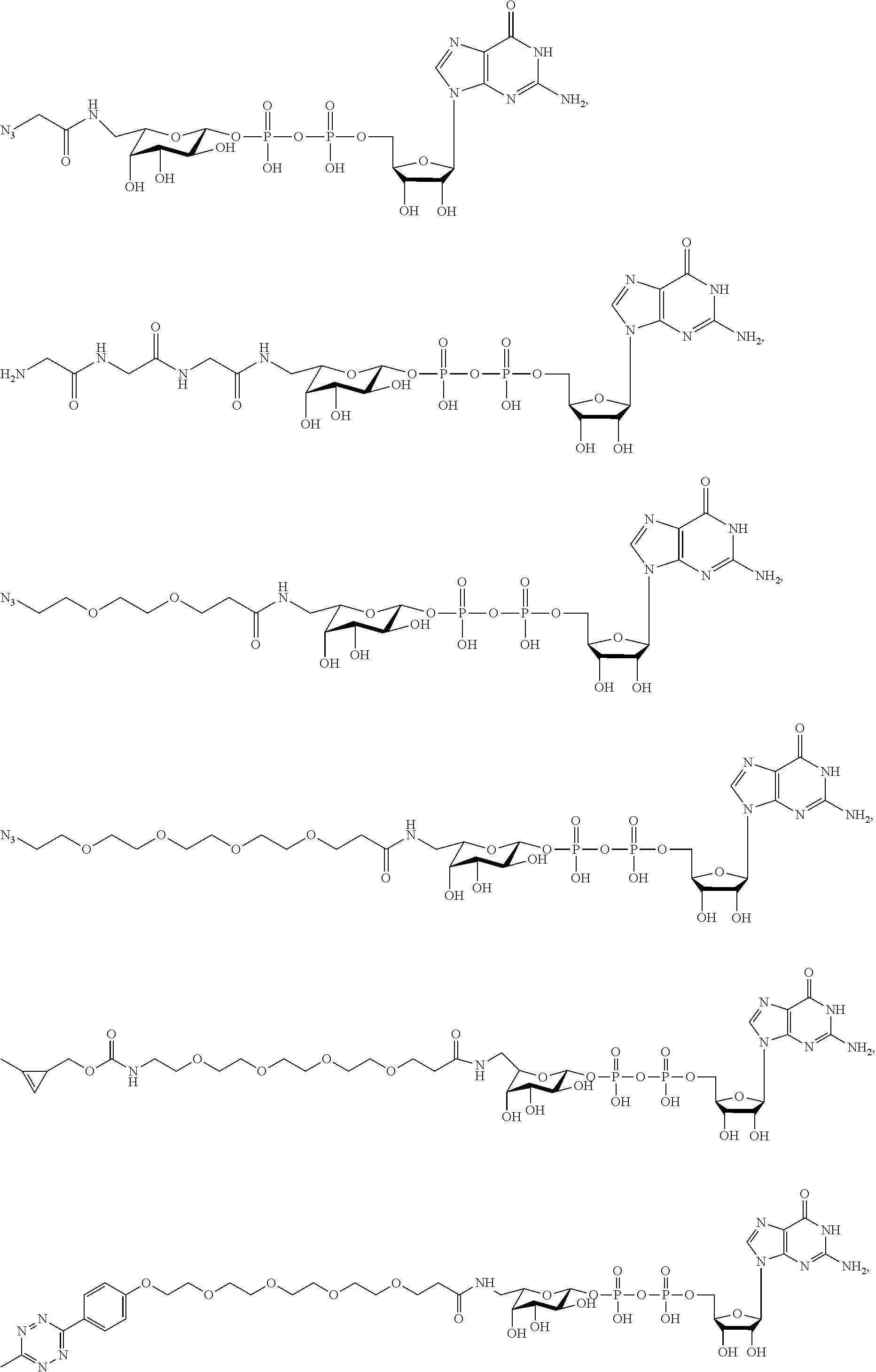

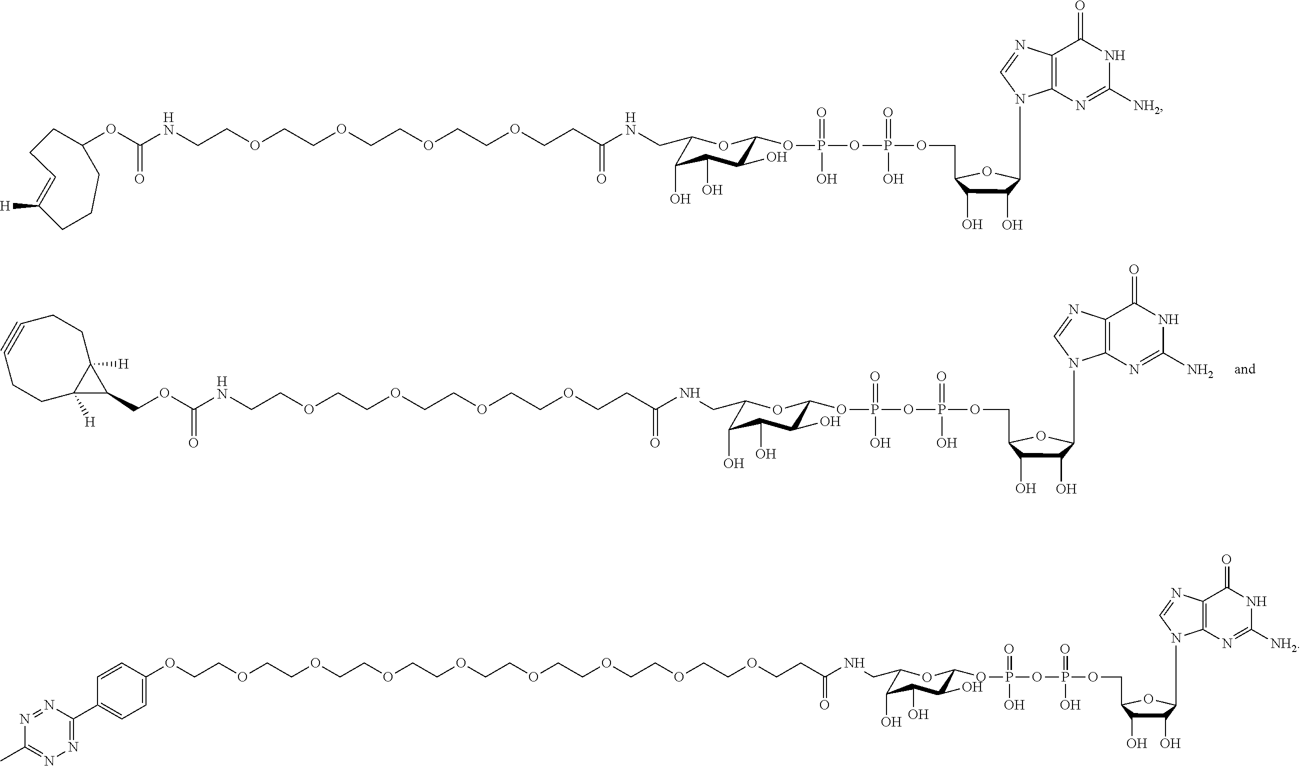

In another aspect, the present disclosure provides a method of making a protein conjugate, which comprises using the compound of the present disclosure. When the protein is an antibody, the protein conjugate has the similar binding affinity towards the corresponding antibody.

Method

In another aspect, the present disclosure provides a method for preparing the protein conjugate. The method comprises step (a) contacting a fucose derivative donor Q-Fuc*′ to a protein comprising an oligosaccharide in the presence of a catalyst, wherein the oligosaccharide comprises -GlcNAc(Fuc)_{b}-Gal, to obtain a protein conjugate comprising

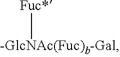

wherein the GlcNAc is directly or indirectly linked to an amino acid of the protein; the Gal is a galactose; the (Fuc) is a fucose, b is 0 or 1; the Fuc*′ comprises a fucose or fucose derivative Fuc linked to a molecule of interest (MOI′); the protein comprises an antigen binding fragment and/or a Fc fragment; the Q-Fuc*′ is a molecule comprises the Fuc*′. The method of the present disclosure may be performed in the presence of a catalyst capable of transferring said Fuc*′ to said GlcNAc of -GlcNAc(Fuc)_{b}-Gal. Several suitable condition for the method according to the invention are known in the art. A suitable condition for a specific method is a catalyst wherefore the fucose or fucose derivative donor in that specific method is a substrate. In the present disclosure, the catalyst may be selected from the group of fucosyltransferase.

In one embodiment, the Fuc*′ of the Q-Fuc*′ may be transferred to the GlcNAc of the -GlcNAc(Fuc)_{b}-Gal of the oligosaccharide comprised by the protein. The fucosyltransferase may be an α-1,3-fucosyltransferase or a catalytic domain thereof. In one embodiment, the fucosyltransferase may be obtained from bacteria (e.g., Helicobacter pylori). In one embodiment, the α-1,3-fucosyltransferase is recombinantly prepared. In some embodiments, the fucosyltransferase is derived from Bacteroides fragilis. In some embodiments, the fucosyltransferase comprises an amino acid sequence as set forth in GenBank Accession no. YP_213065.1, or a functional variant or fragment thereof. In some embodiments, the fucosyltransferase is derived from Helicobacter pylori. In some embodiments, wherein said fucosyltransferase comprises an amino acid sequence as set forth in GenBank accession no. AF008596.1, GenBank accession no. AAD07447.1, GenBank Accession No. AAD07710.1, GenBank accession no. AAF35291.2, GenBank accession no. AAB93985.1, or their functional variant or fragment thereof. For example, the fucosyltransferase may comprise an amino acid sequence as set forth in GenBank Accession No. AAD07710.1, or a functional variant or fragment thereof. For example, the fucosyltransferase may comprise an amino acid sequence as set forth in SEQ ID NO: 3 or 4, or a functional variant or fragment thereof.

For example, the fucosyltransferase may be from bacteria. For example, the step (a) may be performed in the presence of a Hp α-1,3-fucosyltransferase.

In the present disclosure, said Q-Fuc*′ may be a donor and a Fuc*′. The donor may comprise a uridine diphosphate (UDP), a guanosine diphosphate (GDP) or a cytidine diphosphate (CDP).

In the present disclosure, the Q-Fuc*′ may comprise a GDP, the fucose or fucoses derivative, an optionally connector F, an optionally linker L, and an active molecule (e.g. the functional group Y₁ or P), In some embodiments, the Q-Fuc*′ is a GDP-Fuc-(F)ₘ-(L)ₙ-Y₁, wherein said Fuc is a

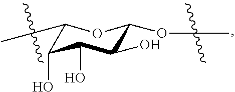

n is 0 or 1, and m is 0 or 1. In some embodiments, the Q-Fuc*′ is a GDP-Fuc-(F)ₘ-(L)ₙ-P, wherein said Fuc is a

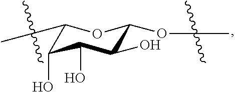

n is 0 or 1, and m is 0 or 1.

In the present disclosure, the P may be conjugated to the protein directly by a glycosyl transfer reaction (a “one-step” process). For example, the Q-Fuc*′ may be a GDP-Fuc-(F)ₘ-(L)ₙ-P, wherein n is 0 or 1, and m is 0 or 1. In the presence of the fucosyltransferase, the -Fuc-(F)ₘ-(L)ₙ-P may be transferred to the GlcNAc of the protein comprising -GlcNAc(Fuc)_{b}-Gal to obtain a protein conjugate comprising

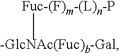

wherein Fuc is

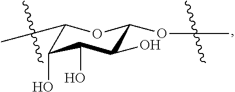

b is 0 or 1, m is 0 or 1, and n is 0 or 1.

For example, the Q-Fuc-(F)ₘ-(L)ₙ-P may have a structure of

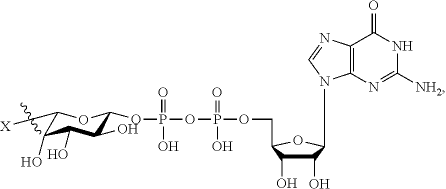

and the X may be selected from the group consisting

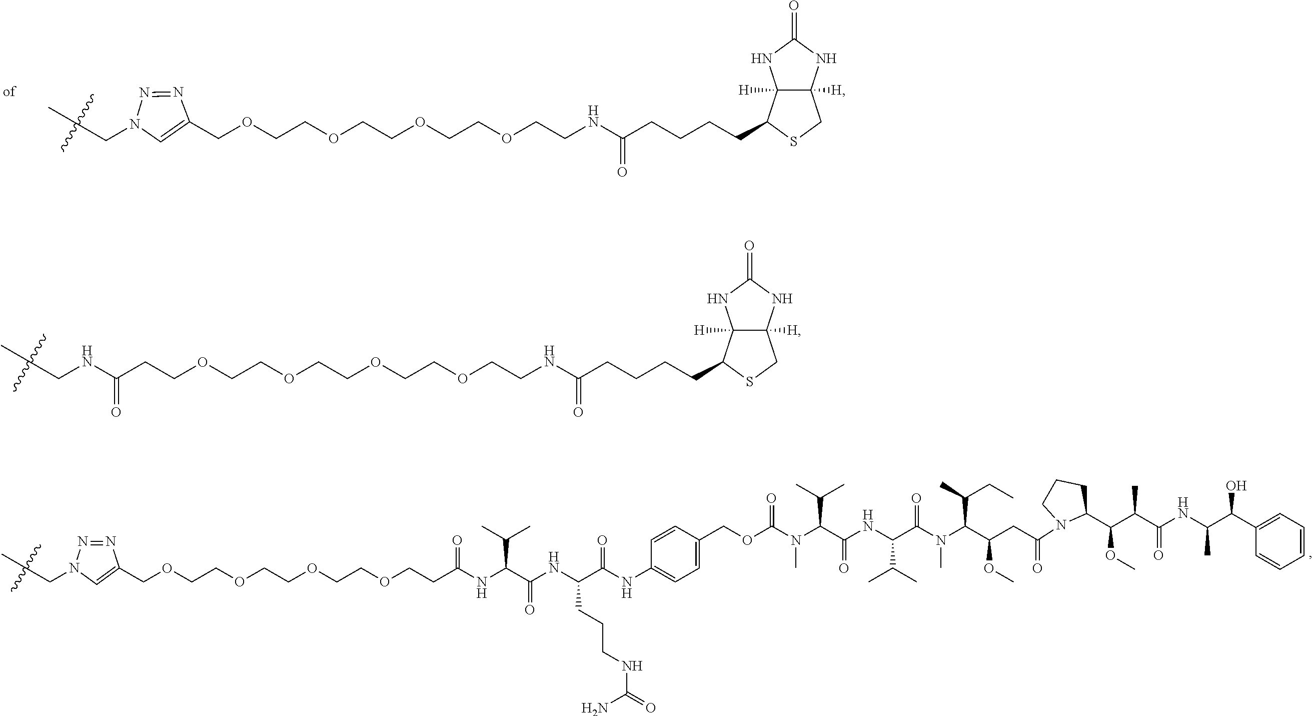

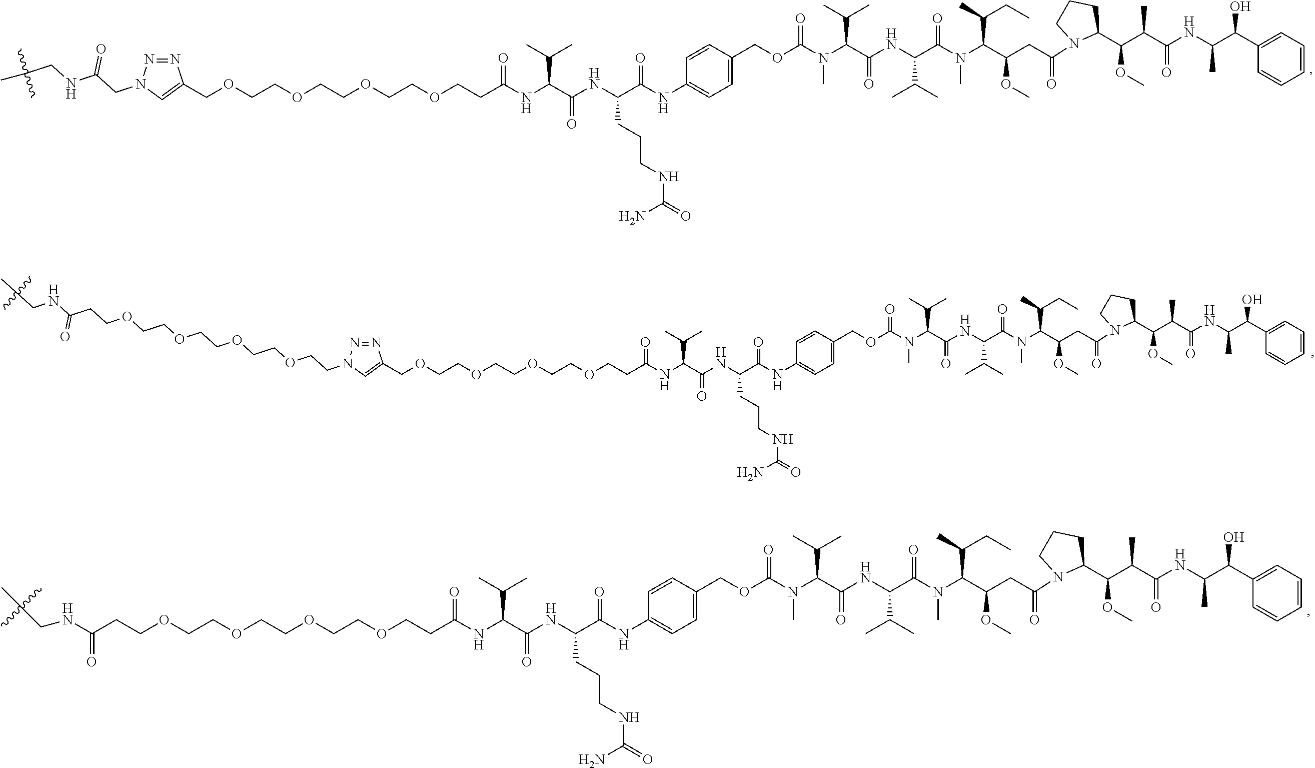

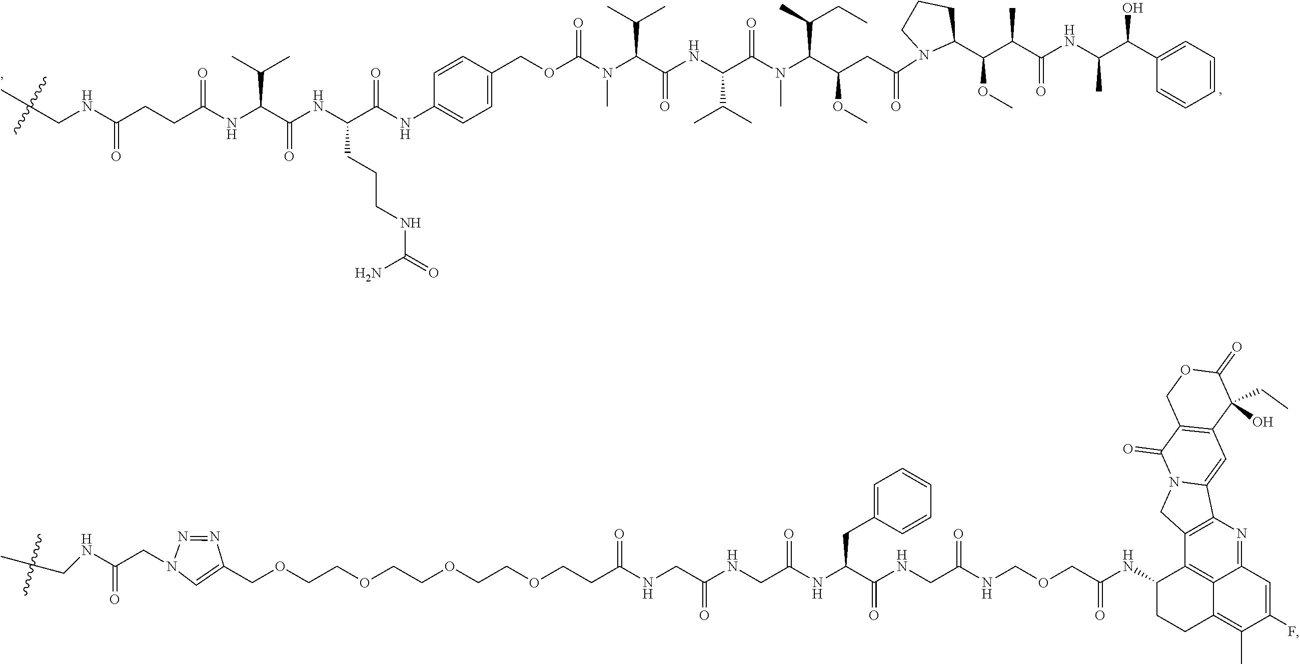 [SMILES: *CNC(=O)CCC(=O)N[C@H](C(=O)N[C@@H](CCCNC(N)=O)C(=O)Nc1ccc(COC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)cc1)C(C)C ; *CNC(=O)Cn1cc(COCCOCCOCCOCCC(=O)NCC(=O)NCC(=O)N[C@@H](Cc2ccccc2)C(=O)NCC(=O)NCOCC(=O)N[C@H]2CCc3c(C)c(F)cc4nc5c(c2c34)Cn2c-5cc3c(c2=O)COC(=O)[C@]3(O)CC)nn1]

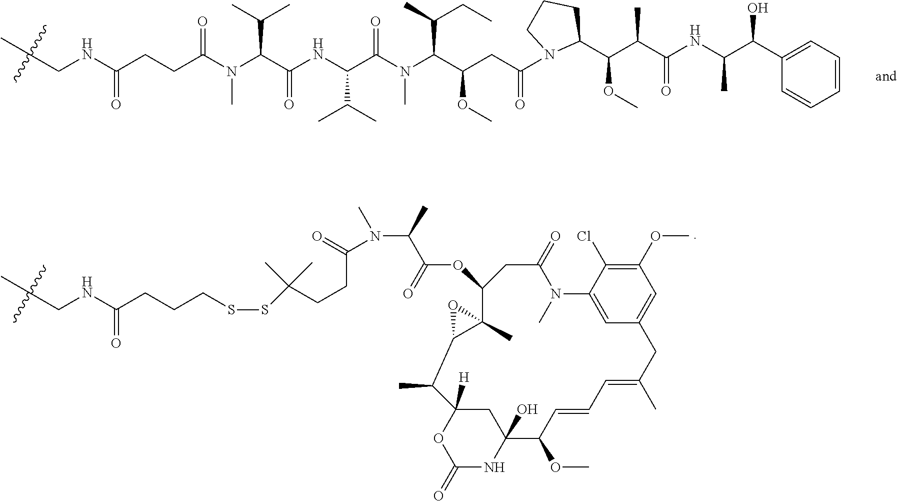

In the present disclosure, in step (a), when the Q-Fuc*′ is a GDP-Fuc-(F)ₘ-(L)ₙ-P, the protein conjugate comprising the

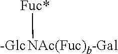

is obtained directly through the glycosyl transfer reaction without a further ligation reaction (the “one-step” process), the Fuc*′ in the Q-Fuc* Fuc*′ and the corresponding Fuc* of the protein conjugate comprising the

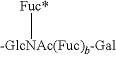

are the same. For example, when the Q-Fuc*′ is a GDP-Fuc-(F)ₘ-(L)ₙ-P, the Fuc*′ in the Q-Fuc*′ and the corresponding Fuc* of the protein conjugate comprising the

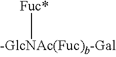

have a same structure of Fuc-(F)ₘ-(L)ₙ-P. Meanwhile, the MOI′ of the Fuc*′ and the MOI of the Fuc* are the same, and the

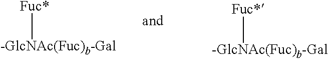

are the same.

In the present disclosure, the P may be conjugated to the protein by a glycosyl transfer reaction and a further ligation reaction (a “two-step” process). For example, the Q-Fuc*′ is a GDP-Fuc-(F)ₘ-(L)ₙ-Y₁, wherein n is 0 or 1, and m is 0 or 1. In the presence of the fucosyltransferase, the Fuc-(F)ₘ-(L)ₙ-Y₁ may be transferred to the GlcNAc of the protein comprising -GlcNAc(Fuc)_{b}-Gal to obtain a protein conjugate comprising

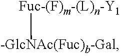

wherein Fuc is

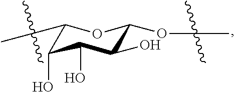

b is 0 or 1, m is 0 or 1, and n is 0 or 1.

For example, the Q-Fuc-(F)ₘ-(L)ₙ-Y₁ may have a structure of

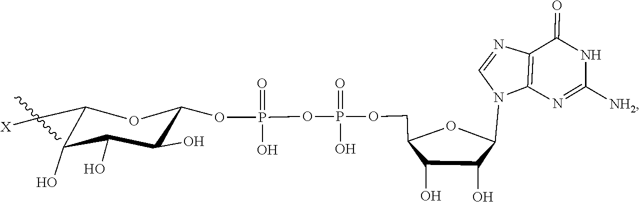

and the X may be selected from the group consisting of

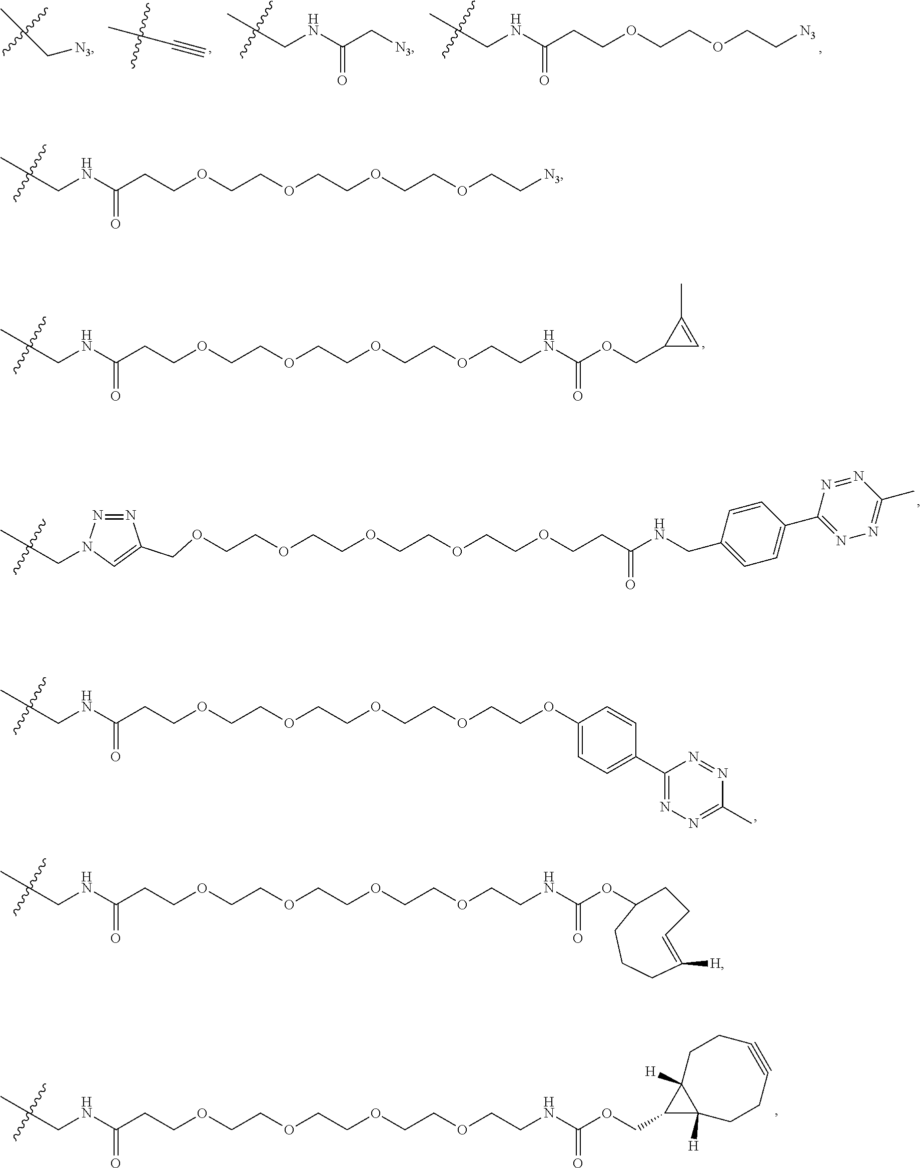

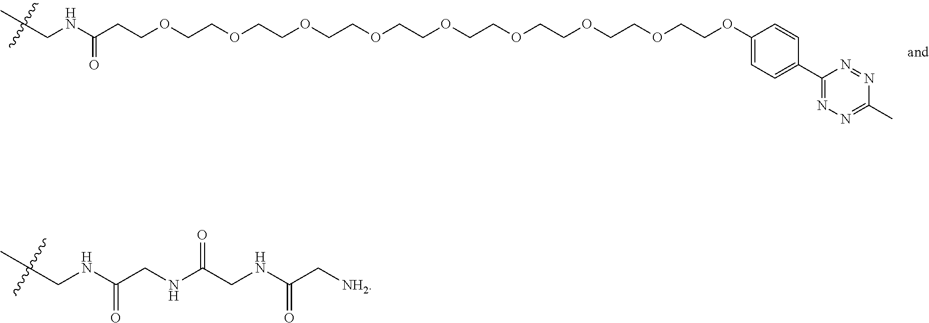 [SMILES: *CNC(=O)CCOCCOCCOCCOCCOCCOCCOCCOCCOc1ccc(-c2nnc(C)nn2)cc1 ; *CNC(=O)CNC(=O)CNC(=O)CN]

In the present disclosure, when the Q-Fuc*′ is a GDP-Fuc-(F)ₘ-(L)ₙ-Y₁, the protein conjugate comprising the

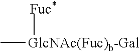

is obtained through the glycosyl transfer reaction without a further ligation reaction, the Fuc*′ in the Q-Fuc*′ and the corresponding Fuc* of the protein conjugate comprising the

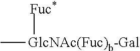

are the same. For example, when the Q-Fuc*′ is a GDP-Fuc-(F)ₘ-(L)ₙ-Y₁ the Fuc*′ in the Q-Fuc*′ and the corresponding Fuc* of the protein conjugate comprising the

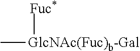

have a same structure of Fuc-(F)ₘ-(L)ₙ-Y₁, Meanwhile, the MOI′ of Fuc*′ and the MOI of the Fuc* are the same, and the

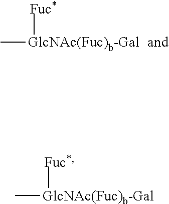

are the same.

The method may further comprises a step (b) contacting the protein conjugate comprising

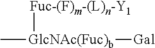

to obtain a protein conjugate comprising

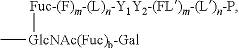

wherein Y₁Y₂ is a remaining group after a ligation reaction between said Y₁ and a functional group Y₂ comprising a functional moiety capable of reacting with Y₁, (Fuc) is a fucose, Fuc is a fucose or fucose derivative, L is a linker, F is a connecter, L′ is a cleavable defined as the same as L, FL′ is a spacer defined as the same as FL, b is 0 or 1, m is 0 or 1, n is 0 or 1, m′ is 0 or 1, and n′ is 0 or 1.

In the present disclosure, step (b) may be performed after step (a). In the present disclosure, there may be a purification process between step (b) and step (a).

In the present disclosure, when the Q-Fuc*′ is a GDP-Fuc-(F)ₘ-(L)ₙ-Y₁, and the protein conjugate comprising the

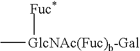

is obtained through a glycotransfer reaction and a further ligation reaction (the “two-step” process), the

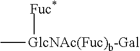

of the protein conjugate is the

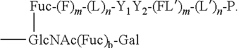

For example, in the “two-step” process, the Fuc*′ of Q-Fuc*′ have a structure of Fuc-(F)ₘ-(L)ₙ-Y₁ while the corresponding Fuc* of the protein conjugate comprising the

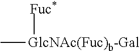

have a structure of Fuc-(F)ₘ-(L)ₙ-Y₁Y₂-(FL′)_{n′}-(L′)_{m′}-P. In this case, the Fuc* and the Fuc*′ are different, and the MOI′ of the Fuc*′ and the MOI of the Fuc* are different.

In the present disclosure, when the protein conjugate comprises the

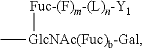

the existence of a connector of

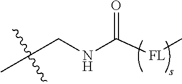

may significantly enhance the reactivities of the functional group Y₁ towards the functional group Y₂, wherein s is 0 or 1. For example, an antibody conjugate comprising the functional group Y₁ with a connector F of

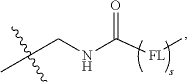

may have significantly enhanced reactivity of Y₁ towards its corresponding functional group Y₂, than an antibody conjugate comprising the same functional group Y₁ but without a connector F of

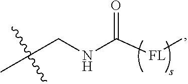

wherein s is 0 or 1. For example, the antibody conjugate comprising the same

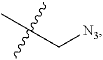 [SMILES: *CN=[N+]=[N-]]

with or without a connector F, have significantly different reactivity towards the DBCO linked with a pharmaceutically active substance P. For example, an antibody-G₂(F)-FAmAz

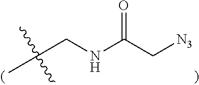 [SMILES: *CNC(=O)CN=[N+]=[N-]]

and an antibody-G₂(F)-FAmP₄Az

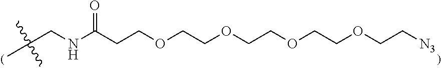 [SMILES: *CNC(=O)CCOCCOCCOCCOCCN=[N+]=[N-]]

may show a much higher reactivity than the antibody-G₂(F)-FAz

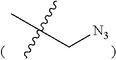 [SMILES: *CN=[N+]=[N-]]

towards DBCO-PEG₄-vc-PAB-MMAE. For another example, an antibody-(Galβ1,4)GlcNAc-FAmAz

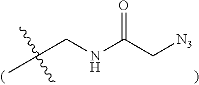 [SMILES: *CNC(=O)CN=[N+]=[N-]]

and antibody-(Galβ1,4)GlcNAc-FAmP₄Az

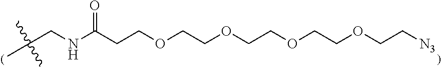 [SMILES: *CNC(=O)CCOCCOCCOCCOCCN=[N+]=[N-]]

may have a significantly higher reactivity than the antibody-(Galβ1,4)GlcNAc-FAz

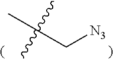 [SMILES: *CN=[N+]=[N-]]

towards DBCO-PEG₄-vc-PAB-MMAE. For example, the trastuzumab-G₂F-FAmAz

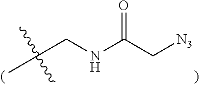 [SMILES: *CNC(=O)CN=[N+]=[N-]]

and the trastuzumab-G₂F-FAmP₄Az

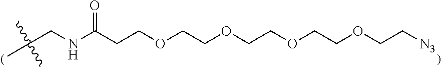 [SMILES: *CNC(=O)CCOCCOCCOCCOCCN=[N+]=[N-]]

showed a much higher reactivity than the trastuzumab-G₂F-FAz

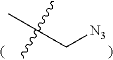 [SMILES: *CN=[N+]=[N-]]

towards DBCO-PEG₄-vc-PAB-MMAE. For another example, the trastuzumab-(Galβ1,4)GlcNAc-FAmAz

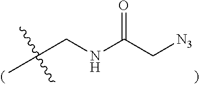 [SMILES: *CNC(=O)CN=[N+]=[N-]]

and the trastuzumab-(Galβ1,4)GlcNAc-FAmP₄Az

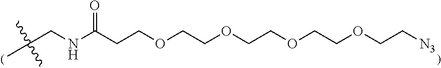 [SMILES: *CNC(=O)CCOCCOCCOCCOCCN=[N+]=[N-]]

showed significantly higher reactivity than the trastuzumab-(Galβ1,4)GlcNAc-FAz

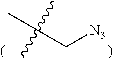 [SMILES: *CN=[N+]=[N-]]

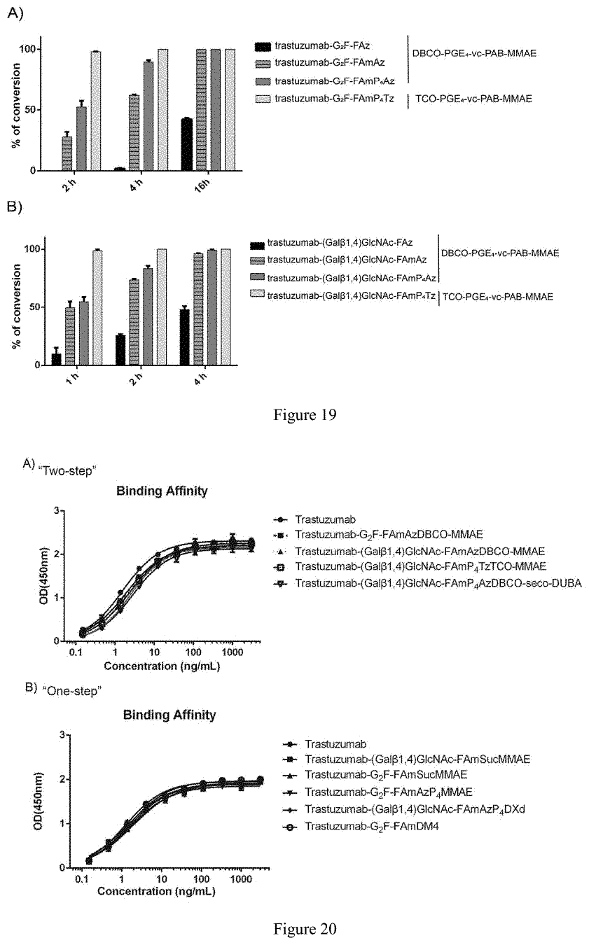

towards DBCO-PEG₄-vc-PAB-MMAE. FIG. 19A showed the comparison of the reactivities of trastuzumab-G₂F-FAz, trastuzumab-G₂F-FAmAz and trastuzumab-G₂F-FAmP₄Az towards DBCO-PEG₄-vc-PAB-MMAE. FIG. 19B showed the comparison of the reactivities of trastuzumab-(Galβ1,4)GlcNAc-FAz, trastuzumab-(Galβ1,4)GlcNAc-FAmAz and trastuzumab-(Galβ1,4)GlcNAc-FAmP₄Az towards DBCO-PEG₄-vc-PAB-MMAE.

In the present disclosure, when a protein conjugate comprises a

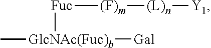

the Y₁ may comprise a

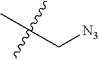 [SMILES: *CN=[N+]=[N-]]

group which could participate in a SPAAC (Strain-promoted azide-alkyne cycloaddition) reaction to for the installation of a pharmaceutically active substance P to the protein. In the present disclosure, when a protein conjugate comprises a

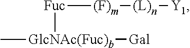

the Y₁ may comprise

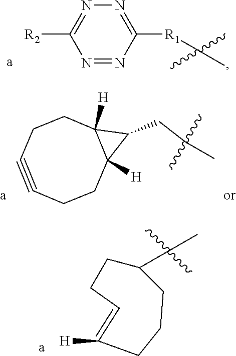

which could participate in an IEDDA (Inverse electron demand Diels-Alder) reaction for the installation of a pharmaceutically active substance P to the protein. The IEDDA reaction is usually much faster than the SPAAC reaction. By using a functional group that could participate in an iEDDA reaction may significantly facilitate the second step in a “two-step” process for making an antibody conjugate or Fc-fusion protein an. For example, it may take less time for an antibody conjugate or Fc-fusion protein conjugate comprising

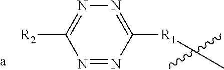

to install a pharmaceutically active substance P than for an antibody conjugate or Fc-fusion protein conjugate comprise

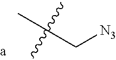 [SMILES: *CN=[N+]=[N-]]

to install a pharmaceutically active substance P. For example, the trastuzumab-G₂F-FAmP₄Tz showed a significantly higher reactivity towards TCO-PEG₄-vc-PAB-MMAE than the trastuzumab-G₂F-FAmP₄Az towards DBCO-PEG₄-vc-PAB-MMAE as shown in FIG. 19A. For another example, the trastuzumab-(Galβ1,4)GlcNAc-FAmP₄Tz showed a significantly higher reactivity towards TCO-PEG₄-vc-PAB-MMAE than the trastuzumab-G₂F-FAmP₄Az towards DBCO-PEG₄-vc-PAB-MMAE as shown in FIG. 19B.

In present disclosure, the protein conjugate prepared from the “two-step” process may contain a remaining group which may not existed in the protein conjugate generated from the “one-step” process. The remaining group may be a hydrophobic group. For example, the protein conjugate with a remaining group (generated from the “two-step” ) may be more hydrophobic than the protein conjugate without a remaining group (generated from the “one-step”). For example, the antibody conjugate with a remaining group (generated from the “two-step”) may be more hydrophobic than the antibody conjugate without a remaining group (generated from the “one-step” process). For example, the trastuzumab-G₂F-FAmAzDBCO-MMAE (generated from the “two-step”) is more hydrophobic than the trastuzumab-G₂F-FAmP₄MMAE (generated from the “one-step” process) as shown in FIG. 21. For example, the trastuzumab-(Galβ1,4)GlcNAc-FAmAzDBCOMMAE (generated from the “two-step”) is more hydrophobic than the trastuzumab-(Galβ1,4)GlcNAc-FAmP₄MMAE (generated from the “one-step” process) as shown in FIG. 21.

In the present disclosure, the step (a) may be performed in a suitable buffer solution, such as for example phosphate, buffered saline (e.g. phosphate-buffered saline, tris-buffered saline), citrate, HEPEs, Tris, Tris-HCl and glycine. Suitable buffers are known in the art. For example, the buffer solution is Tris-HCl buffer containing Mg²⁺. For example, the buffer solution is Tris-HCl buffer containing Mn²⁺. For example, the buffer solution is PBS buffer.

The step (a) may be performed at a temperature in the range of about 0° C. to about 50° C. In some embodiments, the method may be performed at a temperature in the range of about 10° C. to about 45° C. In some embodiments, the method may be performed at a temperature in the range of about 20° C. to about 40° C. In some embodiments, the method may be performed at a temperature in the range of about 20° C. to about 30° C. For example, the method may be performed at a temperature of about 30° C. For example, the method may be performed at a temperature of about 37° C.

The step (a) may be performed at a pH in the range of about 4 to about 10. In some embodiments, the method may be performed at a pH in the range of about 5 to about 9. In some embodiments, the method may be performed at a pH in the range of about 5.5 to about 8.5. In some embodiments, the method may be performed at a pH in the range of about 6 to about 8. In some embodiments, the method may be performed at a pH in the range of about 7 to about 8, for example, in the range of about 7 to about 7.5.

In the present disclosure, the method for preparation a protein conjugate may comprise buffer exchanging of the obtained protein conjugate into a buffer. For example, buffer exchanging of the obtained protein conjugate into a formulation buffer or a storage buffer. The buffer may comprise one or more pharmaceutically acceptable excipients. The excipient may help in improving the bioavailability or stability of the active pharmaceutical ingredient (e.g., the protein conjugate of the present disclosure) during its storage and use.

In the present disclosure, the method may further comprising step (c) contacting a protein comprising an oligosaccharide comprising -GlcNAc(Fuc)_{b} with a UDP-galactose in the presence of a catalyst, to obtain said protein comprising -GlcNAc(Fuc)_{b}-Gal, wherein Gal is a galactose, b is 0 or 1. In the present disclosure, the catalyst may be a β1,4-galactosyltransferase. For example, b is 0. For example, b is 1.

For example, an antibody with heterogenous glycosylation forms of G₀(F), G₁(F), G₂(F) could be transform to an uniform antibody-G₂(F) which contains four -GlcNAc-Gal moieties in an antibody molecule in the presense of a β1,4-galactosyltransferase and UDP-galactose. The antibody-G₂(F) is according to the formula

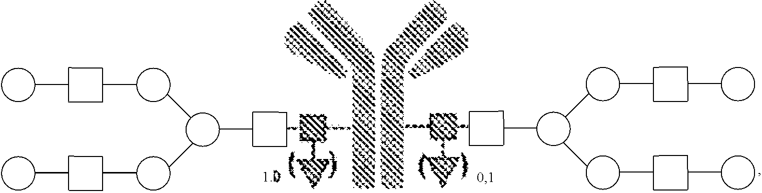

wherein [symbol] is a GlcNAc, [symbol] is a fucose linked the GlcNAc through an α1,6 linkage, [symbol] is a mannose, ◯ is a galactose linked to the GlcNAc through a Galβ1,4GlcNAc linkage, [symbol] is an antibody or Fc-fusion protein, and the oligosaccharide is linked to N297 position of antibody.

For example, an antibody-GlcNAc(Fuc)_{b} could be transformed to an antibody-GlcNAc(Fuc)_{b}-Gal (also named as antibody-(Fuc)_{b}(Galβ1,4)GalNAc) which contains two -GlcNAc(Fuc)_{b}-Gal moieties in an antibody molecule in the presence of a β1,4-galactosyltransferase. The antibody-GlcNAc(Fuc)_{b}-Gal is according to the formula

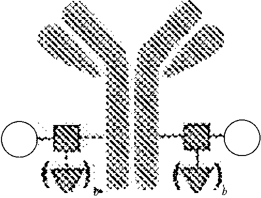

(e.g., the corresponding antibody), wherein [symbol] is a GlcNAc, [symbol] is a fucose linked the GlcNAc through an α1,6 linkage, ◯ is a galactose linked to the GlcNAc through a Galβ1,4GlcNAc linkage, [symbol] is an antibody or Fc-fusion protein, b is 0 or 1 and the GlcNAc is linked to N297 position of antibody. For example, b is 0. For example, b is 1.

In the present disclosure, step (c) may be performed before step (a). In the present disclosure, the method may comprise a purification process between step (c) and step (a). In the present disclosure, the method may not comprise a purification process between step (a) and step (c). In the present disclosure, the step (a) and step (c) may be performed in the same reaction vessel. In the present disclosure, fucosyltransferase, Q-Fuc-(F)ₘ-(L)ₙ-Y₁ or Q-Fuc-(F)ₘ-(L)ₙ-P of step (a) and galactosyltransferase and UDP-galactose in step (c) may be in the same reaction vessel. In the present disclosure, step (a) and step (c) may be performed simultaneously. In the present disclosure, step (a) and step (c) may be performed at the same time. In the present disclosure, step (a) may be performed before step (c) was finished.

In the present disclosure, the method may further comprise a step (d) modifying a protein comprising an oligosaccharide to a protein comprising a core -((Fuc)α1,6) GlcNAc or core -GlcNAc, wherein in the core -((Fuc)α1,6)GlcNAc or core -GlcNAc, the GlcNAc is directly linked to an amino acid of the protein (the amino acid usually is a Asn), and the (Fuc) is linked to the GlcNAc through an α1,6 linkage. The GlcNAc of the core “-((Fuc)α1,6) GlcNAc” is directly linked to an amino acid of the protein. For example, the amino acid of the protein is an Asn. For example, the amino acid of the protein is Asn297. The endoglycosidase may cleave glycan chains from a glycoprotein (e.g. an antibody) and leave a core GlcNAc if the glycoproein doesn't have an core α1,6 fucose linked to the core GlcNAc. The endoglycosidase may cleave glycan chains from a glycoprotein (e.g. an antibody) and leave a core -((Fuc)α1,6)GlcNAc if the glycoproein have an core α1,6 fucose linked to the core GlcNAc. In the present disclosure, the endoglycosidase can modify the oligosaccharide of the antibody or Fc-fusion protein to a -GlcNAc or -((Fuc)α1,6) GlcNAc). In the present disclosure, the endoglycosidase may be an Endo S, an Endo A, an Endo F, an Endo M, an Endo D or their functional mutants or variants, or any combination thereof. In the present disclosure, the endoglycosidase may be an EndoS. For example, the endoglycosidase may have an amino acid sequence as set forth in SEQ ID NO: 6 or 17.

For example, an antibody with heterogenous glycosylation forms (e.g., the corresponding antibody) may be trimmed to an uniform antibody-GlcNAc(Fuc) by using the endoglycosidase. The antibody-((Fuc)α1,6)GlcNAc (i.e. antibody-GlcNAc(Fuc)) is according to the formula

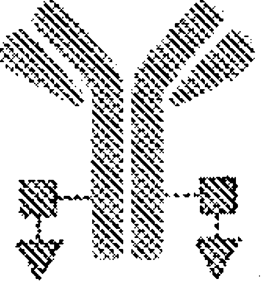

Wherein [symbol] is a GlcNAc, [symbol] is a fucose linked the GlcNAc through an α1,6 linkage, [symbol] is an antibody or Fc-fusion protein, and the GlcNAc is linked to N297 position of the antibody. For example, an antibody with heterogenous glycosylation forms may be trimmed to a uniform antibody-GlcNAc by using the endoglycosidase. The antibody-GlcNAc is according to the formula

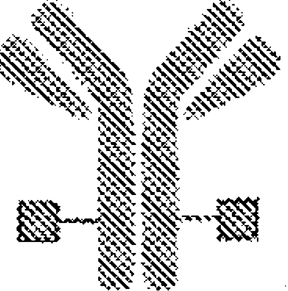

Wherein [symbol] is a GlcNAc, [symbol] is an antibody or Fc-fusion protein, and the GlcNAc is linked to N297 position of the antibody.

In the present disclosure, the method may further comprise a step (e) to remove the core α-1,6 fucose from the protein comprise a core -((Fuc)α1,6)GlcNAc to generate a protein comprising the core -GlcNAc. For example, step (e) may be performed in presence of a core-α1,6 fucosidase. For example, the core-α1,6 fucosidase may be a BfFucH, a fucosidase O, an Alfc, a BKF, a fucosidase O or their functional mutants or variants, or any combination thereof.

For example, the core-α1,6 fucosidase may be Alfc. For example, the core-α1,6 fucosidase may have a protein sequence according to the SEQ ID NO: 7 or SEQ ID NO: 18.

For example, The antibody-GlcNAc(Fuc) could be further trimed to antibody-GlcNAc by using the core-α1,6 fucosidase (e.g. Alfc). The antibody-GlcNAc is according to the formula

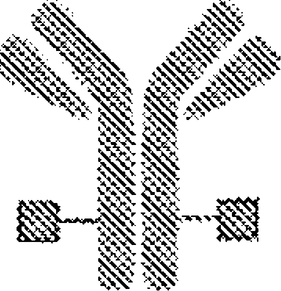

(e.g., the corresponding antibody), Wherein [symbol] is a GlcNAc, [symbol] is an antibody or Fc-fusion protein, and the GlcNAc is linked to N297 position of the antibody.

In the present disclosure, step (e) may be performed between step (d) and step (c). In the present disclosure, there may be a purification process between step (d) and step (e). In the present disclosure, there may not be a purification process between step (d) and step (e). In the present disclosure, there may be a purification process between step (e) and step (c). In the present disclosure, there may not be a purification process between step (e) and step (c)

In the present disclosure, step (d) and step (e) may be performed in the same reaction vessel. In the present disclosure, step (d) and step (e) may be performed simultaneously. For example, an antibody may be modified to antibody-GlcNAc by adding a core-α1,6 fucosidase and an endoglycosidase simultaneously. For example, an antibody may be modified to antibody-GlcNAc by adding a core-α1,6 fucosidase and an endoglycosidase simultaneously in the same reaction vessel.

In the present disclosure, the method may be performed following the order of step (c)-step (a). In the present disclosure, the steps may be performed following the orders of step (c)-step (a)-step (b). In the present disclosure, there may be a purification step between each of the steps. In the present disclosure, in a “one-pot” process, there may not be a purification process between step (c) and step (a). In the present disclosure, in a “one-pot” process, the step (a) and step (c) may be a performed simultaneously in the same reaction vessel.

In the present disclosure, the method may be performed following the order of step (d)-step (c)-step (a). In the present disclosure, the method may be performed following the orders of step (d)-step (c)-step (a)-step (b). In the present disclosure, there may be a purification step between each of the steps. In the present disclosure, in a “one-pot” process, there may not be a purification process between the step (d) and the step (c), and between the step (c) and the step (a). In the present disclosure, in a “one-pot” process, the step (a), step (c) and step (d) may be performed simultaneously in the same reaction vessel.

In the present disclosure, the method may be performed following the order of step (d)-step (e)-step (c)-step (a). In the present disclosure, the method may be performed following the orders of step (d)-step (e)-step (c)-step (a)-step (b). In the present disclosure, there may be a purification step between each of the steps. In the present disclosure, in a “one-pot” process there may not be a purification process between the step (d) and the step (e) between the step (e) and the step (c), and between the step (c) and the step (a). In the present disclosure, in a “one-pot” process, the step (d) and the step (e) may be performed simultaneously in the same vessel before the step (c), and then step (c) and the step (a) were performed simultaneously in the same vessel in which the step (d) and step (e) were performed.

In the present disclosure, the enzymes, the reactants (proteins comprising an oligosaccharide) and the product (modified proteins comprising the oligosaccharide) may be in the same reaction vessel simultaneously.

In the present disclosure, the method may be a “one-pot” process, which comprises contacting the protein comprising the -GlcNAc(Fuc)_{b} with the UDP-galactose, the β1,4-galactosyltransferase, together with the Q-Fuc-(F)ₘ-(L)ₙ-Y₁ or the Q-Fuc-(F)ₘ-(L)ₙ-P and the Hp α1,3-fucosyltransferase in “one-pot” to obtain a protein conjugate comprising

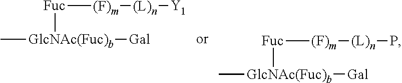

wherein Fuc is the fucose or fucose derivative, F is a connector, L is a linker, Y₁ is a functional group, P is a biologically or pharmaceutically active substance, b is 0 or 1, m is 0 or 1, and n is 0 or 1. For example, b is 0. For example, b is 1. For example, antibody-G₂(F)-Fuc-(F)ₘ-(L)ₙ-P or antibody-G₂(F)-Fuc-(F)ₘ-(L)ₙ-Y₁ could be obtained by contacting an antibody with heterogenous glycosylation forms of G₀(F), G₁(F), G₂(F) with the UDP-galactose, the β1,4-galactosyltransferase, the Q-Fuc-(F)ₘ-(L)ₙ-Y₁ or the Q-Fuc-(F)ₘ-(L)ₙ-P and the Hp α1,3-fucosyltransferase in “one-pot”. During the whole process, only one purification process were performed to obtain the antibody-G₂(F)-Fuc-(F)ₘ-(L)ₙ-P or the antibody-G₂(F)-Fuc-(F)ₘ-(L)ₙ-Y₁

In the present disclosure, the method may be a “one-pot” process, which comprise contacting the protein comprising the -GlcNAc(Fuc)_{b} with the UDP-galactose and the β1,4-galactosyltransferase for some time, followed by directly adding the Q-Fuc-(F)ₘ-(L)ₙ-Y₁ or the Q-Fuc-(F)ₘ-(L)ₙ-P and the the Hp α1,3-fucosyltransferase to the reaction mixture without further purification of the protein from the reaction mixture, to obtain a protein conjugate comprising

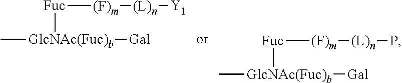

wherein Fuc is the fucose or fucose derivative, F is a connector, L is a linker, Y₁ is a functional group, P is a biologically or pharmaceutically active substance, b is 0 or 1, m is 0 or 1, and n is 0 or 1. For example, b is 0. For example, b is 1. For example, antibody-G₂(F)-Fuc-(F)ₘ-(L)ₙ-P or antibody-G₂(F)-Fuc-(F)ₘ-(L)ₙ-Y₁ could be obtained by contacting an antibody with heterogenous glycosylation forms of G₀(F), G₁(F), G₂(F) with the UDP-galactose and the β1,4-galactosyltransferase, followed by directly adding the Q-Fuc-(F)ₘ-(L)ₙ-Y₁ or the Q-Fuc-(F)ₘ-(L)ₙ-P and the Hp α1,3-fucosyltransferase to the reaction mixture without further purification of the protein from the reaction mixture. During the whole process, only one purification process were performed to obtain the antibody-G₂(F)-Fuc-(F)ₘ-(L)ₙ-P or the antibody-G₂(F)-Fuc-(F)ₘ-(L)ₙ-Y₁.

In the present disclosure, the method may be a “one-pot” process, which comprise contacting a protein comprising an oligosaccharide with the endoglycosidase, the UDP-galactose, the β1,4-galactosyltransferase, the Q-Fuc-(F)ₘ-(L)ₙ-Y₁ or the Q-Fuc-(F)ₘ-(L)ₙ-P and the Hp α1,3-fucosyltransferase in “one-pot” to obtain a protein conjugates comprising

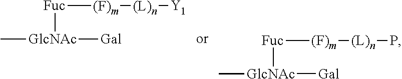

wherein Fuc is the fucose or fucose derivative, F is a connector, L is a linker, Y₁ is a functional group, P is a biologically or pharmaceutically active substance, m is 0 or 1, and n is 0 or 1. For example, antibody-(Galβ1,4)GalNAc-Fuc-(F)ₘ-(L)ₙ-Y₁ or antibody-(Galβ1,4)GalNAc-Fuc-(F)ₘ-(L)ₙ-P could be obtained by contacting an antibody with heterogenous glycosylation forms with the endoglycosidase, the UDP-galactose, the β1,4-galactosyltransferase, the Q-Fuc-(F)ₘ-(L)ₙ-Y₁ or the Q-Fuc-(F)ₘ-(L)ₙ-P and the Hp α1,3-fucosyltransferase in “one-pot”. During the whole process, only one purification process after all the reaction were performed to obtain the antibody-(Galβ1,4)GalNAc-Fuc-(F)ₘ-(L)ₙ-P or antibody-(Galβ1,4)GalNAc-Fuc-(F)ₘ-(L)ₙ-Y₁.

In the present disclosure, the method may be a “one-pot” process, which comprise contacting a protein comprising an oligosaccharide with an endoglycosidase and a core-α1,6 fucosidase for some time, followed by directly adding the UDP-galactose, the β1,4-galactosyltransferase, the Q-Fuc-(F)ₘ-(L)ₙ-Y₁ or the Q-Fuc-(F)ₘ-(L)ₙ-P and the Hp α1,3-fucosyltransferase to the reaction mixture without further purification of the protein from the reaction mixture, to obtain a protein conjugate comprising

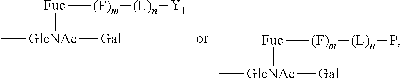

wherein Fuc is the fucose or fucose derivative, F is a connector, L is a linker, Y₁ is a functional group, P is a biologically or pharmaceutically active substance, b is 0 or 1, m is 0 or 1, and n is 0 or 1. For example, b is 0. For example, b is 1. For example, For example, antibody-(Galβ1,4)GalNAc-Fuc-(F)ₘ-(L)ₙ-Y₁ or antibody-(Galβ1,4)GalNAc-Fuc-(F)ₘ-(L)ₙ-P could be obtained by contacting an antibody with heterogenous glycosylation forms the endoglycosidase and the core-α1,6 fucosidase for some time. The reaction were followed by directly adding the UDP-galactose, the β1,4-galactosyltransferase, the Q-Fuc-(F)ₘ-(L)ₙ-Y₁ or the Q-Fuc-(F)ₘ-(L)ₙ-P and the Hp α1,3-fucosyltransferase to the reaction mixture without further purification of the protein from the reaction mixture. During the whole process, only one purification process after all the reaction were performed to obtain the antibody-(Galβ1,4)GalNAc-Fuc-(F)ₘ-(L)ₙ-P or antibody-(Galβ1,4)GalNAc-Fuc-(F)ₘ-(L)ₙ-Y₁.

Multiple rounds of purification of antibodies from the reaction mixture is a laborious task. These “one-pot” method significant simplified the process for the preparation of antibody conjugates.

In another aspect, the present disclosure provides use of the Q-Fuc*′ of the present disclosure in preparation of said protein conjugate.

In the present disclosure, the Q-Fuc*′ is Q-Fuc-(F)ₘ-(L)ₙ-Y₁ or Q-Fuc-(F)ₘ-(L)ₙ-P. In some embodiments, the Q-Fuc*′ is GDP-Fuc-(F)ₘ-(L)ₙ-Y₁ or GDP-Fuc-(F)ₘ-(L)ₙ-P, wherein F is a connector, L is a linker, P is a biologically and/or a pharmaceutically active substance, Y₁ is a functional group, m is 0 or 1 and n is 0 or 1. In some embodiments, the Q-Fuc*′ is GDP-Fuc-F-(L)ₙ-Y₁ or GDP-Fuc-F-(L)ₙ-P, wherein n is 0 or 1. The structure of the connector F may have significant influence on the catalytic efficiency of α-1,3-fucosyltransferase in transferring an active moiety (e.g. Y₁ or P) to the GlcNAc of the comprised by a protein, wherein b is 0 or 1. For example, a GDP-Fuc-F-(L)ₙ-Y₁ or a GDP-Fuc-F-(L)ₙ-P with a connector F comprising a

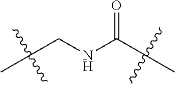 [SMILES: *CNC(*)=O]

the left terminus of the structure is directly linked to the Fuc) may be more efficiently to be transferred to an antibody or a Fc fusion protein comprising the -GlcNAc(Fuc)_{b}-Gal, wherein b is 0 or 1 and n is 0 or 1 (i.e. the GDP-Fuc-F-(L)ₙ-Y₁ or GDP-Fuc-F-(L)ₙ-P comprising a structure of

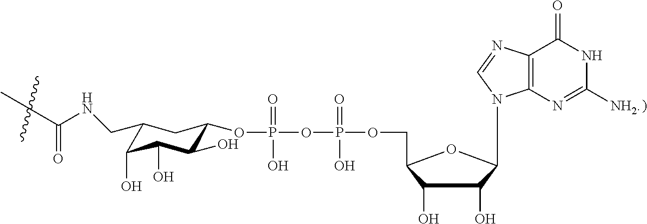

For example, an α-1,3-fucosyltransferase may have significant higher catalytical efficiency towards a GDP-Fuc-(F)ₘ-(L)ₙ-Y₁ or a GDP-Fuc-(F)ₘ-(L)ₙ-P with a connector comprising a

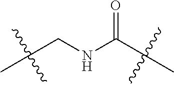 [SMILES: *CNC(*)=O]

the left terminus of the structure is directly linked to the Fuc) than those with a connector comprising a

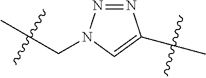 [SMILES: *Cn1cc(*)nn1]

(the left terminus of the structure is directly linked to the Fuc) in transferring an active moiety (e.g. Y₁ or P) to an antibody or a Fc fusion protein comprising the -GlcNAc(Fuc)_{b}-Gal, wherein b is 0 or 1 and n is 0 or 1. That is: An α-1,3-fucosyltransferase may have significant higher catalytical efficiency towards a GDP-Fuc-F-(L)ₙ-Y₁ or a GDP-Fuc-F-(L)ₙ-P comprising a structure of

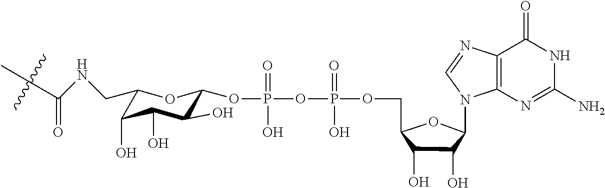

than those comprising a structure of

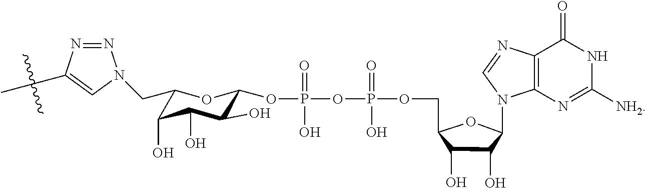

For example, an α-1,3-fucosyltransferase from Helicobacter pylori may display higher catalytical efficiency towards a GDP-Fuc-F-(L)ₙ-Y₁ or a GDP-Fuc-F-(L)ₙ-P comprising a structure of

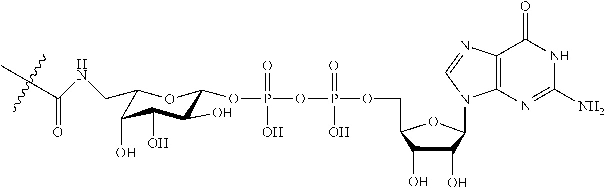

than those comprising a structure of

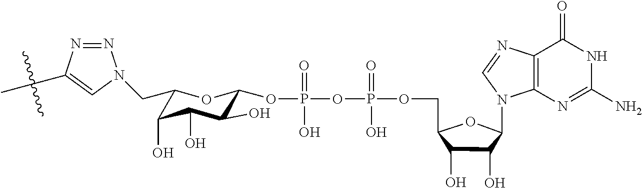

in transferring an active moiety (e.g. Y₁ or P) to an antibody or a Fc fusion protein comprising the -GlcNAc(Fuc)_{b}-Gal, wherein b is 0 or 1 and n is 0 or 1. For example, an α-1,3-fucosyltransferase comprise an amino acid sequence as set forth in SEQ ID NO: 3 or SEQ ID NO: 4 may display significant higher catalytical efficiency towards a GDP-Fuc-F-(L)ₙ-Y₁ or a GDP-Fuc-F-(L)ₙ-P comprising a structure of

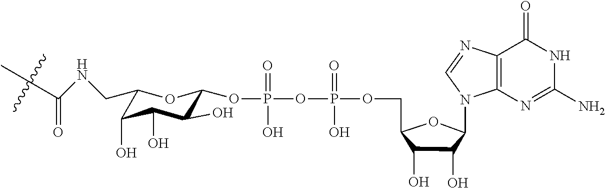

than those comprising a structure of

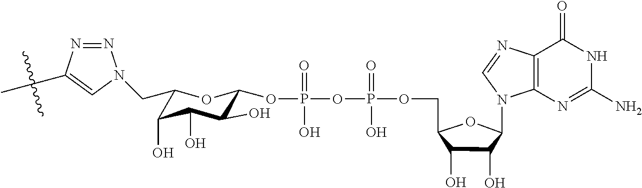

in transferring an active moiety (e.g. Y₁ or P) to an antibody-G₂(F) or an antibody-(Galβ1,4)GlcNAc. For example, the Hp-α(1,3)-FucT (SEQ ID NO: 4) displayed significant higher catalytic efficiency towards the GDP-FAmP₄Biotin

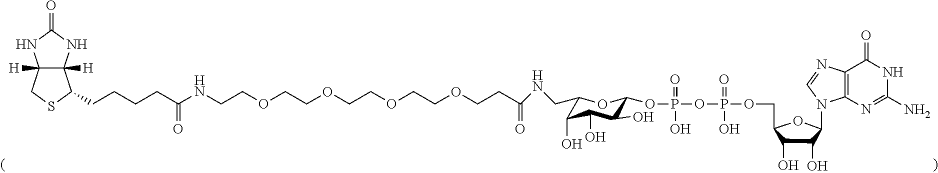

than the GDP-FAzP₄Biotin

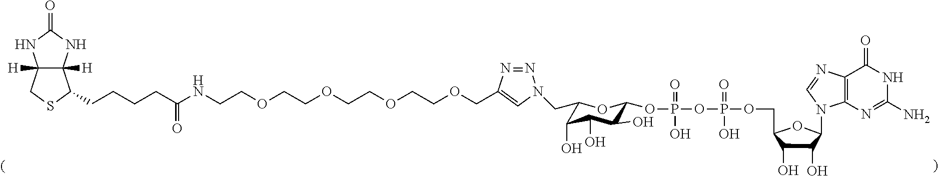

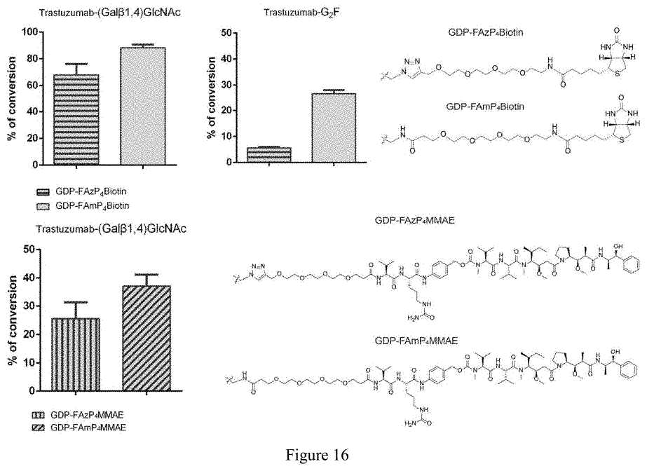

in transferring the Biotin to the trastuzumab-G₂F and the trastuzumab-(Galβ1,4)GlcNAc as show in FIG. 16 A. For example, the Hp-α(1,3)-FucT (SEQ ID NO: 4) displayed significant higher catalytic efficiency towards the GDP-FAmP₄MMAE

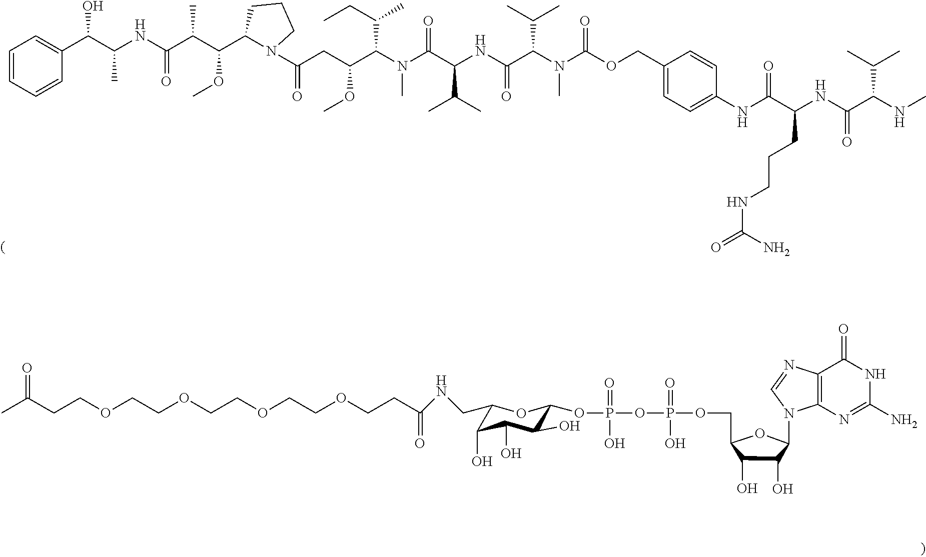

than the GDP-FAzP₄MMAE

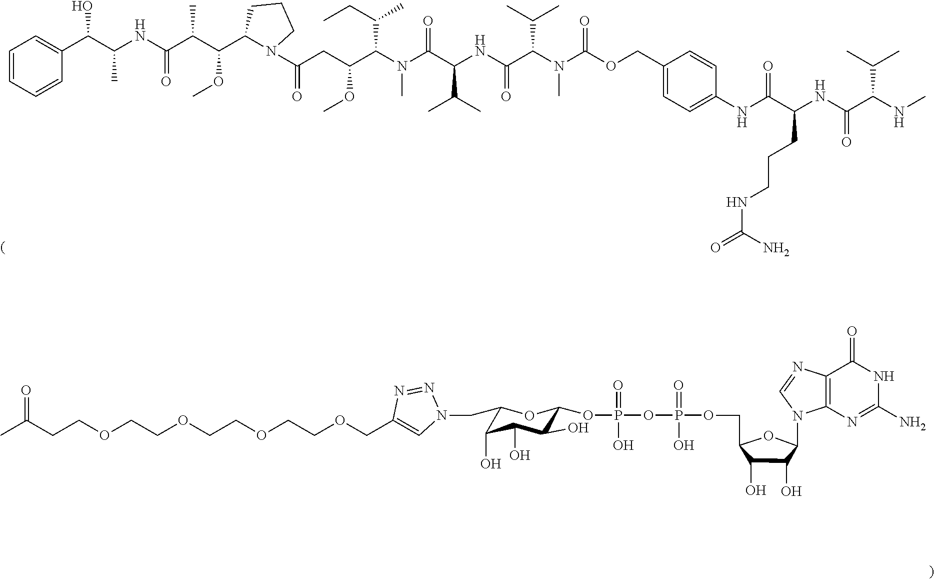

than the in transferring the MMAE to the trastuzumab-(Galβ1,4)GlcNAc as show in FIG. 16 B.

In the present disclosure, the Hp α-1,3-fucosyltransferase may have much higher catalytical efficiency than the Human α-1,3-fucosyltransferase in transferring the Q-Fuc*′ to an antibody or a Fc-fusion protein. For example, a Hp α-1,3-fucosyltransferase comprise an amino acid sequence as set forth in SEQ ID NO: 3 may display much higher efficiency in transferring GDP-(F)ₘ-(L)ₙ-P or GDP-(F)ₘ-(L)ₙ-Y₁ to an antibody or a Fc-fusion protein than a Human α-1,3-fucosyltransferase comprise an amino acid sequence as set forth in SEQ ID NO: 5. For example, the Hp-α(1,3)-FucT (SEQ ID NO: 4) displayed much higher efficiency in transferring GDP-(F)ₘ-(L)ₙ-P or GDP-(F)ₘ-(L)ₙ-Y₁ to an antibody-G₂(F) or an antibody-(Galβ1,4)GlcNAc than the Human FT6 (SEQ ID NO: 5). For example, the Hp-α(1,3)-FucT (SEQ ID NO: 4) displayed much higher efficiency than the Human FT6 (SEQ ID NO: 5) in transferring GDP-FAmP₄Biotin to trastuzumab-G₂F as show in FIG. 18. After 3 hours, the Hp-α(1,3)-FucT achieved 10% of conversion while the Human FT6 achieved undetectable level of conversion. After 16 hours, the Human FT6 only achieved 4% of conversion. In contrast, the Hp-α(1,3)-FucT achieved 69% of conversion.

In the present disclosure, a protein comprising a -GlcNAc-Gal linked directly to an amino acid residue (e.g. Asn) may be more efficiently to be converted to a protein conjugate comprising

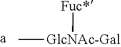

than a protein comprising a -GlcNAc-Gal linked to a saccharide (e.g. mannose) to be converted to a protein conjugate comprising

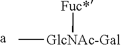

by using a Q-Fuc*′ and an α-1,3-fucosyltransferase. For example, an α-1,3-fucosyltransferase may displayed higher efficiency in transferring GDP-Fuc-(F)ₘ-(L)ₙ-Y₁ or GDP-Fuc-(F)ₘ-(L)ₙ-P to an antibody-(Galβ1,4)GlcNAc than to an antibody-G₂(F). For example, the Hp-α(1,3)-FucT (SEQ ID NO: 4) displayed higher efficiency in transferring GDP-FAmP₄Biotin to the trastuzumab-(Galβ1,4)GlcNAc than to the trastuzumab-G₂F as shown in FIG. 16A. After 2 hours, the trastuzumab-GlcNAc-Gal achieved a 88% of conversion. In contrast, the trastuzumab-G₂F only achieved a 27% of conversion even after 6 hours.

In the present disclosure, a protein comprising a -GlcNAc-Gal may be more efficiently to be converted to a protein conjugate comprising

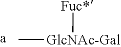

than a protein with

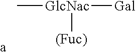

to be converted to a protein conjugate comprising

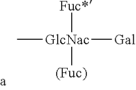

by using a Q-Fuc*′ and an α-1,3-fucosyltransferase. In the present disclosure, a protein comprising a -GlcNAc-Gal directly linked to an Asn may be more efficient to be converted to a protein conjugate comprising

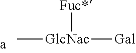

than a protein comprising

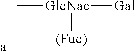

directly linked to an Asn to be converted to a protein conjugate comprising

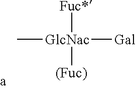

by using a Q-Fuc*′ and an α-1,3-fucosyltransferase. For example, an antibody or a Fc fusion protein comprising a -GlcNAc-Gal directly linked to an Asn may be more efficiently to be converted to an antibody conjugate or a Fc fusion conjugate comprising

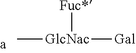

than an antibody or a Fc fusion protein comprising

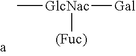

directly linked to an Asn to be converted to an antibody conjugate or a Fc fusion protein conjugate comprising

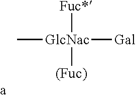

by using a Q-Fuc*′ and an α-1,3-fucosyltransferase. For example, a Hp α-1,3-fucosyltransferase may displayed higher efficiency in transferring GDP-Fuc-(F)ₘ-(L)ₙ-Y₁ or GDP-Fuc-(F)ₘ-(L)ₙ-P to an antibody-GlcNAc-Gal than to an antibody-((Fuc)α1,6)GlcNAc-Gal. For example, it took longer time for the trastuzumab-((Fuc)α1,6)GlcNAc-Gal to achieve a >90% of conversion than for the trastuzumab-GlcNAc-Gal to achieve a >90% of conversion in the presence of the Hp-α(1,3)-FucT (SEQ ID NO 4) and the GDP-FAmSucMMAE.

In another aspect, the present disclosure provides a method for preparation of a composition comprising the protein conjugate.

In some embodiments, the present disclosure provide a method for preparation of a composition comprising the protein conjugate comprising

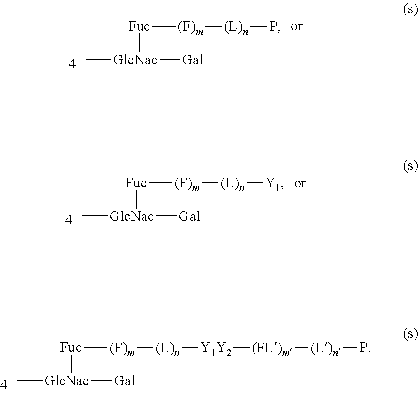

wherein, Fuc is the fucose or fucose derivative, Y₁Y₂ is the remaining group, Y₁ is the functional group, L is the linker, F is the connecter, L′ is the linker defined as the same as the L, FL′ is the spacer defined as the same as the FL, P is the biologically and/or pharmaceutically active substance, m is 0 or 1, n is 0 or 1, m′ is 0 or 1 and n′ is 0 or 1. For example, the protein is an antibody, and the GlcNAc of is linked to the mannose of the

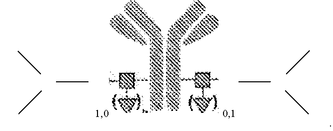

For example, the protein conjugate has the similar binding affinity as the corresponding antibody towards an antigen. For example, the protein conjugate is for treating disease. For example, the protein conjugate is for making an agent for treating disease. For example, the composition has a average MAR of about 2.4-4. For example, the composition has an average MAR of about 2.8-4. For example, the composition has a average MAR of about 3.2-4. For example, the composition has a average MAR of about 3.6-4. For example, the composition has an average MAR of about 3.8-4. For example, the composition has a average MAR of about 4.

In some embodiments, the present disclosure provide a method for preparation of a composition comprising the protein conjugate comprising

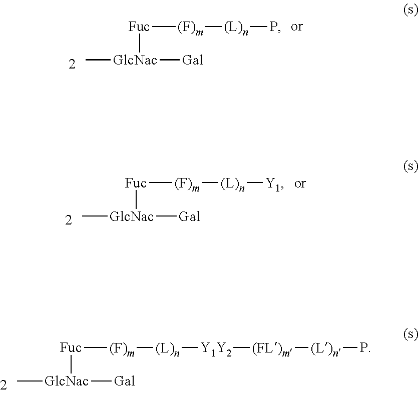

In some embodiments, the present disclosure provide a method for preparation of a composition comprising the protein conjugate comprises

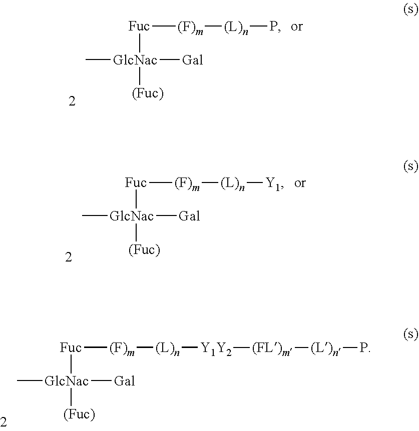

wherein, (Fuc) is the fusoce linked to the GlcNAc through an α1,6 linkage, Fuc is the fucose or fucose derivative, Y₁Y₂ is the remaining group, Y₁ is the functional group, L is the linker, F is the connecter, L′ is the linker defined as the same as the L, FL′ is the spacer defined as the same as the FL, P is the biologically and/or pharmaceutically active substance, m is 0 or 1, n is 0 or 1, m′ is 0 or 1 and n′ is 0 or 1. For example, the protein is an antibody, and the GlcNAc is directly linked to a N297 of the antibody. For example, the protein conjugate has the similar binding affinity as the corresponding antibody towards an antigen. For example, the protein conjugate is for treating disease For example, the protein conjugate is for making an agent for treating disease. For example, the composition has a average MAR of about 0.5-2. For example, the composition has an average MAR of about 1-2. For example, the composition has an average MAR of about 1.5-2. For example, the composition has a average MAR of about 1.8-2. For example, the composition has an average MAR of about 2.

In another aspect, the present disclosure provides a protein conjugate, which is obtained from the method of the present disclosure.

In another aspect, the present disclosure provides a composition, which is obtained from the method of the present disclosure.

In another aspect, the present disclosure provides use of the Q-Fuc*′ of the present disclosure in preparation of said protein conjugate.

In another aspect, the present disclosure provides a pharmaceutical composition, comprising the protein conjugate of the present disclosure and optionally a pharmaceutically acceptable carrier.

In another aspect, the present disclosure provides a pharmaceutical composition, comprising the composition of the present disclosure and optionally a pharmaceutically acceptable carrier.

In addition to the compositions and protein conjugates described above, the present invention also provides a number of methods that can be practiced utilizing the compounds and protein conjugates of the present disclosure. Methods for using the protein conjugate of the present disclosure may comprises: killing or inhibiting the growth or replication of a tumor cell or cancer cell, treating cancer, treating a pre-cancerous condition, killing or inhibiting the growth or replication of a cell that expresses an auto-immune antibody, treating an autoimmune disease, treating an infectious disease, preventing the multiplication of a tumor cell or cancer cell, preventing cancer, preventing the multiplication of a cell that expresses an auto-immune antibody, preventing an autoimmune disease, and preventing an infectious disease. These methods of use comprise administering to an animal such as a mammal or a human in need thereof an effective amount of a protein conjugate.

Pharmaceutical compositions typically must be sterile and stable under the conditions of manufacture and storage. The pharmaceutical composition can be formulated as suitable for administration. The pharmaceutical composition can be formulated as a solution, emulsion, lyophilized formulation, microemulsion, liposome, or other ordered structure suitable to high drug concentration. As used herein, the term “pharmaceutically acceptable carrier” generally refers to a pharmaceutically acceptable adjuvant, excipient or stabilizer, which are non-toxic to the cells or subjects exposed to them at an administrated dose and concentration. Generally, the pharmaceutically acceptable carrier may be an aqueous solution. Examples of a pharmaceutically acceptable carrier may comprise a buffer, an antioxidant, a low molecular weight (less than about 10 residues) polypeptide, a protein, a hydrophilic polymer, a monosaccharide, a disaccharide and other carbohydrates, a chelating agent, a sugar alcohol, a salt-forming counterion, such as sodium; a nonionic surfactant, a preservative, a wetting agent, an emulsifying agent and/or a dispersing agent.

In another aspect, the present disclosure provides a method for preventing or treating disease, comprising administrating the protein conjugate of the present disclosure and/or the pharmaceutical composition of the present disclosure.

In another aspect, the present disclosure provides a method for preventing or treating disease, comprising administrating the composition of the present disclosure and/or the pharmaceutical composition of the present disclosure.

In another aspect, the present disclosure provides the use of the protein conjugate and/or the pharmaceutical composition, in preparation of a medicament for preventing or treating disease.

In another aspect, the present disclosure provides the use of the composition and/or the pharmaceutical composition, in preparation of a medicament for preventing or treating disease.

In another aspect, the present disclosure provides the protein conjugate and/or the pharmaceutical composition, for use in preventing or treating disease.

In another aspect, the present disclosure provides the composition and/or the pharmaceutical composition, for use in preventing or treating disease.

In another aspect, the present disclosure further provides the embodiments as following:
1. A protein conjugate, comprising a first part comprising a N-acetyllactosamine (LacNAc), and a second part comprising a fucose or fucose derivative Fuc and an active moiety, wherein said Fuc is linked with a N-acetylglucosamine (GlcNAc) of said LacNAc via a covalent bond.
2. The protein conjugate of embodiment 1, wherein said first part comprises a N-glycosylation chain, and said LacNAc is located on said N-glycosylation chain.
3. The protein conjugate of any one of embodiments 1-2, wherein said first part comprising a Fc fragment, and said LacNAc is located on a N-glycosylation chain of said Fc fragment.
4. The protein conjugate of embodiment 3, wherein said LacNAc is linked with a mannose of said N-glycosylation chain.
5. The conjugate of embodiment 1, wherein said LacNAc is directly linked to an amino acid of said first part.
6. The conjugate of any one of embodiments 1-5, wherein said LacNAc is a Gal-β(1,4)-GlcNAc and optionally modified with a fucose on the GlcNAc.
7. The conjugate of any one of embodiments 1-6, wherein said first part comprises an isolated protein.
8. The conjugate of any one of embodiments 1-7, wherein said first part comprises an antibody and a fragment with a Fc thereof.
9. The conjugate of any one of embodiments 1-8, wherein said active moiety comprises a molecular weight from 0 to 500,000 Dalton.
10. The conjugate of any one of embodiments 1-9, wherein said active moiety comprises a small molecule with a molecular weight from 0 to 20,000 Dalton, a peptide, a polypeptide, a polymer, a protein, or an oligonucleotide.
11. The conjugate of any one of embodiments 1-10, wherein said active moiety comprises a first reactive group (Y₁).
12. The conjugate of any one of embodiments 1-11, wherein said active moiety comprises a remaining group after reacting said first reactive group (Y₁) with a second reactive group (Y₂).
13. The conjugate of embodiment 12, wherein said Y₂ is linked to an active molecule.
14. The conjugate of embodiments 12-13, wherein said Y₁ and/or Y₂ comprising a bioorthogonal reaction group.
15. The conjugate of embodiments 12-14, wherein said Y₁ and/or Y₂ comprises a bioorthogonal reaction group selected from a group consisting of azide, terminal alkyne, cyclic alkyne, tetrazine, 1,2,4-trazine, terminal alkene, transcyclooctene, cyclopropene, norbornene, keto, aldehyde, aminooxy, thiol and maleimide.
16. The conjugate of embodiments 12-15, wherein said Y₁ and/or Y₂ comprises a bioorthogonal reaction group selected from a group consisting of

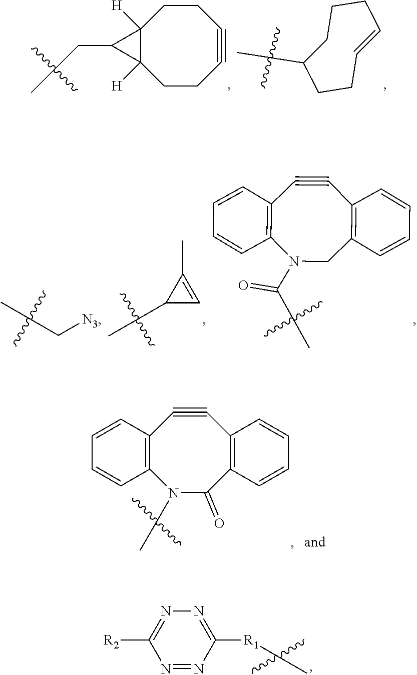

wherein said R₁ and R₂ are independently selected from the group consisting of hydrogen halogen, C₁-C₂₂ alkyl groups, C₅-C₂₂ (hetero)aryl groups, C₇-C₂₂ alkyl(hetero)aryl groups and C₇-C₂₂ (hetero)arylalkyl groups, the alkyl groups optionally being interrupted by one or more hetero-atoms selected from the group consisting of O, N, and S, and wherein the alkyl groups, (hetero)aryl groups, alkyl(hetero)aryl groups and (hetero)arylalkyl groups are independently optionally substitute.
17. The conjugate of any one of embodiments 12-16, wherein said Y₁ comprises

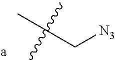 [SMILES: *CN=[N+]=[N-]]

and said Y₂ comprises a

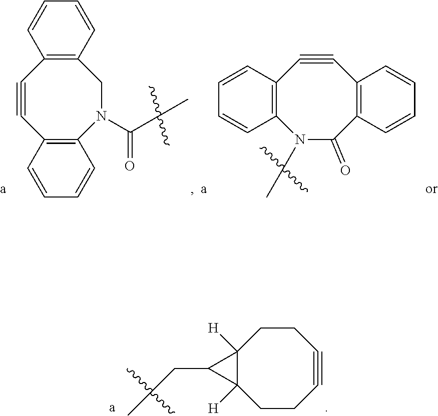 [SMILES: *C(=O)N1Cc2ccccc2C#Cc2ccccc21 ; *N1C(=O)c2ccccc2C#Cc2ccccc21 ; *CC1C2CCC#CCCC12]

18. The conjugate of any one of embodiments 12-17, wherein said Y₁ comprises

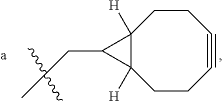 [SMILES: *CC1C2CCC#CCCC12]

and said Y₂ comprise

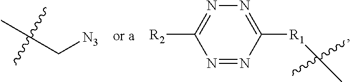

wherein said R₁ and R₂ are independently selected from the group consisting of hydrogen, halogen, C₁-C₂₂ alkyl groups, C₅-C₂₂ (hetero)aryl groups, C₇-C₂₂ alkyl(hetero)aryl groups and C₇-C₂₂ (hetero)arylalkyl groups, the alkyl groups optionally being interrupted by one or more hetero-atoms selected from the group consisting of O, N, and S, and wherein the alkyl groups, (hetero)aryl groups, alkyl(hetero)aryl groups and (hetero)arylalkyl groups are independently optionally substitute.
19. The conjugate of any one of embodiments 12-18, wherein said Y₁ comprises

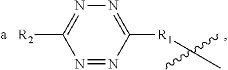

wherein said R₁ and R₂ are independently selected from the group consisting of hydrogen, halogen, C₁-C₂₂ alkyl groups, C₅-C₂₂ (hetero)aryl groups, C₇-C₂₂ alkyl(hetero)aryl groups and C₇-C₂₂ (hetero)arylalkyl groups, the alkyl groups optionally being interrupted by one or more hetero-atoms selected from the group consisting of O, N, and S, and wherein the alkyl groups, (hetero)aryl groups, alkyl(hetero)aryl groups and (hetero)arylalkyl groups are independently optionally substitute, and said Y₂ comprises

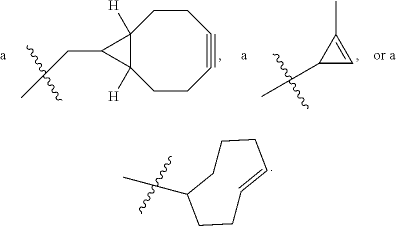

20. The conjugate of any one of embodiments 12-19, wherein said Y₁ comprises

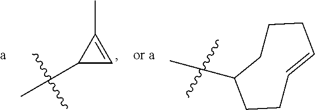

and said Y₂ comprises

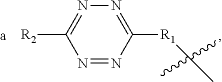

wherein said R₁ and R₂ are independently selected from the group consisting of hydrogen, halogen, C₁-C₂₂ alkyl groups, C₅-C₂₂ (hetero)aryl groups, C₇-C₂₂ alkyl(hetero)aryl groups and C₇-C₂₂ (hetero)arylalkyl groups, the alkyl groups optionally being interrupted by one or more hetero-atoms selected from the group consisting of O, N, and S, and wherein the alkyl groups, (hetero)aryl groups, alkyl(hetero)aryl groups and (hetero)arylalkyl groups are independently optionally substitute.
21. The conjugate of any one of embodiments 1-20 in said second part, wherein said active moiety linked with said Fuc directly or indirectly.
22. The conjugate of any one of embodiments 1-21, wherein said active moiety linked with said Fuc through a linking unit W.
23. The conjugate of embodiment 22, wherein said linking unit W comprises a spacer FL.
24. The conjugate of any embodiments 22-23, wherein said spacer FL comprises a polypeptide, a PEG, an alkyl and/or derivatives thereof.
25. The conjugate of any embodiments 22-24, wherein said linking unit W comprises

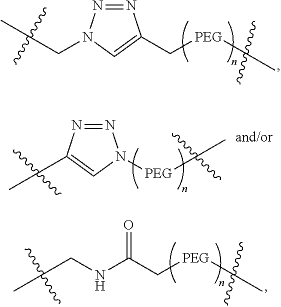

wherein, n is an integer of 0-200.
26. A method of preparation the conjugate of any one of embodiments 1-25, comprising contacting a first molecule comprising a N-acetyllactosamine (LacNAc) with a second molecule (Q-Fuc*′) comprising a fucose or fucose derivative Fuc and an active moiety under a suitable condition capable of transferring said Fuc to a N-acetylglucosamine (GlcNAc) of said LacNAc.
27. The method of any one of embodiment 26, wherein said suitable condition is in presence of a catalyst capable of transferring said Fuc to said GlcNAc of said LacNAc.
28. The method of embodiment 27, wherein said catalyst is a fucosyltransferase.
29. The method of embodiment 28, wherein said fucosyltransferase is an α-1,3-fucosyltransferase.
30. The method of any one of embodiments 28-29, wherein said fucosyltransferase is derived from bacteria, nematodes, trematodes or mammals.
31. The method of any one of embodiments 29-30, wherein said fucosyltransferase is derived from Helicobacter pylori.
32. The method of any one of embodiments 28-31, wherein said fucosyltransferase comprises an amino acid sequence as set forth in GenBank Accession No. AAD07710.1.
33. The method of any one of embodiments 28-30, wherein said fucosyltransferase is derived from human.
34. The method of embodiment 33, wherein said fucosyltransferase comprises an amino acid sequence as set forth in Uniprot Accession No. P51993.
35. The method of any one of embodiments 26-34, wherein said second molecule (Q-Fuc*′) comprises an agent capable of linking with said Fuc.
36. The method of embodiment 35, comprising transferring said Fuc and said active moiety from said agent to said first molecule.
37. The method of any one of embodiments 35-36, wherein said agent comprises a ribonucleotide diphosphate.
38. The method of any one of embodiments 35-37, wherein said agent is selected from uridine diphosphate (UDP), guanosine diphosphate (GDP) and cytidine diphosphate (CDP).
39. The method of any one of embodiments 35-38, wherein said agent comprises guanosine diphosphate (GDP).
40. The method of any one of embodiments 26-39, comprising reacting said Y₁ of conjugate of any one of embodiments 11-20 with said Y₂ of conjugate of any one of embodiments 12-20.
41. Use of the conjugate of any one of embodiments 1-25 in preparation of an antibody-drug conjugate.
42. Use of the conjugate of any one of embodiments 1-25 in preparation of a medicament.
43. The use of embodiment 42, wherein said medicament is used to treat tumor.
44. Pharmaceutical composition, comprising the conjugate of any one of embodiments 1-25 and a pharmaceutically acceptable carrier.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Synthesis of GDP-FAz

GDP-FAz was synthesized according to the reported procedure (Wu P., et al., Proc. Natl. Acad. Sci. USA 2009, 106, 16096), and purified through a Bio-Gel P-2 Gel column (Biorad). HRMS (ESI−) calcd for C₁₆H₂₄N₈O₁₅P₂(M−H⁺) 629.0764, found 629.0785.

### Example 2 Synthesis of GDP-FAm

To a clear solution of 100 mg (0.16 mmol) GDP-FAz in 8.75 mL MeOH/ddH₂O (1:1.5), 5 mg Pd/C (10%) was added. The air atmosphere was changed to H₂ by vacuum and refill, the H₂ pressure was kept at 0.28 MPa. The reaction was allowed for stirred 4 h and filtered through a 0.22 M filter. Rotorvap and lyophilization give the product as a white solid (84.8 mg, yield 88%). HRMS (ESI−) calcd for C₁₆H₂₆N₆O₁₅P₂(M−H⁺) 603.0859, found 603.0874. ¹H NMR (400 MHz, D₂O) δ 8.10 (s, 1H), 5.92 (d, J=6.1 Hz, 1H), 4.97 (t, J=7.6, 1H), 4.76-4.73 (m, 1H), 4.51 (dd, J=5.2, 3.4 Hz, 1H), 4.37-4.34 (m, 1H), 4.23-4.21 (m, 2H), 3.96 (dd, J=9.6, 2.4 Hz, 1H), 3.92-3.91 (m, 1H), 3.70 (dd, J=10.0, 3.3 Hz, 1H), 3.63 (dd, J=10.0, 7.6 Hz, 1H), 3.31 (dd, J=13.4, 9.6 Hz, 1H), 3.24 (dd, J=13.4, 3.1 Hz, 1H).

### Example 3 Synthesis of GDP-FAzP₄Biotin

400 μL GDP-FAz (50 mM in ddH₂O), 400 μL CuSO₄/BTTP (5 mM/10 mM in ddH₂O), 210 μL propargyl-PEG₄-Biotin (Click Chemistry Tools) (100 mM in MeOH), 40 μL ascorbate sodium (250 mM in ddH₂O) and 2.95 mL ddH₂O were mixed together. The reaction was allowed for stirring at r.t. for 5 h and monitored by TLC. Then, 2 mM BCS (bathocuproine sulphonate) was added to quench the reaction and the solvent was removed under reduced pressure. The crude product was further purified through a Prep-HPLC system to give the product as a white solid. (14.4 mg, yield 66%). HRMS (ESI−) calcd for C₃₇H₅₉N₁₁O₂₁P₂S (M−H⁺) 1086.3010, found 1086.3040. ¹H NMR (400 MHz, D₂O) δ 8.15 (s, 1H), 8.11 (s, 1H), 5.89 (d, J=6.0 Hz, 1H), 4.94-4.90 (m, 1H), 4.77-4.76 (m, 1H), 4.73-4.68 (m, 1H), 4.65 (d, J=3.1 Hz, 2H), 4.62-4.59 (m, 1H), 4.58-4.56 (m, 1H), 4.53-4.51 (m, 1H), 4.38 (dd, J=8.0, 4.4 Hz, 1H), 4.34-4.31 (m, 1H), 4.25-4.16 (m, 2H), 4.05-4.02 (m, 1H), 3.82 (s, 1H), 3.72-3.65 (m, 14H), 3.61 (t, J=5.3 Hz, 2H), 3.37 (t, J=5.2 Hz, 2H), 3.30-3.25 (m, 1H), 2.96 (dd, J=13.1, 5.0 Hz, 1H), 2.77-2.72 (m, 1H), 2.24 (t, J=7.3 Hz, 2H), 1.74-1.49 (m, 4H), 1.40-1.32 (m, 2H).

### Example 4 Synthesis of GDP-FAzP₄Tz

To a solution of 500 μL GDP-FAz (50 mM in ddH₂O) in ddH₂O/MeOH (1.45 mL/2.24 mL) were added 500 μL CuSO₄/BTTP (5 mM/10 mM in ddH₂O), 260 μL propargyl-PEG₄-Tz (Click Chemistry Tools) (100 mM in MeOH), and 50 μL ascorbate sodium (250 mM in ddH₂O) were added. The reaction was allowed for stirring at r.t. for 5 h and monitored by TLC. Then, 2 mM BCS was added to quench the reaction and the solvent was removed under reduced pressure. The crude product was further purified through a Prep-HPLC system to give the product as a pink solid (12.5 mg, yield 45%). HIRMS (ESI−) calcd for C₄₀H₅₇N₁₃O₂₁P₂(M−H⁺) 1116.3194, found 1116.3212.

### Example 5 Synthesis of GDP-FAzP₄MMAE

To a solution of 200 μL GDP-FAz (50 mM) in ddH₂O/MeOH (580 μL/790 μL), were added 200 μL CuSO₄/BTTP (5 mM/10 mM), 210 μL propargyl-PEG₄-vc-PAB-MMAE (Levena Biopharma) (50 mM in MeOH), and 20 μL ascorbate (250 mM in ddH₂O) were added. The reaction was allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (7.6 mg, 38%). HRMS (ESI−) calcd for C₉₆H₁₃₆N₁₈O₃₂P₂(M-2H⁺)/2 996.4449, found 996.4463. ¹H NMR (400 MHz, D₂O) δ 8.19 (s, 1H), 8.09 (s, 1H), 7.49-7.47 (m, 2H), 7.38-7.29 (m, 6H), 7.21-7.12 (m, 1H), 5.88 (d, J=5.8 Hz, 1H), 5.25-5.09 (m, 1H), 5.08-4.96 (m, 1H), 4.91 (s, 1H), 4.76-4.75 (m, 1H), 4.70-4.66 (m, 2H), 4.62 (s, 2H), 4.59-4.54 (m, 1H), 4.52-4.50 (m, 1H), 4.47-4.45 (m, 2H), 4.33-4.32 (m, 2H), 4.20-4.15 (m, 5H), 4.03-4.00 (m, 1H), 3.80 (s, 1H), 3.74-3.71 (m, 2H), 3.68-3.58 (m, 16H), 3.49-3.39 (s, 1H), 3.35 (s, 1H), 3.31-3.27 (m, 5H), 3.18 (s, 1H), 3.07-3.06 (m, 3H), 2.92 (d, J=15.3 Hz, 3H), 2.84-2.79 (m, 1H), 2.62-2.37 (m, 4H), 2.20-1.99 (m, 3H), 1.85-1.77 (m, 5H), 1.65-1.51 (m, 4H), 1.37-1.22 (m, 4H), 1.21-1.12 (m, 2H), 1.08 (d, J=6.4 Hz, 2H), 0.96-0.67 (m, 26H), 0.52-0.51 (m, 1H).

### Example 6 Synthesis of GDP-FAmP₄Biotin

To a solution of 500 μL GDP-FAm (100 mM in ddH₂O) in 1.5 mL ddH₂O were added 500 L NaHCO₃ (200 mM), 1.95 mL THF and 550 μL NHS-PEG₄-Biotin (Click Chemistry Tools) (100 mM in THF) were added. The reaction was stirred at r.t. for 4 h and monitored by TLC. The solvent was removed under reduced pressure. The crude product was further purified through a Prep-HPLC system to give the product as a white solid (20.5 mg, 38%). HIRMS (ESI−) calcd for C₃₇H₆₁N₉O₂₂P₂S (M−H⁺) 1076.3054, found 1076.3068. ¹H NMR (400 MHz, D₂O) δ 8.12 (s, 1H), 5.92 (d, J=6.1 Hz, 1H), 4.93 (t, J=7.8 Hz, 1H), 4.78-4.77 (m, 1H), 4.59 (dd, J=7.9, 4.6 Hz, 1H), 4.53 (dd, J=5.2, 3.4 Hz, 1H), 4.39 (dd, J=7.9, 4.4 Hz, 1H), 4.35-4.34 (m, 1H), 4.22 (dd, J=5.4, 3.4 Hz, 2H), 3.87 (d, J=3.1 Hz, 1H), 3.76 (t, J=6.3 Hz, 2H), 3.69-3.66 (m, 14H), 3.63-3.60 (m, 3H), 3.59-3.56 (m, 1H), 3.38 (t, J=5.3 Hz, 2H), 3.32-3.26 (m, 2H), 2.97 (dd, J=13.1, 5.0 Hz, 1H), 2.77 (d, J=13.0 Hz, 1H), 2.56 (t, J=6.2 Hz, 2H), 2.26 (t, J=7.3 Hz, 2H), 1.74-1.51 (m, 4H), 1.42-1.34 (m, 2H).

### Example 7 Synthesis of GDP-FAmP₄Tz

To a solution of 200 μL GDP-FAm (100 mM) in 600 uL ddH₂O were added 200 μL NaHCO₃ (200 mM), 780 μL THF and 220 μL NHS-PEG₄-Tz (Click Chemistry Tools) (100 mM in THF) were added. The reaction was stirred at r.t. for 4 h and monitored by TLC. The solvent was removed under reduced pressure. The crude product was further purified through a Prep-HPLC system to give the product as a pink solid (9.9 mg, yield 48%). HRMS (ESI−) calcd for C₃₆H₅₂N₁₀O₂₁P₂(M−H⁺) 1021.2711, found 1021.2725. ¹H NMR (400 MHz, D₂O) δ 8.17-8.13 (m, 2H), 8.00 (s, 1H), 7.06-7.02 (m, 2H), 5.76 (d, J=5.6 Hz, 1H), 4.91 (t, J=7.7 Hz, 1H), 4.67 (t, J=5.4 Hz, 1H), 4.49 (dd, J=5.1, 3.7 Hz, 1H), 4.30-4.28 (m, 1H), 4.27-4.25 (m, 2H), 4.21-4.19 (m, 2H), 3.95-3.93 (m, 2H), 3.84-3.83 (m, 1H), 3.80-3.78 (m, 2H), 3.74-3.71 (m, 2H), 3.70-3.57 (m, 13H), 3.51 (dd, J=14.1, 4.2 Hz, 1H), 3.24 (dd, J=14.0, 8.6 Hz, 1H), 3.00 (s, 3H), 2.49 (t, J=6.3 Hz, 2H).

### Example 8 Synthesis of GDP-FAmP₈Tz

To a solution of 200 μL GDP-FAm (100 mM) in 600 μL ddH₂O were added 200 μL NaHCO₃ (200 mM), 780 μL THF and 220 μL NHS-PEG₈-Tz (Xi'an Dianhua Biotechnology Co., Ltd) (100 mM in THF) were added. The reaction was stirred at r.t. for 4 h and monitored by TLC. The solvent was removed under reduced pressure. The crude product was further purified through a Prep-HPLC system to give the product as a pink solid (9.2 mg, yield 38%). HRMS (ESI−) calcd for C₄₄H₆₈N₁₀O₂₅P₂(M-2H⁺)/2 598.1844, found 598.1880. ¹H NMR (400 MHz, D₂O) δ 8.28-8.24 (m, 2H), 8.02 (s, 1H), 7.16-7.12 (m, 2H), 5.81 (d, J=6.0 Hz, 1H), 4.92 (t, J=7.8 Hz, 1H), 4.72 (t, J=5.6 Hz, 1H), 4.50 (dd, J=5.2 Hz, 3.5, 1H), 4.32-4.29 (m, 3H), 4.21-4.19 (m, 2H), 3.97-3.95 (m, 2H), 3.85 (d, J=3.0 Hz, 1H), 3.81-3.78 (m, 2H), 3.75-3.59 (m, 32H), 3.27 (dd, J=14.1, 8.7, 1H), 3.02 (s, 3H), 2.53 (t, J=6.2 Hz, 2H).

### Example 9 Synthesis of GDP-FAmP₄BCN

To a solution of 200 μL GDP-FAm (100 mM) in 600 μL ddH₂O were added 200 μL NaHCO₃ (200 mM), 560 μL THF and 440 μL NHS-PEG₄-BCN (Xi'an Dianhua Biotechnology Co., Ltd) (50 mM in THF) were added. The reaction was stirred at r.t. for 4 h and monitored by TLC. The solvent was removed under reduced pressure. The crude product was further purified through a Prep-HPLC system to give the product as a white solid (7.4 mg, yield 36%). HIRMS (ESI−) calcd for C₃₈H₅₉N₇O₂₂P₂(M-2H⁺)/2 512.6522, found 512.6532. ¹H NMR (400 MHz, D₂O) δ 8.17 (s, 1H), 5.92 (d, J=5.9 Hz, 1H), 4.93 (t, J=7.8 Hz, 1H), 4.78-4.75 (m, 1H), 4.52 (dd, J=5.1, 3.5 Hz, 1H), 4.36-4.32 (m, 1H), 4.22 (dd, J=5.4, 3.4 Hz, 2H), 4.14 (d, J=8.2 Hz, 2H), 3.86 (d, J=2.3 Hz, 1H), 3.75 (t, J=6.3 Hz, 2H), 3.69-3.64 (m, 14H), 3.62-3.58 (m, 4H), 3.31 (t, J=5.3 Hz, 2H), 3.29-3.26 (m, 1H), 2.55 (t, J=6.2 Hz, 2H), 2.29-2.15 (m, 6H), 1.54-1.51 (m, 2H), 1.39-1.31 (m, 1H), 0.92 (t, J=9.8 Hz, 2H).

### Example 10 Synthesis of GDP-FAmP₄TCO

To a solution of 400 μL GDP-FAm (100 mM) in 1.4 mL ddH₂O were added 400 uL NaHCO₃ buffer (200 mM), 1.36 mL THF and 440 μL NHS-PEG₄-TCO (Click Chemistry Tools) (100 mM in THF) were added. The reaction was stirred at r.t. for 4 h and monitored by TLC. The solvent was removed under reduced pressure. The crude product was further purified through a Prep-HPLC system to give the product as a white solid (8.2 mg, yield 20%). IRMS (ESI−) calcd for C₃₆H₅₉N₇O₂₂P₂(M−H⁺) 1002.3116, found 1002.3134.

### Example 11 Synthesis of GDP-FAmAz

To a solution of 200 μL GDP-FAm (100 mM) in 600 μL ddH₂O were added 200 μL NaHCO₃ (200 mM), 780 μL THF and 220 μL NHS-azide (Xi'an Dianhua Biotechnology Co., Ltd) (100 mM in THF) were added. The reaction was stirred at r.t. for overnight and monitored by TLC. The solvent was removed under reduced pressure. The crude product was further purified through a Prep-HPLC system to give the product as a white solid (8.7 mg, yield 63%). HIRMS (ESI−) calcd for C₁₈H₂₇N₉O₁₆P₂ (M−H⁺) 686.0978, found 686.1002. ¹H NMR (400 MHz, D₂O) δ 8.10 (s, 1H), 5.92 (d, J=6.0 Hz, 1H), 4.92 (t, J=7.9 Hz, 1H), 4.78-4.76 (m, 1H), 4.52 (dd, J=5.2, 3.4 Hz, 1H), 4.35-4.34 (m, 1H), 4.23-4.21 (m, 2H), 4.00 (s, 2H), 3.87 (d, J=3.2 Hz, 1H), 3.71 (dd, J=8.8, 3.9 Hz, 1H), 3.66 (dd, J=10.0, 3.3 Hz, 1H), 3.62-3.57 (m, 2H), 3.32 (dd, J=14.0, 8.6 Hz, 1H).

### Example 12 Synthesis of GDP-FAmP₂Az

To a solution of 200 μL GDP-FAm (100 mM) in 600 μL ddH₂O were added 200 μL NaHCO₃ buffer (200 mM), then 780 μL THF and 220 μL NHS-PEG₂-azide (Xi'an Dianhua Biotechnology Co., Ltd) (100 mM in THF) were added. The reaction was stirred at r.t. for overnight and monitored by TLC. The solvent was removed under reduced pressure. The crude product was further purified through a Prep-HPLC system to give the GDP-FAmP₂Az as a white solid (5.4 mg, yield 34%). HRMS (ESI−) calcd for C₂₃H₃₇N₉O₁₈P₂(M−H⁺) 788.1659, found 788.1671. ¹H NMR (400 MHz, D₂O) δ 8.10 (s, 1H), 5.91 (d, J=6.0 Hz, 1H), 4.91 (t, J=7.3 Hz, 1H), 4.77-4.68 (m, 1H), 4.52 (t, J=3.8 Hz, 1H), 4.33-4.32 (m, 1H), 4.20-4.08 (m, 2H), 3.85 (d, J=3.1 Hz, 1H), 3.76-3.73 (m, 2H), 3.72-3.62 (m, 8H), 3.61-3.54 (m, 2H), 3.45 (t, J=4.8 Hz, 2H), 3.27 (dd, J=14.0, 8.5 Hz, 1H), 2.53 (t, J=6.0 Hz, 2H).

### Example 13 Synthesis of GDP-FAmP₄Az

To a solution of 200 μL GDP-FAm (100 mM) in 600 μL ddH₂O were added 200 μL NaHCO₃ buffer, then 780 μL THF and 220 μL NHS-PEG₄-azide (Xi'an Dianhua Biotechnology Co., Ltd) (100 mM in THF) were added. The reaction was stirred at r.t. for overnight and monitored by TLC. The solvent was removed under reduced pressure. The crude product was further purified through a Prep HPLC system to give the GDP-FAmP₄Az as a white solid (9.0 mg, yield 51%). HRMS (ESI−) calcd for C₂₇H₄₅N₉O₂₀P₂ (M−H⁺) 876.2183, found 876.2187. ¹H NMR (400 MHz, D₂O) δ 8.14 (s, 1H), 5.90 (d, J=6.0 Hz, 1H), 4.90 (t, J=7.8 Hz, 1H), 4.75 (t, J=5.5 Hz, 1H), 4.50 (dd, J=5.0, 3.5 Hz, 1H), 4.33-4.32 (m, 1H), 4.21-4.19 (m, 2H), 3.84 (d, J=3.2 Hz, 1H), 3.73 (t, J=6.2, 2H), 3.70-3.61 (m, 16H), 3.60-3.53 (m, 2H), 3.47-3.45 (m, 2H), 3.26 (dd, J=14.0, 8.6 Hz, 1H), 2.53 (t, J=6.2 Hz, 2H).

### Example 14 Synthesis of GDP-FAmP₄MCP

To a solution of 220 μL GDP-FAm (100 mM) in 690 μL ddH₂O were added 200 μL NaHCO₃ (200 mM), 690 μL THF and 200 μL NHS-PEG₄-MCP (Xi'an Dianhua Biotechnology Co., Ltd) (100 mM in THF) were added. The reaction was stirred at r.t. for 6 h and monitored by TLC. The solvent was removed under reduced pressure. The crude product was further purified through a Prep-HPLC system to give the desired product as a white solid (7.8 mg, 40%). HRMS (ESI−) calcd for C₃₃H₅₃N₇O₂₂P₂(M−H⁺) 960.2646, found. 960.2638.

### Example 15 Synthesis of GDP-FAmGGG

Cbz-GGG-OH was synthesized according to the reported procedure (Miravet J. F., et al. Eur. J. Org. Chem. 2014, 16, 3372).

Cbz-GGG-NHS. To a solution of 200 mg (0.62 mmol) Cbz-GGG-OH in 1.5 mL dry DMF were added 82 mg (0.71 mmol) NHS and 148 mg (0.82 mmol) EDC·HCl. The reaction was stirred at r.t. for 3 h and monitored by TLC. The solvent was removed under reduced pressure. The residue was resolved in 80 mL DCM, and washed by water, saturated NaHCO₃ solution and brine respectively. The organic layers were dried through Na₂SO₄ and concentrated to afford the crude for the next step without further purification.

GDP-FAmGGG-Cbz. The crude product (Cbz-GGG-NHS) obtained above were resolved in 5.4 mL H₂O followed by adding 40.5 mg (0.48 mmol) NaHCO₃ and 115.0 mg (0.19 mmol) GDP-FAm. The reaction was stirred at r.t. for overnight and monitored by TLC. The product was further purified through a Prep-HPLC system to give GDP-FAmGGG-Cbz as a white powder (135.6 mg. yield 24% in two steps).

GDP-FAmGGG. To a clear solution of 21 mg (0.023 mmol) GDP-FAmGGG-Cbz was added 2 mL H₂O and 15 mg Pd/C (10%). The air atmosphere was change to H₂ by vacuum and refill. The H₂ pressure was kept at 0.28 MPa. The reaction was stirred at r.t. for 1 h and filtered through a 0.22 μm filter. The product was further purified through a Prep-HPLC system to give the GDP-FAmGGG as a white powder (15.2 mg, yield 85%). HIRMS (ESI−) calcd for C₂₂H₃₅N₉O₁₈P₂(M-2H⁺)/2 386.5715, found 386.5717.

### Example 16 Synthesis of GDP-FAmP₄MMAE

To a solution of 200 uL GDP-FAm (100 mM) in 600 uL ddH₂O were added 200 uL NaHCO₃ (200 mM), then 560 uL THF and 440 uL OSu-PEG₄-vc-PAB-MMAE (Levena Biopharma) (50 mM in THF). The reaction was stirred at r.t. for 4 h and monitored by TLC. The solvent was removed under reduced pressure. The crude product was further purified through a Prep-HPLC system to give the desired product as a white solid (13.2 mg. 33%). HIRMS (ESI−) calcd for C₈₆H₁₃₈N₁₆O₃₃P₂(M-2H⁺)/2 991.4471, found 991.4502. ¹H NMR (400 MHz, D₂O) δ 8.19 (s, 1H), 7.48-7.42 (m, 2H), 7.41-7.28 (m, 6H), 7.22-7.16 (m, 1H), 5.90 (d, J=6.0 Hz, 1H), 5.28-5.14 (m, 1H), 5.03 (t, J=11.0 Hz, 1H), 4.90 (t, J=7.8 Hz, 1H), 4.64-4.58 (m, 1H), 4.51-4.49 (m, 1H), 4.44-4.38 (m, 2H), 4.34-4.28 (m, 2H), 4.21-4.16 (m, 3H), 4.11 (t, J=7.2 Hz, 2H), 3.83 (d, J=3.2 Hz, 1H), 3.75-3.70 (m, 4H), 3.69-3.64 (m, 3H), 3.63-3.56 (m, 14H), 3.34 (s, 1H), 3.30-3.25 (m, 6H), 3.19-3.14 (m, 2H), 3.11-3.06 (m, 4H), 2.95-2.83 (m, 4H), 2.62-2.44 (m, 6H), 2.16-1.97 (m, 4H), 1.92-1.71 (m, 6H), 1.65-1.43 (m, 5H), 1.35-1.21 (m, 4H), 1.17-1.14 (m, 3H), 1.06 (d, J=6.7 Hz, 2H), 0.96 (d, J=6.4 Hz, 2H), 0.93-0.89 (m, 8H), 0.83-0.78 (m, 10H), 0.71-0.66 (m, 2H), 0.50-0.48 (m, 2H).

### Example 17 Synthesis of GDP-FAmSucMMAE

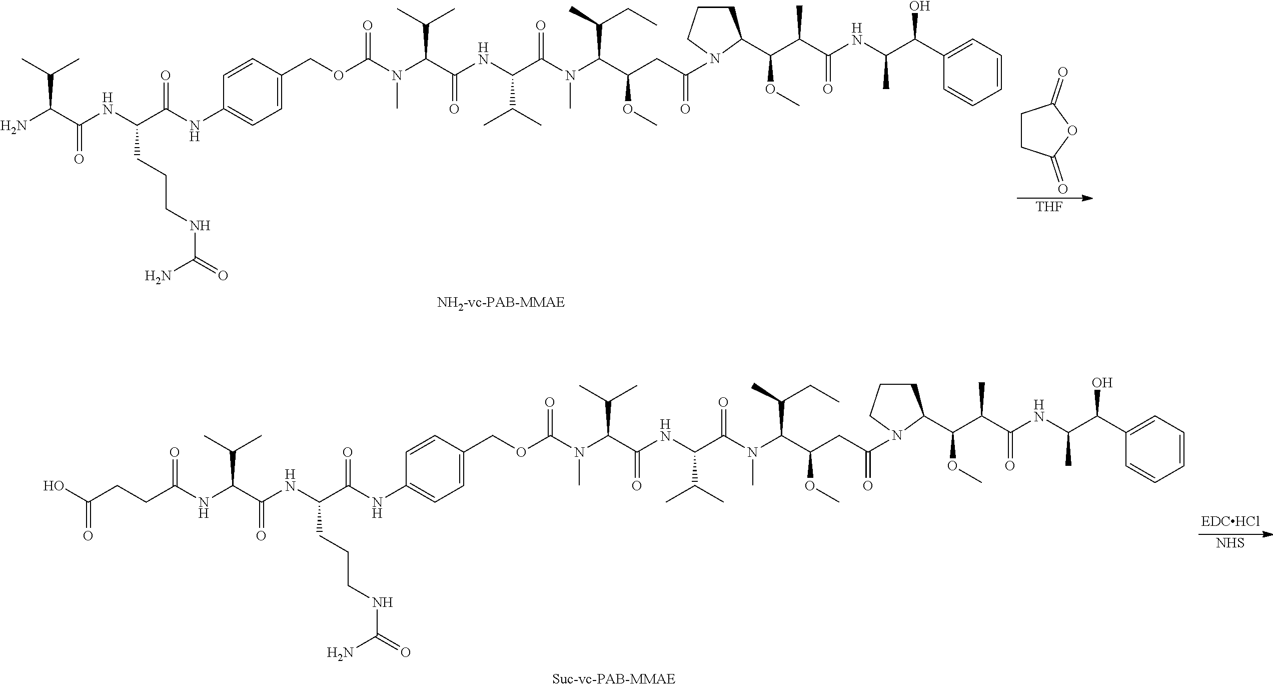

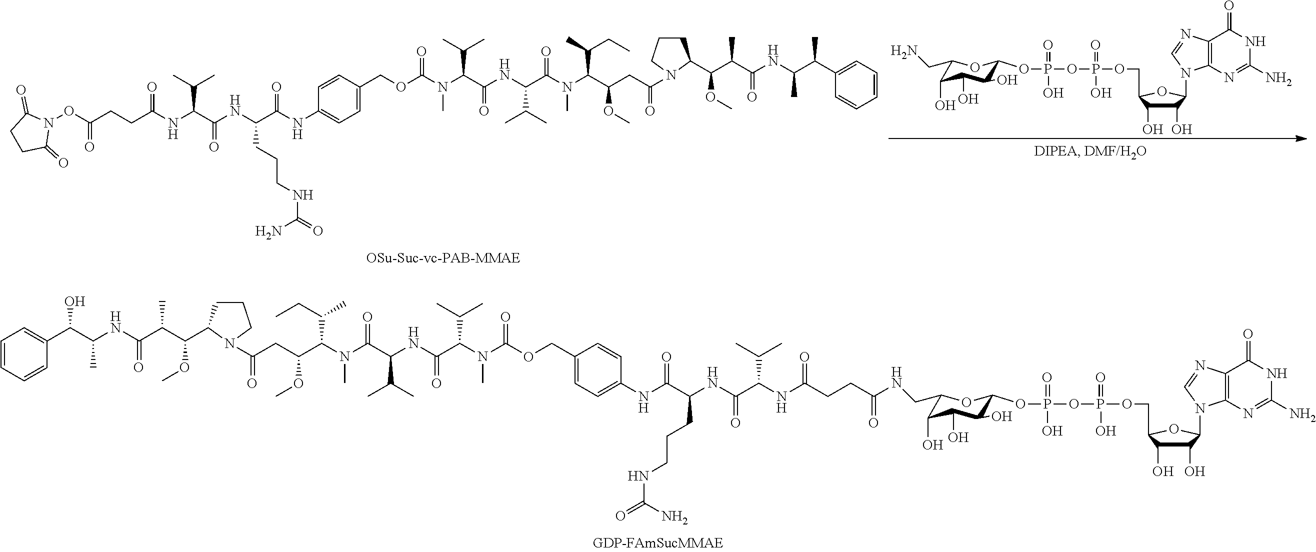

NH₂-vc-PAB-MMNAE was synthesized according to the reported procedure (Tang, F., et al. Org. Biomol. Chem. 2016, 14, 9501).

Suc-vc-PAB-MMAE. To a solution of NH₂-vc-PAB-MMAE (833 mg, 0.74 mmol) in DMF (15 mL) and THE (15 mL) were added Succinic anhydride (120 mg, 1.12 mmol). The mixture was stirred at r.t. for 5 h and monitored by TLC. The product was further purified through a Prep-HPLC system to give the Suc-vc-PAB-MMAE as a white foam (683 mg, yield 75.3%). HRMS (ESI−) calcd for C₆₂H₉₈N₁₀O₁₅ (M−H⁺) 1221.7140, found 1221.7146.

OSu-Suc-vc-PAB-MMAE. To a solution of Suc-vc-PAB-MMAE (683 mg, 0.559 mmol) in DCM (10 mL) and THE (10 mL) were added NHS (644 mg, 5.596 mmol) and EDC·HCl (1284 mg, 6.698 mmol). The mixture was stirred at r.t. for 3 h and monitored by TLC. The product was further purified through a Prep-HPLC system to give the OSu-Suc-vc-PAB-MMAE as a white powder (565 mg, yield 76.6%). HRMS (ESI+) calcd for C₆₆H₁₀₁N₁₁O₁₇ (M+Na⁺) 1342.7269, found 1342.7283.

GDP-FAmSucMMAE. To a solution of GDP-FAm (190 mg, 0.315 mmol) in 30 mL ddH₂O were added 400 uL DIPEA, and then OSu-Suc-vc-PAB-MMAE (346 mg, 0.262 mmol) in 12 mL DMF were added. The mixture was stirred at r.t. for 5 h and monitored by TLC. The product was further purified through a Prep-HPLC system to give the GDP-FAmSucMMAE as a white powder (104.3 mg, yield 22.0%). HRMS (ESI−) calcd for C₇₈H₁₂₂N₁₆O₂₉P₂ (M-2H⁺)/2 903.3947, found 903.3959. ¹H NMR (400 MHz, D₂O) δ 8.16 (s, 1H), 7.48-7.40 (m, 3H), 7.37-7.28 (m, 5H), 7.22-7.14 (m, 1H), 5.9 (d, J=6.0 Hz, 1H), 5.30-5.14 (m, 1H), 5.06-5.00 (m, 1H), 4.76-4.68 (m, 2H), 4.63-4.59 (m, 1H), 4.51-4.49 (m, 1H), 4.46-4.30 (m, 4H), 4.21-4.04 (m, 6H), 3.76-3.63 (m, 2H), 3.57-3.53 (m, 2H), 3.45-3.14 (m, 14H), 3.11-3.05 (m, 4H), 2.94-2.90 (m, 3H), 2.66-2.44 (m, 6H), 2.16-2.01 (m, 3H), 1.94-1.76 (m, 4H), 1.68-1.47 (m, 4H), 1.36-1.14 (m, 8H), 1.06 (d, J=6.6 Hz, 2H), 0.97-0.89 (m, 10H), 0.85-0.77 (m, 10H), 0.66 (t, J=7.8 Hz, 2H), 0.48 (d, J=6.6 Hz, 1H).

### Example 18 Synthesis of GDP-FAmAzP₄MMAE

To a solution of 200 μL GDP-FAmAz (50 mM) in ddH₂O/MeOH (580 μL/790 μL), were added 200 μL CuSO₄/BTTP (5 mM/10 mM), 210 μL propargyl-PEG₄-vc-PAB-MMAE (Levena Biopharma) (50 mM in MeOH), and 20 μL ascorbate (250 mM in ddH₂O) were added. The reaction as allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (16.8 mg, 82%). HIRMS (ESI+) calcd for C₈₈H₁₃₉N₁₉O₃₃P₂(M+2Na⁺)/2 1048.9521, Found 1048.9533.

### Example 19 Synthesis of GDP-FAmP₄AzP₄MMAE

To a solution of 200 μL GDP-FAmP₄Az (50 mM) in ddH₂O/MeOH (580 μL/790 μL), were added 200 μL CuSO₄/BTTP (5 mM/10 mM), 210 μL propargyl-PEG₄-vc-PAB-MMAE (Levena Biopharma) (50 mM in MeOH), and 20 μL ascorbate (250 mM in ddH₂O) were added. The reaction as allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (17.0 mg, 76%). HIRMS (ESI+) calcd for C₉₇H₁₅₇N₁₉O₃₇P₂(M+2Na⁺)/2 1144.0124, Found 1144.0086. ¹H NMR (400 MHz, D₂O) δ 8.16 (s, 1H), 8.04 (s, 1H), 7.49-7.42 (m, 3H), 7.39-7.30 (m, 5H), 7.25-7.21 (m, 1H), 5.91 (d, J=5.9 Hz, 1H), 5.30-5.17 (m, 1H), 5.05 (t, J=10.8 Hz, 1H), 4.95-4.90 (m, 1H), 4.76-4.70 (m, 1H), 4.65 (s, 2H), 4.59 (t, J=5.0 Hz, 2H), 4.52-4.49 (m, 1H), 4.46-4.39 (m, 2H), 4.36-4.32 (m, 1H), 4.26-4.18 (m, 3H), 4.14 (t, J=6.7 Hz, 2H), 3.93 (t, J=5.1 Hz, 2H), 3.85 (d, J=3.0 Hz, 1H), 3.76-3.55 (m, 35H), 3.47-3.42 (m, 1H), 3.37-3.36 (m, 1H), 3.32-3.24 (m, 6H), 3.21-3.17 (m, 1H), 3.13-3.06 (m, 4H), 2.97-2.90 (m, 3H), 2.85-2.67 (m, 1H), 2.66-2.38 (m, 6H), 2.35-1.99 (m, 4H), 1.93-1.76 (m, 5H), 1.65-1.51 (m, 5H), 1.31-1.20 (m, 4H), 1.18-1.14 (m, 2H), 1.08 (d, J=6.8 Hz, 2H), 0.99 (d, J=6.4 Hz, 2H), 0.94-0.90 (m, 8H), 0.87-0.78 (m, 12H), 0.73-0.67 (m, 2H), 0.52-0.51 (m, 1H).

### Example 20 Synthesis of GDP-FAmAzP₄DXd

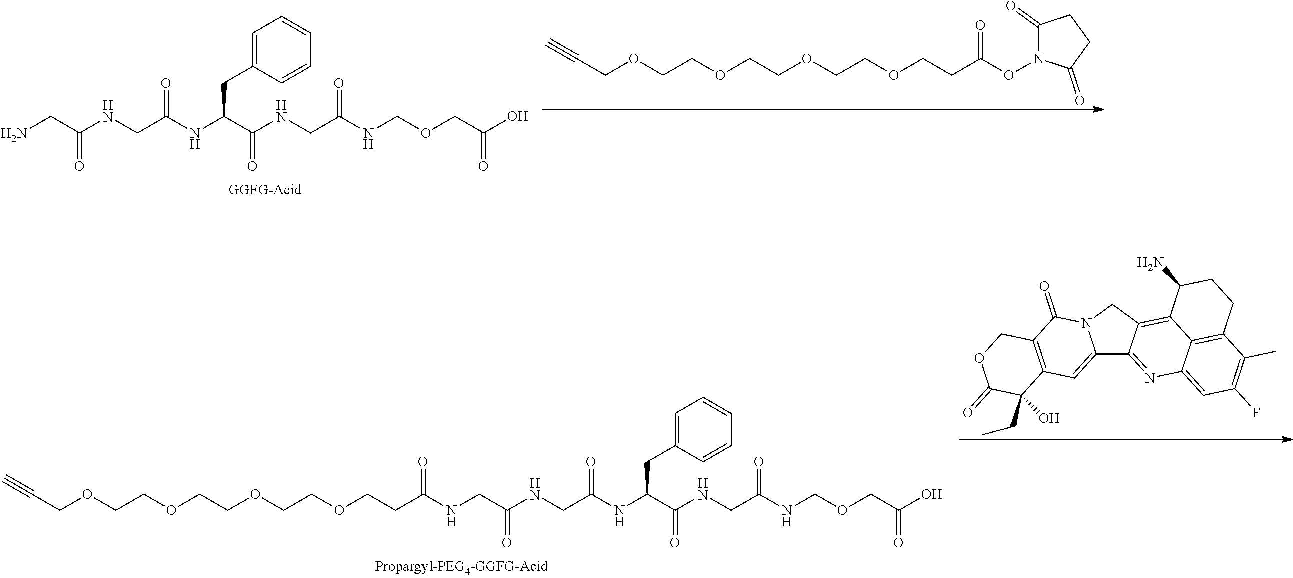 [SMILES: NCC(=O)NCC(=O)N[C@@H](Cc1ccccc1)C(=O)NCC(=O)NCOCC(=O)O ; C#CCOCCOCCOCCOCCC(=O)ON1C(=O)CCC1=O ; C#CCOCCOCCOCCOCCC(=O)NCC(=O)NCC(=O)N[C@@H](Cc1ccccc1)C(=O)NCC(=O)NCOCC(=O)O ; CC[C@@]1(O)C(=O)OCc2c1cc1n(c2=O)Cc2c-1nc1cc(F)c(C)c3c1c2[C@@H](N)CC3]

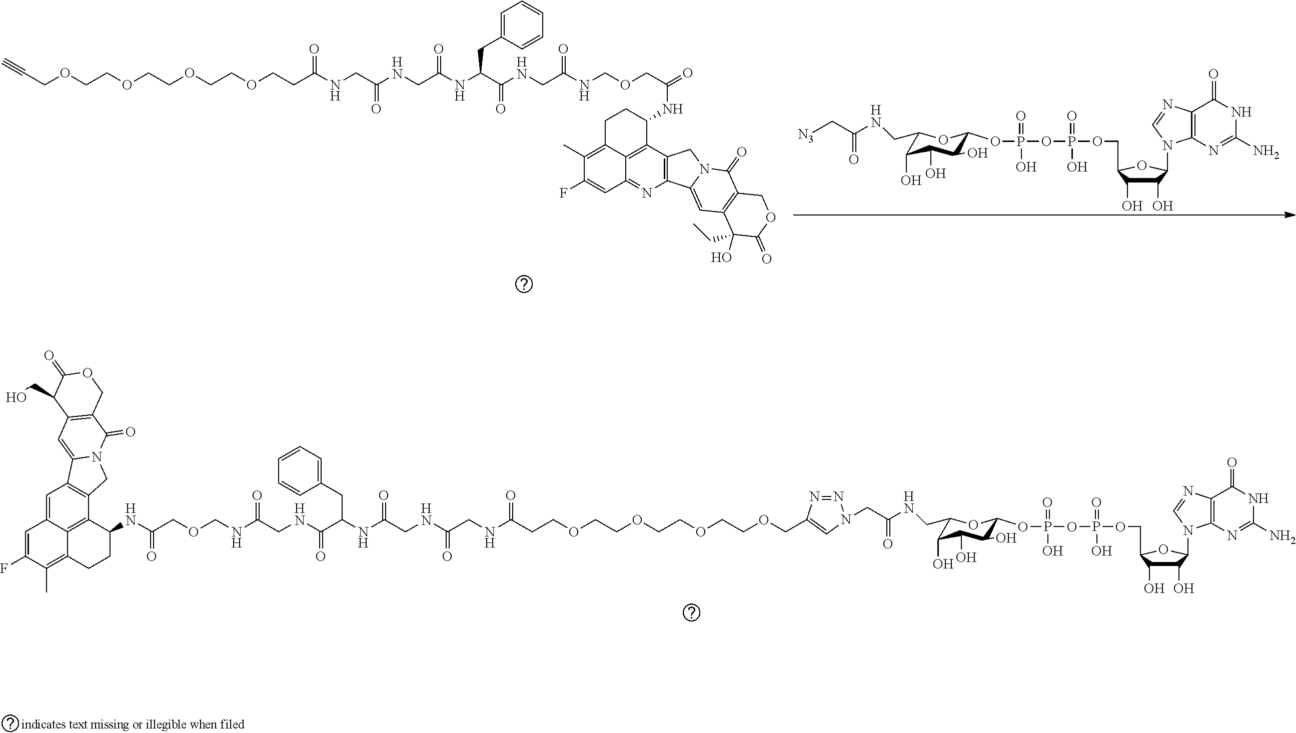

GGFG-Acid was synthesized according to the reported procedure (Yamaguchi, T., et al., EP3677589A1).

Propargyl-PEG₄-GGFG-Acid. To a solution of GGFG-Acid (98.4 mg, 0.23 mmol) in DMF (5 ml) were added DIPEA (0.2 ml) and propargyl-PEG₄-OSu (99.6 mg, 0.28 mmol). The mixture was stirred at r.t. for overnight and monitored by TLC. The crude product was further purified through a Prep-HPLC system to give the desired product as a white solid (114.8 mg, 75.0%). HRMS (ESI−) calcd for C₃₀H₄₃N₅O₁₂ (M−H⁺) 664.2835, found 664.2808.

Propargyl-PEG₄-GGFG-DXd. To a solution of propargyl-PEG₄-GGFG Acid (66.6 mg, 0.1 mmol) in DMF (5 ml) were added DIPEA (0.1 ml), Exatecan (43.5 mg, 0.1 mmol) and PyBOP (104.9 mg, 0.2 mmol). The mixture was stirred at r.t. for 2 h and monitored by TLC. The crude product was further purified through a Prep-HPLC system to give the desired product as a light-yellow solid (70.6 mg, 65.2%). HRMS (ESI+) calcd for C₅₄H₆₃FN₈O₁₅ (M+Na⁺) 1105.4289, found 1105.4255.

GDP-FAmAzP₄DXd. To a solution of 200 μL GDP-FAmAz (50 mM) in ddH₂O/MeOH (580 μL/790 μL), were added 200 μL CuSO₄/BTTP (5 mM/10 mM), 210 μL propargyl-PEG₄-GGFG-DXd (50 mM in MeOH), and 20 μL ascorbate (250 mM in ddH₂O) were added. The reaction was allowed for stirring at r.t. for 5 h and monitored by TLC. Then, 2 mM BCS was added to quench the reaction and the solvent was removed under reduced pressure. The crude product was further purified through a Prep-HPLC system to give the product as a white solid (12.1 mg, 68.5%). HRMS (ESI−) calcd for C₇₂H₉₀FN₁₇O₃₁P₂(M-2H⁺)/2 883.7651, found 883.7651. ¹H NMR (400 MHz, D₂O) δ 7.97 (s, 2H), 7.21 (s, 1H), 7.11-7.03 (m, 4H), 6.86 (d, J=7.0 Hz, 2H), 5.68 (d, J=5.7 Hz, 1H), 5.59-5.55 (m, 1H), 5.45-5.40 (m, 1H), 5.29-5.25 (m, 1H), 5.20 (s, 1H), 4.95-4.91 (m, 2H), 4.70-4.53 (m, 7H), 4.46 (t, J=4.1 Hz, 1H), 4.37-4.31 (m, 2H), 4.26-4.17 (m, 4H), 3.86-3.57 (m, 26H), 3.34-3.28 (m, 1H), 3.10-3.06 (m, 1H), 2.97-2.88 (m, 1H), 2.78-2.73 (m, 1H), 2.56-2.50 (m, 3H), 2.39-2.29 (m, 1H), 2.09 (s, 3H), 1.93-1.82 (m, 2H), 0.94 (t, J=7.3, 3H).

### Example 21 GDP-FAmDM4

To a solution of 200 uL GDP-FAm (100 mM) was added 600 uL ddH₂O, 200 uL NaHCO₃ (200 mM), 560 uL THF and 440 uL OSu-SPDB-DM4 (Levena Biopharma) (50 mM in THF). The reaction was stirred at r.t. for 4 h and monitored by TLC. The solvent was removed under reduced pressure. The crude product was further purified through a Prep-HPLC system to give the desired product as a white solid (9.2 mg. 62%). HIRMS (ESI−) calcd for C₅₈H₈₄ClN₉O₂₆P₂S₂ (M-2H⁺)/2 740.6994, Found: 740.7004. ¹H NMR (400 MHz, D₂O) δ 8.20 (s, 1H), 7.13 (s, 1H), 6.62-6.54 (m, 3H), 5.90 (d, J=5.6 Hz, 1H), 5.65-5.60 (m, 1H), 5.38-5.37 (m, 1H), 4.93 (t, J=7.8 Hz, 1H), 4.71 (t, J=5.4 Hz, 1H), 4.60-4.57 (m, 1H), 4.52-4.50 (m, 1H), 4.31 (s, 1H), 4.25-4.22 (m, 3H), 3.94 (s, 3H), 3.87-3.86 (m, 1H), 3.70-3.48 (m, 7H), 3.35 (s, 3H), 3.30-3.26 (m, 1H), 3.23-3.18 (m, 4H), 3.08 (d, J=9.4 Hz, 1H), 2.83 (s, 3H), 2.69-2.62 (m, 1H), 2.52-2.49 (m, 3H), 2.39-2.37 (m, 1H), 2.26-2.23 (m, 3H), 1.88-1.79 (m, 2H), 1.74-1.67 (m, 2H), 1.59-1.51 (m, 4H), 1.36-1.33 (m, 1H), 1.26 (d, J=6.6 Hz, 3H), 1.21-1.19 (m, 6H), 1.11 (s, 3H), 0.78 (s, 3H).

### Example 22 Synthesis of TCO-PEG₄-vc-PAB-MMAE

To a solution of NH₂-vc-PAB-MMAE (30.0 mg, 0.027 mmol) in DMF (1.5 mL) were added DIPEA (100 μL) and NHS-PEG₄-TCO (Xi'an Dianhua Biotechnology Co., Ltd) (16.5 mg, 0.032 mmol). The mixture was stirred at r.t. for overnight and monitored by TLC. The product was further purified through a Prep-HPLC system to give the TCO-PEG₄-vc-PAB-MMAE as a white powder (21.7 mg, yield 53%). HIRMS (ESI−) calcd for C₇₈H₁₂₇N₁₁O₁₉ (M−H⁺) 1520.9237, found 1520.9277.

### Example 23 Synthesis of Az-PEG₄-vc-PAB-MMAE

To a solution of NH₂-vc-PAB-MMAE (30.0 mg, 0.027 mmol) in DMF (1.5 mL) were added DIPEA (100 μL) and NHS-PEG₄-azide (Xi'an Dianhua Biotechnology Co., Ltd) (12.4 mg, 0.032 mmol). The mixture was stirred at r.t. for overnight and monitored by TLC. The product was further purified through a Prep-HPLC system to give the Az-PEG₄-vc-PAB-MMAE as a white powder (25.7 mg, yield 67%). HRMS (ESI+) calcd for C₆₉H₁₁₃N₁₃O₁₇ (M+Na⁺) 1418.8270, found 1418.8262.

### Example 24 Synthesis of DBCO-PEG₄-vc-PAB-seco-DUBA (24-12)

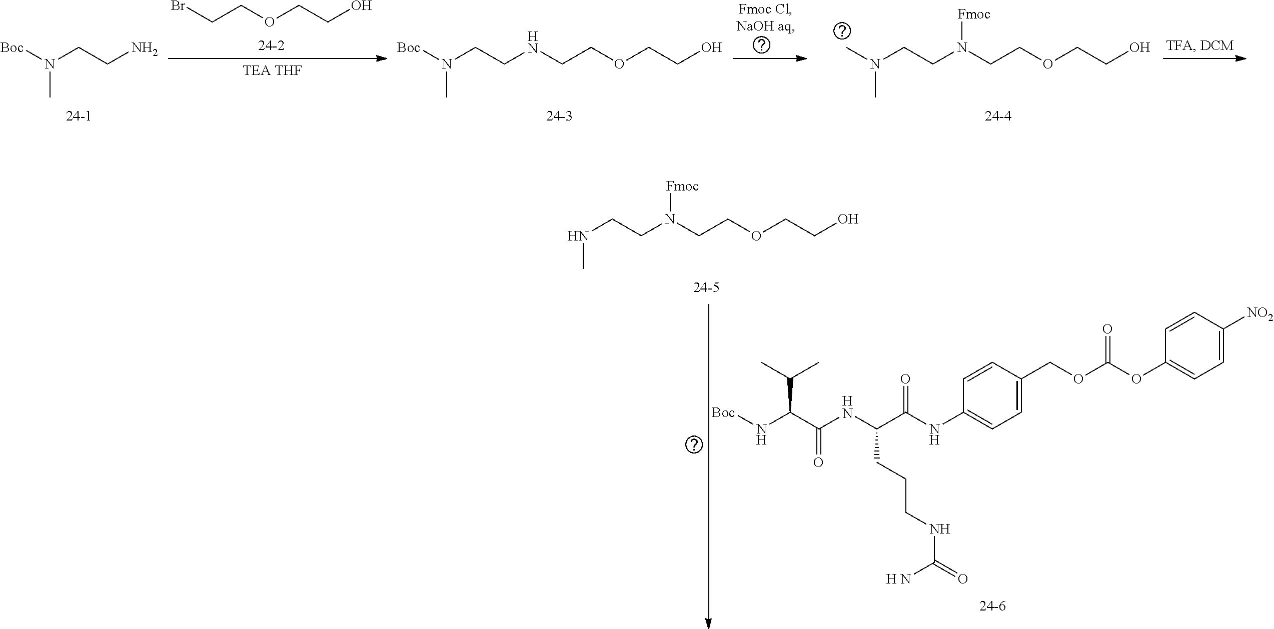

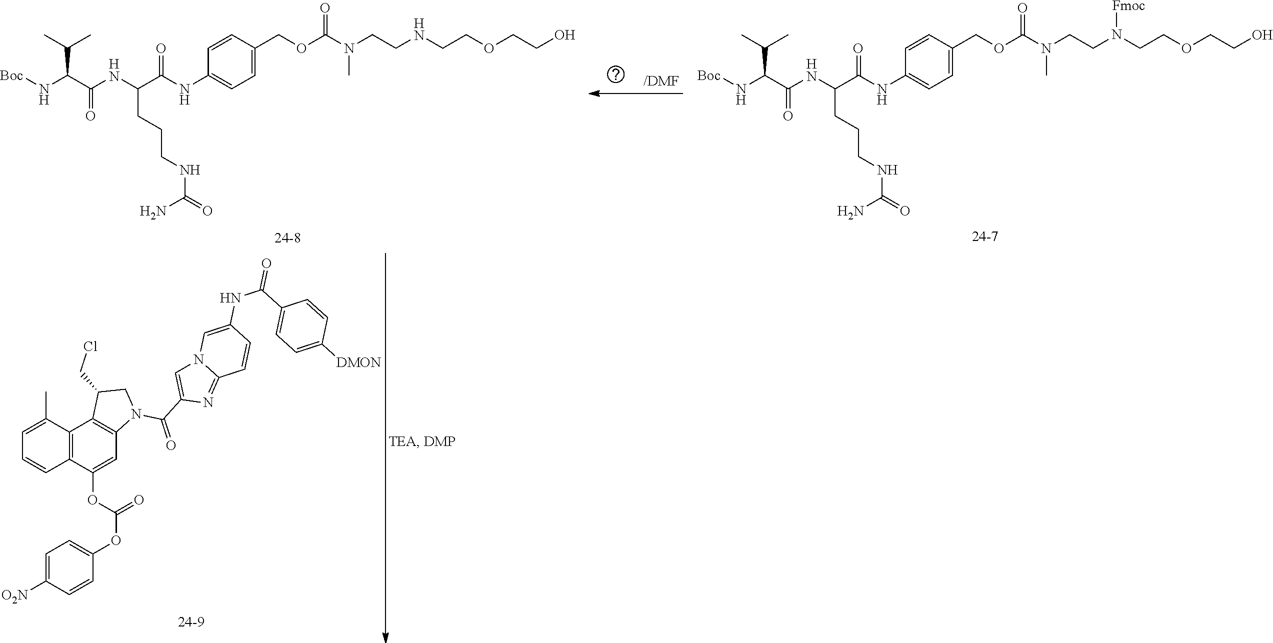

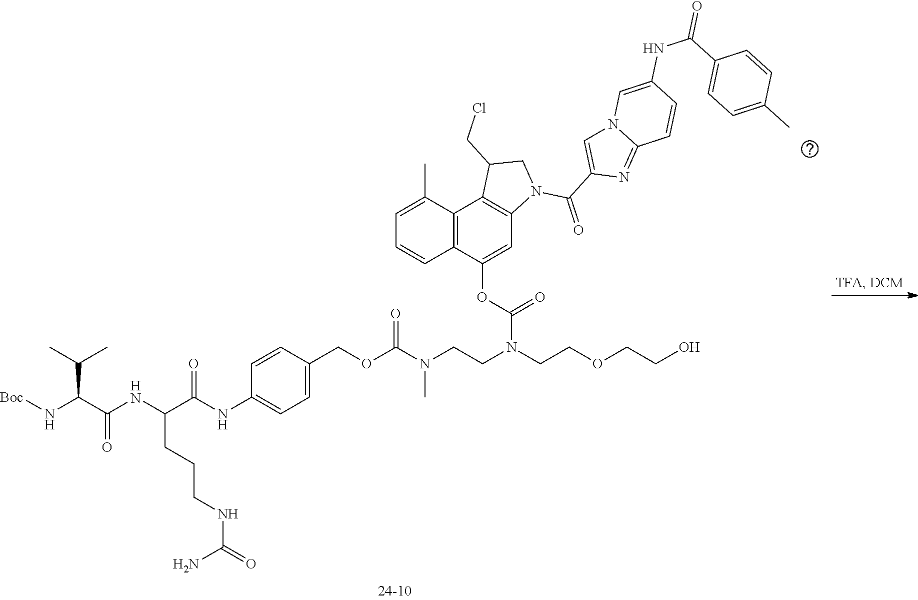 [SMILES: Cc1ccc(C(=O)Nc2ccc3nc(C(=O)N4CC(CCl)c5c4cc(OC(=O)N(CCOCCO)CCN(C)C(=O)OCc4ccc(NC(=O)C(CCCNC(N)=O)NC(=O)[C@@H](NC(=O)OC(C)(C)C)C(C)C)cc4)c4cccc(C)c54)cn3c2)cc1]

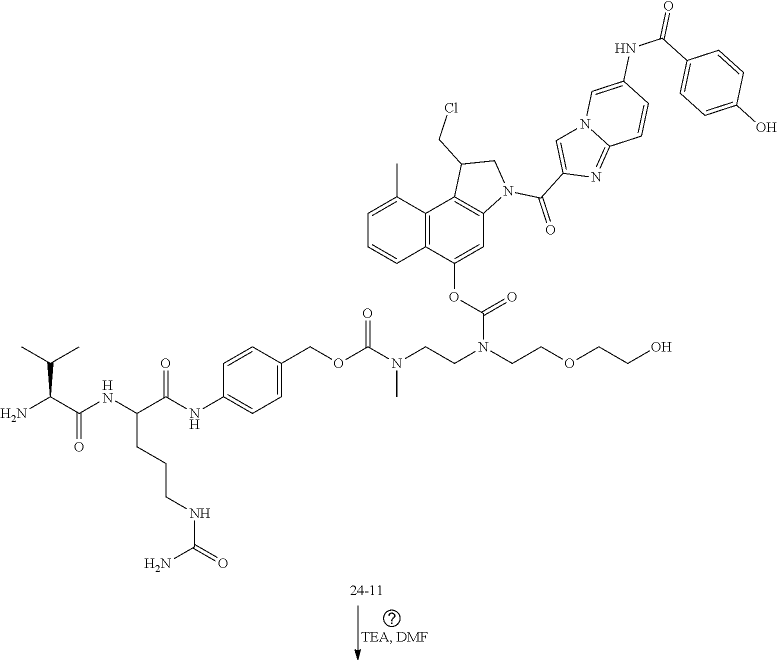 [SMILES: Cc1cccc2c(OC(=O)N(CCOCCO)CCN(C)C(=O)OCc3ccc(NC(=O)C(CCCNC(N)=O)NC(=O)[C@@H](N)C(C)C)cc3)cc3c(c12)C(CCl)CN3C(=O)c1cn2cc(NC(=O)c3ccc(O)cc3)ccc2n1]

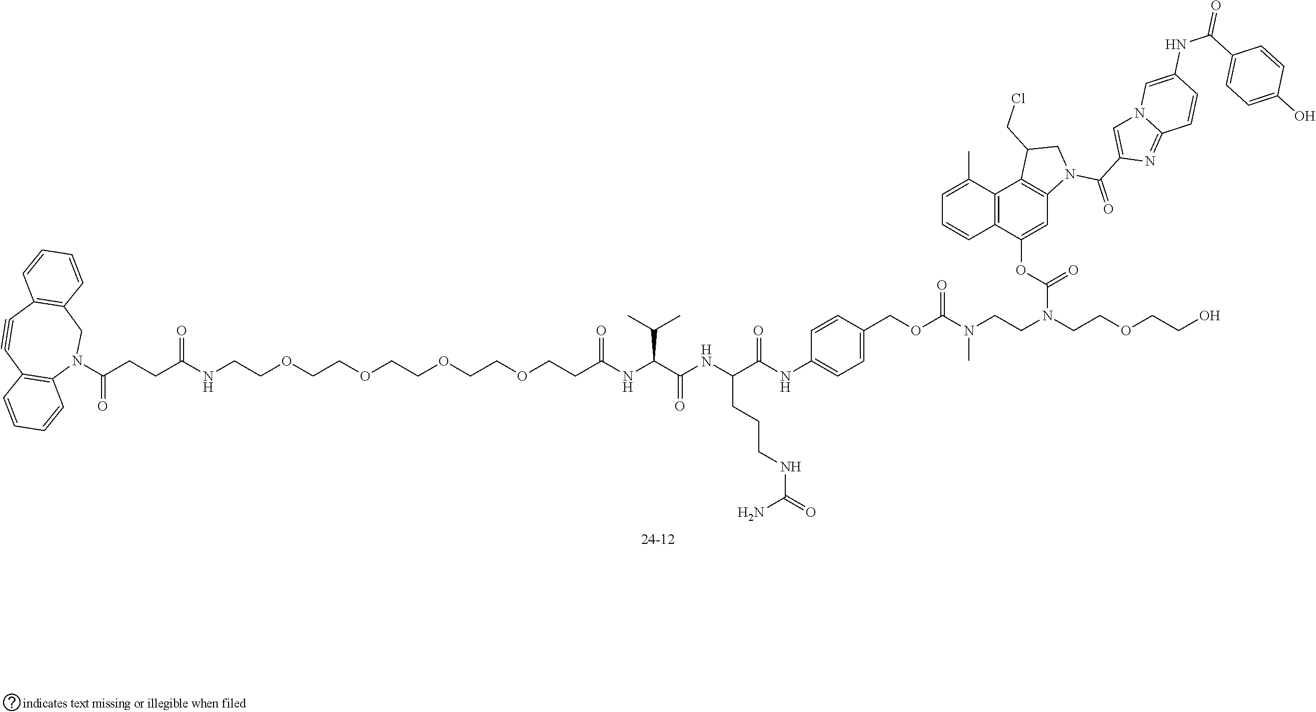 [SMILES: Cc1cccc2c(OC(=O)N(CCOCCO)CCN(C)C(=O)OCc3ccc(NC(=O)C(CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc3c(c12)C(CCl)CN3C(=O)c1cn2cc(NC(=O)c3ccc(O)cc3)ccc2n1]

DBCO-PEG₄-vc-PAB-seco-DUBA was synthesized according to the route listed above.

Tert-butyl(2-((2-(2-hydroxyethoxy)ethyl)amino)ethyl)(methyl)carbamate (24-3). To a solution of tert-butyl(2-aminoethyl)(methyl)carbamate (24-1) (5.2 g, 30 mmol) in THE (60 mL) were added 5 g TEA. Then 2-(2-bromoethoxy)ethanol (24-2) (1.7 g, 10 mmol) was dropped to the mixture stepwise. The mixture was stirred at r.t. for 5 h and monitored by TLC. The solvent was removed under reduced pressure to give the crude product 24-3.

Tert-butyl(2-((((9H-fluoren-9-yl)methoxy)carbonyl)(2-(2-hydroxyethoxy)ethyl)amino)ethyl)(methyl)carbamate (24-4). To a solution of all of the crude product 24-3 in THF (30 mL) and NaOH aq. (80 mL, 1N) were added Fmoc-Cl (7.8 g, 30.2 mmol) at r.t. The mixture was stirred at r.t. for 1 h and monitored by TLC. The solvent was then removed under reduced pressure. The residue was dissolved in 200 ml ethyl acetate and wash with washed with saturated NaHCO₃ solution and water respectively, followed by dried with Na₂SO₄. The crude product was further purified through a column chromatography to yield the 24-4 (3.5 g, yield 72% in two steps) as a light-yellow liquid.

(9H-fluoren-9-yl)methyl(2-(2-hydroxyethoxy)ethyl)(2-(methylamino)ethyl)carbamate (24-5). To a solution of 24-4 (3.4 g, 7.0 mmol) in DCM (15 mL) were added 20 ml TFA. The mixture was stirred at r.t. for 4 h and monitored by TLC. The solvent was removed under reduced pressure. The crude product was further purified through a column chromatography to yield 24-5 (0.88 g, yield 33%) as a colorless liquid. RMS (ESI+) calcd for C₂₂H₂₈N₂O₄ (M+H⁺) 385.2122, found 385.2109.

4-((S)-2-((S)-2-((tert-butoxycarbonyl)amino)-3-methylbutanamido)-5-ureidopentanamido)benzyl (2-((2-(2-hydroxyethoxy)ethyl)amino)ethyl)(methyl) carbamate (24-8). To a solution of 24-6 Boc-vc-PAB-PNP (Tsbiochem) (1.5 g, 2.3 mmol) in DMF (5 mL) were added DIPEA (1.3 mL) and 24-5 (880 mg, 2.3 mmol). The mixture was stirred at r.t. for overnight and monitored by TLC. Then 3 ml piperidine was added to the mixture and stirred for another 4 h. The product was further purified through a Prep-HPLC system to give the 24-8 as a white solid (736 mg. yield 48%). RMS (ESI−) calcd for C₃₁H₅₃N₇O₉ (M−H⁺) 666.3832, found 666.3837.

4-((S)-2-((S)-2-((tert-butoxycarbonyl)amino)-3-methylbutanamido)-5-ureidopentanamido)benzyl(2-(((((S)-1-(chloromethyl)-3-(6-(4-(methoxymethoxy)benzamido)imidazo[1,2-a]pyridine-2-carbonyl)-9-methyl-2,3-dihydro-1H-benzo[e]indol-5-yl)oxy)carbonyl)(2-(2-hydroxyethoxy)ethyl)amino)ethyl)(methyl)carbamate (24-10). The PNP-seco-DUBA (24-9) was synthesized according to the reported procedure (Beusker P. H., et al., Mol. Pharmaceutics 2015, 12, 1813). To a solution of 24-9 (125 mg, 0.17 mmol) in DMF (5 mL) were added 130 μL TEA and 136 mg 24-8 (0.20 mmol). The mixture was stirred at r.t. for overnight and monitored by TLC. The product was further purified through a Prep-HPLC system to give the 24-10 as a white solid (71 mg. yield 33%). HRMS (ESI−) calcd for C₆₃H₇₈ClN₁₁O₁₅ (M−H⁺) 1262.5295, found 1262.5287.

4-((S)-2-((S)-2-amino-3-methylbutanamido)-5-ureidopentanamido)benzyl(2-(((((S)-1-(chloromethyl)-3-(6-(4-hydroxybenzamido)imidazo[1,2-a]pyridine-2-carbonyl)-9-methyl-2,3-dihydro-1H-benzo[e]indol-5-yl)oxy)carbonyl)(2-(2-hydroxyethoxy)ethyl)amino)ethyl)(methyl)carbamate (24-11). To a solution of 24-10 (71 mg, 0.056 mmol) in DCM (2 mL) were added 3 ml TFA. The mixture was stirred at r.t. for 3 h and monitored by TLC. The solvent was removed under reduced pressure to afford the 24-11 as a crude product (58 mg) without further purification.

DBCO-PEG₄-vc-PAB-seco-DUBA (24-12). To a solution of the crude product 24-11 (25 mg) in DMF (1.5 mL) were added 100 μL TEA and 20 mg NHS-PEG₄-DBCO ester (Xi'an Dianhua Biotechnology Co., Ltd) (0.03 mmol). The mixture was stirred at room temperature overnight and monitored by TLC. The product was further purified through a Prep-HPLC system. Concentration and lyophilization to give 24-12 as a white powder (15.2 mg). HRMS (ESI−) calcd for C₈₆H₁₀₀ClN₁₃O₁₉ (M−H⁺) 1653.6908, found 1653.6948.

### Example 25 Synthesis of GDP-FAmSucMMAE (No Cleavable Linker)

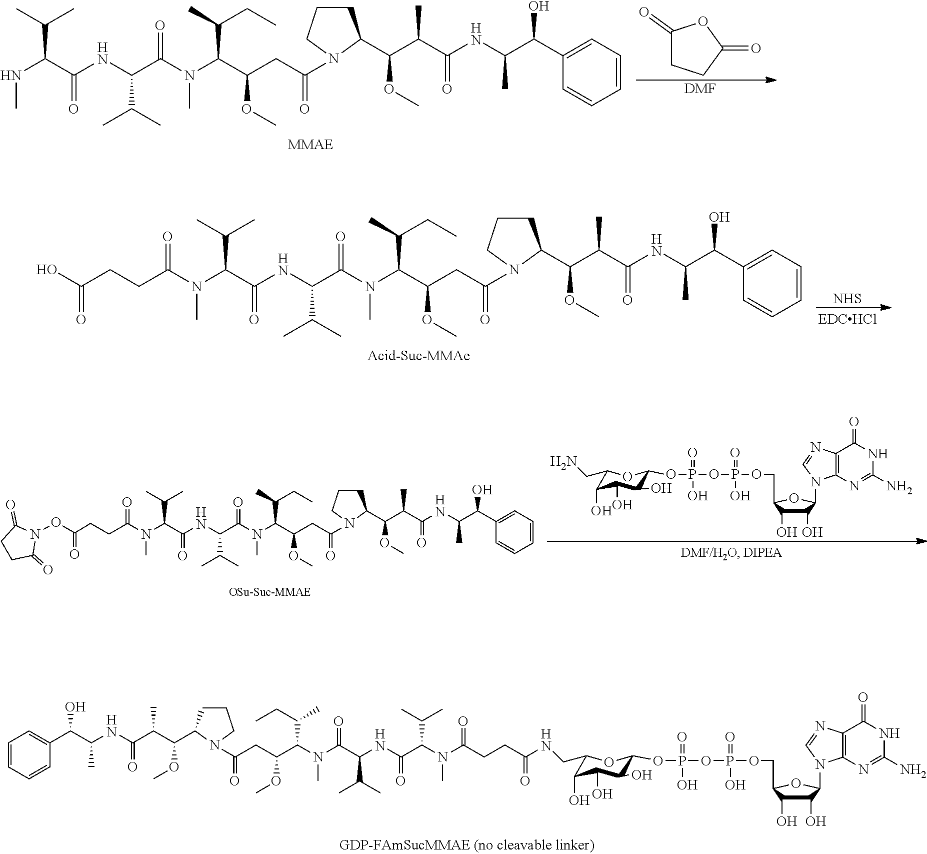

GDP-FAmSucMMAE (no cleavable linker) was synthesized according to the route list above.

Acid-Suc-MMAE. To a solution of MMAE (59 mg, 0.082 mmol) in DMF (4 mL) was added Succinic anhydride (24.7 mg, 0.25 mmol). The mixture was stirred at r.t. overnight and monitored by TLC. The product was further purified by Prep-HPLC system to give the Acid-Suc-MMAE as a white powder (52 mg, yield 77.4%).

OSu-Suc-MMAE. To a solution of Acid-Suc-MMAE (80.5 mg, 0.098 mmol) in DCM (4 mL) was added NHS (45.3 mg, 0.394 mmol) and EDC·HCl (113.2 mg, 0.59 mmol). The mixture was stirred at r.t. for 3 h and monitored by TLC. The product was further purified by Prep-HPLC system to give the OSu-Suc-MMAE as a white powder (68 mg, yield 75.6%).

GDP-FAmSucMMAE (no cleavable linker). To a solution of GDP-FAm (30 mg, 0.05 mmol) in 3 mL ddH₂O was added 26 uL DIPEA, and then OSu-Suc-MMAE (90.9 mg, 0.099 mmol) in 3 mL DMF was added. The mixture was stirred at r.t. overnight and monitored by TLC. The product was further purified by Prep-HPLC system to give the GDP-FAmSucMMAE (no cleavable linker) as a white powder (10 mg, yield 14.3%). ¹H NMR (400 MHz, D₂O) δ 8.17 (s, 1H), 7.31-7.22 (m, 5H), 5.85 (d, J=6.0 Hz, 1H), 4.84 (t, J=8.0 Hz, 1H), 4.65-4.55 (m, 1H), 4.53-4.35 (m, 4H), 4.30-4.20 (m, 2H), 4.15-3.98 (m, 4H), 3.78-3.77 (m, 1H), 3.63-3.58 (m, 2H), 3.56-3.55 (m, 1H), 3.53-3.51 (m, 1H), 3.35-2.30 (m, 1H), 3.27-3.12 (m, 10H), 3.06-3.04 (m, 2H), 2.98-2.96 (m, 2H), 2.86-2.79 (m, 1H), 2.72-2.57 (m, 3H), 2.50-2.35 (m, 4H), 2.26-1.92 (m, 4H), 1.75-1.51 (m, 4H), 1.46-1.48 (m, 1H), 1.22-1.21 (m, 2H), 1.16-1.14 (m, 1H), 1.07-1.06 (m, 1H), 1.00-0.98 (m, 2H), 0.93-0.70 (m, 20H).

### Example 26 Preparation of Antibody-G₂F Using Human β(1,4)-GalT1(Y285L)

Antibodies (10 mg/mL) were incubated with UDP-galactose (5 mM) and human β(1,4)-GalT1(Y285L) (SEQ ID NO: 1) (0.5 mg/mL) in 25 mM Tris-HCl buffer (pH 8.0) with 10 mM MnCl₂ at 30° C. for 12 to 36 hours. The reaction mixture was purified with protein A resin (Genescript) to give the antibody-G₂F. Mass spectral analysis showed the full conversion to trastuzumab-G₂F (found as 148711 Da), bevacizumab-G₂F (found as 149853 Da) and rituximab-G₂F (found as 147741 Da) respectively.

### Example 27 Generation of Trastuzumab-G₂F Using Bovine β(1,4)-GalT1(Y289L)

Trastuzumab (10 mg/mL) was incubated with UDP-galactose (5 mM) and bovine β(1,4)-GalT1(Y289L)(SEQ ID NO: 2) (0.5 mg/mL) in 25 mM Tris-HCl buffer (pH 8.0) with 10 mM MnCl₂ at 30° C. for overnight. The modified trastuzumab was purified with protein A resin. Mass spectral analysis showed the formation of one major peak (found as 148713 Da).

### Example 28 Generation of Trastuzumab-G₂F-Az Conjugates

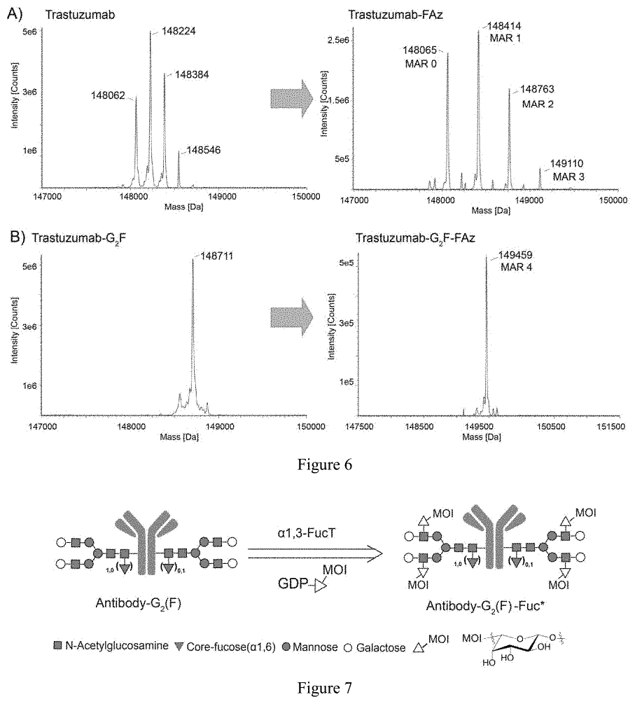

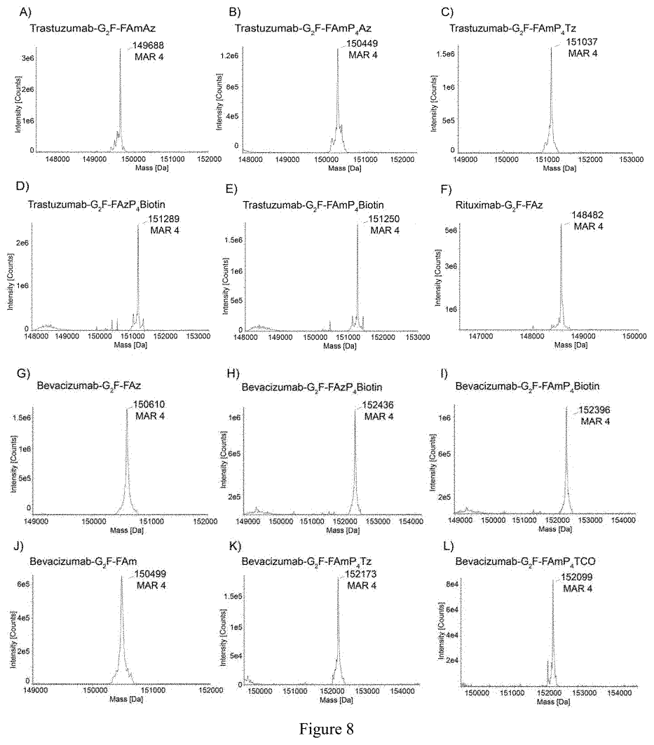

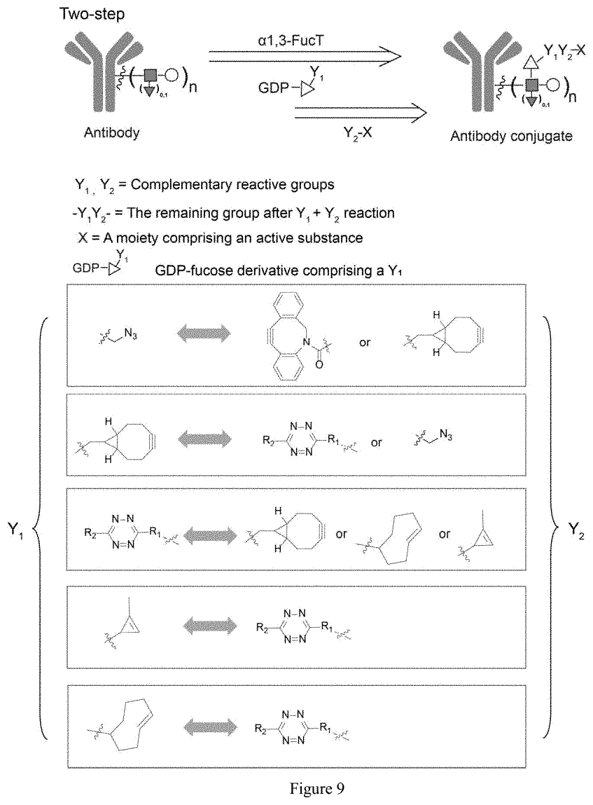

Trastuzumab-G₂F (5 mg/mL) was incubated with GDP-FAz or GDP-FAmAz or GDP-FAmP₄Az (5 mM) and Hp-α(1,3)-FucT (SEQ ID NO: 4) (0.5 mg/mL) in 50 mM Tris-HCl buffer (pH 7.5) with 20 mM MgCl₂ at 37° C. for overnight to 48 h. The reaction mixture was purified with protein A resin to give the trastuzumab-G₂F-Az conjugates. Mass spectral analysis showed complete conversion to trastuzumab-G₂F-FAz (found as 149459 Da, MAR 4) (FIG. 6B), trastuzumab-G₂F-FAmAz (found as 149688, MAR 4) (FIG. 8A) and trastuzumab-G₂F-FAmP₄Az (found as 150449, MAR 4) (FIG. 8B) respectively.

### Example 29 Generation of Trastuzumab-FAz

Trastuzumab (5 mg/mL) was incubated with GDP-FAz (5 mM) and Hp-α(1,3)-FucT (SEQ ID NO: 4) (0.5 mg/mL) in 50 mM Tris-HCl buffer (pH 7.5) with 20 mM MgCl₂ at 37° C. for overnight. The reaction mixture was purified with protein A resin to give the trastuzumab-FAz conjugates (a mixture of MAR 0, MAR 1, MAR 2 and MAR 3) (FIG. 6A). The composition of conjugates has an average MAR below 1.2.

### Example 30 Generation of Trastuzumab-G₂F-FAz Using Human FT6

Trastuzumab-G₂F (5 mg/mL) was incubated with GDP-FAz (5 mM) and human FT6 (SEQ ID NO: 5) (0.8 mg/mL) in 50 mM Tris-HCl buffer (pH 7.5) with 20 mM MgCl₂ at 37° C. for 48 h. The reaction mixture was purified with protein A resin to give the trastuzumab-G₂F-FAz conjugates. Mass spectral analysis showed formation of one major peak (found as 149461 Da, MAR 4).

### Example 31 “One-Pot” Synthesis of Trastuzumab-G₂F-FAz

Trastuzumab (5 mg/mL) was incubated with UDP-galactose (5 mM), GDP-FAz (5 mM), human β(1,4)-GalT1(Y285L) (0.5 mg/mL), Hp-α(1,3)-FucT (0.5 mg/mL) in 25 mM Tris-HCl buffer (pH 7.5) with 20 mM MgCl₂ and 10 mM MnCl₂ at 30° C. overnight. The modified trastuzumab was purified with protein A resin. Mass spectral analysis showed formation of one major peak (found as 149461 Da, MAR 4).

### Example 32 Generation of Trastuzumab-G₂F-FAzP₄Biotin

Trastuzumab-G₂F (5 mg/mL) was incubated with GDP-FAzP₄Biotin (5 mM) and Hp-α(1,3)-FucT (0.5 mg/mL) in 50 mM Tris-HCl buffer (pH 7.5) with 20 mM MgCl₂ at 37° C. for 72 hours. The reaction mixture was purified with protein A resin to give the trastuzumab-G₂F-FAzP₄Biotin. Mass spectral analysis showed the formation of one major peak (found as 151289 Da, MAR 4) (FIG. 8D) with four FAzP₄Biotin groups added to one trastuzumab-G₂F molecule. The composition of conjugates has an average MAR of 3.6-4.0.

### Example 33 Generation of Trastuzumab-G₂F-FAmP₄Biotin

Trastuzumab-G₂F (5 mg/mL) was incubated with GDP-FAmP₄Biotin (5 mM) and Hp-α(1,3)-FucT (0.5 mg/mL) in 50 mM Tris-HCl buffer (pH 7.5) with 20 mM MgCl₂ at 37° C. for 48 hours. The reaction mixture was purified with protein A resin to give the trastuzumab-G₂F-FAmP₄Biotin. Mass pectral analysis showed the formation of one major peak (found as 151250 Da) (FIG. 8E) with four FAmP₄Biotin groups added to one trastuzumab-G₂F molecule. The composition of conjugates has an average MAR of 3.6-4.0, in which more than 90% of the conjugates have a MAR of 4.

### Example 34 Generation of Trastuzumab-G₂F-FAmP₄Tz

Trastuzumab-G₂F (5 mg/mL) was incubated with GDP-FAmP₄Tz (5 mM) and Hp-α(1,3)-FucT (0.5 mg/mL) in 50 mM Tris-HCl buffer (pH 7.5) with 20 mM MgCl₂ at 37° C. for 48 hours. The reaction mixture was purified with protein A resin to give the trastuzumab-G₂F-FAmP₄Tz. Mass spectral analysis showed the formation of one major peak (found as 151037 Da) (FIG. 8C) with four FAmP₄Tz groups added to one trastuzumab-G₂F molecule. The composition of conjugates have an average MAR of 3.6-4.0, in which more than 90% of the conjugates have a MAR of 4.

### Example 35 Generation of Bevacizumab-G₂F-FAz

Bevacizumab-G₂F (5 mg/mL) was incubated with GDP-FAz (5 mM) and Hp-α(1,3)-FucT (0.5 mg/mL) in 50 mM Tris-HCl buffer (pH 7.5) with 20 mM MgCl₂ at 37° C. overnight. The reaction mixture was purified with Protein A to give the bevacizumab-G₂F-FAz. Mass spectral analysis showed the complete conversion to bevacizumab-G₂F-FAz (found as 150610 Da (FIG. 8G) with four FAz groups added to one bevacizumab-G₂F molecule. The composition of conjugates has an average MAR of 3.6-4.0, in which more than 90% of the conjugates have a MAR of 4.

### Example 36 Generation of Bevacizumab-G₂F-FAzP₄Biotin

Bevacizumab-G₂F (5 mg/mL) was incubated with GDP-FAzP₄Biotin (5 mM) and Hp-α(1,3)-FucT (0.5 mg/mL) in 50 mM Tris-HCl buffer (pH 7.5) with 20 mM MgCl₂ at 37° C. for 72 hours. The reaction mixture was purified with protein A resin to give the bevacizumab-G₂F-FAzP₄Biotin. Mass spectral analysis showed the formation of one major peak (found as 152436 Da) (FIG. 8H) with four FAzP₄Biotin groups added to one bevacizumab-G₂F molecule. The composition of conjugates has an average MAR of 3.6-4.0, in which more than 90% of the conjugates have a MAR of 4.

### Example 37 Generation of Bevacizumab-G₂F-FAmP₄Biotin

Bevacizumab-G₂F (5 mg/mL) was incubated with GDP-FAmP₄Biotin (5 mM) and Hp-α(1,3)-FucT (0.5 mg/mL) in 50 mM Tris-HCl buffer (pH 7.5) with 20 mM MgCl₂ at 37° C. for 48 hours. The reaction mixture was purified with protein A to give the bevacizumab-G₂F-FAmP₄Biotin. Mass spectral analysis showed the formation of one peak product (found as 152396 Da)(FIG. 8I) with four FAmP₄Biotin groups added to one bevacizumab-G₂F molecule. The composition of conjugates has an average MAR of 3.6-4.0, in which more than 90% of the conjugates have a MAR of 4.

### Example 38 Generation of Bevacizumab-G₂F-FAm

Bevacizumab-G₂F (5 mg/mL) was incubated with GDP-FAm (5 mM) and Hp-α(1,3)-FucT (0.5 mg/mL) in 50 mM Tris-HCl buffer (pH 7.5) with 20 mM MgCl₂ at 37° C. overnight The reaction mixture was purified with protein A resin to give the bevacizumab-G₂F-FAm. Mass spectral analysis showed the complete conversion to bevacizumab-G₂F-FAm (found as 150499 Da) (FIG. 8J) with four FAm groups added to one bevacizumab-G₂F molecule.

### Example 39 Generation of Bevacizumab-G₂F-FAmP₄Tz

Bevacizumab-G₂F was subjected to the process described in example 34. Mass spectral analysis showed the formation of one major peak (found as 152173 Da) (FIG. 8K) with four FAmP₄Tz groups added to one bevacizumab-G₂F molecule. The composition of conjugates has an average MAR of 3.6-4.0, in which more than 90% of the conjugates have a MAR of 4.

### Example 40 Generation of Bevacizumab-G₂F-FAmP₄TCO

Bevacizumab-G₂F (5 mg/mL) was incubated with GDP-FAmP₄TCO (1 mM) and Hp-α(1,3)-FucT (0.5 mg/mL) in 50 mM Tris-HCl buffer (pH 7.5) with 20 mM MgCl₂ at 37° C. for 48 hours. The reaction mixture was purified with protein A resin to give the bevacizumab-G₂F-FAmP₄TCO. Mass spectral analysis showed the formation of one major peak (found as 152099 Da)(FIG. 8L) with four FAmP₄TCO groups added to one bevacizumab-G₂F molecule. One more minor peak appeared due to the fragmentation of TCO linkage during MS spectrometry. Similar fragmentation appeared for following antibody-conjugates containing the TCO linkage. The composition of conjugates has an average MAR of 3.6-4.0, in which more than 90% of the conjugates have a MAR of 4.

### Example 41 Generation of Rituximab-G₂F-FAz

Rituximab-G₂F was subjected to the process described in example 28. Mass spectral analysis showed the formation of one major peak (found as 148482 Da) (FIG. 8F) with four FAz groups added to one rituximab-G₂F molecule. The composition of conjugates has an average MAR of 3.6-4.0, in which more than 90% of the conjugates have a MAR of 4.

### Example 42 Generation of Trastuzumab-G₂F-AzDBCO-GGG Conjugates

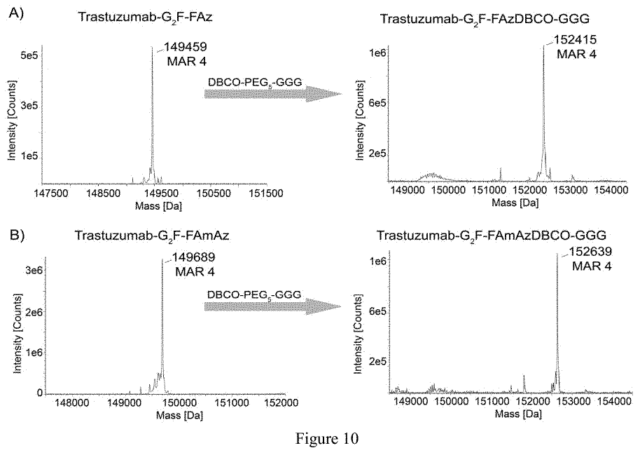

Azido groups modified trastuzumab-G₂F (trastuzumab-G₂F-FAz and trastuzumab-G₂F-FAmAz, 1 mg/mL) were incubated with 100 μM DBCO-PEG₅-GGG in PBS buffer at r.t. overnight. Then the reaction mixture was purified with protein A resin to give the trastuzumab-G₂F-FAzDBCO-GGG and trastuzumab-G₂F-FAmAzDBCO-GGG conjugates. (FIG. 10)

### Example 43 Generation of Trastuzumab-G₂F-FAzP₄MMAE

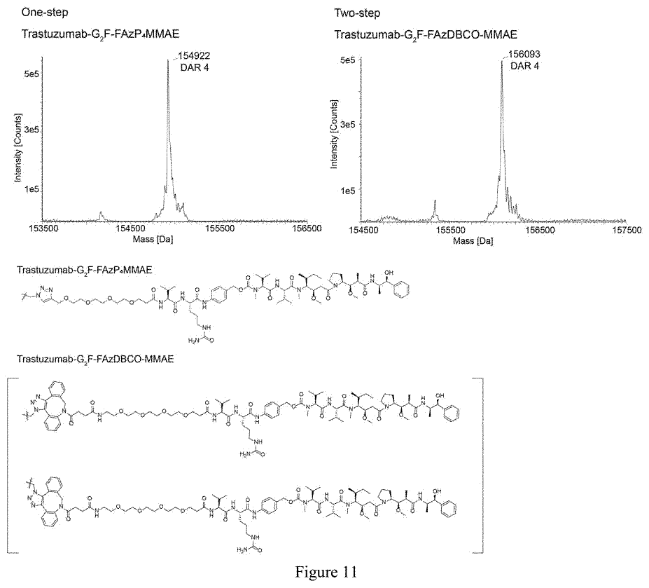

Trastuzumab-G₂F (3 mg/mL) was incubated with GDP-FAzP₄MMAE (5 mM) and Hp-α(1,3)-FucT (0.5 mg/mL) in 50 mM Tris-HCl buffer (pH 7.5) with 20 mM MgCl₂ at 37° C. for 72 hours. The reaction mixture was purified with protein A resin to give the trastuzumab-G₂F-FAzP₄MMAE. Mass spectral analysis showed the formation of one major peak (found as 154922 Da) with four MMAE added to one trastuzumab-G₂F molecule (FIG. 11). A minor peak (found as Da) was resulted from the PABA linker fragmentating in the MS spectormetry, similar fragments appeared in the following antibody-drug conjugates containing the PABA linker. The composition of conjugates has an average DAR of 3.6-4.0, in which more than 90% of the conjugates have a DAR of 4.

### Example 44 Generation of Trastuzumab-G₂F-FAzDBCO-MMAE

Trastuzumab-G₂F-FAz (1.5 mg/mL) was incubated with DBCO-PEG₄-vc-PAB-MMAE (150 μM)(Levena Biopharma) in PBS (pH 7.4) with 10% DMSO at r.t. for 48 hours. The reaction mixture was purified with protein A resin to give the trastuzumab-G₂F-FAzDBCO-MMAE. Mass spectral analysis showed one major peak (found as 156093 Da) with four MMAE added to one trastuzumab-G₂F-FAz molecule (FIG. 11). The composition of conjugates has an average DAR of 3.6-4.0, in which more than 90% of the conjugates have a DAR of 4.

### Example 45 In Vitro Efficacy of Trastuzumab-G₂F-FAzP₄MMAE and Trastuzumab-G₂F-FAzDBCO-MMAE on SK-Br-3 (Her2+) and MDA-MB-231 (Her2−) Cells Respectively

SK-Br-3 (Her2+) and MDA-MB-231 (Her2−) cells were cultured in McCoy's 5A medium (Gibco) and DMEM (Gibco) supplemented with 10% FBS (Invitrogen) respectively. The cells were plated in 96-well plates with 5000 cells per well and were incubated for 24 hours at 37° C. and 5% CO₂. After remove of the culture medium, the antibody samples (trastuzumab, trastuzumab-G₂F-FAzP₄MMAE and trastuzumab-G₂F-FAzDBCO-MMAE) were added to culturing medium to a series of final concentrations (100 nM, 10 nM, 1 nM, 0.5 nM, 0.1 nM, 0.05 nM, 0.01 nM, 0.001 nM and 0 nM) and added to the plates respectively. The cells were incubated for 72 h at 37° C. and 5% CO₂ and subjected to a CellTiter-Glo® Luminescent Cell Viability Assay (Promega) to measure the cell viability. Both the trastuzumab-G₂F-FAzP₄MMAE and trastuzumab-G₂F-FAzDBCO-MMAE showed high potent of killing cells towards Her2-positive cell lines SK-Br-3, but not of the Her2-negative cell line MDA-MB-231 (FIG. 12).

### Example 46 Generation of Trastuzumab-((Fuc)α1,6)(Galβ1,4)GlcNAc

Trastuzumab (10 mg/mL) was incubated with EndoS (SEQ ID NO: 6) (0.05 mg/mL) in 50 mM Tris-HCl pH 8.0 for 1 hours at 37° C. and then purified with Protein A. Mass spectral analysis showed the formation of one major peak trastuzumab-((Fuc)α1,6)GlcNAc (found as 145867 Da). Trastuzumab-((Fuc)α1,6)GlcNAc (10 mg/mL) was further incubated with UDP-Galactose (5 mM) and human β(1,4)-GalT1(Y285L) (0.5 mg/mL) in 10 mM MnCl₂ and 25 mM Tris-HCl pH 8.0 for 48 hours at 30° C. The reaction mixture was purified with protein A resin to give the trastuzumab-((Fuc)α1,6)(Galβ1,4)GlcNAc. Mass spectral analysis showed the formation of one major peak (found as 146192 Da) with two galactoses added to one trastuzumab-((Fuc)α1,6)GlcNAc molecule.

### Example 47 Generation of Trastuzumab-((Fuc)α1,6)(Galβ1,4)GlcNAc-FAz

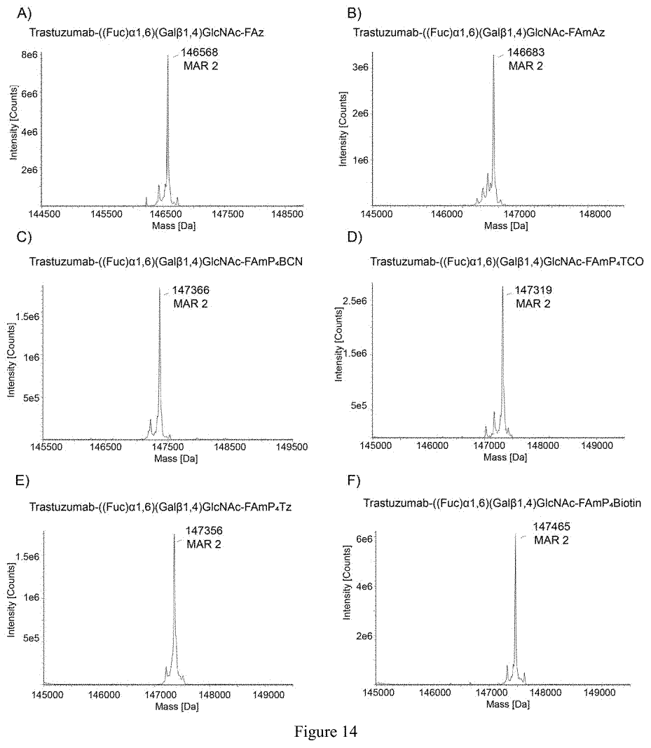

Trastuzumab-((Fuc)α1,6)(Galβ1,4)GlcNAc was subjected to the process described in example 28. Mass spectral analysis showed the formation of one major peak (found as 146568 Da, MAR 2)(FIG. 14A). The composition of conjugates has an average MAR of 1.8-2.0, in which more than 90% of the conjugates have a MAR of 2.

### Example 48 Generation of Trastuzumab-((Fuc)α1,6)(Galβ1,4)GlcNAc-FAmAz

Trastuzumab-((Fuc)α1,6)(Galβ1,4)GlcNAc was subjected to the process described in example 28. Mass spectral analysis showed the formation of one major peak (found as 146683 Da, MAR 2)(FIG. 14B). The composition of conjugates has an average MAR of 1.8-2.0, in which more than 90% of the conjugates have a MAR of 2.

### Example 49 Generation of Trastuzumab-((Fuc)α1,6)(Galβ1,4)GlcNAc-FAmP₄BCN

Trastuzumab-((Fuc)α1,6)(Galβ1,4)GlcNAc (5 mg/mL) was incubated with GDP-FAmP₄BCN (5 mM) and Hp-α(1,3)-FucT (0.5 mg/ml) in 20 mM MgCl₂ and 50 mM Tris-HCl pH 7.5 for 48 hours at 37° C. The reaction mixture was purified with protein A resin to give the trastuzumab-((Fuc)α1,6)(Galβ1,4)GlcNAc-FAmP₄BCN. Mass spectral analysis showed the formation of one major peak (147366 Da, MAR 2))(FIG. 14C). The composition of conjugates has an average MAR of 1.8-2.0, in which more than 90% of the conjugates have a MAR of 2.

### Example 50 Generation of Trastuzumab-((Fuc)α1,6)(Galβ1,4)GlcNAc-FAmP₄TCO

Trastuzumab-((Fuc)α1,6)(Galβ1,4)GlcNAc (5 mg/mL) was subjected to the process described in example 40. Mass spectral analysis showed the formation of one major peak (found as 147319 Da, MAR 2) (FIG. 14D). The composition of conjugates has an average MAR of 1.8-2.0, in which more than 90% of the conjugates have a MAR of 2.

### Example 51 Generation of Trastuzumab-((Fuc)α1,6)(Galβ1,4)GlcNAc-FAmP₄Tz

Trastuzumab-((Fuc)α1,6)(Galβ1,4)GlcNAc was subjected to the process described in example 34. Mass spectral analysis showed the formation of one major peak (found as 147356 Da, MAR 2)(FIG. 14E). The composition of conjugates has an average MAR of 1.8-2.0, in which more than 90% of the conjugates have a MAR of 2.

### Example 52 Generation of Trastuzumab-((Fuc)α1,6)(Galβ1,4)GlcNAc-FAmP₄Biotin

Trastuzumab-((Fuc)α1,6)(Galβ1,4)GlcNAc was subjected to the process described in example 33. Mass spectral analysis showed the formation of one major peak (found as 147465 Da, MAR 2) (FIG. 14F). The composition of conjugates has an average MAR of 1.8-2.0, in which more than 90% of the conjugates have a MAR of 2.

### Example 53 Generation of Antibody-(Galβ1,4)GlcNAc

Antibodies (10 mg/mL) were incubated with EndoS (0.05 mg/mL) and Alfc (1.5 mg/mL) (SEQ ID NO: 7) in 50 mM Tris-HCl pH 8.0 for 24 hours at 37° C. and then purified with Protein A. Mass spectral analysis showed the formation of trastuzumab-GlcNAc (found as 145583 Da), rituximab-GlcNAc (found as 144599 Da) and hRS7-GlcNAc (found as 145426 Da) respectively. Antibody-GlcNAc (10 mg/mL) was further incubated with UDP-galactose (5 mM) and human β(1,4)-GalT1(Y285L) (0.5 mg/mL) in 10 mM MnCl₂ and 25 mM Tris-HCl pH 8.0 for 24 hours at 30° C. The reaction mixture was purified with protein A resin. Mass spectral analysis showed complete conversion to trastuzumab-(Galβ1,4)GlcNAc (found as 145907 Da), rituximab-(Galβ1,4)GlcNAc (found as 144926 Da) and hRS7-(Galβ1,4)GlcNAc (found as 145750 Da) respectively.

### Example 54 Generation of Antibody-(Galβ1,4)GlcNAc-Az

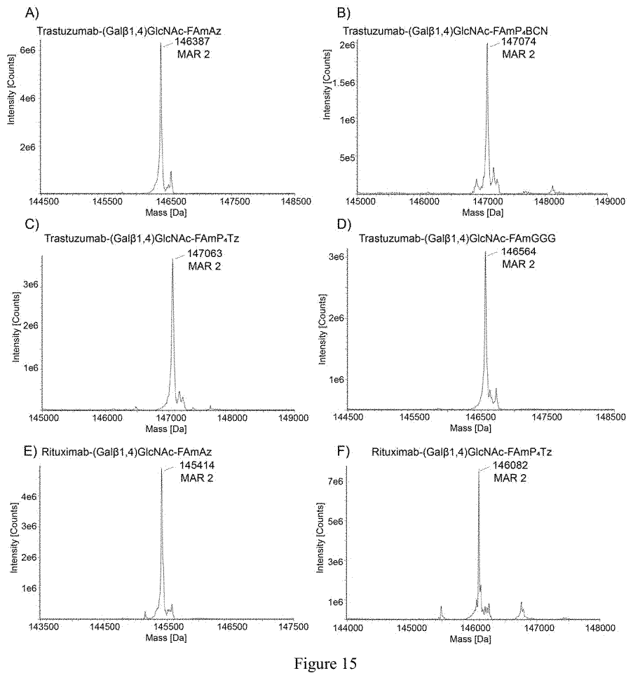

Antibody-(Galβ1,4)GlcNAc was subjected to the process described in example 28. Mass spectral analysis showed complete conversion to trastuzumab-(Galβ1,4)GlcNAc-FAz (found as 146289 Da, MAR 2), trastuzumab-(Galβ1,4)GlcNAc-FAmAz (found as 146387 Da, MAR 2) (FIG. 15A), trastuzumab-(Gal1,4)GlcNAc-FAmP₄Az (found as 146777, MAR 2), rituximab-(Galβ1,4)GlcNAc-FAz (found as 145300 Da, MAR 2), rituximab-(Galβ1,4)GlcNAc-FAmAz (found as 145414 Da, MAR 2)(FIG. 15E) and hRS7-(Galβ1,4)GlcNAc-FAmAz (found as 146239 Da, MAR 2), respectively. All the compositions of conjugates have an average MAR of 1.8-2.0, in which more than 90% of the conjugates have a MAR of 2.

### Example 55 Generation of Trastuzumab-(Galβ1,4)GlcNAc-FAmP₄BCN

Trastuzumab-(Galβ1,4)GlcNAc (5 mg/mL) was subjected to the process described in example 49. Mass spectral analysis showed the formation of one major peak (found as 147074 Da, MAR 2) (FIG. 15B). The composition of conjugates has an average MAR of 1.8-2.0, in which more than 90% of the conjugates have a MAR of 2.

### Example 56 Generation of Antibody-(Galβ1,4)GlcNAc-FAmP₄Tz

Antibody-(Galβ1,4)GlcNAc (5 mg/mL) was subjected to the process described in example 34. Mass spectral analysis showed the formation of one major peak trastuzumab-(Galβ1,4)GlcNAc-FAmP₄Tz (found as 147063 Da, MAR 2)(FIG. 15C), rituximab-(Galβ1,4)GlcNAc-FAmP₄Tz (found as 146082 Da, MAR 2) (FIG. 15F) respectively.

### Example 57 Generation of Trastuzumab-(Galβ1,4)GlcNAc-FAmGGG

Trastuzumab-(Galβ1,4)GlcNAc (5 mg/mL) was incubated with GDP-FAmGGG (5 mM) and Hp-α(1,3)-FucT (0.5 mg/ml) in 20 mM MgCl₂ and 50 mM Tris-HCl pH 7.5 for 24 hours at 37° C. The reaction mixture was purified with protein A resin to give the trastuzumab-(Galβ1,4)GlcNAc-FAmGGG. Mass spectral analysis showed the formation of one major peak (found as 146564 Da, MAR 2) (FIG. 15D). The composition of conjugates has an average MAR of 1.8-2.0, in which more than 90% of the conjugates have a MAR of 2.

### Example 58 Comparison of the Catalytic Efficiency of Hp-α(1,3)-FucT Towards GDP-FAzX Derivatives and GDP-FAmX Derivatives

Trastuzumab-G₂F (2 mg/mL) was incubated with GDP-FAzP₄Biotin (1 mM) or GDP-FAmP₄Biotin (1 mM) and Hp-α(1,3)-FucT(SEQ ID NO: 4) (0.5 mg/mL) in 50 mM Tris-HCl buffer (pH 7.5) with 5 mM MgCl₂ at 30° C. for 6 hours. Trastuzumab-(Galβ1,4)GlcNAc (2 mg/mL) was incubated with GDP-FAzP₄Biotin (1 mM) or GDP-FAmP₄Biotin (1 mM) and Hp-α(1,3)-FucT(SEQ ID NO: 4) (0.5 mg/mL) in 50 mM Tris-HCl buffer (pH 7.5) with 5 mM MgCl₂ at 30° C. for 2 hours. Trastuzumab-(Galβ1,4)GlcNAc (2 mg/mL) was incubated with GDP-FAzP₄MMAE (1 mM) or GDP-FAmP₄Biotin (1 mM) and Hp-α(1,3)-FucT (0.5 mg/mL) in 50 mM Tris-HCl buffer (pH 7.5) with 5 mM MgCl₂ at 30° C. for 2 hours. The reaction mixture was purified with protein A resin and analyzed by LC-MS respectively. For trastuzumab-G₂F, % of conversion=average MAR/4*100%. For trastuzumab-(Galβ1,4)GlcNAc, % of conversion=average MAR/2*100%. The results showed that Hp-α(1,3)-FucT displayed significant higher catalytic efficiency towards the GDP-FAmX derivatives than the GDP-FAzX derivatives in transferring active molecule to the antibody-G₂F and the antibody-(Galβ1,4)GlcNAc (FIG. 16).

### Example 59 Comparison of the Catalytic Efficiency of Hp-α(1,3)-FucT and Human FT6 Towards GDP-FAmP₄Biotin on Trastuzumab-G₂F

Trastuzumab-G₂F (2 mg/mL) was incubated with GDP-FAmP₄Biotin (1 mM) and Hp-α(1,3)-FucT(SEQ ID NO: 4) (0.5 mg/mL) or human FT6 (SEQ ID NO: 5)(0.5 mg/mL) in 40 mM Tris-HCl buffer (pH 7.5) with 20 mM MgCl₂ at 30° C. for 6 hours and 16 hours. The reaction mixture was purified with protein A resin and analyzed by LC-MS respectively. For trastuzumab-G₂F, % of conversion=average MAR/4*100%. The results showed that Hp-α(1,3)-FucT display dramatically higher catalytic efficiency towards the GDP-FAmP₄Biotin in transferring the FAmP₄Biotin to the antibody-G₂F than Human FT6 (FIG. 18). After 3 hours, the Hp-α(1,3)-FucT achieved 10% of conversion while the Human FT6 achieved undetectable level of conversion. After 16 hours, the Hp-α(1,3)-FucT achieved 69% of conversion while the Human FT6 only achieved 4% of conversion.

### Example 60 Generation of Antibody-G₂F-FAmAzDBCO-MMAE

Antibody-G₂F-FAmAz (1.5 mg/mL) was incubated with DBCO-PEG₄-vc-PAB-MMAE (100 μM)(Levena Biopharma) in PBS (pH 7.4) with 10% DMSO at r.t. for 16 hours. The reaction mixture was purified with protein A resin. Mass spectral analysis the formation of one major peak trastuzumab-G₂F-FAmAzDBCO-MMAE (found as 156322 Da, DAR 4) (FIG. 17A) and rituximab-G₂F-FAmAzDBCO-MMAE (found as 155341 Da, DAR 4) (FIG. 17C), respectively. All the compositions of conjugates have an average DAR of 3.6-4, in which more than 90% of the conjugates have a DAR of 4.

### Example 61 Generation of Rituximab-G₂F-FAzDBCO-MMAE

Rituximab-G₂F-FAz (1.5 mg/mL) was subjected to the process described in example 60. Mass spectral analysis showed the formation of one major peak rituximab-G₂F-FAzDBCO-MMAE (found as 155113 Da, DAR 4). The composition of conjugates has an average DAR of 3.6-4, in which more than 90% of the conjugates have a DAR of 4.

### Example 62 Generation of Trastuzumab-G₂F-FAmAzDBCO-MMAF

Trastuzumab-G₂F-FAmAz (1.5 mg/mL) was incubated with DBCO-PEG₄-vc-PAB-MMAF (100 μM)(Levena Biopharma) in PBS (pH 7.4) with 10% DMSO at r.t. for 16 hours. The reaction mixture was purified with protein A resin. Mass spectral analysis showed the formation of one major peak trastuzumab-G₂F-FAmAzDBCO-MMAF (found as 156378 Da, DAR 4) (FIG. 17B). The composition of conjugates has an average DAR of 3.6-4, in which more than 90% of the conjugates have a DAR of 4.

### Example 63 Generation of Antibody-(Galβ1,4)GlcNAc-FAmAzDBCO-MMAE

Antibody-(Galβ1,4)GlcNAc-FAmAz (1.5 mg/mL) was subjected to the process described in example 60. Mass spectral analysis showed the formation of one major peak trastuzumab-(Galβ1,4)GlcNAc-FAmAzDBCO-MMAE (found as 149708 Da, DAR 2) (FIG. 17D) and hRS7-(Galβ1,4)GlcNAc-FAmAzDBCO-MMAE (found as 149555 Da, DAR 2) (FIG. 17J), respectively. All the compositions of conjugates have an average DAR of 1.8-2.0, in which more than 90% of the conjugates have a DAR of 2.

### Example 64 Generation of Trastuzumab-(Galβ1,4)GlcNAc-FAmAzDBCO-MMAF

Trastuzumab-(Galβ1,4)GlcNAc-FAmAz (1.5 mg/mL) was subjected to the process described in example 62. Mass spectral analysis showed the formation of one major peak trastuzumab-(Galβ1,4)GlcNAc-FAmAzDBCO-MMAF (found as 149736 Da, DAR2)(FIG. 17E) with two MMAF added to one trastuzumab molecule. The composition of conjugates has an average DAR of 1.8-2, in which more than 90% of the conjugates have a DAR of 2.

### Example 65 Generation of Trastuzumab-(Galβ1,4)GlcNAc-FAmP₄AzDBCO-seco-DUBA

Trastuzumab-(Galβ1,4)GlcNAc-FAmP₄Az (1.5 mg/mL) was incubated with DBCO-PEG₄-vc-PAB-seco-DUBA (100 μM) in PBS (pH 7.4) with 45% propanediol at r.t. for 16 hours. The reaction mixture was purified with protein A resin. Mass spectral analysis showed the formation of one major peak trastuzumab-(Galβ1,4)GlcNAc-FAmP₄AzDBCO-seco-DUBA (found as 150081 Da, DAR2) with two seco-DUBA added to one trastuzumab molecule (FIG. 17F). The composition of conjugates has an average DAR of 1.8-2.0, in which more than 90% of the conjugates have a DAR of 2.

### Example 66 Generation of Trastuzumab-(Galβ1,4)GlcNAc-FAmAzBCN-MMAE

Trastuzumab-(Galβ1,4)GlcNAc-FAmAz (1.5 mg/mL) was incubated with BCN-PEG₄-vc-PAB-MMAE (100 μM) (BroadPharm) in PBS (pH 7.4) with 10% DMSO at r.t. for 16 hours. The reaction mixture was purified with protein A resin. Mass spectral analysis showed the formation of one major peak trastuzumab-(Galβ1,4)GlcNAc-FAmAzBCN-MMAE (found as 149486 Da, DAR2) with two MMAE added to one trastuzumab molecule (FIG. 17G). The composition of conjugates has an average DAR of 1.8-2, in which more than 90% of the conjugates have a DAR of 2.

### Example 67 Generation of Trastuzumab-(Galβ1,4)GlcNAc-FAmP₄BCNAz-MMAE

Trastuzumab-(Galβ1,4)GlcNAc-FAmP₄BCN (1.5 mg/mL) was incubated with Az-PEG₄-vc-PAB-MMAE (100 μM) in PBS (pH 7.4) with 10% DMSO at r.t. for 16 hours. The reaction mixture was purified with protein A resin. Mass spectral analysis showed the formation of one major peak trastuzumab-(Galβ1,4)GlcNAc-FAmP₄BCNAz-MMAE (found as 149863 Da, DAR2) with two MMAE added to one trastuzumab molecule (FIG. 17H). The composition of conjugates has an average DAR of 1.8-2.0, in which more than 90% of the conjugates have a DAR of 2.

### Example 68 Generation of Trastuzumab-(Galβ1,4)GlcNAc-FAmP₄TzTCO-MMAE

Trastuzumab-(Galβ1,4)GlcNAc-FAmP₄Tz (1.5 mg/mL) was incubated with TCO-PEG₄-vc-PAB-MMAE (100 μM) in PBS (pH 7.4) with 10% DMSO at r.t. for 2 hours. The reaction mixture was purified with protein A resin. Mass spectral analysis showed the formation of one major peak trastuzumab-(Galβ1,4)GlcNAc-FAmP₄TzTCO-MMAE (found as 150051 Da, DAR2) with two MMAE added to one trastuzumab molecule. One more minor peak appeared (found as 148637 Da) due to the fragmentation of TCO linkage during MS spectrometry (FIG. 17I). The composition of conjugates has an average DAR of 1.8-2.0, in which more than 90% of the conjugates have a DAR 2.

### Example 69 “One-Step” Generation of Trastuzumab-Drug Conjugates

Trastuzumab-G₂F (3 mg/mL) or trastuzumab-(Galβ1,4)GlcNAc (3 mg/mL) was incubated with GDP-Fuc derivatives obtained from Examples 16-21 and 25 (GDP-FAmP₄MMAE, GDP-FAmSucMMAE, GDP-FAmAzP₄MMAE, GDP-FAmP₄AzP₄MMAE, GDP-FAmAzP₄DXd, GDP-FAmDM4, or GDP-FAmSucMMAE (no cleavable linker)) (5 mM) and Hp1,3-FucT (0.5 mg/mL) in 50 mM Tris-HCl buffer (pH 7.5) with 20 mM MgCl₂ at 37° C. for 24 to 48 h. The reaction mixture was purified with protein A resin to give the trastuzumab-drug conjugates. Mass spectral analysis showed the formation of one major peak trastuzumab-G₂F-FAmP₄MMAE (found as 154879 Da, DAR4) (FIG. 17K), trastuzumab-G₂F-FAmSucMMAE (found as 154177 Da, DAR4) (FIG. 17L), trastuzumab-G₂F-FAmAzP₄MMAE (found as 155147 Da, DAR4) (FIG. 17M), trastuzumab-G₂F-FAmP₄AzP₄MMAE (found as 155908 Da, DAR4) (FIG. 17N), trastuzumab-G₂F-FAmAzP₄DXd (found as 153967 Da, DAR4)(FIG. 17Q), trastuzumab-G₂F-FAmDM4 (found as 152877 Da, DAR4) (FIG. 17R) trastuzumab-(Galβ1,4)GlcNAc-FAmP₄MMAE (found as 148989 Da, DAR2) (FIG. 17O), trastuzumab-(Galβ1,4)GlcNAc-FAmSucMMAE (found as 148634 Da, DAR2) (FIG. 17P), and trastuzumab-(Galβ1,4)GlcNAc-FAmAzP₄DXd (found as 148561 Da, DAR2)(FIG. 17S), trastuzumab-(Galβ1,4)GlcNAc-FAmSucMMAE (no cleavable linker) (found as 147873 Da, DAR2), respectively. All the compositions of the trastuzumab-G₂F-Fuc* conjugates have an average DAR of 3.6-4, in which more than 90% of the conjugates have a DAR of 4. All the compositions of the trastuzumab-(Galβ1,4)GlcNAc-Fuc* conjugates have an average DAR of 1.8-2.0, in which more than 90% of the conjugates have a DAR of 2.

### Example 70 “One-Step” Generation of hRS7-(Galβ1,4)GlcNAc-Drug Conjugates

The hRS7-(Galβ1,4)GlcNAc (3 mg/mL) was subjected to the process described in example 69. Mass spectral analysis showed the formation of one major peak hRS7-(Galβ1,4)GlcNAc to hRS7-(Galβ1,4)GlcNAc-FAmSucMMAE (found as 148484 Da, DAR2) (FIG. 17T), and hRS7-(Galβ1,4)GlcNAc-FAmAzP₄MMAE (found as 148969 Da, DAR2) (FIG. 17U) respectively. The composition of conjugates has an average DAR of 1.8-2.0, in which more than 90% of the conjugates have a DAR of 2.

### Example 71 Generation of Trastuzumab-(Fucα1,6)(Galβ1,4)GlcNAc-FAmSucMMAE

Trastuzumab-((Fuc)α1,6)(Galβ1,4)GlcNAc (5 mg/mL) was incubated with GDP-FAmSucMMAE (5 mM) and Hp-α(1,3)-FucT (0.5 mg/mL) in 50 mM Tris-HCl buffer (pH 7.5) with 20 mM MgCl₂ at 37° C. for 72 hours. The reaction mixture was purified with protein A to give the trastuzumab-((Fuc)α1,6)(Gal1,4)GlcNAc-FAmSucMMAE. Mass spectral analysis showed the formation of one major peak (found as 148940 Da, DAR2) with two MMAE added to one trastuzumab-((Fuc)α1,6)(Galβ1,4)GlcNAc molecule. The composition of conjugates has an average DAR of 1.8-2.0, in which more than 90% of the conjugates have a DAR of 2.

### Example 72 the Reactivity of Antibody-FAz, Antibody-FAmAz and Antibody-FAmP₄Az Towards DBCO-PEG₄-Vc-PAB-MMAE and the Reactivity of Antibody-FAmP₄Tz Towards TCO-PEG₄-vc-PAB-MMAE

Trastuzumab-G₂F-FAz, trastuzumab-G₂F-FAmAz, trastuzumab-G₂F-FAmP₄Az (2 mg/mL) was incubated with DBCO-PEG₄-vc-PAB-MMAE (133 μM), and trastuzumab-G₂F-FAmP₄Tz (2 mg/mL) was incubated with TCO-PEG₄-vc-PAB-MMAE (133 μM), in PBS (pH 7.4) with 10% DMSO at r.t. for 2, 4 and 16 hours respectively. Trastuzumab-(Galβ1,4)GlcNAc-FAz, trastuzumab-(Galβ1,4)GlcNAc-FAmAz and trastuzumab-(Galβ1,4)GlcNAc-FAmP₄Az was incubated with DBCO-PEG₄-vc-PAB-MMAE (80 μM), and trastuzumab-(Galβ1,4)GlcNAc-FAmP₄Tz (2 mg/mL) was incubated with TCO-PEG₄-vc-PAB-MMAE (80 μM), in PBS (pH 7.4) with 10% DMSO at r.t. for 1, 2 and 4 hours. For trastuzumab-G₂F-drug conjugates, % of conversion=average DAR/4*100%. For trastuzumab-(Galβ1,4)GlcNAc-drug conjugates, % of conversion=average DAR/2*100%. The results demonstrated the trastuzumab-G₂F-FAmP₄Az and the trastuzumab-G₂F-FAmAz showed significant higher reactivity than the trastuzumab-G₂F-FAmP₄Az towards the DBCO-PEG₄-vc-PAB-MMAE. Meanwhile, the reaction rate of TCO-PEG₄-vc-PAB-MMAE towards trastuzumab-G₂F-FAmP₄Tz was much faster than the DBCO-PEG₄-vc-PAB-MMAE towards all three kinds of trastuzumab-G₂F-Az conjugates (FIG. 19A). Similar results were observed in trastuzumab-(Galβ1,4)GlcNAc-FAz, trastuzumab-(Galβ1,4)GlcNAc-FAmAz, trastuzumab-(Galβ1,4)GlcNAc-FAmP₄Az and trastuzumab-(Galβ1,4)GlcNAc-FAmP₄Tz (FIG. 19B).

### Example 73 “One-Pot” Synthesis of Trastuzumab-G₂F-Fuc*

Trastuzumab (5 mg/mL) was incubated with UDP-galactose (5 mM), with GDP-Fuc derivatives (5 mM) (GDP-FAmAz, GDP-FAmP₄Tz, GDP-FAmSucMMAE or GDP-FAmAzDXd), human β(1,4)-GalT1(Y285L) (0.5 mg/mL), Hp-α(1,3)-FucT (0.7 mg/mL) in 25 mM Tris-HCl buffer (pH 7.0) with 20 mM MgCl₂ and 10 mM MnCl₂ at 30° C. for overnight to 72 hours. The modified trastuzumab was purified with protein A resin. Mass spectral analysis showed formation of one major peak trastuzumab-G₂F-FAmAz (found as 151041 Da, MAR 4), trastuzumab-G₂F-FAmP₄Tz (found as 154177 Da, MAR 4), trastuzumab-G₂F-FAmSucMMAE (found as 154033 Da, DAR 4), trastuzumab-G₂F-FAmAzDXd (found as 154033 Da, DAR 4). All the compositions of the trastuzumab-G₂F-Fuc* conjugate have an average MAR or DAR of 3.6-4, in which more than 90% of the conjugates have a MAR or DAR of 4.

### Example 74 “One-Pot” Synthesis of Trastuzumab-(Galβ1,4)GlcNAc-Fuc*

Trastuzumab (10 mg/mL) was incubated with EndoS (0.05 mg/mL) and Alfc (1.5 mg/mL) in 50 mM Tris-HCl pH 7.0 for 24 hours at 37° C., and then incubated with UDP-galactose (5 mM), GDP-Fuc derivatives (5 mM) (GDP-FAmAz, GDP-FAmP₄Tz, GDP-FAmSucMMAE or GDP-FAmAzDXd), human β(1,4)-GalT1(Y285L) (0.5 mg/mL), Hp-α(1,3)-FucT (0.7 mg/mL) in 25 mM Tris-HCl buffer (pH 7.0) with 20 mM MgCl₂ and 10 mM MnCl₂ at 30° C. for overnight to 48 hours. The modified trastuzumab was purified with protein A resin. Mass spectral analysis showed formation of one major peak trastuzumab-(Galβ1,4)GlcNAc-FAmAz (found as 146388 Da, MAR 2), trastuzumab-(Galβ1,4)GlcNAc-FAmP₄Tz (found as 147060 Da, MAR 2), trastuzumab-(Galβ1,4)GlcNAc-FAmSucMMAE (found as 148634 Da, DAR 2), trastuzumab-(Galβ1,4)GlcNAc-FAmAzDXd (found as 148561 Da, DAR 2). All the compositions of the trastuzumab-(Galβ1,4)GlcNAc-Fuc* conjugates have an average MAR or DAR of 1.8-2.0, in which more than 90% of the conjugates have a MAR or DAR of 2.

### Example 75 Intact Protein Mass Analysis

For LC-MS analysis, the purified proteins were analyzed on an Xevo G2-XS QTOF MS System (Waters Corporation) equipped with an electrospray ionization (ESI) source in conjunction with Waters Acuqity UPLC I-Class plus. Separation and desalting were carried out on a waters ACQUITY UPLC Protein BEH C4 Column (300 Å, 1.7 μm, 2.1 mm×100 mm). Mobile phase A was 0.1% formic acid in water and mobile phase B was acetonitrile with 0.1% formic acid. A constant flow rate of 0.2 ml/min was used. Data were analysed using Waters Unify software. Mass spectral deconvolution was performed using a Unify software (version 1.9.4, Waters Corporation). Some of the results were shown in FIGS. 6, 8, 10, 11, 14, 15 and 17.

FIG. 6 shows the MS analysis of A) the transform of trastuzumab (148062 Da GoF, 148224 Da GoF-G₁F, 148384 Da G₁F-G₁F or GoF-G₂F and 148546 Da G₁F-G₂F) to trastuzumab-FAz (148065 Da MAR 0, 148414 Da MAR 1, 148763 Da MAR 2 and 149110 Da MAR 3). B) the transform of tratuzumab-G₂F (148711 Da) to tratuzumab-G₂F-FAz (149459 Da MAR 4). MAR: MOI to antibody ratio.

FIG. 8 shows MS analysis of antibody-G₂F-Fuc* conjugates generated by treating G₂F-antibodies with Hp-α(1,3)-FucT and GDP-Fuc derivatives. A) trastuzumab-G₂F-FAmAz (found as 149688 Da, MAR 4). B) trastuzumab-G₂F-FAmP₄Az (found as 150449 Da, MAR 4). C) trastuzumab-G₂F-FAmP₄Tz (found as 151037 Da, MAR 4). D) trastuzumab-G₂F-FAzP₄Biotin (found as 151289 Da, MAR 4). E) trastuzumab-G₂F-FAmP₄Biotin (found as 151250 Da, MAR 4). F) rituximab-G₂F-FAz (found as 148482 Da, MAR 4). G) bevacizumab-G₂F-FAz (found as 150610, MAR 4). H) bevacizumab-G₂F-FAzP₄Biotin (found as 152436 Da, MAR 4). I) bevacizumab-G₂F-FAmP₄Biotin (found as 152396, MAR 4). J) bevacizumab-G₂F-FAm (found as 150499 Da, MAR 4). K) bevacizumab-G₂F-FAmP₄Tz (found as 152173, MAR 4). L) bevacizumabG₂F-FAmP₄TCO (found as 152099, MAR 4). MAR: MOI to antibody ratio.

FIG. 10 MS-analysis of trastuzumab-G₂F-GGG conjugates. A) trastuzumab-G₂F-FAzDBCO-GGG (found as 152415 Da, MAR 4). B) trastuzumab-G₂F-FAmAzDBCO-GGG (found as 152639 Da, MAR 4). MAR: MOI to antibody ratio.

FIG. 11 shows the MS analysis of trastuzumab-G₂F-MMAE conjugates prepared from “one-step” and “two-step” process respectively. For the “one-step” process, trastuzumab-G₂F were modified directly with GDP-FAzP₄MMAE to generated the trastuzumab-G₂F-FAzP₄MMAE (found as 154922, DAR 4). For the “Two-step” process, trastuzumab-G₂F were first modified with GDP-FAz to generate the trastuzumab-G₂F-FAz (found as 149459, MAR 4) followed by reacting with DBCO-PEG₄-vc-PAB-MMAE to generate the Trastuzumab-G₂F-FAzDBCO-MMAE (found as 156093, DAR 4).

FIG. 14 shows the MS analysis of antibody-((Fuc)α1,6)(Galβ1,4)GlcNAc-Fuc* conjugates by treating antibody-((Fuc)α1,6)(Galβ1,4)GlcNAc with Hp-α(1,3)-FucT and GDP-Fuc derivatives. A) trastuzumab-((Fuc)α1,6)(Galβ1,4)GlcNAc-FAz (found as 146568, MAR 2). B) trastuzumab-((Fuc)α1,6)(Galβ1,4)GlcNAc-FAmAz (found as 146683, MAR 2). C) trastuzumab-((Fuc)α1,6)(Galβ1,4)GlcNAc-FAmP₄BCN (found as 147366, MAR 2). D) trastuzumab-((Fuc)α1,6)(Galβ1,4)GlcNAc-FAmP₄TCO (found as 147319, MAR 2). E) trastuzumab-((Fuc)α1,6)(Galβ1,4)GlcNAc-FAmP₄Tz (found as 147356, MAR 2). F) trastuzumab-((Fuc)α1,6)(Galβ1,4)GlcNAc-FAmP₄Biotin (found as 147465, MAR 2). MAR: MOI to antibody ratio.

FIG. 15 shows the MS analysis of antibody-(Galβ1,4)GlcNAc-Fuc* conjugates by treating antibody-(Galβ1,4)GlcNAc with Hp-α(1,3)-FucT and GDP-Fuc derivatives. A) trastuzumab-(Galβ1,4)GlcNAc-FAmAz (found as 146387, MAR 2). B) trastuzumab-(Gal1,4)GlcNAc-FAmP₄BCN (found as 147074, MAR 2). C) trastuzumab-(Gal1,4)GlcNAc-FAmP₄Tz (found as 147063, MAR 2). D) trastuzumab-(Galβ1,4)GlcNAc-FAmGGG (found as 146564, MAR 2). E) rituximab-(Galβ1,4)GlcNAc-FAmAz (found as 145414, MAR 2). F) rituximab-(Galβ1,4)GlcNAc-FAmP₄Tz (found as 146082, MAR 2). MAR: MOI to antibody ratio.

FIG. 17 shows the MS analysis of antibody-drug conjugates prepared from “one-step” and “two-step” process respectively. For the “two-step” process: A) trastuzumab-G₂F-FAmAzDBCO-MMAE (found as 156322, DAR 4). B) trastuzumab-G₂F-FAmAzDBCO-MMAF (found as 156378, DAR 4). C) rituximab-G₂F-FAmAzDBCO-MMAE (found as 155341, DAR 4). D) trastuzumab-(Galβ1,4)GlcNAc-FAmAzDBCO-MMAE (found as 149708, DAR 2). E) trastuzumab-(Galβ1,4)GlcNAc-FAmAzDBCO-MMAF (found as 149736, DAR 2). F) trastuzumab-(Galβ1,4)GlcNAc-FAmP₄AzDBCO-seco-DUBA (found as 150081, DAR 2). G) trastuzumab-(Galβ1,4)GlcNAc-FAmAzBCN-MMAE (found as 149486, DAR 2). H) trastuzumab-(Galβ1,4)GlcNAc-FAmP₄BCNAz-MMAE (found as 149863, DAR 2). I) trastuzumab-(Galβ1,4)GlcNAc-FAmP₄TzTCO-MMAE (found as 150051, DAR 2). J) hRS7-(Galβ1,4)GlcNAc-FAmAzDBCO-MMAE (found as 149555, DAR 2). For the “one-step” process: K) trastuzumab-G₂F-FAmP₄MMAE (found as 154879, DAR 4). L) trastuzumab-G₂F-FAmSucMMAE (found as 154177, DAR 4). M) trastuzumab-G₂F-FAmAzP₄MMAE (found as 155147, DAR 4). N) trastuzumab-G₂F-FAmP₄AzP₄MMAE (found as 155908, DAR 4). O) trastuzumab-(Galβ1,4)GlcNAc-FAmP₄MMAE (found as 148989, DAR 2). P) trastuzumab-(Galβ1,4)GlcNAc-FAmSucMMAE (found as 148634, DAR 2). Q) trastuzumab-G₂F-FAmAzP₄DXd (found as 153967, DAR 4). R) trastuzumab-G₂F-FAmDM4 (found as 152877, DAR 4). S) trastuzumab-(Galβ1,4)GlcNAc-FAmAzP₄DXd (found as 148561, DAR 2). T) hRS7-(Galβ1,4)GlcNAc-FAmSucMMAE (found as 148484, DAR 2). U) hRS7-(Galβ1,4)GlcNAc-FAmAzP₄MMAE (found as 148969, DAR 2).

### Example 76 Binding Affinity Assay

Recombinant Her2 extracellular domain (HER2, novoprotein) was diluted to a final concentration of 250 ng/mL with coating buffer and plated on 96-well plates (100 μL/well) at 4° C. for overnight. After removing the coating solution, the plates were blocked with 3% (v/v) bovine serum albumin in PBS for 2 h at 37° C. After washing with PBST (PBS containing 0.03% tween-20) for 3 times, trastuzumab (positive control) and the “two-step” produced trastuzumab-drug conjugates (trastuzumab-G₂F-FAmAzDBCO-MMAE, trastuzumab-(Galβ1,4)GlcNAc-FAmAzDBCO-MMAE, trastuzumab-(Galβ1,4)GlcNAc-FAmP₄TzTCO-MMAE, trastuzumab-(Galβ1,4)GlcNAc-FAmP₄AzDBCO-seco-DUBA) were added to PBST (with 1% (v/v) bovine serum albumin in PBS) to a series of final concentrations (3000 ng/mL, 1000 ng/mL, 333.33 ng/mL, 111.11 ng/mL, 37.04 ng/mL, 12.35 ng/mL, 4.12 ng/mL, 1.37 ng/mL, 0.46 ng/mL, 0.15 ng/mL, 0 ng/mL) and added to the plates respectively. After incubating for 1.5 h, the plates were washed 3 times with PBST, then horseradish peroxidase (HRP)-conjugated goat anti-human IgG antibody was added to each well and incubated for 1 h at 37° C. Finally, each well was washed with PBST for 3 times, and then tetramethyl benzidine substrate was cotreated to produce color for visualization. The reaction in each well was stopped by adding 100 μL of 3 M HCl after 15 min of incubation at r.t. The absorbance was read at 450 nm on a Synergy™ LX plate reader. Trastuzumab (positive control) and the “one-step” produced trastuzumab-drug conjugates (trastuzumab-(Galβγ1,4)GlcNAc-FAmSucMMAE, trastuzumab-G₂F-FAmSucMMAE, trastuzumab-G₂F-FAmAzP₄MMAE, trastuzumab-(Galβ1,4)GlcNAc-FAmAzP₄DXd, trastuzumab-G₂F-FAmDM₄) were subjected to the process described above. The result showed a similar HER2-binding affinity between trastuzumab and trastuzumab-drug conjugates (FIG. 20).

### Example 77 HIC-HPLC Assay

Some trastuzumab-drug conjugates were evaluated by HIC-HPLC analysis using the Agilent 1260 HPLC system with a TSKgel Butyl-NPR column (4.6 mm×35 mm, 2.5 m; TOSOH) under the following conditions: (1) buffer A: 20 mM sodium phosphate, 1.5 M ammonium sulfate (pH 6.9); (2) buffer B: 75% (v/v) 20 mM sodium phosphate, 25% (v/v) isopropanol (pH 6.9); (3) flow rate: 0.4 mL/min; (4) gradient: from 100% buffer A to 100% buffer B (over 1-13 min); and (5) column temperature was 25° C. HIC-HPLC analysis showed high homogeneity of trastuzumab-drug conjugates (FIG. 21).

### Example 78 In Vitro Plasma Stability Assay

Human plasma was treated with protein A resin to removal the IgG. Then the depleted IgG plasma was filter sterilized by 0.22 μM filter. The trastuzumab-G₂F-FAmAzDBCO-MMAE, trastuzumab-(Galβ1,4)GlcNAc-FAmAzDBCO-MMAE, trastuzumab-G₂F-FAmSucMMAE, trastuzumab-(Galβ1,4)GlcNAc-FAmP₄MMAE were incubated with the plasma to a final concentration of 100 μg/mL at 37° C. and 5% CO₂. Samples was taken at 0, 2, 4, 6, 8 days and purified with protein A followed by MS analysis. MS analysis showed that the peaks corresponding to the antibody drug conjugates did not decrease in time and no new peak corresponding to degradation products could be detected, indicating that the conjugates were stable in human plasma for at least 8 days (FIG. 22).

### Example 79 In Vitro Efficacy of Some Trastuzumab-MMAE or Trastuzumab-MMAF Conjugates on SK-Br-3, BT-474 and MDA-MB-231 Cells

SK-Br-3 (Her2+) and BT-474(Her2+) were cultured in RPMI 1640 medium supplemented with 10% FBS (Gibco). MDA-MB-231 (Her2−) cells were cultured in DMEM (Gibco) supplemented with 10% FBS (Gibco). The cells were plated in 96-well plates with 5000 cells per well and were incubated for 24 hours at 37° C. and 5% CO₂. After removing of the culture medium, samples Kadcyla®, trastuzumab-G₂F-FAmAzDBCO-MMAE, trastuzumab-G₂F-FAmAzDBCO-MMAF, trastuzumab-(Galβ1,4)GlcNAc-FAmAzDBCO-MMAE, trastuzumab-(Galβ1,4)GlcNAc-FAmP₄MMAE and rituximab-G₂F-FAzDBCO-MMAE were added to the culture medium to a series of final concentrations (100 nM, 10 nM, 1 nM, 0.5 nM, 0.1 nM, 0.05 nM, 0.01 nM, 0.001 nM and 0 nM) and added to the plates respectively. The cells were incubated for 72 h at 37° C. and 5% CO₂ and subjected to a CellTiter-Glo® Luminescent Cell Viability Assay (Promega) to measure the cell viability. The trastuzumab-G₂F-FAmAzDBCO-MMAE, trastuzumab-G₂F-FAmAzDBCO-MMAF, trastuzumab-(Galβ1,4)GlcNAc-FAmAzDBCO-MMAE and trastuzumab-(Galβ1,4)GlcNAc-FAmP₄MMAE showed high potent of killing cells towards Her2 positive cell lines, but not of the Her2 negative cell line MDA-MB-231 (FIG. 23).

### Example 80 In Vitro Efficacy of Trastuzumab-(Galβ1,4)GlcNAc-FAmP₄AzDBCO-Seco-DUBA on SK-Br-3, NCI-N87 and BT-474 Cells

SK-Br-3 (Her2+), NCI-N87(Her2+) and BT-474(Her2+) were cultured in RPMI 1640 medium supplemented with 10% FBS (Gibco). MDA-MB-231 (Her2−) cells were cultured in DMEM (Gibco) supplemented with 10% FBS (Gibco). The cells were plated in 96-well plates with 5000 cells per well and were incubated for 24 hours at 37° C. and 5% CO₂. After removing of the culture medium, the sample was added to the culture medium to a series of final concentrations (100 nM, 10 nM, 1 nM, 0.5 nM, 0.1 nM, 0.05 nM, 0.01 nM, 0.001 nM and 0 nM) and added to the plates respectively. The cells were incubated for 72 h at 37° C. and 5% CO₂ and subjected to a CellTiter-Glo® Luminescent Cell Viability Assay (Promega) to measure the cell viability. trastuzumab-(Galβ1,4)GlcNAc-FAmP₄AzDBCO-seco-DUBA showed high potent of killing cells towards Her2 positive cell lines, but not of the Her2 negative cell line MDA-MB-231 (FIG. 24).

### Example 81 In Vitro Efficacy of Some Trastuzumab-Drug Conjugates on NCI-N87 and MDA-MB-231 Cells

NCI-N87 (Her2+) cells were cultured in RPMI 1640 medium supplemented with 10% FBS (Gibco). MDA-MB-231 (Her2−) cells were cultured DMEM (Gibco) supplemented with 10% FBS (Gibco). The cells were plated in 96-well plates with 3000 cells per well and incubated for 24 hours at 37° C. and 5% CO₂. After removing of the culture medium, samples trastuzumab, trastuzumab-G₂F-FAmSucMMAE, trastuzumab-(Galβ1,4)GlcNAc-FAmSucMMAE, trastuzumab-G₂F-FAmAzDBCO-MMAE, trastuzumab-G₂F-FAmAzP₄DXd and trastuzumab-G₂F-FAmDM4 were added to the culture medium to a series of final concentrations (100 nM, 10 nM, 1 nM, 0.1 nM, 0.05 nM, 0.01 nM, 0.001 nM, 0.0001 nM, 0.00001 nM and 0 nM) and added to the plates respectively. The cells were incubated for 6 days at 37° C. and 5% CO₂ and subjected to a CellTiter-Glo® Luminescent Cell Viability Assay (Promega) to measure the cell viability. The trastuzumab-G₂F-FAmSucMMAE, trastuzumab-(Galβ1,4)GlcNAc-FAmSucMMAE, trastuzumab-G₂F-FAmAzDBCO-MMAE, trastuzumab-G₂F-FAmAzP₄DXd and trastuzumab-G₂F-FAmDM4 showed high potent of killing cells towards Her2 positive cell lines, but not of the Her2 negative cell line MDA-MB-231 (FIG. 25).

### Example 82 In Vitro Efficacy of hRS7-(Galβ1,4)GlcNAc-FAmSucMMAE

JIMT-1 (Trop2 high expression) and MDA-MB-231 (Trop2 low expression) cells were cultured in DMEM (Gibco) supplemented with 10% FBS (Gibco). The cells were plated in 96-well plates with 5000 cells per well and were incubated for 24 hours at 37° C. and 5% CO₂. After removing of the culture medium, hRS7 and hRS7-(Galβ1,4)GlcNAc-FAmSucMMAE were added to the culturing medium to a series of final concentrations (100 nM, 10 nM, 1 nM, 0.5 nM, 0.1 nM, 0.05 nM, 0.01 nM, 0.001 nM and 0 nM) and added to the plates respectively. The cells were incubated for 72 h at 37° C. and 5% CO₂ and subjected to a CellTiter-Glo® Luminescent Cell Viability Assay (Promega) to measure the cell viability. The hRS7-(Galβ1,4)GlcNAc-FAmSucMMAE showed high potent of killing cells towards Trop2-high expression cell line JIMT-1, but not of the Trop2-low expression cell line MDA-MB-231 (FIG. 26).

### Example 83 In Vivo Efficacy of Trastuzumab-G₂F-FAmSucMMAE on a Nude Mouse Human Gastric NCI-N87 Xenograft Model

Female BALB/c nude mice (4-5-week-old) were inoculated with 1×10⁶ NCI-N87 (Her2+) cells which were resuspended in 50% PBS (pH 7.4) and 50% matrigel (BD). When the average tumor size reached 150-200 mm³, the PBS, trastuzumab (5 mg/kg), Kadcyla® (5 mg/kg) and trastuzumab-G₂F-FAmSucMMAE (5 mg/kg) were injected to different groups (n=6 mice per group) through the tail vein for one time respectively. The total length of the animal study was 35 days, and the tumor size and body weight of the mice were monitored twice per week throughout the study period. Tumor volumes were determined according to the formula: tumor volume (mm³)=π×long diameter×(short diameter)²/6. Trastuzumab-G₂F-FAmSucMMAE showed high efficacy of inhibiting tumor growth towards NCI-N87 tumor (FIG. 27). All animal studies were conducted in accordance with Institutional Animal Care and Use Committee guidelines and were performed at Hangzhou Medical College.

### Example 84 In Vivo Efficacy of hRS7-(Galβ1,4)GlcNAc-FAmSucMMAE on a Nude Mouse Human Breast Cancer JIMT-1 Xenograft Model

Female BALB/c nude mice (4-5-week-old) were inoculated with 1×10⁶ JIMT-1 (trop2 high expression) cells which were resuspended in 50% PBS (pH7.4) and 50% matrigel (BD). When the average tumor size reached 150-200 mm³, the PBS, hRS7 (5 mg/kg) and hRS7-(Galβ1,4)GlcNAc-FAmSucMMAE (5 mg/kg) were injected to different groups (n=6 mice per group) through the tail vein for one time respectively. The total length of the animal study was 37 days, and the tumor size and body weight of the mice were monitored twice per week throughout the study period. Tumor volumes were determined according to the formula: tumor volume (mm³)=π×long diameter×(short diameter) 2/6. The hRS7-(Galβ1,4)GlcNAc-FAmSucMMAE showed high efficacy of inhibiting tumor growth towards JIMT-1 tumor (FIG. 28). All animal studies were conducted in accordance with Institutional Animal Care and Use Committee guidelines and were performed at Hangzhou Medical College.

### Example 85 Cloning and Expression of Human β1,4-GalT1(Y285L)

The human β1,4-GalT1 (Uniprot accession number P15291) mutant Y285L gene was synthesized in PUC57 vector at Genscript, the human GalT Y285L mutant genes were amplified from this construct containing the sequence encoding the catalytic domain (63-398), by the overlap extension PCR method. the first insert DNA fragment was amplified with a pair of primers: Fw: (AAAAAGCAGGCTCTGAAAACTTGTACTTTCAAGGCGGCTCG (SEQ ID NO: 19)) and Rw: (TTGTACAAGAAAGCTGGGTCCTAGCTCGGTGTCCCGATGTC (SEQ ID NO: 20)). The vector DNA fragment was amplified from Mammalian Expression Vector PGEN2 DEST (Nat Chem. Biol. 2018, 14, 156) with a pair of primers: Oligo vector Fw: (GACCCAGCTTTCTTGTACAAAGTG (SEQ ID NO: 21)) and Oligo vector Rw: (GTTTTCAGAGCCTGCTTTTTTGT (SEQ ID NO: 22)). The GalT Y285L mutant DNA fragment was cloned to Vector PGEN2 DEST by using Exnase®II (Vazyme: C112-01). The expression and purification of human GalT1(Y285L) (SEQ ID NO: 1) were performed according to the reported procedure by Moremen K. W et al. (Moremen K. W et al., Nat. Chem. Biol. 2018, 14, 156).

### Example 86 Cloning, Expression and Purification of Bovine P1,4-GalT1(Y289L), Hp-α(1,3)-FucT, EndoS, Alfc and Human FT6

The cloning, expression and purification of bovine β1,4-GalT1(Y289L)(SEQ ID NO: 2), Streptococcus pyogenes EndoS (SEQ ID NO: 6), Lactobacillus casei α-1,6-fucosidase (AlfC) (SEQ ID NO: 7), Helicobacter pylori Hp-α(1,3)-FucT (SEQ ID NO: 4) and human FT6 (SEQ ID NO: 5) were performed according to the reported procedure by Qasba, P. K et al. (Prot. Expr. Fur. 2003, 30, 219) (J. Biol. Chem. 2002, 277, 20833), by Collin, M. et al. (EMBO J. 2001, 20, 3046; Infect. Immun. 2001, 69, 7187), by Wang L., et al. (Methods Mol. Biol. 2018, 19, 367), by Wu P. (Proc. Natd. Acad. Sci. USA 2009, 106, 16096) and by Moremen K. W et al. (Nat Chem. Biol. 2018, 14, 156), respectively.

### Example 87 Cloning, Expression and Purification of hRS7

The sequence of hRS7 antibody light chain and heavy chain were referenced to the patent (U.S. Pat. No. 7,238,785 B2). The gene encoding the light chain and the heavy chain of hRS7 were synthesized and clone into a PPT5 vector respectively by Genescript. Then, FreeStyle 293F cells were grown to a density of ˜2.5×10⁶ cells/ml and transfected by direct addition of 0.37 μg/ml and 0.66 μg/ml of the light chain and heavy chain expression plasmid DNA, and 2.2 μg/ml polyethylenimine (linear 25 kDa PEI, Polysciences, Inc, Warrington, Pa.) to the suspension cultures. The cultures were diluted 1:1 with Freestyle 293 expression medium containing 4.4 mM valproic acid (2.2 mM final) 24 h after transfection, and protein production was continued for another 4-5 d at 37° C. After protein production, the antibodies were purified through the protein A agarose following the manufacturer's instructions.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A protein conjugate, comprising a protein and an oligosaccharide, wherein said oligosaccharide comprises a structure of 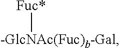
wherein:
said GlcNAc is directly or indirectly linked to an amino acid of said protein;
said Gal is a galactose;
said (Fuc) is a fucose, b is 0 or 1;
said Fuc* comprises a fucose or a fucose derivative and a molecule of interest (MOI);
said protein comprises an antigen binding fragment and/or a Fc fragment.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The protein conjugate of claim 1, wherein said MOI of said Fuc* comprises a F, F is a connector, and F is a 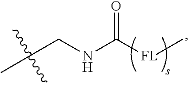
wherein said FL is a spacer and s is 0 or 1.

58. (canceled)

59. (canceled)

60. (canceled)

61. (canceled)

62. (canceled)

63. (canceled)

64. (canceled)

65. (canceled)

66. The protein conjugate of claim 1, wherein said Fuc* is Fuc-(F)ₘ-(L)ₙ-Y₁, or Fuc-(F)ₘ-(L)ₙ-P, wherein Fuc is said fucose or fucose derivative of the Fuc*, F is a connector, L is a linker, P is a biologically and/or pharmaceutically active substance, Y₁ is a functional group, m is 1, n is 0 or 1; wherein F is 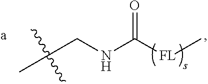
wherein said FL is a spacer and s is 0 or 1, and wherein Y comprises a functional moiety selected from the group consisting of

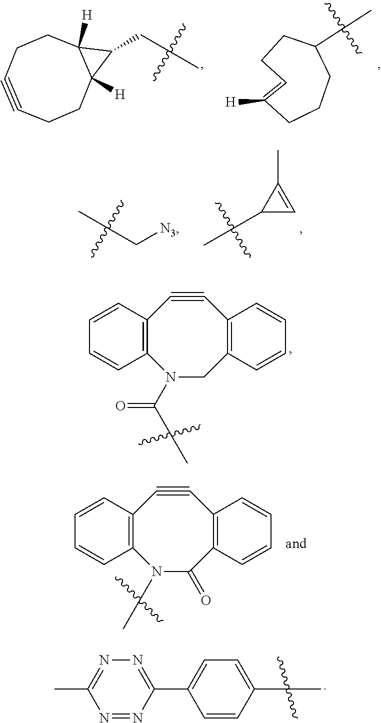

67. (canceled)

68. The protein conjugate of claim 1, wherein said Fuc is according to the formula 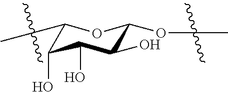
and said Fuc* is according to the formula

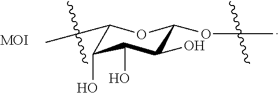

69. (canceled)

70. (canceled)

71. (canceled)

72. The protein conjugate of claim 1, the protein conjugate comprises 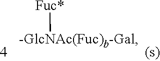
and is according to the formula 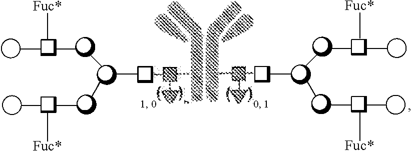
wherein [symbol] is a GlcNAc, [symbol] is the fucose of (Fuc) linked a GlcNAc through an α1,6 linkage, [symbol] is a mannose, ◯ is a galactose linked to a GlcNAc through a Galβ1,4GlcNAc linkage, Fuc* is linked to the GlcNAc through an Fuc*α1,3GlcNAc linkage, and [symbol] is an antibody or a Fc-fusion protein.

73. The protein conjugate of claim 1, the protein conjugate comprises 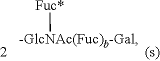
and is according to formula 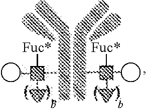
wherein [symbol] is a GlcNAc, [symbol] is the fucose of (Fuc) linked the GlcNAc through an α1,6 linkage, ◯ is a galactose linked to a GlcNAc through a Galβ1,4GlcNAc linkage, Fuc* is linked to the GlcNAc through an Fuc*α1,3GlcNAc linkage, [symbol] is an antibody or a Fc-fusion protein and b is 0 or 1.

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. A method for preparing a protein conjugate, comprising a step (a): contacting a fucose derivative donor Q-Fuc*′ to a protein comprising an oligosaccharide in the presence of a catalyst,
wherein said oligosaccharide comprises -GlcNAc(Fuc)_{b}-Gal, to obtain a protein conjugate comprising 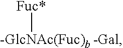
wherein:
said GlcNAc is directly or indirectly linked to an amino acid of said protein;
said Gal is a galactose;
said (Fuc) is a fucose, b is 0 or 1;
said Fuc*′ comprises a fucose or fucose derivative Fuc and a molecule of interest (MOI′);
said protein comprises an antigen binding fragment and/or a Fc fragment;
said Q-Fuc*′ is a molecule comprises said Fuc*′.

88. (canceled)

89. (canceled)

90. (canceled)

91. (canceled)

92. (canceled)

93. (canceled)

94. (canceled)

95. (canceled)

96. (canceled)

97. (canceled)

98. The method of 87, wherein said GlcNAc in said oligosaccharide is directly linked to an Asn residue of said protein.

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. The method of claim 87, wherein said MOI′ of Fuc* comprises an active moiety; wherein said active moiety of said MOI′ comprises a functional group Y₁ capable of participating in a ligation reaction; wherein said Y₁ comprises a functional moiety selected from the group consisting of 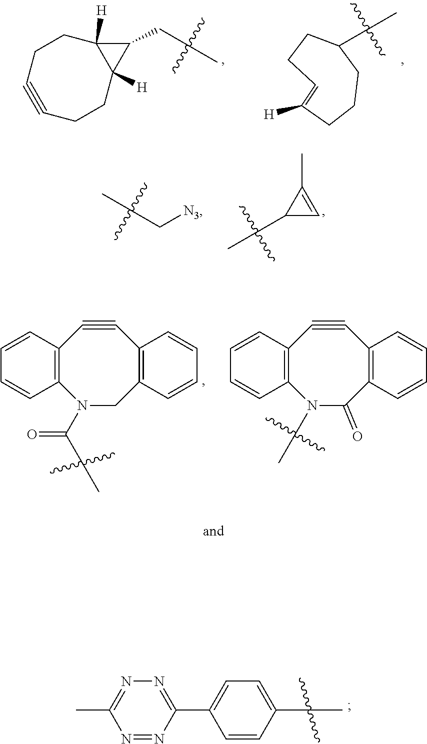
and
wherein step (a) comprising contacting said Q-Fuc*′ with said protein to obtain a protein conjugate comprising 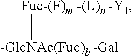
wherein Q-Fuc*′ is Q-Fuc-(F)ₘ-(L)ₙ-Y₁, Fuc is said fucose or fucose derivative of the Fuc*′, F is a connector, L is a linker, Y₁ is a functional group, b is 0 or 1, m is 0 or 1, and n is 0 or 1.

123. (canceled)

124. (canceled)

125. (canceled)

126. (canceled)

127. (canceled)

128. (canceled)

129. The method of claim 87, wherein said MOI′ of Fuc* comprises an active moiety; wherein said active moiety of said MOI′ comprises a P, and P is a biologically and/or pharmaceutically active substance; and
wherein step (a) comprising contacting said Q-Fuc*′ with sa protein to obtain a protein conjugate comprising 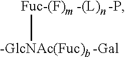
wherein Q-Fuc*′ is Q-Fuc-(F)ₘ-(L)ₙ-P, Fuc is the fucose or fucose derivative of the Fuc*′, F is a connector, L is a linker, P is a biologically and/or pharmaceutically active substance, b is 0 or 1, m is 0 or 1, and n is 0 or 1.

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. The method of claim 87, wherein said MOI′ of Fuc* comprises an active moiety; wherein said active moiety of said MOI′ comprises a functional group Y₁ capable of participating in a ligation reaction; wherein step (a) comprising contacting said Q-Fuc*′ with said protein to obtain a protein conjugate comprising 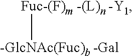
wherein Q-Fuc*′ is Q-Fuc-(F)ₘ-(L)ₙ-Y₁, Fuc is said fucose or fucose derivative of the Fuc*′, F is a connector, L is a linker, Y₁ is a functional group, b is 0 or 1, m is 1, and n is 0 or 1; wherein said connector F is 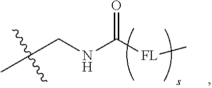
wherein said FL is a spacer and s is 0 or 1.

153. (canceled)

154. The method of 87, wherein said MOP′ of Fuc* comprises an active moiety; wherein said active moiety of said MOP′ comprises a P, and P is a biologically and/or pharmaceutically active substance; wherein step (a) comprising contacting said Q-Fuc*′ with said protein to obtain a protein conjugate comprising 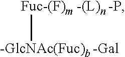
wherein Q-Fuc*′ is Q-Fuc-(F)ₘ-(L)ₙ-P, Fuc is the fucose or fucose derivative of the Fuc*′, F is a connector, L is a linker, P is a biologically and/or pharmaceutically active substance, b is 0 or 1, m is 1, and n is 0 or 1; wherein said connector F is 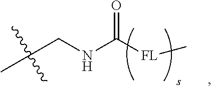
wherein said FL is a spacer and s is 0 or 1.

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. The method of claim 87, wherein said MOP′ of Fuc* comprises an active moiety; wherein said active moiety of said MOP′ comprises a functional group Y₁ capable of participating in a ligation reaction, wherein step (a) comprising contacting said Q-Fuc*′ with said protein to obtain a protein conjugate comprising 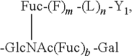
wherein Q-Fuc*′ is Q-Fuc-(F)ₘ-(L)ₙ-Y₁, Fuc is said fucose or fucose derivative of the Fuc*′, F is a connector, L is a linker, Y₁ is a functional group, b is 0 or 1, m is 1, and n is 0 or 1; wherein said Q-Fuc*′ is according to the formula 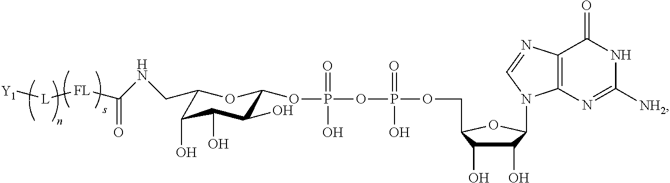
wherein said the FL is a spacer, s is 0 or 1.

168. (canceled)

169. (canceled)

170. (canceled)

171. The method of claim 87, wherein said MOP′ of Fuc* comprises an active moiety; wherein said active moiety of said MOP′ comprises a P, and P is a biologically and/or pharmaceutically active substance; wherein step (a) comprising contacting said Q-Fuc*′ with said protein to obtain a protein conjugate comprising 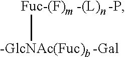
wherein Q-Fuc*′ is Q-Fuc-(F)ₘ-(L)ₙ-P, Fuc is the fucose or fucose derivative of the Fuc*′, F is a connector, and L is a linker, P is a biologically and/or pharmaceutically active substance, b is 0 or 1, m is 1, and n is 0 or 1; wherein said Q-Fuc*′ has a structure of 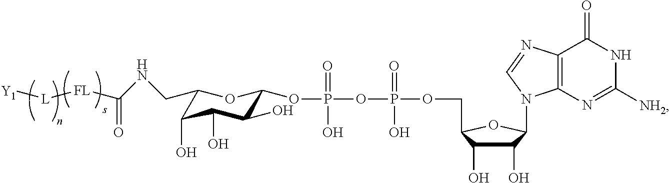
wherein said the FL is a spacer, s is 0 or 1.

172. (canceled)

173. (canceled)

174. The method of claim 171, wherein said Q-Fuc*′ is selected from the group consisting of

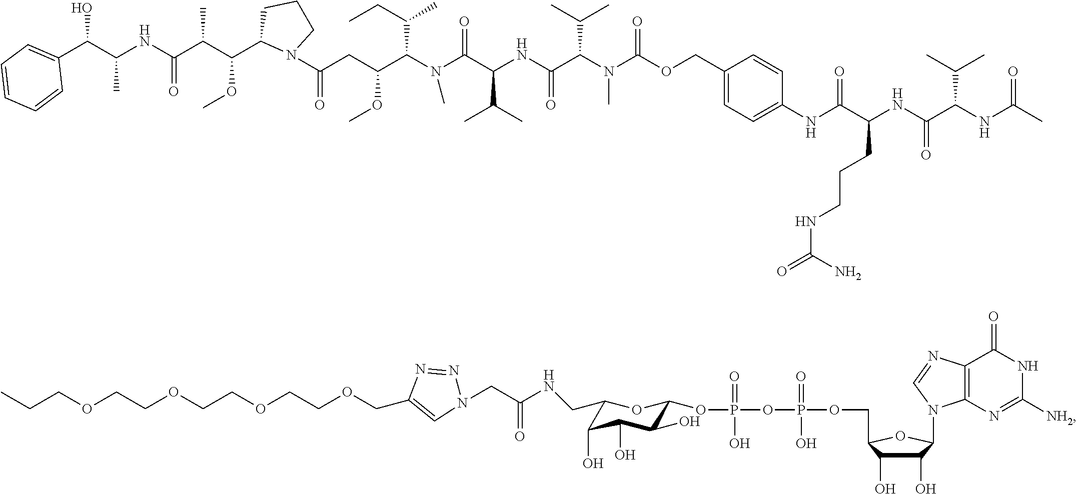

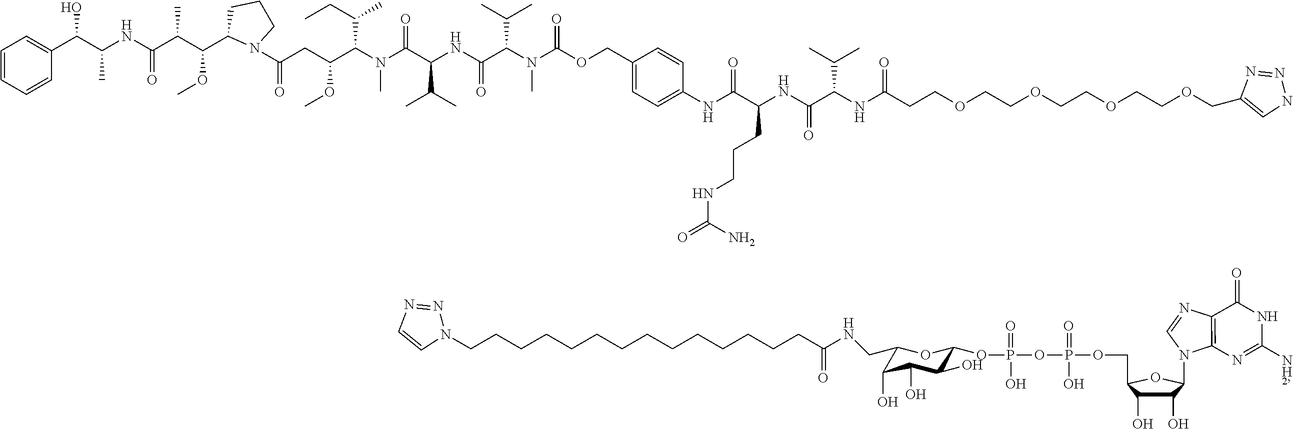

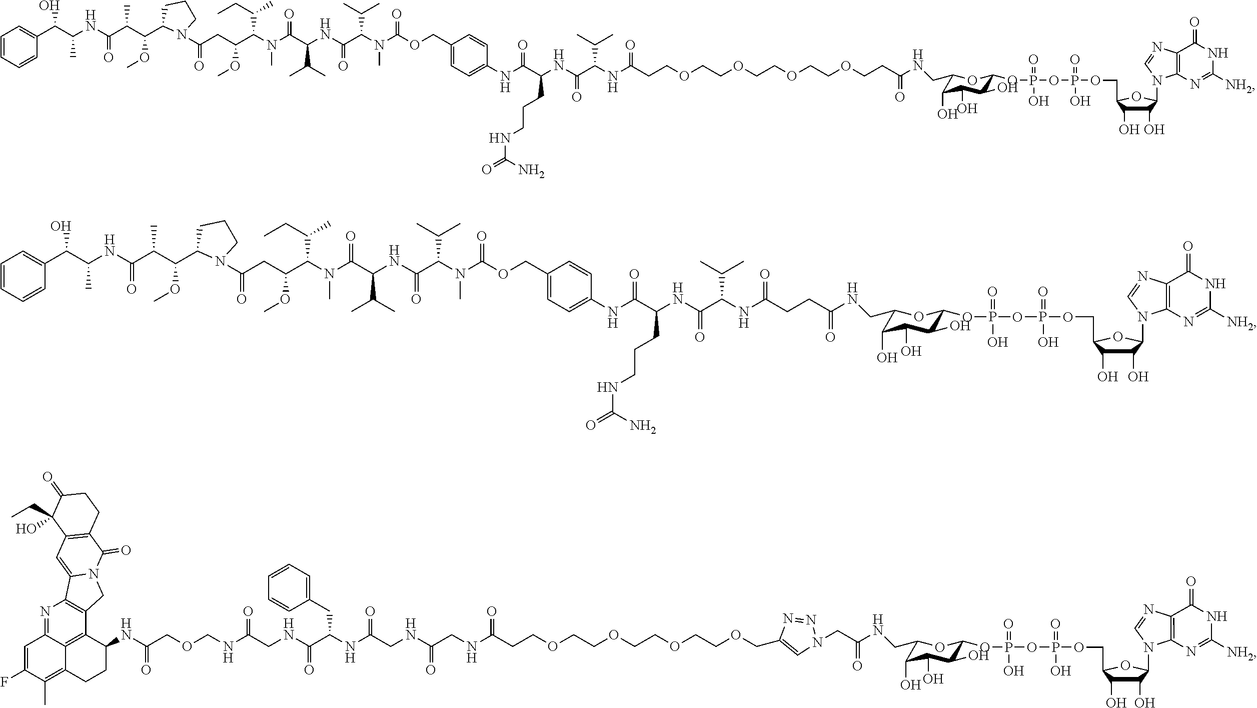

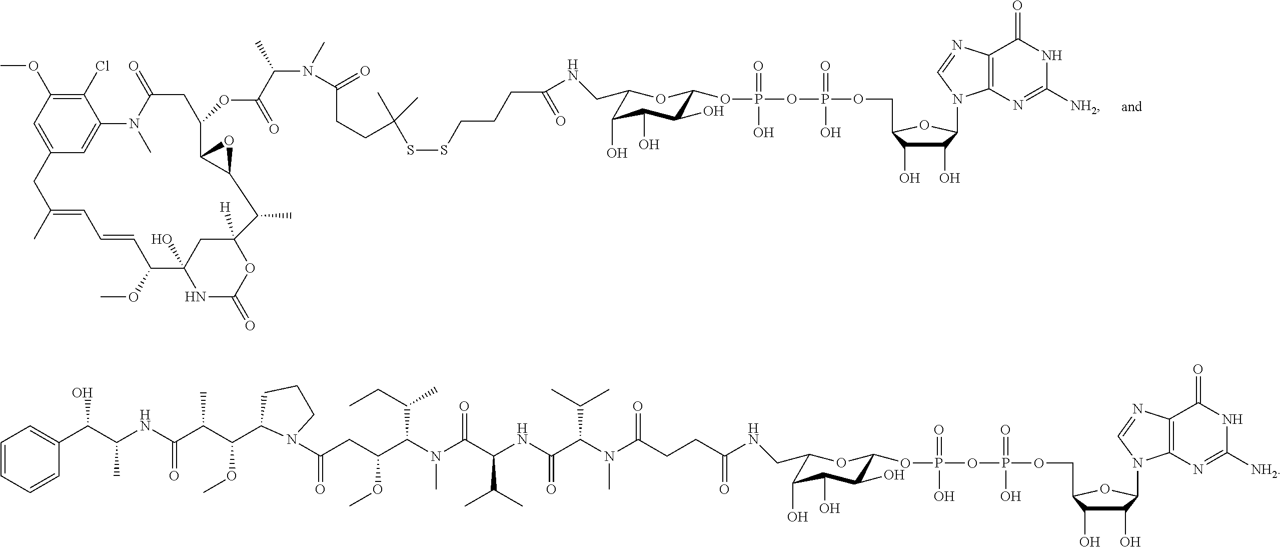

175. (canceled)

176. (canceled)

177. (canceled)

178. (canceled)

179. The method of claim 87,
wherein said protein comprising the oligosaccharide is according to the formula 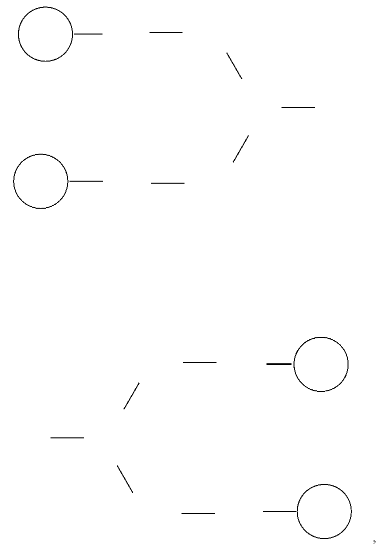
wherein [symbol] is a GlcNAc, [symbol] is the fucose of (Fuc) linked the GlcNAc through an α1,6 linkage, [symbol] is a mannose, ◯ is a galactose linked to the GlcNAc through a Galβ1,4GlcNAc linkage, and [symbol] is an antibody or a Fc-fusion protein.

180. (canceled)

181. The method of claim 87,
wherein said protein comprising the oligosaccharide is according to the formula 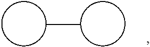
wherein [symbol] is a GlcNAc, [symbol] is the fucose of (Fuc) linked the GlcNAc through an α1,6 linkage, ◯ is a galactose linked to the GlcNAc through a Galβ1,4GlcNAc linkage, [symbol] is an antibody or a Fc-fusion protein, and b is 0 or 1.

182. (canceled)

183. (canceled)

184. (canceled)

185. (canceled)

186. (canceled)

187. (canceled)

188. (canceled)

189. (canceled)

190. (canceled)

191. (canceled)

192. (canceled)

193. (canceled)

194. The method of claim 87, further comprising a step (d) modifying a protein comprising an oligosaccharide to a protein comprises a core -GlcNAc(Fuc)_{b}, wherein b is 0 or 1; said step (d) is performed in the presence of an endoglycosidase or a functional variant or fragment thereof; and further comprising a step (c): contacting a protein comprising an oligosaccharide comprising the -GlcNAc(Fuc)ₙ with a UDP-galactose in the presence of a catalyst, to obtain said protein comprising the -GlcNAc(Fuc)ₙ-Gal, wherein Gal is a galactose, (Fuc) is a fucose and b is 0 or 1;
wherein said fucose of said (Fuc) is linked to said GlcNAc through an α1,6 linkage.

195. (canceled)

196. (canceled)

197. (canceled)

198. (canceled)

199. The method of claim 87, further comprising a step (d) modifying a protein comprising an oligosaccharide to a protein comprises a core -GlcNAc(Fuc)_{b}, wherein b is 0 or 1; said step (d) is performed in the presence of a endoglycosidase or a functional variant or fragment thereof; and further comprising a step (e) is performed in the presence of a core-α1,6 fucosidase or a functional variant or fragment thereof, to modify a protein comprising the core -GlcNAc(Fuc)ₙ to a protein comprises a core -GlcNAc; and further comprising a step (c): contacting a protein comprising an oligosaccharide comprising the -GlcNAc(Fuc)_{b} with a UDP-galactose in the presence of a catalyst, to obtain said protein comprising the -GlcNAc(Fuc)_{b}-Gal, wherein Gal is a galactose, (Fuc) is a fucose and b is 0; wherein said fucose of said (Fuc) is linked to said GlcNAc through an α1,6 linkage.

200. (canceled)

201. (canceled)

202. (canceled)

203. (canceled)

204. (canceled)

205. (canceled)

206. (canceled)

207. (canceled)

208. (canceled)

209. (canceled)

210. (canceled)

211. (canceled)

212. (canceled)

213. (canceled)

214. (canceled)

215. (canceled)

216. (canceled)

217. (canceled)

218. A protein conjugate, which is obtained with the method according to claim 87, and a pharmaceutical composition, comprising said protein conjugate.

219. (canceled)

220. A pharmaceutical composition, comprising the protein conjugate of claim 1, and optionally a pharmaceutically acceptable carrier.

221. (canceled)

222. A method for preventing or treating disease, comprising administrating the protein conjugate of claim 1, and/or the pharmaceutical composition comprising said protein conjugate.

223. A method for preventing or treating disease, comprising administrating the protein conjugate of claim 87, and/or the pharmaceutical composition comprising said protein conjugate.

224. (canceled)

225. (canceled)

226. (canceled)


SEQUENCE LISTING (23 sequences)

SEQ ID NO: 1 - protein, 336 aa, Artificial Sequence
  Human beta1,4-GalT1 (63-398; Y285L)
GSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQLFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 2 - protein, 287 aa, Artificial Sequence
  Bovine beta1,4-GalT1(Y289L) (130-402; Y289L; C342T) with a N-terminal tag
MASMTGGQQMGRGSSLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 3 - protein, 392 aa, Artificial Sequence
  Helicobacter pylori alpha(1,3)- fucosyltransferase (1-392)
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNY

SEQ ID NO: 4 - protein, 400 aa, Artificial Sequence
  Hp alpha(1,3)-fucosyltransferase (1-392) fusing with a C-terminal His tag
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNYLEHHHHHH

SEQ ID NO: 5 - protein, 321 aa, Artificial Sequence
  human FT6 catalytic domain (40-359) with a N terminal G
GDPTVYPNGSRFPDSTGTPAHSIPLILLWTWPFNKPIALPRCSEMVPGTADCNITADRKVYPQADAVIVHHREVMYNPSAQLPRSSRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFKNSLHPDYITEKLWRNALEAWAVPVVLGPSRSNYERFLPPDAFIHVDDFQSPKDLARYLQELDKDHARYLSYFRWRETLRPRSFSWALAFCKACWKLQEESRYQTRGIAAWFT

SEQ ID NO: 6 - protein, 1006 aa, Artificial Sequence
  Streptococcus pyogenes Endoglycosidase S (37-995) fusing with a N-terminal tag
MHHHHHHSSGLVPRGSGMKETAAAKFERQHMDSPDLGTGGGSGIEGREEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 7 - protein, 561 aa, Artificial Sequence
  Lactobacillus casei alpha1,6-fucosidase (1-344) fusing with a CPD tag and a his tag in the C-terminus
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANRALADGKILHNQNVNSWGPITVTPTTDGGETRFDGQIIVQMENDPVVAKAAANLAGKHAESSVVVQLDSDGNYRVVYGDPSKLDGKLRWQLVGHGRDHSETNNTRLSGYSADELAVKLAKFQQSFNQAENINNKPDHISIVGCSLVSDDKQKGFGHQFINAMDANGLRVDVSVRSSELAVDEAGRKHTKDANGDWVQKAENNKVSLSWDAQGHHHHHH

SEQ ID NO: 8 - protein, 214 aa, Artificial Sequence
  trastuzumab LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, Artificial Sequence
  trastuzumab HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  rituximab LC
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 451 aa, Artificial Sequence
  rituximab HC
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  bevacizumab LC
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 453 aa, Artificial Sequence
  bevacizumab HC
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Artificial Sequence
  hRS7 LC
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 450 aa, Artificial Sequence
  hRS7 HC
VQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 273 aa, Artificial Sequence
  Bovine beta1,4-GalT1(Y289L) (130-402; Y289L; C342T) without a N-terminal tag
SLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 17 - protein, 959 aa, Artificial Sequence
  Streptococcus pyogenes Endoglycosidase S (37-995)
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 18 - protein, 344 aa, Artificial Sequence
  Lactobacillus casei alpha1,6-fucosidase (1-344)
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 19 - DNA, 41 bp, Artificial Sequence
  Forward primer
aaaaagcaggctctgaaaacttgtactttcaaggcggctcg

SEQ ID NO: 20 - DNA, 41 bp, Artificial Sequence
  Reverse primer
ttgtacaagaaagctgggtcctagctcggtgtcccgatgtc

SEQ ID NO: 21 - DNA, 24 bp, Artificial Sequence
  Oligo vector forward primer
gacccagctttcttgtacaaagtg

SEQ ID NO: 22 - DNA, 23 bp, Artificial Sequence
  Oligo vector reverse primer
gttttcagagcctgcttttttgt

SEQ ID NO: 23 - protein, 6 aa, Artificial Sequence
  C-termianl LPETGG tag, LPETGG-COOH
LPETGG